(19) United States
(12) Patent Application Publication
(10) Pub. No.: US 2026/0108571 A1
(43) Pub. Date: Apr. 23, 2026

(54) MYCOBACTERIOPHAGE COMPOSITIONS AND RELATED METHODS

(71) Applicant: Oregon State University, Corvallis, OR (US)

(72) Inventor: Lia Danelishvili, Corvallis, OR (US)

(73) Assignee: Oregon State University, Corvallis, OR (US)

(21) Appl. No.: 19/363,116

(22) Filed: Oct. 20, 2025

Related U.S. Application Data
(60) Provisional application No. 63/710,533, filed on Oct. 22, 2024.

(51) Int. Cl. A61K 35/76; A61P 31/06; C12N 7/00

(52) U.S. Cl. CPC A61K 35/76; A61P 31/06; C12N 7/00; C12N 2795/10021; C12N 2795/10032

(57) ABSTRACT

Disclosed are isolated bacteriophages having lytic activity against a broad range of mycobacterial species, as well as phage cocktails comprising different mixtures of the isolated bacteriophages. The disclosed bacteriophages include Pill582, and variants thereof, having the ability to stimulate TLR2 signaling and limit intracellular bacterial survival within macrophages through the signalosome involving NLRP3 inflammasome activation leading to the caspase 1 activation and the production of pro-inflammatory cytokine IL-1β. Also disclosed are compositions comprising the isolated bacteriophages or phage cocktails, including pharmaceutical compositions and compositions further comprising one or more non-phage therapeutic agents. The disclosed bacteriophage and phage cocktail compositions are useful in methods for treating mycobacterial infections and associated disease conditions.

### CROSS-REFERENCE TO RELATED APPLICATION

This application claims the benefit of U.S. Patent Application No. 63/710,533, filed Oct. 22, 2024, expressly incorporated herein by reference in its entirety.

### STATEMENT REGARDING SEQUENCE LISTING

The Sequence Listing XML associated with this application is provided in XML format and is hereby incorporated by reference into the specification. The name of the XML file containing the sequence listing is 3014-P43US_Subst_Sequence_Listing.xml. The XML file is 181,567 bytes; was created on Dec. 29, 2025; and is being submitted electronically via Patent Center.

### BACKGROUND

An environmental Mycobacterium abscessus (MAB) is emerging as a significant human pathogen largely affecting individuals with lung conditions such as cystic fibrosis, emphysema, chronic obstructive pulmonary disease, and bronchiectasis. See Abdelaal et al., Microorganisms 10, 2022. MAB infections are also reported in children and individuals over 60 years of age without predisposing factors. See Lamb et al., J. Pediatric. Infect. Dis. Soc. 7:e131-e144, 2018; González-Martínez et al., Encyclopedia of Infection and Immunity 1:703-718, 2022. The immunocompromised patients commonly develop chronic pulmonary infections, leading to high treatment failure rates. See Chen et al., Front. Microbiol. 10:1977, 2019; Yang et al., Antibiotics (Basel) 11, 2022. While patients receive a prolonged course of the combination antibiotic regimens, MAB treatment is still challenging as the pathogen possess the natural resistance to many antimicrobials that are used in clinics. See Nessar et al., J. Antimicrob. Chemother. 67:810-818, 2012. Therefore, it is common in clinics to observe patients who either do not respond to therapy or respond only partially to it with subsequent re-activation of the disease.

There is a growing interest in the use of bacteriophages (phages) as a novel treatment strategy against multi-drug resistant pathogens (see Kortright et al., Cell Host Microbe 25:219-232, 2019) to save lives of patients with chronic and disseminated infections (see Lin et al., World J. Gastrointest. Pharmacol. Ther. 8:162-173, 2017). The substantial research indicates that phage-antibiotic combination therapy can display synergy and be clinically more effective over the use of single agents. See Oechslin et al., J. Infect. Dis. 215:703-712, 2017; Comeau et al., PLOS One 2:e799, 2007; Kamal and Dennis, Appl. Environ. Microbiol. 81:1132-1138, 2015; Torres-Barcelo et al., Trends. Microbiol. 24:249-256, 2016. Recent reports highlight favorable clinical outcomes among NTM patients (see Dedrick et al., Clin. Infect. Dis. 76:103-112, 2023) and in individuals with cystic fibrosis colonized by Pseudomonas aeruginosa, following participation in a phase 1b/2 trial of phage therapy (see Tamma et al., Trials 23:1057, 2022; Rappo et al., Open Forum Infect. Dis. 10, 2023).

Phage therapy is emerging as a promising alternative strategy for hard-to-treat bacterial infections. Yet, its therapeutic application in humans remains greatly constrained, primarily due to gaps in our understanding of fundamental phage properties within the human host. Recent studies present compelling evidence that phages possess a spectrum of characteristics beyond merely lysing bacteria. For example, they are capable of stimulating innate immune responses through mechanisms such as phagocytosis and cytokine secretion, and they can also modulate mammalian host immunity by regulating gene expression. These multifaceted attributes likely play a significant role in determining the success of phage therapy in vivo. See Jonczyk-Matysiak et al., Viruses 9, 2017.

Phages alone can adhere to mucosal surfaces and interact with epithelial cells and phagocytes. See Kurzepa et al., Clin. Exp. Med. 9:93-100, 2009; Carroll-Portillo and Lin, Microorganisms 7, 2019; Barr et al., Proc. Natl. Acad. Sci. USA 110:10771-10776, 2013. Eukaryotic cells express number of pattern recognition receptors (PRRs) (see Gordon, Cell 111:927-930, 2002; Opitz et al., Thromb. Haemost. 98:319-326, 2007; Kawai and Akira, Cell Death Differ. 13:816-825, 2006) allowing for the possibility of phage recognition at multiple locations within the host. Phages are capable stimulating PRRs because they contain ligands for TLRs 7, 8, 9, RIG-I-like receptors, and components that can exert TLR2 and TLR4 activity. See Jonczyk-Matysiak et al., supra; Carroll-Portillo et al., supra; Sartorius et al., EMBO Mol. Med. 7:973-988, 2015. The interactions between phage-derived ligands and the host PRRs have been identified to contribute to the efficacy of phage therapy. See Krut and Bekeredjian-Ding, J. Immunol. 200:3037-3044, 2018.

In addition, studies have demonstrated that phages can elicit an antibody response in immune cells, indicating their considerable potential not only as therapeutic treatments but also as vaccines. See Hashiguchi et al., Biochem. Biophys. Res. Commun. 402:19-22, 2010. Phages can boost immunity against invading pathogens during their attachment to mucin glycoproteins of mucosal surfaces of the gut or respiratory tract. See Van Belleghem et al., Sci. Rep. 7:8004, 2017. This interaction occurs via the immunoglobulin-like (Ig-like) protein domains that are present on the phage capsid. The Ig-like fold, a structural motif found in both antibodies and T-cell receptors, is recognized for its role in facilitating interactions with the adaptive immune system. See Bork et al., J. Mol. Biol. 242:309-320, 1994. Therefore, the presence of this domain positively selects and enhances phage adherence to mucus while also mediating a non-host-derived immunity. See Barr et al., 2013, supra; Barr et al., Proc. Natl. Acad. Sci. USA 112:13675-13680, 2015. Phages can also trigger an antiviral immune response in a TLR2-dependent manner via the repeated subunit pattern that most viral capsids contain. See Mishra et al., Int. J. Microbiol. 2020:8844963, 2020; Shepardson et al., mBio 8, 2017. In vivo studies utilizing the empty capsid of phage P22 have demonstrated increased bacterial clearance in mice alongside with the high gene expression levels of TLR2 and TLR6 in both whole-lung samples and purified alveolar macrophages. See Shepardson et al., supra. The research involving TLR2 deficient mice provides evidence suggesting that the immune-enhancing effects of phage P22 are entirely dependent on the TLR2 signaling and indicating a potential interaction between the viral capsid and the TLR2 receptor itself. Certain phages can affect the downstream of the TLR pathway to incur immunomodulatory effects. For instance, in vivo studies using phage M13 demonstrate the MyD88 pathway dependent strong IgG antibody responses in the absence of TLR2-, TLR4- and TLR7 genes in the knockout mice model. See Hashiguchi et al., supra. Phages depleted of their genetic material as well as heat-inactivated phages are also capable of eliciting immunomodulatory effects, suggesting that phage proteins are largely responsible for the immune interactions. See Carroll-Portillo et al., supra.

Furthermore, the transcriptome analysis of human peripheral blood mononuclear cells (PBMCs) reveal that phages are capable of inducing cytokine signatures. See Van Belleghem et al., supra. Reports on phage modulation of cytokine activity by blood mononuclear cells also show that application of highly purified phages with or without endotoxins can elicit both the anti-inflammatory (up-regulation of IL-1RN, SOCS3) and pro-inflammatory effects (up-regulation of IL-1α, IL-1β, TNFα). See id. In addition, phages can reduce the levels of LPS-induced inflammatory cytokines such as TNF-α, IL1β, IL-6, IL-8 and IL-10 in MAC-T bovine mammary epithelial cells, while the sole administration of phages does not provoke changes in the cytokine levels. See Zhang et al., Front. Microbiol. 9:1614, 2018. Most studies describing phage-induced anti-inflammatory effects in vivo report concurrent and efficient clearance of bacterial infections to be the treatment outcome. See Zimecki et al., BMC Microbiol. 9:169, 2009; Secor et al., Infect. Immun. 85, 2017. Interestingly, intraperitoneal administration of T4 phage has been reported to inhibit the endotoxin-induced innate signaling of reactive oxygen species (ROS) in leukocytes, and suggesting phage use in sepsis patients having high susceptibility to excessive ROS and oxidative cell death. See Miedzybrodzki et al., Virus Res. 131:233-242, 2008.

M. avium subsp. paratuberculosis (MAP) is the causative agent of Johne's disease, and a prevalent and economically significant infection in dairy cows. See Stabel, J. Dairy Sci. 81:283-288, 1998; Ott et al., Prev. Vet. Med. 40:179-192, 1999; Mortier et al., Prev. Vet. Med. 121:189-198, 2015. MAP is highly infectious and easily spread from contaminated environmental sources to healthy animals. It is challenging to control MAP transmission in the dairy herd environment due to long silent latency period of infection, the high rate of bacterial shedding from cows with advanced disease, a lack of reliable low-cost diagnostics, and a lack of effective preventive and treatment methods. See Idris et al., Animals (Basel) 12, 2021; Timms et al., J. Microbiol. Methods 85:1-8, 2011; Gill et al., J. Food Prot. 74:480-499, 2011. One promising new solution for control, treatment, and prophylaxis of MAP infections is to employ phages.

In recent years, phage therapies have been proposed for use in animal health, including veterinary applications, animal production environments, and more specifically in the dairy production chain for prophylaxis, therapeutics, environmental decontamination, and dairy product bio-preservation. See Colavecchio and Goodridge, Microbiol. Spectr. 5, 2017; Smith et al., J. Gen. Microbiol. 133:1127-1135, 1987; Smith et al., J. Gen. Microbiol. 133:1111-1126, 1987. Phage use in livestock has been mainly directed towards foodborne zoonotic infectious agents such as Campylobacter, Salmonella, E. coli, and Listeria. Multiple studies have explored the use of phages against Campylobacter in the poultry industry, and against Salmonella and pathogenic E. coli in the pig industry. See Smith and Huggins, J. Gen. Microbiol. 129:2659-2675, 1983; Firlieyanti et al., Int. J. Food Microbiol. 237:121-127, 2016; Zhang et al., J. Anim. Sci. Biotechnol. 6:39, 2015; Wall et al., Appl. Environ. Microbiol. 76:48-53, 2010. The efficacy and the therapeutic use of phages in animals to control pathogenic bacterial infections is well supported in the literature.

The majority of mycobacteriophages have been isolated in Mycobacterium smegmatis host. See Hatfull, Microbiol. Spectr. 6, 2018; Hatfull et al., J. Virol. 86:2382-2384, 2012. However, limited studies report the discovery of phages active against MAP, as isolating such phages is extremely challenging due to the complexity of working with slow-growing bacteria and their tendency to clump. Consequently, the potential use of these phages in livestock and veterinary medicine has not yet been explored.

Despite the advances in the use of phages in animals to control pathogenic bacterial infections, a need exists for bacteriophage compositions useful in livestock and veterinary medicine. The present disclosure seeks to fulfill this need and provide further related advantages.

### SUMMARY

In one aspect, the disclosure provides a phage cocktail comprising at least two bacteriophages selected from the group consisting of:
a first isolated bacteriophage having a genome that has at least 95% sequence identity with the genome of bacteriophage Pill582, deposited under ATCC Patent Deposit Designation No. PTA-127809, wherein said first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages;
a second isolated bacteriophage having a genome that has at least 95% sequence identity with the genome of bacteriophage Ore278, deposited under ATCC Patent Deposit Designation No. PTA-127810, wherein said second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and
a third isolated bacteriophage having a genome that has at least 95% sequence identity with the genome of bacteriophage Minn381, deposited under ATCC Patent Deposit Designation No. PTA-127811, wherein said third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

In certain embodiments, the phage cocktail comprises at least two bacteriophages selected from the group consisting of:
a first isolated bacteriophage having a genome that has at least 95% sequence identity with the nucleotide sequence shown in SEQ ID NO:1, deposited under ATCC Patent Deposit Designation No. PTA-127809, wherein said first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages;
a second isolated bacteriophage having a genome that has at least 95% sequence identity with the nucleotide sequence shown in SEQ ID NO:2, wherein said second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and
a third isolated bacteriophage having a genome that has at least 95% sequence identity with the nucleotide sequence shown in SEQ ID NO:3, wherein said third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

In certain of these embodiments, the first isolated bacteriophage is Pill582 or progeny thereof.

In certain of these embodiments, the second isolated bacteriophage is Ore278 or progeny thereof.

In certain of these embodiments, the third isolated bacteriophage is Minn381 or progeny thereof.

In certain embodiments, the phage cocktail comprises the first, second, and third isolated bacteriophages.

In certain embodiments, the phage cocktail comprises:
a first isolated bacteriophage having a genome that has at least 95% sequence identity with the genome of bacteriophage Pill582, deposited under ATCC Patent Deposit Designation No. PTA-127809, wherein said first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; and at least one bacteriophage selected from the group consisting of:

(i) a second isolated bacteriophage having a genome that has at least 95% sequence identity with the genome of bacteriophage Ore278, deposited under ATCC Patent Deposit Designation No. PTA-127810, wherein said second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and
(ii) a third isolated bacteriophage having a genome that has at least 95% sequence identity with the genome of bacteriophage Minn381, deposited under ATCC Patent Deposit Designation No. PTA-127811, wherein said third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

In certain embodiments, the phage cocktail comprises:
a first isolated bacteriophage having a genome that has at least 95% sequence identity with the nucleotide sequence shown in SEQ ID NO:1, deposited under ATCC Patent Deposit Designation No. PTA-127809, wherein said first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; and at least one bacteriophage selected from the group consisting of:
(i) a second isolated bacteriophage having a genome that has at least 95% sequence identity with the nucleotide sequence shown in SEQ ID NO:2, wherein said second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and
(ii) a third isolated bacteriophage having a genome that has at least 95% sequence identity with the nucleotide sequence shown in SEQ ID NO:3, wherein said third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

In another aspect, the disclosure provides compositions comprising a phage cocktail as described herein.

In certain embodiments, the composition comprises a phage cocktail as described herein and a pharmaceutically acceptable carrier.

In other embodiments, the composition comprises a phage cocktail as described herein and an anti-mycobacterial antibiotic.

In certain embodiments, the anti-mycobacterial antibiotic is selected from the group consisting of clarithromycin, azithromycin, rifampin, rifabutin, ethambutol, streptomycin, amikacin, cefoxitin, and ciprofloxacin.

In certain of these embodiments, the composition comprises at least two anti-mycobacterial antibiotics.

In a further aspect, the disclosure provides a method for treating a mycobacterial infection. In certain embodiments, the method comprises administering to a subject having the mycobacterial infection an effective amount of an isolated bacteriophage as described herein.

In certain of these embodiments, the mycobacterial infection is an infection with a mycobacterium selected from the group consisting of Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

In certain embodiments of the method, the subject is human.

In certain of these methods, the mycobacterial infection is a pulmonary infection. In certain of these embodiments, the mycobacterial infection is associated with a pulmonary disease in the subject. Representative pulmonary diseases include cystic fibrosis, emphysema, chronic obstructive pulmonary disease (COPD), and bronchiectasis.

In other of these embodiments, the mycobacterial infection is a gastrointestinal infection.

In further of these embodiments, the mycobacterial infection is an infection with Mycobacterium abscessus or Mycobacterium avium. In certain of these embodiments, the mycobacterial infection is an infection with Mycobacterium avium subspecies paratuberculosis or Mycobacterium bovis.

In other embodiments of the method, the subject is a non-human animal.

In certain of these methods, the non-human animal is a ruminant. In certain of these embodiments, the ruminant is a bovine.

In certain of these methods, the mycobacterial infection is a pulmonary infection. In other of these methods, the mycobacterial infection is a gastrointestinal infection.

In certain of these methods, the mycobacterial infection is infection with Mycobacterium avium subspecies paratuberculosis. In certain embodiments, the mycobacterial infection is associated with Johne's disease in the subject. In certain embodiments, the mycobacterial infection is a disseminated infection.

In another aspect, the disclosure provides a method for treating a mycobacterial infection that is a combination therapy comprising administering to a subject having the mycobacterial infection an effective amount of an isolated bacteriophage as described herein and an effective amount of an anti-mycobacterial antibiotic.

In certain embodiments, the anti-mycobacterial antibiotic is clarithromycin, azithromycin, rifampin, rifabutin, ethambutol, streptomycin, amikacin, cefoxitin, or ciprofloxacin.

In certain of these embodiments, the combination therapy comprises administering at least two anti-mycobacterial antibiotics.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

As used herein, the term “bacteriophage” or “phage” refers to a functional phage particle comprising a nucleic acid genome packaged in a proteinaceous envelope or capsid.

The term “lytic activity” means the property of a bacteriophage to cause lysis of a bacterial cell as determined by a plaque assay. The lytic activity of a bacteriophage can be tested on different mycobacterial species according to plaque assay techniques generally known in the art. For example, to assess the lytic activity of a bacteriophage on Mycobacterium smegmatis mc² 155, the host bacteria are prepared at a concentration of 10⁸ colony-forming units (CFU) per milliliter. This bacterial suspension is then overlaid on mycobacterial growth agar using a layer of molten top agar. Subsequently, the bacteriophage is spot-plated in a series of dilutions onto the surface of the agar. The plates are incubated for 3 to 4 days to allow for phage infection and lysis of the host cells. The presence of clear zones, or plaques, on the bacterial lawn indicates areas where the bacteriophage has lysed the bacterial cells, thereby demonstrating its lytic activity.

The term “isolated,” as used herein in reference to a bacteriophage, means a bacteriophage removed from its original environment in which it naturally occurs. The removed bacteriophage is cultivated, purified, and cultured separately from the environment in which it was located. Thus, an isolated phage in this instance does not contain any significant amounts of other phages.

The term “progeny” or “isolated progeny,” as used herein, means replicates of the original phage, including descendants of the phage created by serial passage of the isolated phage or by other means known in the art, and/or phages having a substantially equivalent Restriction Fragment Length Polymorphism (RFLP) profile to the deposited phage. “Restriction Fragment Length Polymorphism profile” or “RFLP profile” as used herein means an EcoRI, HindIII, BamHI, and/or XhoI restriction digestion profile of the phage. The term “substantially equivalent RFLP profile” as used herein means a profile showing acceptable variability between genomes of identical propagated organisms of isolated progeny as described by Tenover et al., J. Clin. Microbiol. 33:2233-2239, 1995.

The term “variant” of a reference bacteriophage means a bacteriophage having variation(s) in the genomic sequence and/or polypeptide(s) encoded thereby as compared to the reference bacteriophage. Such variants may have different phenotypic characteristics such as a different bacterial host range compared to the reference bacteriophage. In a particular aspect, a variant may be obtained by directed evolution (also called phage training), which allows the variant to acquire a lytic activity on one or several bacterial strains. Variants typically exhibit the same morphology compared to the reference bacteriophage. Variants typically comprise, e.g., silent mutations, conservative mutations, minor deletions, and/or minor replications of genetic material. In some embodiments, variants according to the present disclosure retain any observable characteristic or property that is dependent upon the genome of the reference bacteriophage such as, e.g., phenotypic characteristics of the bacteriophage and/or lytic activity against the mycobacterial strains. Preferred variants have no more than 5% nucleotide variation as compared to the genome of the reference bacteriophage, more preferably no more than 4% nucleotide variation, even more preferably no more than 2% nucleotide variation. Alternatively, or in combination, variants have preferably no more than 5% amino acid variation in a coded polypeptide sequence as compared to a polypeptide of the reference bacteriophage.

The term “phage cocktail,” as used herein, means a combination of at least two phages, each of which has been isolated from the environment from which it was originally found or has been produced by means of a technical process such as genetic engineering or serial passage techniques.

The term “pharmaceutically acceptable” as used herein in reference to a material (e.g., carrier, excipient, or diluent) refers to a material that is compatible for use in a human or animal subject. Such includes physiologically acceptable solutions or vehicles that are harmless or do not cause any significant specific or non-specific immune reaction to an organism or do not abrogate the biological activity of the active compound. For formulation of the composition into a liquid preparation, saline, sterile water, Ringer's solution, buffered physiological saline, albumin infusion solution, dextrose solution, maltodextrin solution, glycerol, ethanol, and mixtures thereof may be used as a pharmaceutically acceptable excipient or carrier. If necessary, other conventional additives such as thickeners, diluents, buffers, preservatives, surface active agents, antioxidants, and bacteriostatic agents may be added. Further, diluents, dispersants, surfactants, binders, and lubricants may be additionally added to the composition to prepare injectable formulations such as aqueous solutions, suspensions, and emulsions, oral formulations such as pills, capsules, granules, or tablets, or powdered formulations, and aerosolized formulations such as liquids or powders.

The term “effective amount,” in the context of treatment of a mycobacterial infection by administration of a bacteriophage or phage cocktail to a subject as described herein, refers to an amount of such phage or phage cocktail that is sufficient to inhibit the occurrence or ameliorate one or more symptoms of the mycobacterial infection, including one or more symptoms of a disease or disorder associated with (e.g., caused or exacerbated by) the mycobacterial infection. An effective amount of a bacteriophage or phage cocktail composition is administered according to the methods of the present invention in an “effective regime.” The term “effective regime” refers to a combination of amount of the composition being administered and dosage frequency adequate to accomplish treatment or prevention of the mycobacterial infection. As used herein, “treat,” “treatment,” “therapy,” “therapeutic,” and the like refer to both therapeutic and prophylactic applications.

The term “patient” or “subject,” in the context of treating a mycobacterial infection, or a disease or disorder associated with such infection, refers to an animal, which may be human or non-human (e.g., non-human mammals). In some variations, a non-human animal subject is a ruminant (e.g., a bovine).

The term “combination therapy” refers to a therapeutic regime that involves the provision of at least two distinct therapies to achieve an indicated therapeutic effect. In the particular context of the present disclosure, the term means a therapeutic regime comprising administration of a bacteriophage or phage cocktail as described herein and at least one other therapy such as, e.g., an anti-mycobacterial antibiotic and/or an immunomodulatory agent. The distinct therapies constituting a combination therapy may be delivered, e.g., as simultaneous, overlapping, or sequential dosing regimens. In the context of the administration of two or more chemically distinct agents, it is understood that the active agents may be administered as part of the same composition or as different compositions. When administered as separate compositions, the compositions comprising the different active ingredients may be administered at the same or different times, by the same or different routes, using the same or different dosing regimens, all as the particular context requires and as determined by the attending physician.

The term “sequence identity” as used herein is determined by comparing two optimally aligned sequences over a comparison window, where the fragment of the polynucleotide in the comparison window may comprise additions or deletions (e.g., gaps or overhangs) as compared to the reference sequence, which does not comprise additions or deletions, for optimal alignment of the two sequences. The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (Add. Appl. Math. 2:482, 1981), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by inspection.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings.

FIGS. 1A-1J show ultrathin TEM sections of THP-1 human macrophages exposed with phages over 5 h. Confluent monolayers of THP-1 cells were exposed to phages (10¹⁰ pfu/ml) for 20 min (FIGS. 1A and 1B), 2 h (FIGS. 1C and 1D) and 5 h (FIGS. 1E and 1F). At each time point, cells were washed three times with PBS, fixed in the Karnovsky fixative and processed for TEM. The scale bar, ranging between 500 nm and 3 μm, is provided beneath each micrograph for reference. Four ultrathin section images depict the fusion of vacuoles within THP-1 human macrophages following a 2-hour exposure with phages (FIGS. 1G-1J). The merging sites are indicated by arrows.

FIGS. 2A and 2B compare quantification of intracellular bacteria in response to phage exposure to THP-1 cells. Pre-treatment assay (FIG. 2A): lytic phages were pre-exposed to THP-1 cells (10⁵ cells/well) at a phage-to-cell ratio of 10:1 for 1 h, followed by three gentle washes with HBSS. Next, cells were infected with MAB inoculum in HBSS supplemented 0.1% Tween 80 at MOI of 1 bacteria to 1 cell for 2 h. Sample lysates were obtained at day 3 post-infection for bacterial CFU quantification. Post-treatment assay (FIG. 2B): THP-1 cell monolayers (10⁵ cells/well) in 96-well plates were initially infected with a MOI of 1 bacterium to 1 cell for 2 h. Next, the cells were exposed to phages at a 10:1 ratio for 1 h. The monolayers were washed with HBSS, replenished with fresh culture media, and incubated at 37° C. with 5% CO₂ for up to 3 days. Monolayers were lysed at day 3 post-infection for bacterial CFU quantification.

FIGS. 3A-3D show the impact of TLR2/TLR4 signaling activation on MAB infection. FIG. 3A compares The intracellular growth dynamics of MAB in TLR2 and TLR4-stimulated THP-1 cells before and after bacterial infection. The bacterial survival rates were assessed through viable CFU counts at 1 hour, 24 hours, and 72 hours after MAB infection. The data is presented as the mean±standard deviation (SD) of two independent experiments, each conducted in triplicate. *p<0.05, **p<0.01 denote statistical significance between the TLR-treated groups and the MAB infection alone group at the corresponding time points. Following the 2 h MAB infection of THP-1 monolayers, either TLR2/4 inhibitor oxPAPC (FIG. 3B), TLR2 inhibitor TL2-C29 (FIG. 3C), or TLR4 inhibitor CLI-095 (FIG. 3D) from InvivoGen was added to THP-1 cell monolayers for 30 min and then exposed to phages at 10:1 ratio. Intracellular MAB growth was evaluated at day 3 post-infection where the control group did not receive antagonist treatment. *p<0.05 and **p<0.01, denote statistical significance between the TLR-treated and non-treated corresponding groups. The data is presented as the mean±standard deviation (SD) of these experiments in three technical replicates.

FIGS. 4A-4D show the evaluation of intracellular growth of MAB morphotypes in THP-1 cells. Macrophages were infected with either MAB-S or MAB-R at 1:1 MOI for 2 h and then either left untreated (control group) or exposed with TLR2 agonist or treated with Ph17 for 1 h (experimental groups) (FIG. 4A). THP-1 cells were lysed in 0.1% of TritonX-100 and via mechanical scraping at 3-day post-infection, and each well were serially diluted and plated on 7H10 agar plates for CFU quantification. **p<0.01, denote statistical significance between the TLR2 or Ph17 treated and only infection groups of MAB-S or MAB-R at day 3. The data is presented as the mean±standard deviation (SD) of three experiments in four technical replicates. THP-1 cell monolayers were infected with either MAB-S or MAB-R at 1:1 MOI for 2 h and either incubated with TLR2 agonist or treated with Ph17 for 1 h (FIG. 4B). At day 3 post-infection, supernatants were removed and processed separately from the cell lysates for CFU counts. Each sample (supernatant as well as cell lysate) was serially diluted and plated on 7H10 agar plates. Bacterial colonies were counted after 5 days of incubation at 37° C. **p<0.01, denote statistical significance between extracellular and intracellular MAB counts within the same experimental group. The data is presented as the mean±standard deviation (SD) of three experiments in three technical replicates. Ultrathin sections of THP-1 human macrophages were obtained after infection with MAB-S (FIG. 4C) or MAB-R (FIG. 4D) at 1:1 MOI for 2 h and then treating with Ph17 at 10:1 ratio for 1 h. Three hours later, cells were washed, fixed in the Karnovsky fixative, and processed. Four images for each MAB morphotypes are presented. Vacuole merging sites are indicated by arrows. B for Bacteria and Ph-Phage.

FIGS. 5A-5D show the NLRP3 inflammasome and ROS activation in MAB infected and Ph17 treated macrophages. Gene expression of NLRP3 (FIG. 5A), NF-kB (FIG. 5B), and GILZ (FIG. 5C) were measured by qRT-PCR at 24 h post-infection with MAB-S. TLR2 treated group served as a positive control and MAB-S only infection as a negative control. Selected gene transcript mRNA levels are presented as relative expression to β actin. ROS levels were measured over 24 h of bacterial infection within the same experimental and control groups using CellROX Green at 485/520 nm according to manufacturer's protocol (FIG. 5D). Results are shown for 5 h time point where most changes in ROS are detected.

FIGS. 6A and 6B show the NLRP3 inflammasome activation leads to elevated production of IL-1β and caspase 1 activation in MAB-S infected and Ph17 treated THP-1 cells. FIG. 6A compares IL-1β concentration at 5 and 24 hours for infection/treatment groups. Supernatants were obtained from macrophages infected with either live or heat-killed MAB-S or treated with Ph17 without bacterial infection at 5 h and 24 h time point. The supernatant obtained from THP-1 without infection or treatment served as a baseline control for the corresponding timepoints. Supernatants from MAB-S infected/TLR2 exposed and MAB-S/Ph17 treated cells were collected and measured IL-1β secretion levels with ELISA. The data are normalized with IL-1β secretion levels of uninfected/untreated THP-1 cells. *p<0.05 and **p<0.01 denote statistical significance between the MAB-infected group and both the MAB/TLR2 and MAB/Ph17 treated groups at the corresponding time points. The data is presented as the mean±SD of three independent experiments each performed in four technical replicates. FIG. 6B are Western blot images comparing caspase 1 activation for infection/treatment groups: top panel, β-actin; bottom panel, caspase 1 and pro-caspase 1. Cell lysates were obtained from the same experimental and control groups at 24 h and precleared total protein samples were subjected for Western Blot analysis for caspase-1 (pro form at 45 kDa; active form at 10 kDa). The experiments were performed three times, and immunoblot images were recorded on the Odyssey Imager (Li-Cor).

FIGS. 7A-7C show phage host range and lytic efficiency in MAP strains. Twenty-three phages were retested against MAP K10 and five additional clinical isolates using the double-layer agar plate method (FIG. 7A). While the bacteria formed a dense lawn, selected MAP-specific phages produced clear plaques and growth inhibition zones, confirming their lytic activity. Plates were incubated for up to eight weeks or until distinct plaques were observed. FIG. 7B is a heat map summarizes the activity of twenty-three phages against 6 MAP isolates, assessed through liquid culturing methods. Phage efficacy was quantified by viable bacterial CFU counts, and the percentage of bacterial survival was calculated relative to control cultures without phage exposure. Selected phages were further tested through titration to assess their virulence in titrations across different MAP isolates (FIG. 7C).

FIG. 8 compares the lytic activity of MAP-specific phages across clinically relevant mycobacterial species. The heat map summarizes the lytic activity of twenty-three phages tested against 17 clinical isolates of various non-tuberculous mycobacterial species, as well as M. bovis BCG and M. tuberculosis (M. tb) strains. Lytic activity was determined using a plaque assay with the double-layer agar method at a phage concentration of 10⁷ PFU.

FIG. 9 compares the effective inhibition by phage of MAP colonization and invasion in epithelial cells. MDBK cell monolayers were pre-treated with phages at a concentration of 10⁹ PFU for 30 min before infection with MAP at a MOI of 10. Control wells, without phage pre-exposure, were included for comparison. At 8 h and 24 h post-infection, cells were lysed with 0.1% Triton X-100, serially diluted, and MAP counts were assessed by CFU enumeration following 4-5 weeks of incubation on Middlebrook 7H10 agar plates. The significance was determined using student's t-test (*p<0.05, **p<0.01) within the treatment groups over time and between control and experimental groups. Data represent the mean±SD of three independent biological replicates, each performed in quadruplicate.

### DETAILED DESCRIPTION

The present disclosure is based, at least in part, on the isolation, characterization, and testing of new bacteriophages having lytic activity against mycobacterial species, phage cocktail compositions comprising different mixtures of the isolated bacteriophages, and use of the bacteriophage and phage cocktail compositions in methods for treating mycobacterial infections. The disclosure relates particularly to three strains of isolated bacteriophages, and variants thereof, that are capable of infecting and lysing a wide range of mycobacterial species, which are responsible for significant infections in humans as well as non-human animals, including infections associated with pulmonary and gastrointestinal conditions. As described herein, at least one of these strains is also shown to stimulate the innate immune responses limiting intracellular bacterial survival within professional phagocytes. These mycobacteriophage strains, referred to herein as Pill582, Ore278, and Minn381, have been deposited with the American Type Culture Collection, where they received ATCC Patent Deposit Designation Nos. PTA-127809, PTA-127810, and PTA-127811, respectively.

In one aspect, the present disclosure provides an isolated bacteriophage having a genome that has at least 95% sequence identity with the genome of bacteriophage Pill582, deposited under ATCC Patent Deposit Designation No. PTA-127809, wherein the isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages. In some embodiments, the isolated bacteriophage is Pill582 or progeny thereof.

In another aspect, the present disclosure provides an isolated bacteriophage having a genome that has at least 95% sequence identity with the genome of bacteriophage Ore278, deposited under ATCC Patent Deposit Designation No. PTA-127810, wherein the isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In some embodiments, the isolated bacteriophage is Ore278 or progeny thereof.

In a further aspect, the present disclosure provides an isolated bacteriophage having a genome that has at least 95% sequence identity with the genome of bacteriophage Minn381, deposited under ATCC Patent Deposit Designation No. PTA-127811, wherein the isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In some embodiments, the isolated bacteriophage is Minn381 or progeny thereof.

In another aspect, the present disclosure provides an isolated bacteriophage having a genome that has at least 95% sequence identity with the nucleotide sequence shown in SEQ ID NO:1, SEQ ID NO:2, or SEQ ID NO:3, wherein the isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In some embodiments, the genome has the nucleotide sequence shown in SEQ ID NO:1, or at least 95% sequence identity with the nucleotide sequence shown in SEQ ID NO:1. In other embodiments, the genome has the nucleotide sequence shown in SEQ ID NO:2, at least 95% sequence identity with the nucleotide sequence shown in SEQ ID NO:2. In further embodiments, the genome has the nucleotide sequence shown in SEQ ID NO:3, at least 95% sequence identity with the nucleotide sequence shown in SEQ ID NO:3.

In yet another aspect, the present disclosure provides a phage cocktail comprising at least two bacteriophages selected from (i) a first isolated bacteriophage having a genome that has at least 95% sequence identity with the genome of bacteriophage Pill582, deposited under ATCC Patent Deposit Designation No. PTA-127809, wherein the first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; (ii) a second isolated bacteriophage having a genome that has at least 95% sequence identity with the genome of bacteriophage Ore278, deposited under ATCC Patent Deposit Designation No. PTA-127810, wherein the second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and (iii) a third isolated bacteriophage having a genome that has at least 95% sequence identity with the genome of bacteriophage Minn381, deposited under ATCC Patent Deposit Designation No. PTA-127811, wherein the third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In some embodiments, the first isolated bacteriophage is Pill582 or progeny thereof; the second isolated bacteriophage is Ore278 or progeny thereof; and/or the third isolated bacteriophage is Minn381 or progeny thereof. In certain variations, the phage cocktail comprises the first, second, and third isolated bacteriophages.

In a related aspect, the present disclosure provides a phage cocktail comprising at least two bacteriophages selected from (i) a first isolated bacteriophage having a genome that has at least 95% sequence identity with the genome of bacteriophage Pill582, deposited under ATCC Patent Deposit Designation No. PTA-127809, wherein the first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; (ii) a second isolated bacteriophage having a genome that has at least 95% sequence identity with the nucleotide sequence shown in SEQ ID NO:2, wherein the second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and (iii) a third isolated bacteriophage having a genome that has at least 95% sequence identity with the nucleotide sequence shown in SEQ ID NO:3, wherein the third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In some embodiments, the first isolated bacteriophage is Pill582 (SEQ ID NO: 1) or progeny thereof; the genome of the second isolated bacteriophage has the nucleotide sequence shown in SEQ ID NO:2; and/or the genome of the third isolated bacteriophage has the nucleotide sequence shown in SEQ ID NO:3. In certain variations, the phage cocktail comprises the first, second, and third isolated bacteriophages.

In another related aspect, the present disclosure provides a phage cocktail comprising a first isolated bacteriophage having a genome that has at least 95% sequence identity with the genome of bacteriophage Pill582, deposited under ATCC Patent Deposit Designation No. PTA-127809, wherein the first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; and at least one bacteriophage selected from (i) a second isolated bacteriophage having a genome that has at least 95% sequence identity with the genome of bacteriophage Ore278, deposited under ATCC Patent Deposit Designation No. PTA-127810, wherein the second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and (ii) a third isolated bacteriophage having a genome that has at least 95% sequence identity with the genome of bacteriophage Minn381, deposited under ATCC Patent Deposit Designation No. PTA-127811, wherein the third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In some embodiments, the first isolated bacteriophage is Pill582 or progeny thereof; the second isolated bacteriophage is Ore278 or progeny thereof; and/or the third isolated bacteriophage is Minn381 or progeny thereof. In certain embodiments of the phage cocktail, the genome of the first isolated bacteriophage has the nucleotide sequence shown in SEQ ID NO:1. In certain embodiments of the phage cocktail, the genome of the second isolated bacteriophage has the nucleotide sequence shown in SEQ ID NO:2. In certain embodiments of the phage cocktail, the genome of the third isolated bacteriophage has the nucleotide sequence shown in SEQ ID NO:3. In certain embodiments of the phage cocktail, the phage cocktail comprises the first, second, and third isolated bacteriophages.

In still another related aspect, the present disclosure provides a phage cocktail comprising a first isolated bacteriophage having a genome that has at least 95% sequence identity with the genome of bacteriophage Pill582, deposited under ATCC Patent Deposit Designation No. PTA-127809, wherein the first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; and at least one bacteriophage selected from (i) a second isolated bacteriophage having a genome that has at least 95% sequence identity with the nucleotide sequence shown in SEQ ID NO:2, wherein the second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and (ii) a third isolated bacteriophage having a genome that has at least 95% sequence identity with the nucleotide sequence shown in SEQ ID NO:3, wherein the third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In some embodiments, the first isolated bacteriophage is Pill582 (SEQ ID NO:1) or progeny thereof; the genome of the second isolated bacteriophage has the nucleotide sequence shown in SEQ ID NO:2; and/or the genome of the third isolated bacteriophage has the nucleotide sequence shown in SEQ ID NO:3.

In another aspect, the present disclosure provides a composition comprising an isolated bacteriophage or phage cocktail as described herein and a pharmaceutically acceptable carrier. In certain embodiments, the composition further comprises an anti-mycobacterial antibiotic. Representative anti-mycobacterial antibiotics include clarithromycin, azithromycin, rifampin, rifabutin, ethambutol, streptomycin, amikacin, cefoxitin, and ciprofloxacin. In certain of these embodiments, the composition comprises at least two anti-mycobacterial antibiotics.

In still another aspect, the present disclosure provides a method for treating a mycobacterial infection. The method generally includes administering to a subject having the mycobacterial infection an effective amount of an isolated bacteriophage or phage cocktail as described herein. In some embodiments, the mycobacterial infection comprises infection with a mycobacterium selected from Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In certain embodiments, the mycobacterial infection is a disseminated and/or chronic infection.

In certain embodiments of a method for treating a mycobacterial infection as described herein, the subject is human. In some embodiments, the mycobacterial infection comprises a pulmonary infection; in some such embodiments, the mycobacterial infection is associated with a pulmonary disease in the subject such as, e.g., a pulmonary disease selected from cystic fibrosis, emphysema, chronic obstructive pulmonary disease (COPD), and bronchiectasis. In other embodiments, the mycobacterial infection comprises a gastrointestinal infection. In certain, non-mutually exclusive variations, the mycobacterial infection comprises infection with Mycobacterium abscessus or Mycobacterium avium.

In some embodiments of a method for treating a mycobacterial infection as above, the subject is an animal. In certain variations, the mycobacterial infection comprises infection with Mycobacterium avium subspecies paratuberculosis or Mycobacterium bovis. In other, non-mutually exclusive embodiments, the animal is a ruminant such as, e.g., a bovine. In yet other, non-mutually exclusive embodiments, the mycobacterial infection comprises a pulmonary infection or a gastrointestinal infection. In some embodiments wherein the mycobacterial infection comprises infection with Mycobacterium avium subspecies paratuberculosis in a ruminant (e.g., a bovine), the mycobacterial infection is associated with Johne's disease in the subject.

In certain embodiments of a method as above, the method is a combination therapy further comprising administering to the subject an effective amount of an anti-mycobacterial antibiotic. Suitable anti-mycobacterial antibiotics include clarithromycin, azithromycin, rifampin, rifabutin, ethambutol, streptomycin, amikacin, cefoxitin, and ciprofloxacin. In some variations, the combination therapy comprises administering at least two anti-mycobacterial antibiotics.

The above aspects of the disclosure will become evident upon reference to the following detailed description.

Pill582 (also referred to herein as “Ph17”; see, e.g., Example 1 and related Figures) is lytic at least against Mycobacterium abscessus, Mycobacterium avium subspecies hominissuis, and Mycobacterium fortuitum. In addition, as further shown in Example 1, infra, Pill582 stimulates TLR2 signaling in THP-1 human macrophages. THP2 signaling plays an essential role in the innate immune activation of phagocytic cells that defend from pathogens. Activation of this signaling attenuates mycobacteria (many species including Mycobacterium tuberculosis) in phagocytic cells and in vivo helps to kill bacteria through the immune system.

For example, with regard to Mycobacterium abscessus (also referred to herein as “MAB”), both smooth and rough phenotypes of MAB have significance in disease pathogenicity. When macrophages are infected with the smooth phenotype of Mycobacterium abscessus (MAB-S), MAB-S has a mechanism to block TLR2 signaling. However, when infected THP-1 macrophages are exposed to the Pill582 phage, the cells engulf the phage, which stimulates TLR2 signaling. This activation initiates the macrophages' innate killing mechanisms such as NLRP3 inflammasome and ROS activation. Consequently, MAB-S is killed indirectly by the macrophage's phage-induced killing mechanisms. This is supported by the observation that phage and bacterial vacuoles do not fuse within cells; they remain in separate vacuoles. See Example 1. In some examples, the disclosed bacteriophages, including Pill582 and functional variants thereof, are characterized by their ability to stimulate TLR2 signaling and thereby enhance macrophage antimicrobial activity through signalosome-mediated NLRP3 inflammasome activation, leading to caspase-1 activation and subsequent production of the pro-inflammatory cytokine IL-1β, ultimately limiting intracellular bacterial survival.

When macrophages are infected with the rough phenotype of Mycobacterium abscessus (MAB-R), MAB-R will initiate TLR2 signaling but it escapes in the cytosol before lysosomes are fused with bacterial vacuoles. However, when infected THP-1 macrophages are exposed to the Pill582 phage, the cells engulf the phage, which stimulates TLR2 signaling before MAB-R escapes in the cytosol. The present inventors have observed fusion events between phage-containing and bacterial vacuoles, most likely because both Pill582 and MAB-R vacuoles are initiated from TLR2 signaling. This allows the phage to directly encounter and infect the bacteria, thereby reducing bacterial numbers through direct phage-mediated killing.

Ore278 and Minn381 are lytic at least against Mycobacterium avium subspecies paratuberculosis (causative of Johne's diseases in ruminants and sometimes responsible for Crohn's disease), Mycobacterium abscessus, Mycobacterium avium subspecies hominissuis, Mycobacterium bovis BCG, Mycobacterium tuberculosis, Mycobacterium fortuitum, Mycobacterium kansasii, and Mycobacterium gordonae. These phages also retain activity at 42° C., at pH 5.0, and in cow rumen. These latter properties are particularly relevant to, e.g., treating Mycobacterium avium subspecies paratuberculosis infection in agricultural animals by oral administration: because infection occurs in the small intestine, phages must bypass cow and other animal rumen as well as withstand conditions that are created in the gastrointestinal tract.

In addition, Ore278 synergizes Mycobacterium abscessus killing by, e.g., amikacin, cefoxitin, and ciprofloxacin when use in combination, with results that are superior to phage or antibiotic treatment alone. Furthermore, Ore278 is active in Mycobacterium abscessus of different physiological states. For example, Ore278 is active against M. abscessus grown in 7H9-base minimal broth supplemented with the following different carbon sources: (i) 5% OADC 10% glycerol, (ii) 10% glycerol, or (iii) 20 mM glucose. This characteristic is relevant, e.g., to use of the phage in treating mycobacterial infections in vivo, since when bacteria infect a host, it changes its physiology and surface targets (versus growth culture media) and, if phage targets are not expressed by bacteria in the host, no matter how phage is effective in vitro, it will not have a therapeutic effect in the clinic.

The Pill582 genome is about 41.4 kb in size and has the nucleotide sequence shown in SEQ ID NO:1. The Ore278 genome is about 71.7 kb in size and has the nucleotide sequence shown in SEQ ID NO:2. The Minn381 genome is about 56.4 kb in size and has the nucleotide sequence shown in SEQ ID NO:3.

In addition to the isolated bacteriophages Pill582, Ore278, or Minn381 and isolated progeny thereof, the present disclosure provides variants of Pill582, Ore278, or Minn381 having a genome that has at least 90% or at least 95% sequence identity with its respective genome and which retains lytic activity against mycobacterial species as summarized above. Such variants of Pill582 typically also retain the ability to stimulate TLR2 signaling in THP-1 human macrophages.

Accordingly, in some aspects, the present disclosure provides an isolated bacteriophage having a genome that has at least 90% sequence identity with the genome of bacteriophage Pill582, deposited under ATCC Patent Deposit Designation No. PTA-127809, wherein the isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages. In certain embodiments, the bacteriophage genome has at least 91%, at least 92%, at least 93%, at least 94%, at least 95%, at least 96%, at least 97%, at least 98%, or at least 99% sequence identity with the genome of bacteriophage Pill582. In other embodiments, the bacteriophage genome has at least 99.1%, at least 99.2%, at least 99.3%, at least 99.4%, at least 99.5%, at least 99.6%, at least 99.6, at least 99.7%, at least 99.8%, at least 99.9%, or 100% sequence identity with the genome of bacteriophage Pill58. In some embodiments, the isolated bacteriophage is Pill582 or progeny thereof.

In some aspects, the present disclosure provides an isolated bacteriophage having a genome that has at least 90% sequence identity with the genome of bacteriophage Ore278, deposited under ATCC Patent Deposit Designation No. PTA-127810, wherein the isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In certain embodiments, the bacteriophage genome has at least 91%, at least 92%, at least 93%, at least 94%, at least 95%, at least 96%, at least 97%, at least 98%, or at least 99% sequence identity with the genome of bacteriophage Ore278. In other embodiments, the bacteriophage genome has at least 99.1%, at least 99.2%, at least 99.3%, at least 99.4%, at least 99.5%, at least 99.6%, at least 99.6, at least 99.7%, at least 99.8%, at least 99.9%, or 100% sequence identity with the genome of bacteriophage Ore278. In some embodiments, the isolated bacteriophage is Ore278 or progeny thereof.

In some aspects, the present disclosure provides an isolated bacteriophage having a genome that has at least 90% sequence identity with the genome of bacteriophage Minn381, deposited under ATCC Patent Deposit Designation No. PTA-127811, wherein the isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In certain embodiments, the bacteriophage genome has at least 91%, at least 92%, at least 93%, at least 94%, at least 95%, at least 96%, at least 97%, at least 98%, or at least 99% sequence identity with the genome of bacteriophage Minn381. In other embodiments, the bacteriophage genome has at least 99.1%, at least 99.2%, at least 99.3%, at least 99.4%, at least 99.5%, at least 99.6%, at least 99.6, at least 99.7%, at least 99.8%, at least 99.9%, or 100% sequence identity with the genome of bacteriophage Minn381. In some embodiments, the isolated bacteriophage is Minn381 or progeny thereof.

In some aspects, the present disclosure provides an isolated bacteriophage having a genome that has at least 90% sequence identity with the nucleotide sequence shown in SEQ ID NO:1, SEQ ID NO:2, or SEQ ID NO:3, wherein the isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In certain embodiments, the bacteriophage genome has at least 91%, at least 92%, at least 93%, at least 94%, at least 95%, at least 96%, at least 97%, at least 98%, or at least 99% sequence identity with the nucleotide sequence shown in SEQ ID NO:1, SEQ ID NO:2, or SEQ ID NO: 3. In other embodiments, the bacteriophage genome has at least 99.1%, at least 99.2%, at least 99.3%, at least 99.4%, at least 99.5%, at least 99.6%, at least 99.6, at least 99.7%, at least 99.8%, at least 99.9%, or 100% sequence identity with the nucleotide sequence shown in SEQ ID NO:1, SEQ ID NO:2, or SEQ ID NO:3. In some embodiments, the isolated bacteriophage has the nucleotide sequence shown in SEQ ID NO:1, SEQ ID NO:2, or SEQ ID NO:3.

The bacteriophages of the invention can be prepared by standard culture, isolation and purification methods. For example, host mycobacteria (e.g., M. smegmatis) are incubated with a sample of a bacteriophage, and then plated in a medium, for example top agar medium, to obtain phage plaques. Next, phage buffer is overlaid on the agar to recover bacteriophage suspension, which is filtered to remove bacterial cell lysate. One or more cycles of selective propagation of bacteriophages of the invention may be performed to obtain phage in high titration. Following filtration, phage is tested for presence of lytic activity.

The titration of bacteriophages in a suspension and the visualization of plaque morphology of bacteriophages of the invention may then be assessed by known methods such as, for example, by plaque counting. Additionally, processing bacteriophages of the invention in various forms (liquid, lyophilized, etc.) for short-, long-, freeze- or any other kind of storage can be carried out by any suitable method well-known in the art.

The lytic activity of the bacteriophages of the invention can be assessed by methods well-known in the art such as, e.g., a plaque assay (also known as double agar method), based on the growing of bacteriophage with potential host bacteria and followed by assessing their ability to kill the host bacterial cell. In the plaque assay method, the bacteriophage induces lysis of target mycobacteria strains after a period of incubation in soft agar medium, resulting in zones of clearing on the plate known as plaques.

The bacteriophages of the invention may be cultured, expanded, isolated, purified, and used in, e.g., phage therapy of mycobacterial infections and mycobacteria-mediated disorders, as disclosed herein. Furthermore, bacteriophage variants retaining a phenotypic character (e.g., lytic activity) of Pill582, Ore278, or Minn381 can be produced and/or isolated by techniques known in the art.

In some aspects, the present invention provides a phage cocktail comprising at least two phages as described above. For example, in certain variations, a phage cocktail includes at least two bacteriophages selected from (i) a first isolated bacteriophage having a genome that has at least 90% or at least 95% sequence identity with the genome of bacteriophage Pill582 (SEQ ID NO:1), deposited under ATCC Patent Deposit Designation No. PTA-127809, wherein the first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; (ii) a second isolated bacteriophage having a genome that has at least 90% or at least 95% sequence identity with the genome of bacteriophage Ore278, deposited under ATCC Patent Deposit Designation No. PTA-127810, or having a genome that has at least at least 90% or at least 95% sequence identity with the nucleotide sequence shown in SEQ ID NO:2, wherein the second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and (iii) a third isolated bacteriophage having a genome that has at least 90% or at least 95% sequence identity with the genome of bacteriophage Minn381, deposited under ATCC Patent Deposit Designation No. PTA-127811, or having a genome that has at least at least 90% or at least 95% sequence identity with the nucleotide sequence shown in SEQ ID NO: 3, wherein the third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae. In some preferred embodiments, a phage cocktail includes the first isolated bacteriophage and at least one of the second and third isolated bacteriophages. In some embodiments, a phage cocktail composition includes the first, second, and third isolated bacteriophages.

As previously indicated, the isolated bacteriophages and phage cocktails disclosed herein can be used to provide therapy for the treatment of mycobacterial infections. Such infections may include, for example, pulmonary infections, gastrointestinal infections, infections of the skin and soft tissues, bacteremia, and central nervous system infections, to name a few. The infection to be treated may be, for example, disseminated and/or chronic. The phage and phage cocktail compositions are particularly useful, e.g., in treating mycobacterial infections associated with diseases or disorders for which mycobacterial pathogens may be a causative or exacerbating agent. Such diseases or disorders can include, for example, pulmonary disease (e.g., cystic fibrosis, emphysema, chronic obstructive pulmonary disease, and bronchiectasis), gastrointestinal disease (e.g., Johne's disease in ruminants, or gastric diseases in humans and other subjects), and diseases or disorders related to skin and soft tissues (e.g., peritonitis). The phage and phage cocktail compositions are also particularly useful, e.g., for treating immunocompromised individuals having an increased risk for infection with mycobacteria (e.g., solid organ transplant recipients having depressed cell-mediated immunity, for whom mycobacterial infections can become chronic and require ongoing treatment). In certain variations, the mycobacterial infection to be treated comprises an infection with a mycobacterium selected from Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

For therapeutic use, a bacteriophage or phage cocktail composition as described herein is delivered in a manner consistent with conventional methodologies associated with management of the infection (including any associated disease or disorder) for which treatment is sought. In accordance with the disclosure herein, an effective amount of the bacteriophage or phage cocktail is administered to a subject in need of such treatment for a time and under conditions sufficient to prevent or treat the mycobacterial infection.

The bacteriophage treatment may be formulated as a concentrate composition or a ready-to-use composition. A bacteriophage or phage cocktail composition may be freeze-dried or spray-dried for storage, if desired. Upon reconstitution, the phage titer can be verified using phage titration protocols and host bacteria. One of skill in the art would be capable of determining bacteriophage titers using widely known bacteriophage assay techniques. See, e.g., Davis et al., Microbiology, 3rd Ed., Harper & Row, Hagerstown, Md. (1980), pp. 874-877, 880-883.

For administration, a bacteriophage or phage cocktail in accordance with the present invention is formulated as a pharmaceutical composition. A pharmaceutical composition comprising a bacteriophage or phage cocktail as described herein can be formulated according to known methods to prepare pharmaceutically useful compositions, whereby the therapeutic agent is combined in a mixture with a pharmaceutically acceptable carrier. A composition is said to be a “pharmaceutically acceptable carrier” if its administration can be tolerated by a recipient patient. Pharmaceutically acceptable carriers include any of the standard pharmaceutical carriers, such as, for example, a phosphate buffered saline solution, water, emulsions (e.g., such as an oil/water or water/oil emulsions), and various types of wetting agents. Other suitable carriers are well-known to those in the art. See, e.g., Gennaro (ed.), Remington's Pharmaceutical Sciences (Mack Publishing Company, 19th ed. 1995). Formulations may further include one or more excipients, preservatives, solubilizers, buffering agents, albumin to prevent protein loss on vial surfaces, etc.

Pharmaceutical compositions containing bacteriophages disclosed herein can be presented in a dosage unit form and can be prepared by any suitable method. A pharmaceutical composition should be formulated to be compatible with its intended route of administration. The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form will depend upon the intended mode of administration and therapeutic application.

A pharmaceutical composition comprising a bacteriophage or phage cocktail of the present invention is administered to a subject in an effective amount. The bacteriophage or phage cocktail composition may be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, the bacteriophage or phage cocktail may be administered to a subject in a single bolus delivery, via continuous delivery (e.g., continuous transdermal delivery) over an extended time period, or in a repeated administration protocol (e.g., on an hourly, daily, or weekly basis).

In some embodiments, such as in the case of a pulmonary infection, a pharmaceutical composition comprising a bacteriophage or phage cocktail of the present invention is formulated for delivery to the lung by nebulization using a nebulizing device. Nebulization may be carried out with a portable inhaler or with an add-on nebulizer to a medical mechanical ventilator. A phage or phage cocktail solution volume (e.g., from about 1 to about 20 mL) may be nebulized at various time intervals and during the treatment period. Before starting the first nebulization and according to the pathology, lung washing may be performed with the same phage or phage cocktail solution.

For administration to non-human animal subjects (e.g., ruminants), in addition to administration as generally discussed above, isolated bacteriophage or phage cocktail may be combined with animal feed or water and administered orally to an animal. The phage animal feed formulation can be provided to animals at selected time intervals and then replaced with regular animal feed as desired. Alternatively, bacteriophage or phage cocktail in an acceptable carrier (e.g., water or saline) may be instilled into the nose of an animal with, for example, a syringe with a nasal spray tip in a suitable volume (e.g., about 1 mL). Bacteriophage or phage lysate can also be administered in an acceptable carrier via a stomach tube in any suitable volume (e.g., about 50 mL). In yet another alternative, bacteriophage or phage lysate can be formulated in an aerosol by combining the phage in a carrier (e.g., water or saline) or in a propellant (e.g., trichloromonofluoromethane, dichlorodifluoromethane, or oleic acid). Aerosol formulations of the phage or phage cocktail can be administered in the nasal passage or oral cavity of the animal.

Dosage can be determined on a specific basis for each specific type of phage in each specific type of infection in each specific host. Thus, those skilled in the art will recognize that the effective dosage will vary depending on the type of infection and virulence of the phage(s). The concentration of bacteriophage or phage cocktail employed for treatment may be determined using phage titration protocols known in the art.

Determination of effective dosages is typically based on animal model studies followed up by human clinical trials and is guided by determining effective dosages and administration protocols that significantly reduce the occurrence or severity of the subject disease or disorder in model subjects. Effective doses of the compositions of the present invention vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Typically, dosage regimens are adjusted to provide an optimum therapeutic response, i.e., to optimize safety and efficacy. Accordingly, a therapeutically effective amount is also one in which any undesired collateral effects are outweighed by beneficial effects.

In some embodiments, a bacteriophage or phage cocktail composition to obtain lytic activity in vivo includes a concentration of bacteriophage from about 10² to about 10¹² Plaque Forming Units (PFU)/mL (e.g., about 10³, about 10⁴, about 10⁵, about 10⁶, about 10⁷, about 10⁸, about 10⁹, or about 10¹⁰, or about 10¹¹ PFU/mL). In some embodiments, the bacteriophage concentration is at least about 10⁴ PFU/mL, at least about 10⁵ PFU/mL, or at least about 10⁶ PFU/mL. In some embodiments, the bacteriophage concentration ranges from about 10⁶ to about 10¹⁰ PFU/mL or about 10⁷ to about 10¹¹ PFU/mL.

In certain embodiments of a method for treating a mycobacterial infection as described herein, the method is a combination therapy. In some such embodiments, the combination therapy further includes administration of one or more non-phage therapeutic agents useful for treatment of a mycobacterial infection or a disease or disorder associated with the infection. In more particular variations, a non-phage therapeutic agent for use in a combination therapy is an anti-mycobacterial antibiotic. Particularly suitable anti-mycobacterial antibiotics include clarithromycin, azithromycin, rifampin, rifabutin, ethambutol, streptomycin, amikacin, cefoxitin, and ciprofloxacin, to name a few. Other non-phage therapeutic agents that may be used in combination with the bacteriophages or phage cocktails of the present disclosure include, for example, anti-inflammatory agents (e.g., corticosteroids) and immunomodulatory agents (e.g., arginine, 1,25-dihydroxyvitamin D3 (vitD), or histone deacetylase (HDAC) inhibitors such as, for example, sodium phenylbutyrate (PBA)).

In related aspects, the present invention provides compositions for treating mycobacterial infections as described above. In some embodiments, a composition for treating a mycobacterial infection includes (i) an isolated bacteriophage or phage cocktail as described herein and (ii) a pharmaceutically acceptable carrier. In some embodiments, a composition for treating a mycobacterial infection includes (i) an isolated bacteriophage or phage cocktail as described herein and (ii) a non-phage therapeutic agent (e.g., an anti-mycobacterial antibiotic, anti-inflammatory agent, or immunomodulatory agent).

The following describes phage-mediated TLR2-signaling results in attenuation of intracellular Mycobacterium abscessus within macrophages (see Example 1 below), particularly screening a phage library and investigating whether mycobacterial specific phages can activate innate immune responses limiting intracellular bacterial survival within professional phagocytes and elucidate mechanisms behind it.

Mycobacteriophages Engage with Phagocytic Cells

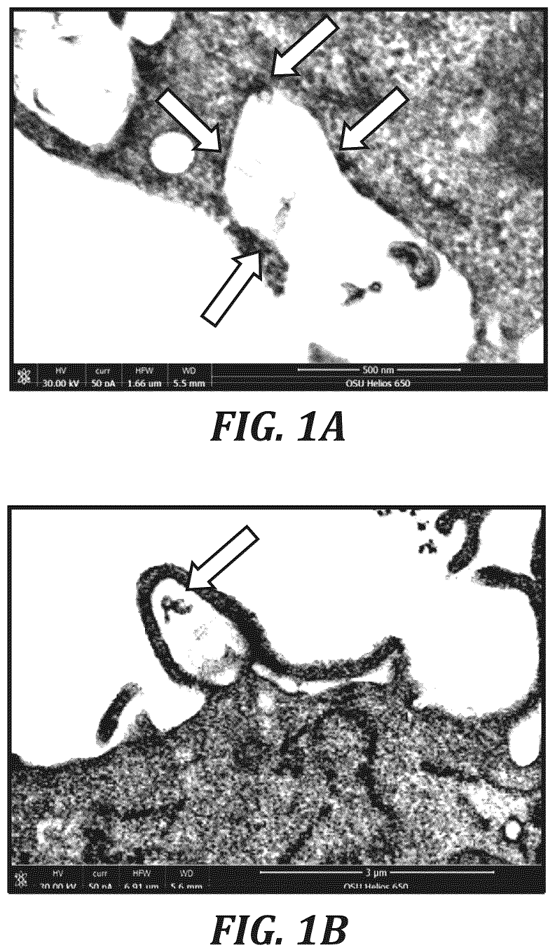

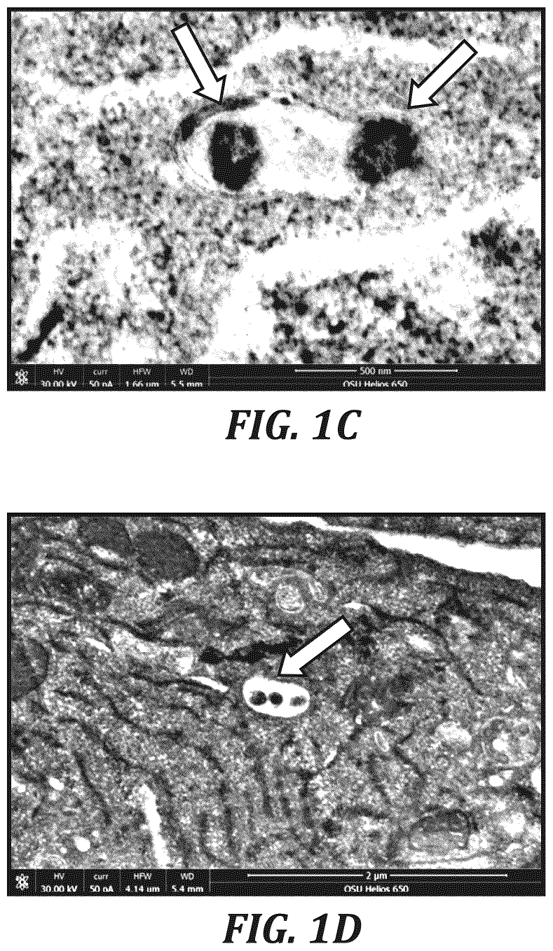

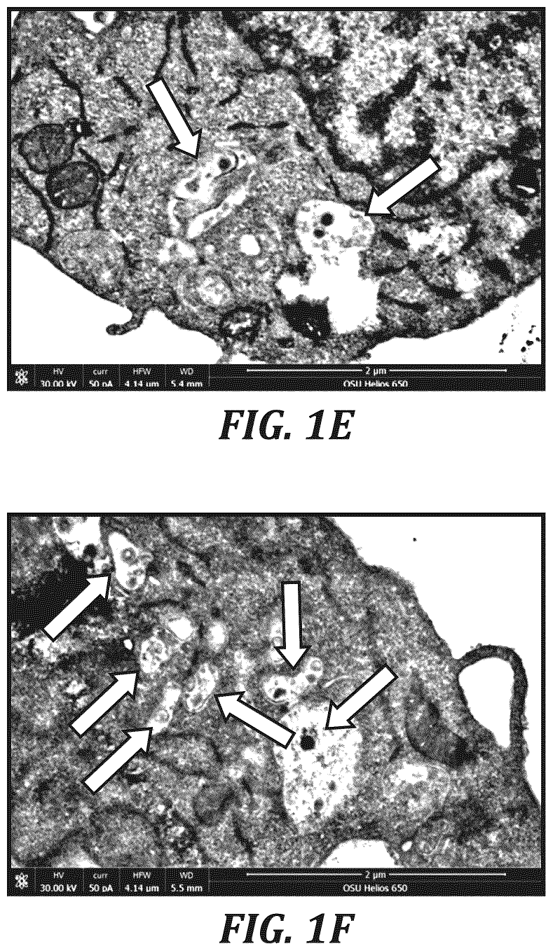

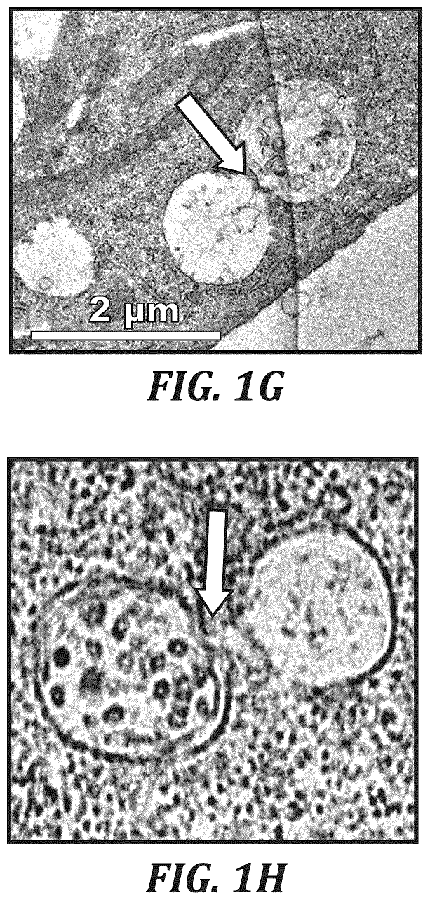

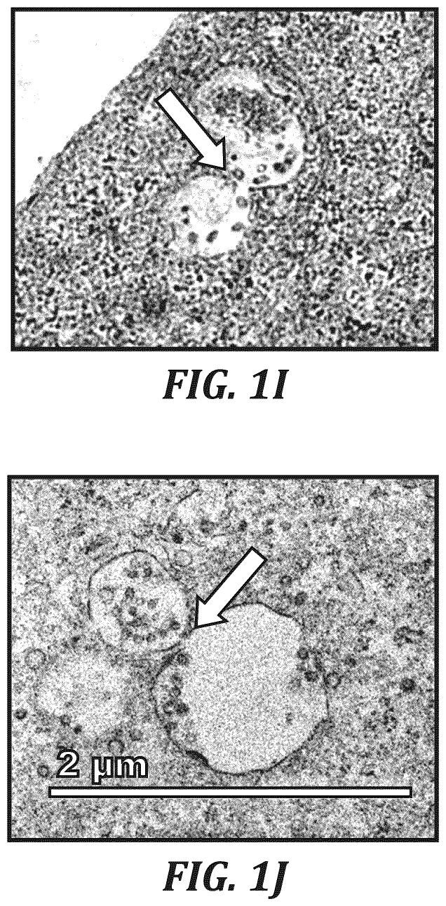

To determine if mycobacteriophages adhere and stimulate the phagocytosis process in human macrophages, a lytic mycobacteriophage was exposed to THP-1 cells and phage uptake process was monitored over 24 h. The transmission electron microscopy (TEM) micrographs, displayed in FIGS. 1A-1J, reveal that macrophages engulf phages through the endocytosis process (FIGS. 1A and 1B) and, later, transported to the cytosol where they reside within vacuoles formed from the plasma membrane during the internalization process (FIGS. 1C-1F). Interestingly, it was also observed that when multiple phage vacuoles are formed in macrophages during phage uptake by phagocytic cells, the fusion between these vacuoles take place (FIGS. 1G-1J).

Identification of Phages Associated with Modulation of Intracellular MAB Growth within THP-1 Cells

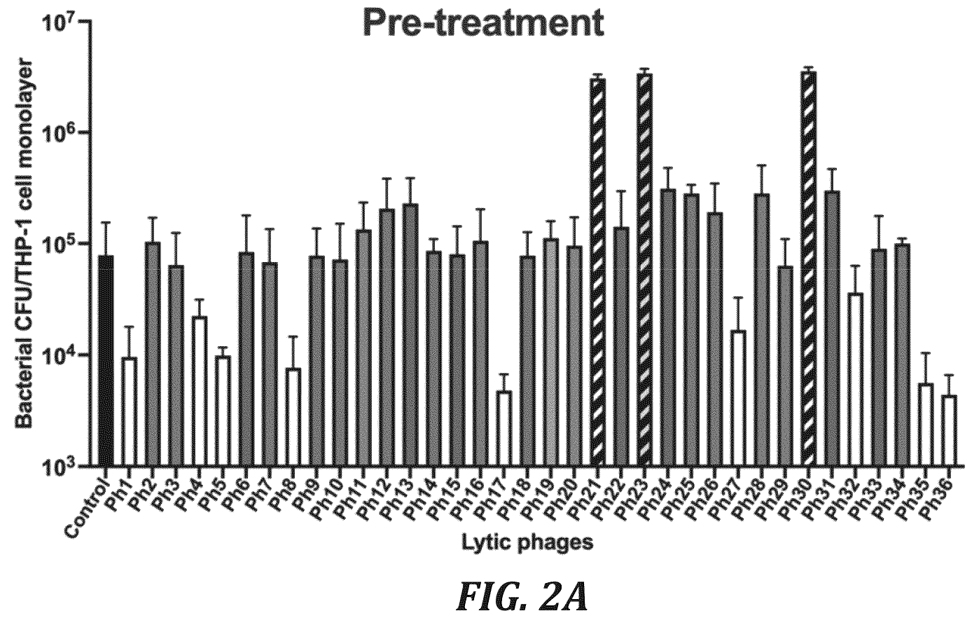

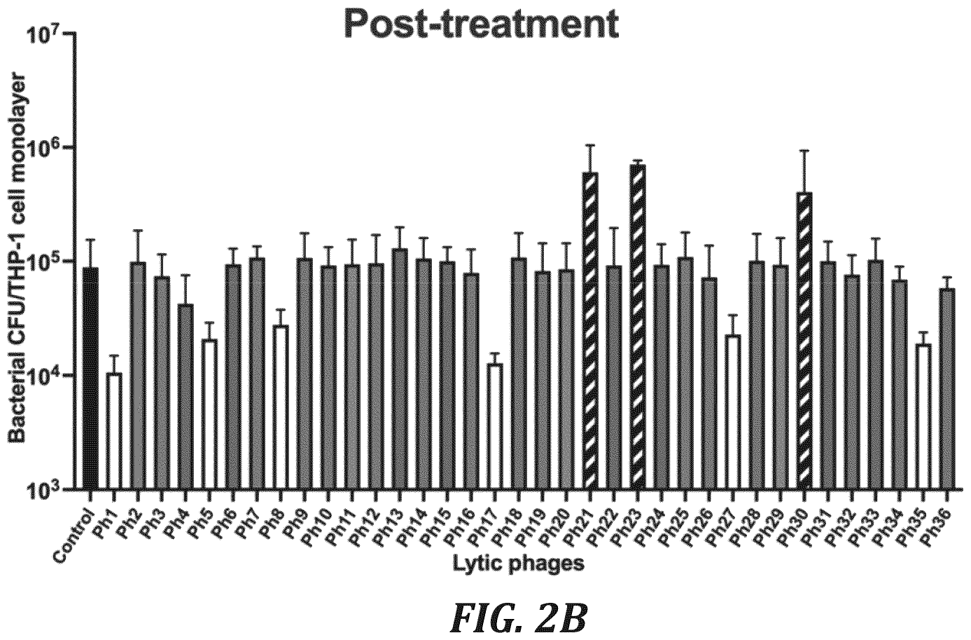

To investigate whether phages can stimulate innate immune responses resulting in phagocyte-mediated clearance of intracellular MAB, experiments were conducted using two distinct approaches. In the first setup, THP-1 cells were pre-treated with thirty-six lytic phages before MAB infection (FIG. 2A). While in the second assay, macrophage monolayers were initially infected with MAB and then subjected to phage treatment (FIG. 2B). Presented herein are findings on six phages that promote macrophage responses limiting intracellular MAB growth in both experimental setting. Notably, also identified are three phages that contribute to macrophage response facilitating better growth of intracellular MAB when compared to the control group with no phage exposure (FIGS. 2A and 2B).

### Activation of TLR2 and TLR4 Signaling Enhance Innate Killing Mechanisms Against MAB

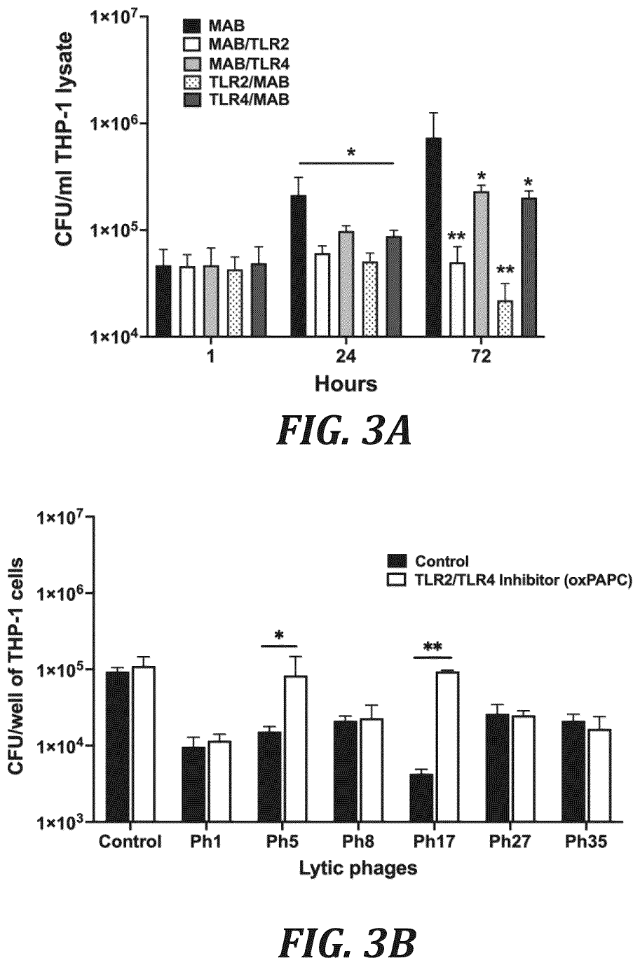

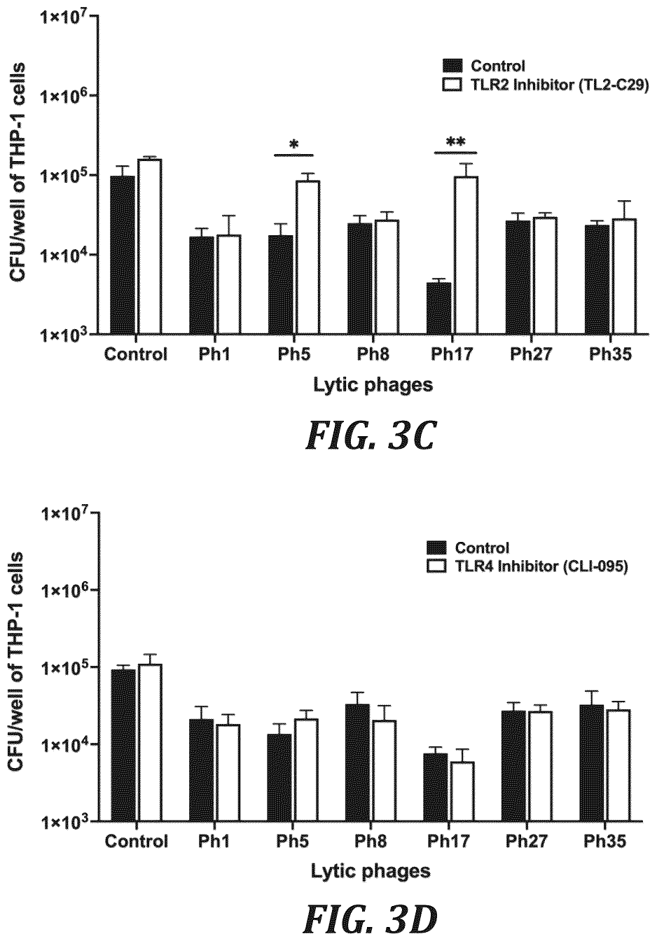

The present disclosure demonstrates that when TLR2 and TLR4 signaling is activated in THP-1 macrophages via TLR2 (HKLM, freeze-dried heat-killed preparation of Listeria monocytogenes) and TLR4 (LPS-EK, lipopolysaccharide from E. coli) agonists, it leads to significant attenuation of intracellular MAB growth when compared with the MAB infection group alone (FIG. 3A). Bacterial growth inhibition is evident whether agonists are used before or after MAB infection of THP-1 macrophages, but the most substantial decrease in bacterial Colony Forming Units (CFUs) occurs when cells are pre-exposed to TLR agonists before infection (FIG. 3A). These results suggest that stimulation of TLR signaling in presence of MAB infection triggers innate immune mechanisms that play a pivotal role in limiting intracellular bacterial proliferation within phagocytic cells.

Moreover, because phages elicit a wide range of immunomodulatory properties, the present disclosure tested whether six selected phages known to contribute to MAB attenuation in macrophages as shown in FIGS. 2A and 2B, might be also implicated in TLR2 or TLR4 activation. The results reveal that when TLR2 and TLR4 signaling are blocked using a receptor specific antagonist, two out of six phages are unable to effectively eliminate intracellular MAB in only TLR2-inhibited cells (FIGS. 3B-3D). Although no changes were noted during TLR4 pathway inhibition (FIG. 3D), blocking the TLR2 signaling in the Ph5 and Ph17 phage treated groups reversed outcomes, closely reflecting those of the non-phage control group (FIGS. 3B and 3C). Results suggest that the uptake of these two phages likely transpire via a TLR2-mediated pathway.

Evaluation of Ph17 Treatment on Intracellular MAB Growth with Smooth (MAB-S) and Rough (MAB-R) Morphotypes

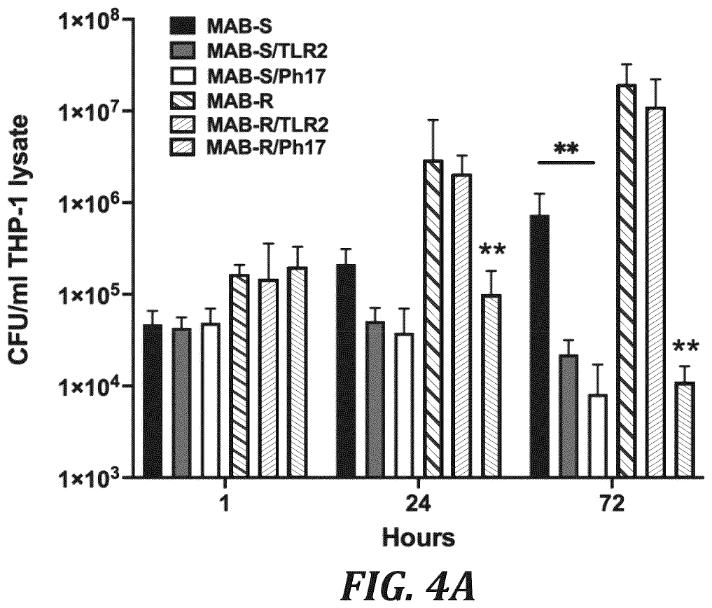

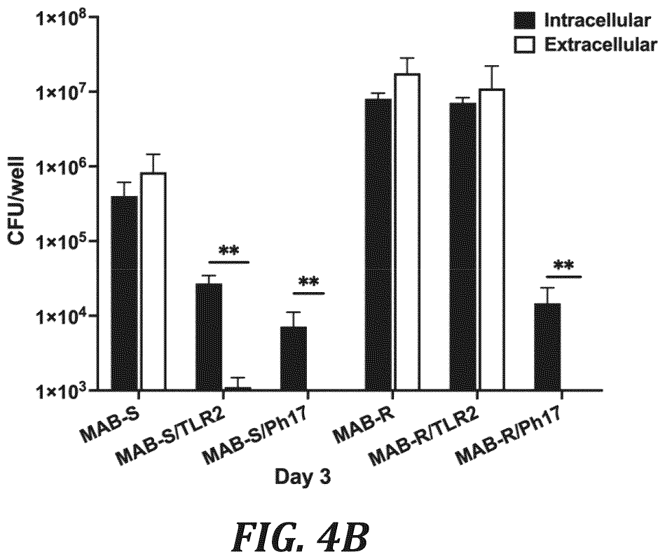

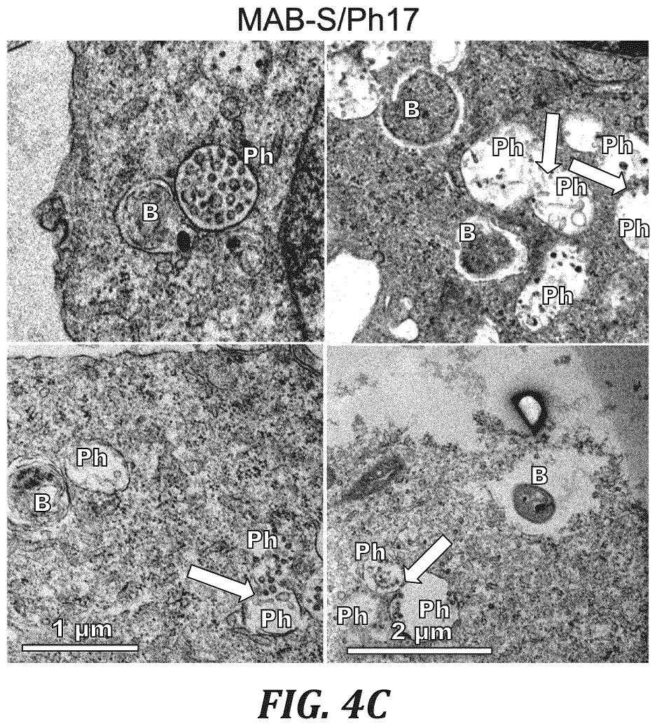

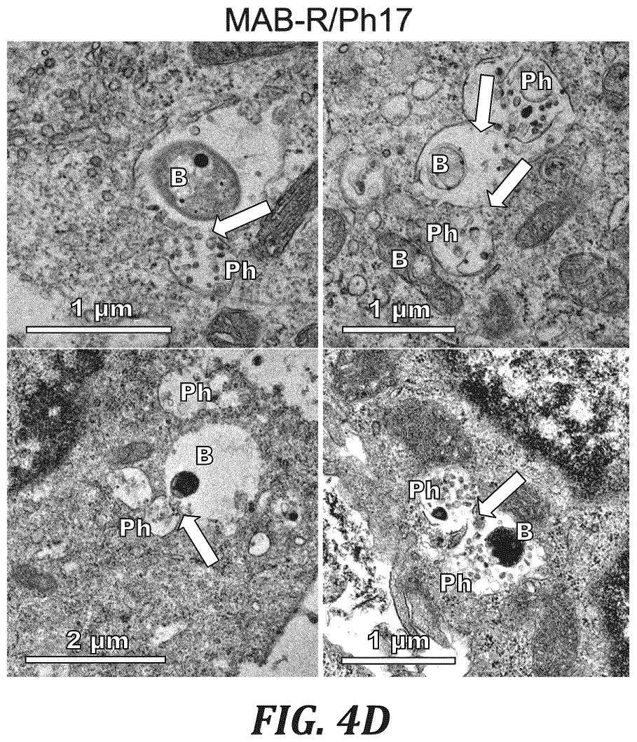

THP-1 cells were treated with TLR2 agonist or Ph17 after infecting monolayers with either MAB-S or MAB-R morphotypes. Macrophage monolayers with only bacterial infection served as a control. The results demonstrate that while activation of TLR2 signaling using the TLR2 agonist leads to significant attenuation of intracellular MAB-S growth, it did not have similar effect on MAB-R where bacteria grew at a much higher rate than MAB-S control group without TLR2 activation (FIG. 4A). In contrast, the inhibition of intracellular bacterial growth was evident in both MAB-S and MAB-R infection groups that were treated with Ph17 phage as shown for 24 h and 72 h post-infection time-points (FIG. 4A).

Next, to understand the MAB-S or MAB-R growth dynamics during Ph17 treatment, extracellular and intracellular bacterial loads were quantified and compared to TLR2 agonist exposed and bacteria only infection groups at day 3 of post-infection. As shown in FIG. 4B, TLR2 activation in MAB-R infection group has no inhibitory effect on intracellular bacteria and large number of extracellular MAB-R is recovered in the supernatant fraction, whereas the TLR2 activation in MAB-S infected group reduced bacterial loads in both intracellular and extracellular fractions. Furthermore, the results demonstrate that Ph17 treatment of THP-1 cells infected either MAB-S or MAB-R significantly reduces both bacterial loads in the intracellular fraction and no bacteria is recovered in the extracellular faction in both experimental groups of the phage treatment (FIG. 4B).

### Limiting the Intracellular Growth of MAB-S and MAB-R Morphotypes by Ph17 Via Two Distinct Mechanisms

To determine a cellular mechanism activated by Ph17 treatment and limiting intracellular mycobacterial growth within phagocytic cells, macrophage infection experiments were repeated with MAB-S and MAB-R clones and subjected for TEM analysis after three hours of phage treatment. The TEM images of THP-1 cells infected with MAB-S (known to inhibit TLR2 signaling) and subsequently treated with Ph17 (stimulating TLR2 signaling), reveal a distinct separation of phage and bacterial vacuoles (FIG. 4C). This finding suggests attenuation of MAB-S via innate killing mechanism likely through phage-induced TLR2 activation pathway. MAB-R is recognized for its ability to trigger TLR2 signaling while evading macrophage innate mechanisms by escaping into the cytosol. Contrary to MAB-S, TEM images reveal a phenomenon where fusion of vacuoles containing MAB-R and Ph17 take place (FIG. 4D). These results suggest the pathogen elimination primarily via direct killing by lytic phages before MAB-R escape into the cytosol.

### Phage-Enhanced Activation of NLR Family Pyrin Domain Containing 3 (NLRP3) Inflammasome and Mitochondrial Oxidative Stress (ROS) in MAB Infected Macrophages

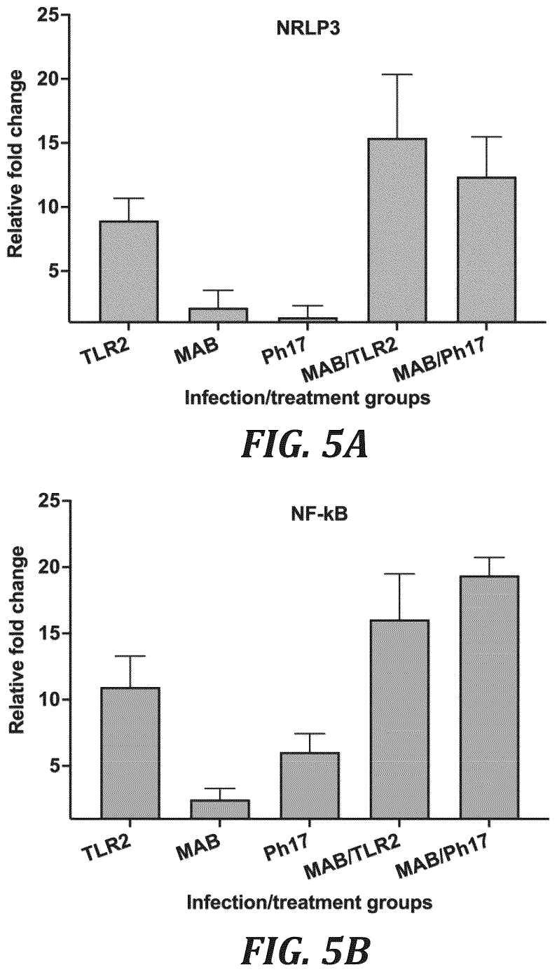

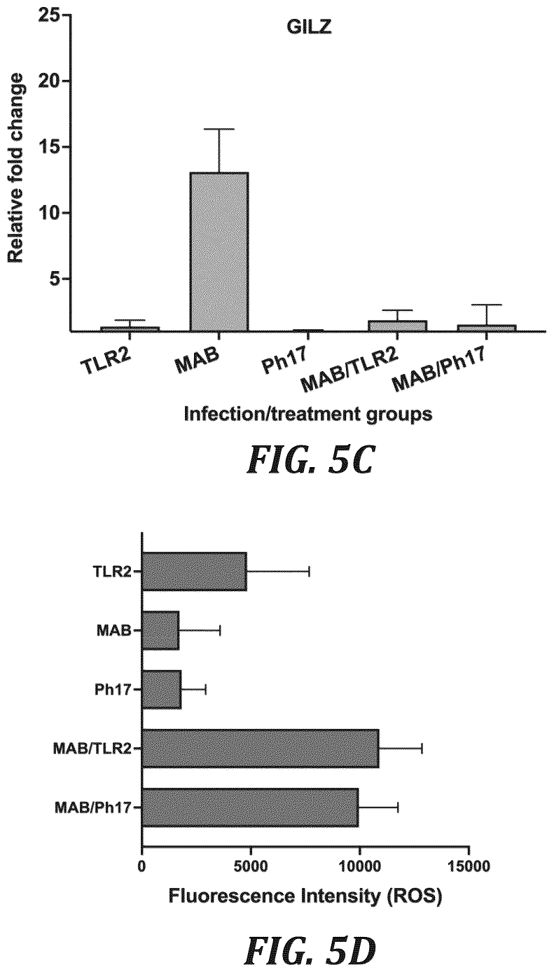

Modulation of inflammasome and ROS has been demonstrated to effect MAB virulence in phagocytic cells. See Kim et al., PLOS Pathog. 16:e1008294, 2020; Bernut et al., Cell Rep. 26:1828-1840 e1824, 2019. The expression levels of NLRP3 and Nuclear Factor Kappa B (NF-kB) genes were evaluated in MAB-S infected THP-1 cells. In addition, the Glucocorticoid-induced leucine zipper (GILZ) gene expression levels were also assessed as it directly interacts with and prevents NF-κB-mediated activation of transcription (see Di Marco et al., Nucleic Acids Res. 35:517-528, 2007) as well as downregulates TLR2 expression and inhibit bactericidal activity in macrophages. The real-time PCR analysis of MAB-S infected THP-1 cells demonstrates the high expression levels of NLRP3 and NF-kB1 genes in TLR2 activated and Ph17 post-treatment groups when compared with the MAB-S control group (FIGS. 5A and 5B). In contrast, the high expression of GILZ in MAB-S infected cells corelates with downregulation of NLRP3 and NF-κB in TLR2 activated cells as well as in MAB infection/Ph17 or MAB infection/TLR2 co-treatment groups (FIG. 5C). While increased level of NF-kB1 is seen in the THP-1 macrophage treatment group solely administered with Ph17 when compared with MAB infection group, significant upregulation in NLRP3 gene expression was not observed within this group unless it is coupled with MAB infection (FIGS. 5A and 5B).

TLR2 signaling plays critical role in oxidative stress, and because ROS contributes to immune resistance against MAB infection, whether phage treatment of MAB-S infected macrophages was also associated with increased levels of mitochondrial damage was examined using CellROX green fluorogenic probe. Results indicate that ROS levels notably increased in TLR2 activated or Ph17 treated macrophages during MAB infection when compared with MAB infection or phage alone groups (FIG. 5D). Collectively, the data suggest that activation of TLR2 signaling either through TLR2 agonist or Ph17 treatment stimulates innate immune responses beneficial for the host cells against MAB-S infection.

### Phage-Enhanced Immune Activation Leads to Increased IL-1β Secretion and Elevated Production of Caspase-1 in MAB Infected Macrophages

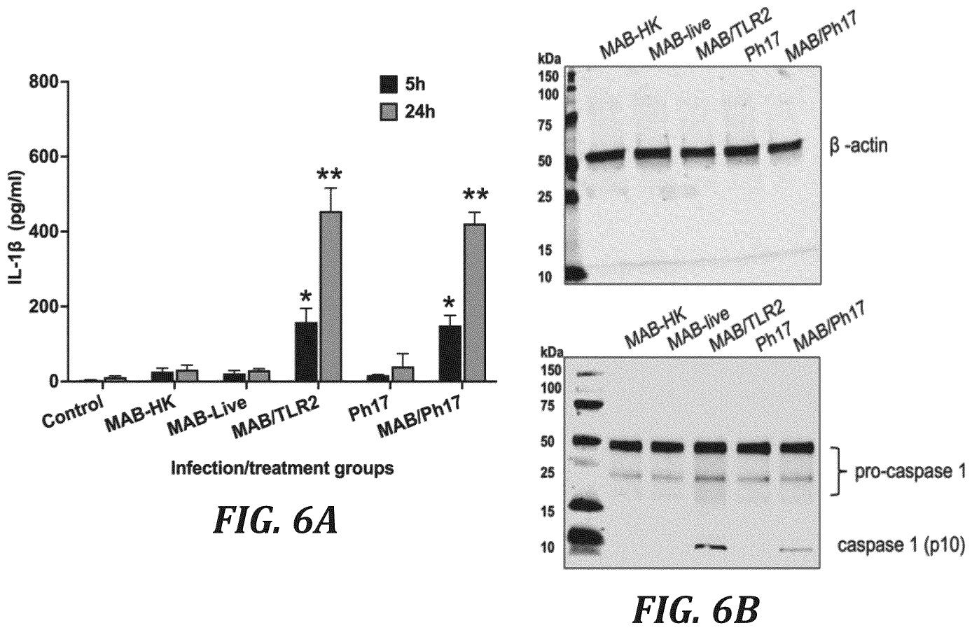

Because the significant expression levels of NLRP3 and ROS were observed in MAB-S infected macrophages during Ph17 exposure, the IL-1β secretion levels in supernatants of THP-1 cells as well as the mature caspase 1 protein levels were measured via the Western blot analysis on precleared cell lysates. Elevated production of IL-1β in MAB infected/Ph17 treatment group was found when compared to bacterial infection or phage treatment alone groups, suggesting that IL-1β secretion is stimulated in TLR2 activated phagocytic cells by TLR2 agonist or Ph17 and merely during MAB-S infection (FIG. 6A). These changes increased over time and displaying significantly higher levels of IL-1β secretion at 24 h time point compared to 5 h. Notably, cells treated solely with Ph17 did not show any elevated levels of IL-1β.

A similar trend was observed for a mature caspase-1 protein in the MAB-S infected/Ph17 treated group at 24 h when compared to controls (FIG. 6B). The elevated production of mature caspase-1 (p10) further supports the findings that ROS and inflammasome activation leads to increased IL-1β secretion and enhanced production of caspase-1 (p10). The fact that the significant levels of IL-1β production and enrichment in caspase-1 protein were detected in the MAB-S infected/Ph17 treated macrophages in comparison to MAB-S only infection indicate that these changes are stimulated by phages.

MAB belongs to the most difficult to treat mycobacterial infection. See Lee et al., Emerg. Infect. Dis. 21:1638-1646, 2015. The treatment outcomes in clinics are unpredictable and, in many cases, ineffective. See Chen et al., Front. Microbiol. 10:1977, 2019; Nessar et al., J. Antimicrob. Chemother. 67:810-818, 2012. Increased antibiotic resistance and subsequent failure of clinically available antibiotics have fueled discussions to develop an alternative therapeutic strategy of eliminating bacteria by lytic phages. However, current understanding of interactions among phages, mycobacteria, and the mammalian host immune system remains limited. As described herein, the immunomodulatory characteristics of mycobacteriophages were investigated, and phages were identified that are capable of stimulating innate immune defenses of phagocytic cells leading to attenuation of intracellular MAB.

Recent studies provide evidence that phages facilitate innate immune activation through two primary mechanisms: either through direct interface with the cell membrane receptors, which subsequently enhance phagocytosis and the cytokine/chemokine secretion by mononuclear phagocytes or via their impact on bacteria. See Jonczyk-Matysiak et al., Viruses 9, 2017; Kurzepa et al., Clin. Exp. Med. 9:93-100, 2009; Carroll-Portillo and Lin, Microorganisms 7, 2019. More specifically, phage-mediated lysis of bacteria has been demonstrated to activate an innate immunity in vivo (see Tiwari et al., J. Microbiol. 49:994-999, 2011), underscoring the significance of the synergy between phage lytic activity and subsequent stimulation of the host immune system for effective therapeutic outcomes (see Leung and Weitz, J. Theor. Biol. 429:241-252, 2017; Roach et al., Cell Host Microbe 22:38-47 e34, 2017). Some of the results derived from mathematical modeling indicate that phages alone are insufficient to eradicate the entire bacterial population, emphasizing the necessity of collaboration with the immune system for successful phage therapy. See Leung and Weitz, supra. Interestingly, phage therapy has demonstrated improved animal survival using non-lysing (endolysin-deficient) phages over lytic phages (see Paul et al., BMC Microbiol. 11:195, 2011), suggesting that effective pathogen clearance can be achieved through the activation of host immune defenses. Moreover, a subset of non-lytic phages equipped with capsular depolymerases can enhance immune-mediated bacterial killing in vivo by releasing these enzymes. See Mushtaq et al., J. Antimicrob. Chemother. 56:160-165, 2005; Lin et al., Front. Microbiol. 8:2257, 2017; Lin et al., Viruses 10, 2018.

Initially, to validate the ability of mycobacteriophages to bind to and induce the phagocytosis process in human macrophages, transmission electron microscopy was conducted. These experiments allowed capture of the processes of lytic phage uptake by THP-1 cells over time. The TEM micrographs presented in FIGS. 1A-1J illustrate the early stages of phage engulfment by phagocytes via endocytosis, as well as later events where phage vacuoles, originating from the plasma membrane during the internalization process, are directed to the cytosol. Interestingly, it was also observed that when multiple phage vacuoles are formed, the fusion between these phage vacuoles take place likely driven by a shared cellular mechanism initiated during phagocytosis process at the plasma membrane.

To identify if phages can stimulate innate immune responses in phagocytic cells restricting the intracellular MAB growth within THP-1 cells, a screening of a mycobacteriophage library was conducted. A two-stage approach was employed involving pre- and post-treatment strategies, utilizing a total of thirty-six lytic phages. The findings on six phages described herein that demonstrated the ability to enhance the clearance of MAB by macrophages in both experimental settings. The data collectively shows that certain phages can boost macrophage defenses, enabling them to overcome processes suppressed by the intracellular pathogen and attenuate its virulence mechanisms. In contrast, three phages associated with unfavorable outcomes were also identified, as they increased MAB growth in THP-1 cells even beyond what was observed in the bacterial control group with no phage exposure.

A growing body of literature exploring phage interactions with the immune system reveals a complex interplay between phages and the host immune system, extending beyond their direct antibacterial effects. Phages harbor ligands for TLRs (TLR7, TLR8, TLR9), RIG-I-like receptors, and components capable of activating TLR2 and TLR4 activity. See Jonczyk-Matysiak et al., supra; Carroll-Portillo and Lin, supra; Sartorius et al., EMBO Mol. Med. 7:973-988, 2015. The interplay between phage-derived ligands and PRRs contributes to the efficacy of phage therapy. See Krut and Bekeredjian-Ding, J. Immunol. 200:3037-3044, 2018. Conversely, extensive research on mycobacterial recognition by TLRs underscores the pivotal role of TLR2 and TLR4 as critical PRRs in triggering innate immune responses that can impact intracellular bacterial survival in the host. See Ryffel et al., Tuberculosis (Edinb) 85:395-405, 2005; Kim et al., Infect. Immun. 83:1556-1567, 2015; Le Moigne et al., Front. Cell Infect. Microbiol. 10:432, 2020; Hu and Spaink, Biology (Basel) 11, 2022; Park et al., Immune Netw. 21:e40, 2021. The findings described herein indicate that the activation of TLR2 and TLR4 signaling with agonists significantly inhibits MAB growth within THP-1 human macrophages. Given that certain phages contain ligands capable of directly activating TLR2 and TLR4 signaling (see Jonczyk-Matysiak et al., supra; Carroll-Portillo and Lin, supra; Sartorius et al., supra), they may contribute to the attenuation of MAB within phagocytic cells. Therefore, six selected phages, which were associated with attenuation of intracellular MAB, were assessed for their potential to activate TLR2 and TLR4 pathways. This analysis revealed that Ph5 and Ph17 phages are associated with the stimulation of TLR2 signaling.

It is well established that MAB-S can escape the TLR2-signaling through “masking” the underlining bioactive cell wall components with glycopeptidolipids (GPLs) in comparison to MAB-R that provoke TLR2 response. See Davidson et al., PLOS One 6:e29148, 2011. Studies also suggest a vaccine potential of TLR2-activating the cell wall fraction purified from MAB-R variant that provides protection against MAB-S challenge in mice. See Le Moigne et al., supra. The findings described herein also support this data and indicate that while MAB-S avoids TLR2 activation, when TLR2 signaling is stimulated via agonist or exposing to certain phages, the intracellular pathogen is unable to counteract macrophage defenses effectively. Given that TLR2 signaling plays a crucial role in stimulating immune responses that can inhibit mycobacterial survival in the host (see Noss et al., J. Immunol. 167:910-918, 2001; Gilleron et al., J. Biol. Chem. 278:29880-29889, 2003; Lien et al., J. Biol. Chem. 274:33419-33425, 1999; Bulut et al., J. Biol. Chem. 280:20961-20967, 2005), and TLRs also trigger signaling cascades leading to the activation of NRLP3 inflammasome, NF-κB, and pro-inflammatory cytokines (see Jo et al., Cell. Microbiol. 9:1087-1098, 2007), a quantitative gene expression analysis was conducted using the qRT-PCR for these targets. Results revealed significant expression levels of these genes in the MAB-S infected and phage-treated group versus MAB-S only infection group, providing strong support for activation of TLR2 signaling by phages.

Furthermore, the regulatory role of GILZ in inflammation, macrophage polarization and apoptosis in vitro and in vivo (see Vago et al., Pharmacol. Res. 158:104842, 2020), defense mechanisms such as phagocytosis and bactericidal activity is well established (see Hoppstadter et al., Front. Immunol. 9:3111, 2018). The positive regulation of GILZ gene has been demonstrated in PBMCs obtained from patients with moderate Mycobacterium tuberculosis (Mtb) pulmonary infections, when compared to controls and less severe cases (see Le Moigne et al., supra). The GILZ directly interacts with and prevents NF-κB-mediated activation of transcription (see Di Marco et al., supra) and can downregulate TLR2 expression as well (see Ricci et al., Cells 10, 2021). The qRT-PCR analysis suggests that MAB promotes an environment where GILZ is upregulated as a mechanism to reduce the innate immune response after the initial stages of infection. The high expression of GILZ in MAB-S infected cells corelates with downregulation of NRLP3 and NF-κB. In contrast, the activation of TLR2 either through the agonist or phages correlates with downregulation of GILZ and with high levels of NRLP3 and NF-κB in MAB-S infected cells. This finding further supports the notion that Ph17 exposure to phagocytic cells stimulates TLR2 signaling.

The research data indicate that greater GILZ expression aligns with reduced ROS production. See Ricci et al., J. Leukoc. Biol. 105:187-194, 2019. In neutrophils lacking GILZ gene, there is a pronounced cell activation alongside disrupted NOX2 regulation, which is an integral factor in ROS release during immune responses. Consequently, infected cells exhibit amplified ROS responses, underscoring the pivotal role of GILZ as a regulator and inhibitor of ROS. In addition, considering that GILZ can modulate TLR2 signaling, and given the critical role of PRR in the oxidative stress as well (see Gao et al., Sci. Rep. 5:13004, 2015; Gatto et al., PLOS One 10:e0117977, 2015; Abdul-Cader et al., Arch. Virol. 161:2075-2086, 2016; Marcato et al., Oral Microbiol. Immunol. 23:353-359, 2008; Mukherjee et al., Braz. J. Infect. Dis. 20:193-204, 2016) coupled with the established contribution of ROS to immune resistance against MAB-S versus MAB-R infection (see Ahn et al., Front. Immunol. 12:738070, 2021; Bogdanovski et al., Access Microbiol. 2:acmi000154, 2020; Goldbart et al., BMJ Case Rep. 14, 2021; Chau et al., Eur. Respir. J. 54, 2019; Ghaffari et al., Eur. Respir. J. 52, 2018), ROS levels in TLR2-stimulated and phage-treated macrophages were investigated. The findings described herein suggest that the stimulation of TLR2 signaling in THP-1 cells by phages leads to the activation of NRLP3 and NF-κB, as well as ROS production, and this response impairs the intracellular growth of MAB-S. Consistent with the above findings, when MAB-S infected THP-1 macrophages are treated with either a TLR2 agonist or Ph17 phage, it leads to the activation of caspase 1 and a subsequent rise in IL-1β secretion. Overall, the results underscore the ability of Ph17 phage to stimulate TLR2 signaling and elicit NLRP3 inflammasome activation that result in overcoming the immune suppression mechanisms instigated by the pathogen.

The present disclosure demonstrates that while activation of TLR2 signaling with TLR2 agonist leads to significant attenuation of MAB-S growth in THP-1 cells, it does not have an impact on MAB-R growth. Moreover, the results obtained from the quantification of extracellular fraction of both morphotypes are in agreement with findings by other groups that TLR-2 activation is a pathogenicity mechanism of MAB-R to escape from cells and, thus, killing by phagocytes (see Le Moigne et al., supra). However, attenuation of both MAB-S and MAB-R infection is evident when macrophages are treated with Ph17. Collectively, these findings suggest that the activation of TLR2 signaling by Ph17 in the presence of MAB-S infection initiates innate immune mechanisms crucial for restraining intracellular bacterial proliferation within phagocytic cells. However, the reduction in MAB-R under Ph17 treatment appears to involve mechanisms distinct from TLR2 activation.

Therefore, transmission electron microscopy was performed and images uncovered a phenomenon wherein Ph17 orchestrates the intracellular elimination of MAB-R via vacuole fusion mechanisms, presenting a novel finding not previously reported in the literature. This is likely attributed to the fact that MAB-R (see Le Moigne et al., supra) and Ph17 (described herein) both induce the TLR2 signaling, and a shared mechanism allows vacuoles to fuse within the same phagocytes. Thus, the decrease in bacterial population is primarily attributed to the direct eradication of intracellular pathogens by lytic phages and MAB-R cytosol escape mechanism is effectively abolished as a result. In contrast, vacuoles of MAB-S and Ph17 remain intact likely because, as a virulence mechanism, MAB-S actively suppress TLR2 signaling whereas Ph17 drives the TLR2, and these vacuoles do not fuse. Consequently, intracellular pathogen is eliminated via triggering the macrophage innate immune defenses as demonstrated by the activation of ROS and NLRP3 inflammasome in MAB-S infected and Ph17 treated cells.

In summary, the present disclosure reveals that lytic phages can evoke both beneficial and detrimental cell-mediated responses, which could impact MAB pathogenicity and overall success of phage therapy. As described herein, phages have profound effects on the infection outcome not only through the direct lysis of bacterial host but also through activation of innate immune defenses of phagocytic cells. More specifically, phages with TLR2 immune activation properties can be leveraged for the development of the host-directed antibacterial therapy against both MAB-S and MAB-R morphotypes, thereby aiding in the optimization of phage therapy approaches for effective elimination of intracellular pathogen. This aspect is particularly significant when addressing treatment challenges of persistent mycobacterial infections. Additionally, this innovative strategy holds the potential to contribute to robust clinical practices in phage therapy.

The identification of Mycobacterium avium subsp. paratuberculosis lytic phages with extensive host range across rapid- and slow-growing pathogenic mycobacteria is described in Example 2.

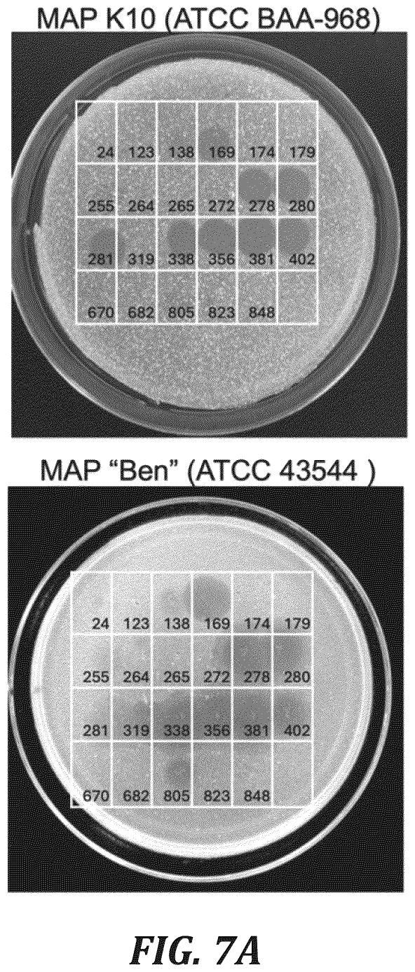

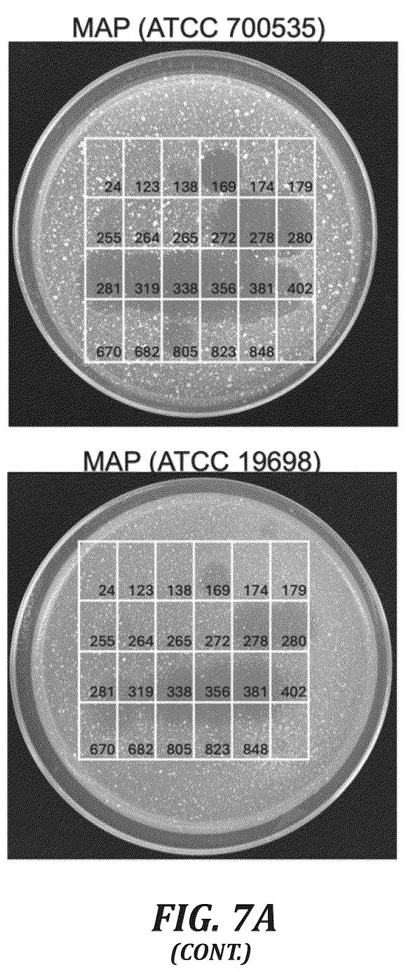

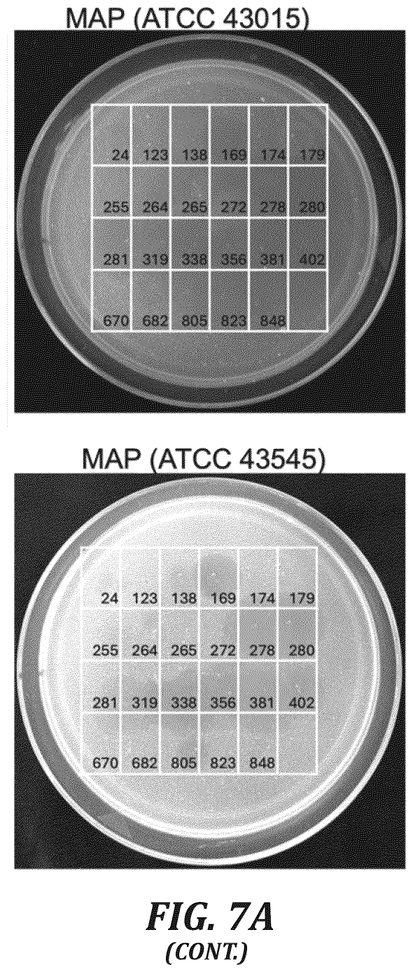

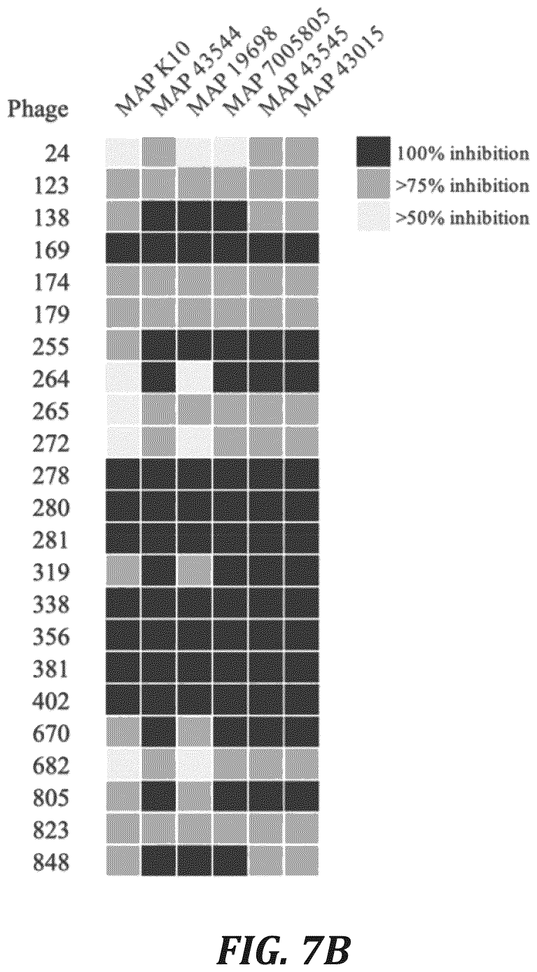

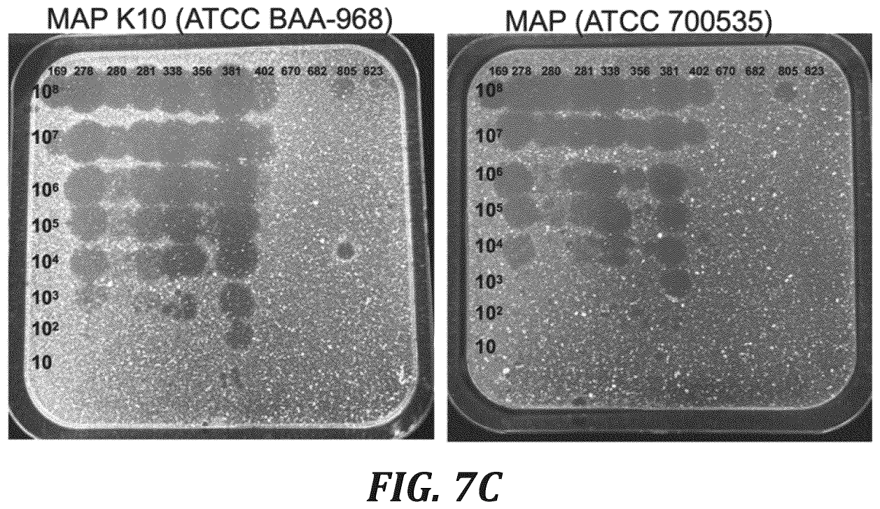

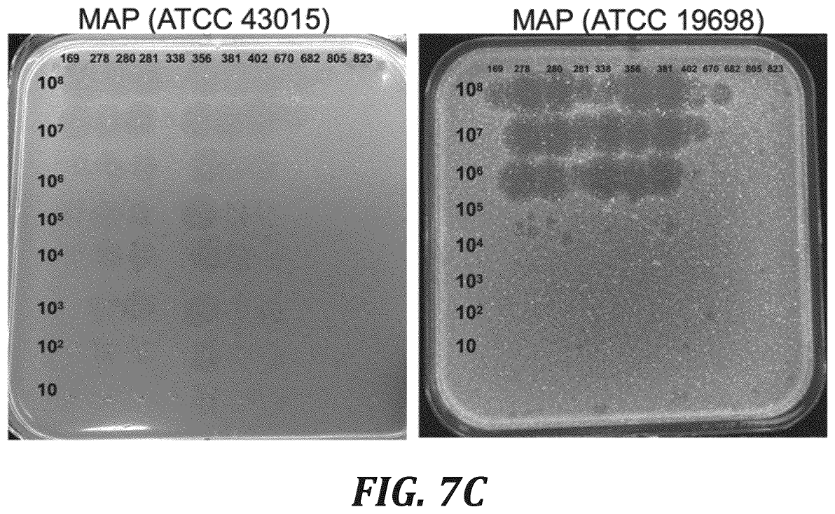

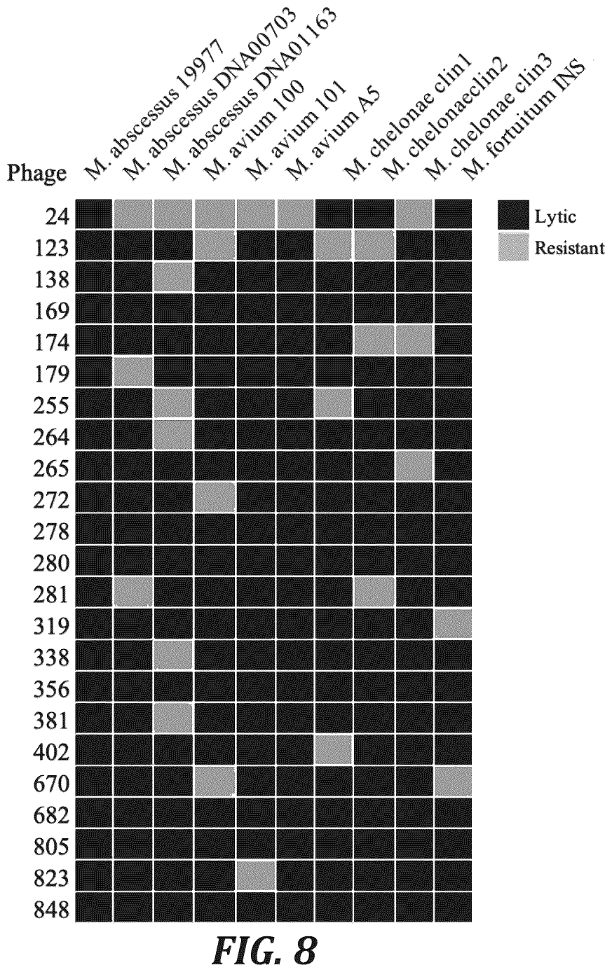

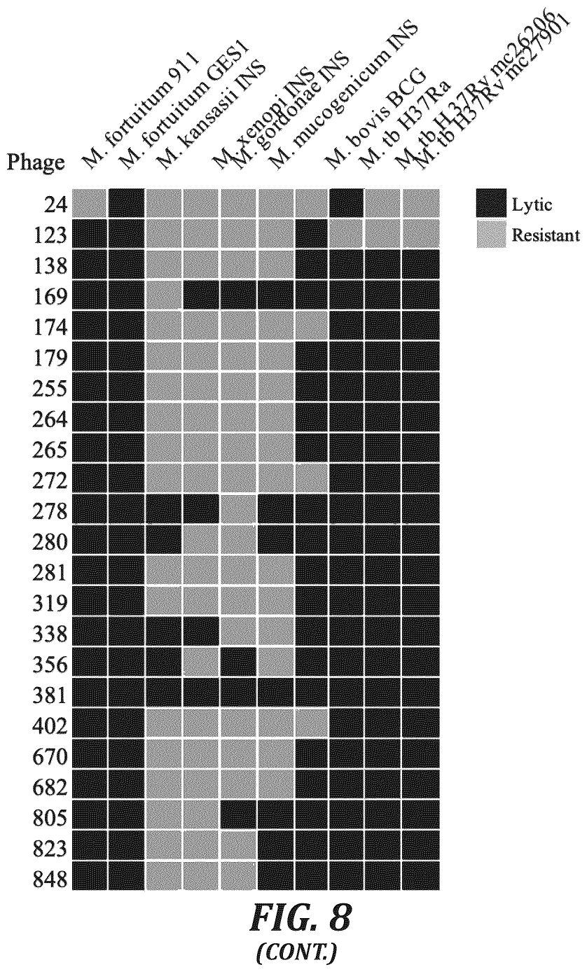

The characterization of the host range of MAP-specific phages is described in Example 3 and the results summarized in FIGS. 7A-7C and 8.

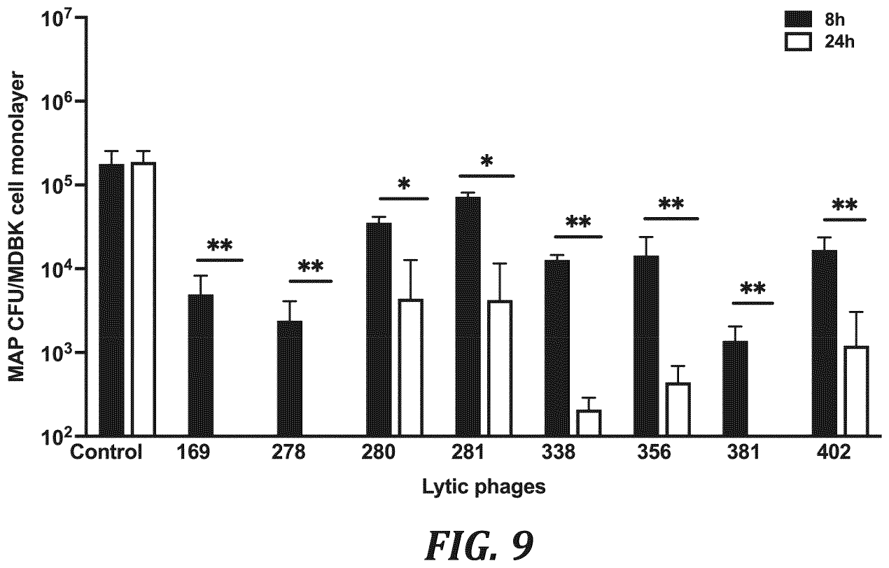

The prevention of MAP colonization and invasion in tissue culture of MAP-specific phages is described in Example 4 and the results summarized in FIG. 9.

The invention is further illustrated by the following non-limiting examples.

### Example 1

Phage-Mediated TLR2-Signaling Results in Attenuation of Intracellular Mycobacterium abscessus within Macrophages

Given the broad spectrum of immunomodulatory properties (beneficial or unfavorable) exhibited by phages, and the limited understanding of the clinical significance of interactions between mycobacteriophages and host immunity, this example is directed to screening a phage library and investigating whether mycobacterial specific phages can activate innate immune responses limiting intracellular bacterial survival within professional phagocytes and elucidate mechanisms behind it.

### Bacterial Strains and Growth Conditions

Mycobacterium abscessus subsp. abscessus strain 19977 with smooth morphology (referred as MAB or MAB-S) was acquired from the American Type Culture Collection (ATCC, Manassas, VA, USA). Bacteria were cultured on 7H10 Middlebrook agar or in 7H9 Middlebrook broth (Hard Diagnostic, Santa Maria, CA, USA) supplemented with 5% oleic acid and albumin dextrose and catalase (OADC, Hardy Diagnostics, Santa Maria, CA, USA) and 0.5% glycerol, but without Tween 80. In addition, MAB 19977 clone was isolated with rough morphology (hereafter referred to as MAB-R) through passaging bacteria in macrophages and selecting MAB-R clones for infection studies.

Bacterial inoculums for tissue culture infections were prepared in the mycobacteriophage buffer (MP buffer) consisting of 10 mM Tris pH 7.6, 100 mM NaCl, 10 mM MgSO₄, 2 mM CaCl₂. See Jacobs et al., Methods Enzymol. 204:537-555, 1991.

The single cell suspensions of approximately 3×10⁸ CFU/ml of the mid-log phase growth (3-5 days) were prepared by sonication and adjusting inoculums to McFarland Standard 1.0. In addition, bacterial serial dilutions were plated on 7H10 agar for CFU counts to accurately record MAB concentrations.

### Differentiation of THP-1 Monocytes and Growth Condition

THP-1 human monocytes (ATCC TIB-202) were maintained in 75 cm² tissue culture flasks and grown by incubating cells at 37° C. and 5% CO₂ in the RPMI 1640) media supplemented with 10% Fetal Bovine Serum (FBS, Gemini). The concentration of THP-1 monocytes was determined by counting cells using the hemocytometer. The phorbol 12-myristate 13-acetate (PMA, Sigma) was added to a final concentration of 50 ng/ml of cells and appropriate volumes were dispersed to each well of 96- or 24-well tissue culture plates. After 24 h incubation, monolayers were replenished with new RPMI 1640 media. Cells were rested additional 48 h before phage treatment or bacterial infection experiments were carried out.

### Macrophage Infection and TLR Activation

The THP-1 macrophage monolayers were infected at Multiplicity of Infection (MOI) 1 bacteria to 1 cell for 2 h at 37° C. and 5% CO₂. After the infection period, the cells were washed gently three times with Hanks' Balanced Salt Solution (HBSS). Next, cells were washed with HBSS. Some wells were immediately lysed to establish the MAB invasion rates, while rest of monolayers were incubated in fresh RPMI 1640 media for 24 h or 72 h time-points. Cells were lysed in 0.1% of Triton X-100 and via mechanical scraping. Lysates were serially diluted in HBSS and plated on Middlebrook 7H10 agar plates for CFU counts of viable bacteria.

In TLR activation experiments, THP-1 macrophage monolayers were either (a) pre-treated with TLR2 and TLR4 agonists for 1 h before MAB infection (as described above) or (b) THP-1 cells were first infected and then exposed to TLR2 and TLR4 agonists for 1 h. The human TLR1-9 agonist kit was purchased from the InvivoGen. The TLR2 agonist, the Heat Killed Listeria Monocytogenes (HKLM) was used at a working concentration of 10⁸ cells/ml, and the TLR4 agonist, Lipopolysaccharide E. Coli (LPS-EK) was used at a working concentration of 10 ng/ml. After each treatment, cells were washed three times with HBSS.

### Evaluation of Phage Effects on THP-1 Macrophages to Influence the Intracellular Load of MAB

The mycobacteriophage library has been previously generated in M. smegmatis host and stored at ≥10¹⁰ Plaque Forming Units (PFU)/ml. See Kurzepa et al., Clin. Exp. Med. 9:93-100, 2009. Briefly, the stock phage preparations were cleared by ultrafiltration in 100 kDa membranes (Sigma-Aldrich) to concentrate samples as well as to remove any possible contaminant bacterial factors and endotoxins (typically <30 kDa). Due to the fact that the 100 kDa membrane pore size provides an equivalent to a spherical particle with a 3-nm diameter, it allows to crossflow any smaller material but retain phages in the cartridge. Erickson, Biol. Proced. Online 11:32-51, 2009. The final phage titers were determined through phage serial dilutions and top agar overlay method on M. smegmatis. The working plates of the phage lysates have been generated in 96-well plates at 10⁹ PFU/ml. In addition, selected phages of this study were purified using a commercially available kit (EndoTrap® HD, Biovendor) according to the instructions of the manufacturer.

The THP-1 macrophage monolayers (about 5×10⁵ cells/well) were established in 96-well plates as described above. Two phage treatment conditions (pre- and post-treatment) were tested as follows: as a first step, 36 phages (out of the library of 300 mycobacteriophages, see Gorzynski et al., Biomedicines 11, 2023) consisting of lytic phages against MAB host were pre-exposed to THP-1 cells at 10:1 phage to cell ratio for 1 h and after washed gently three times with HBSS. To prevent any interference from remaining extracellular phages in the tissue culture well, if any, MAB inoculums were prepared in HBSS with 0.1% Tween 80 to prevent the phage adsorption during bacterial exposure with macrophages (see Kurzepa et al., supra) and preserving the integrity of the experimental conditions. Macrophages were infected at MOI of 1:1 and lysed as detailed above. The cell lysates were obtained immediately after bacterial infection as a baseline and at day 3 post-infection.

In the phage post-treatment experiments, THP-1 cell monolayers (about 5×10⁵ cells/well) in 96-well plates were first infected with MOI of 1 bacterium to 1 cell for 2 h and then exposed to phages for 1 h at 10:1 phage to cell ratio. After washing cells three times with HBSS, some cells were lysed as a baseline and rest incubated at 37° C. and 5% CO₂ for up to 3 days, at which point all THP-1 cell monolayers were lysed and intracellular bacteria were quantified with CFU counts on 7H10 agar plates.

All THP-1 macrophage infection and phage treatment assays were carried out in three technical replicates and repeated four times. MAB infection without any phage treatment served as an intracellular bacterial growth control in these experiments.

### Assessing TLR2 Activation by Phages During MAB Infection

Initially, TLR activation was assessed using the oxidized 1-palmitoyl-2-arachidonyl-sn-glycero-3-phosphorylcholine from InvivoGen (oxPAPC), a modified low-density lipoprotein of mammalian cell membrane, which acts as a dual inhibitor for TLR2 and TLR4 signaling. However, small-molecule inhibitors TL2-C29 at 50 UM and CLI-095 at 10 ng/ml (InvivoGen) were later used to selectively target either TLR2 or TLR4 signaling, respectively. THP-1 cell monolayers (˜5×10⁵ cells/well) were established in 96-well plates and infected with MAB for 2 h at MOI of 1 bacterium to 1 cell. Monolayers were washed 3 times gently and incubated with TLR antagonists before phage treatment. After 30 min inhibition, phages were added to cells (10:1 ratio) for 1 h. The cell monolayers were either lysed immediately to measure the invasion rates or incubated up to 3 days at which time point THP-1 macrophages were lysed and MAB were plated on 7H10 agar plates for CFU quantification. The experimental group without antagonist treatment served as a control. These experiments were carried out in three technical replicates and repeated three times.

Assessing Phage Antimicrobial Effects on Intracellular MAB with Smooth and Rough Morphotypes

MAB-S and MAB-R clones of genetically identical strain were used to infect THP-1 macrophages at 1:1 MOI for 2 h and then exposed to a Ph17 phage (also referred to herein as Pill582) at 10:1 ration for 1 h. Macrophage cell lysates were obtained at zero and 3-day time points, serially diluted and plated on 7H10 agar plates for CFU quantification alongside with a MAB intracellular growth control with no phage exposure.

To obtain samples for TEM analysis, the above experiments were repeated in 25 cm² tissue culture flasks and THP-1 macrophages were detached from the plastic through TrypLE (Sigma-Aldrich) treatment for 5 min and then gently scraping cells. Macrophages were washed once in HBSS and then fixed at 4° C. in the Karnovsky fixative consisting of 5% glutaraldehyde, 4% formaldehyde, 80 mM cacodylate buffer [pH 7.3] and 5 mM CaCl₂). Samples were obtained at several time points. After removing fixative via microcentrifugation at 10,000 g for 10 min, samples were treated with cold 1% osmium tetroxide for 90 min and dehydrated in an acetone gradient made with an ascending order of series of concentrations 10, 30, 50, 70, 90, 100 and 100%, respectively. Next, macrophages were fixed in the epoxy resin. Ultrathin sections (80 mm) were cut out of blocks, mounted on grids and stained using uranyl acetate and lead citrate. TEM micrographs were taken using the FEI Titan 80-200 Chemi STEMT transmission electron microscope (Thermo Fisher Scientific) operating at 200 kV at the Oregon State University Electron Microscopy facility.

RNA Extraction and Quantitative Real-Time PCR (qRT-PCR)

RNA extraction was done using the RNeasy Micro Kit from QIAGEN. The THP-1 cell monolayers (˜5×10⁶ cells/well) were established in 24-well plates, infected with MAB (MOI of 1:1 for 2 h) and exposed with Ph17 (MOI of 10:1 for 1 h). At 5 h and 24 h post-infection, supernatants were collected for cytokine analysis and saved at −80° C. However, only macrophage monolayers of 24 h infection were processed for RNA extraction (QIAGEN) and gene expression analysis as per manufacturers' instructions (BioRad). Briefly, 350 μl RTL buffer was added to wells and cells were mechanically scraped using pipette tip to allow for easier lysing of cells. The equal amount of 70% ethanol was added to each well and transferred into the RNeasy columns for centrifugation at 10,000 g for 30 seconds. After treating samples with RW1 and RPE buffers, RNA was eluted from cartridges with 30 μl nuclease-free water and stored at −80° C. Later, these samples were subjected for the DNase I treatment, and the purity and concentration were measured using Nanodrop.

The cDNAs were synthesized using the iScript cDNA synthesis kit according to the protocol established by the manufacturer's instructions (BioRad). Briefly, total RNAs were normalized to 200 ng/mL using nuclease-free water. The reaction with 1 μl of iScript reverse transcriptase and 4 μl of iScript reaction mix containing a blend of oligo (dT) and random hexamer primers were generated in 20 μl volume and incubated in a thermocycler according to the iScript cDNA synthesis protocol. Generated cDNAs were used to assess the relative gene expression of NF-κB1 and NLRP3 genes using the SYBR Green I assay kit using the following PCR setting: 5 min at 95° C. and 40 cycles of 30 s at 95° C., 30 s at 60° C., and 30 s at 72° C., carried out in the iCycler iQ (BioRad). The gene specific forward and reverse primers for NF-κB1 were 5′-AACAGAGAGGATTTCGTTTCCG-3′ (SEQ ID NO:4) and 5′-TTTGACCTGAGGGTAAGACTTCT-3′ (SEQ ID NO:5), for NLRP3 5′-TGCCCG TCTGGGTGAGA-3′ (SEQ ID NO:6) and 5′-CCGGTGCTCCTTGATGAGA-3′ (SEQ ID NO: 7), and for GILZ 5′-CAGTCCAAGCTCGATTGCC-3′ (SEQ ID NO:8) and 5′-CTGCCGAAAGTTGCTCACT-3′ (SEQ ID NO:9), respectively. The mRNA expression levels were determined using a comparative quantification method based on the Ct value that is the faction of the cycle number at which the fluorescence reaches 10 times the standard deviation baseline value. The data was normalized from the reference β-actin gene in the corresponding samples using 5′-CATGTACGTTGCTATCCAGGC-3′ (SEQ ID NO: 10) forward and 5′-CTCCTT AATGTCACGCACGAT-3′ (SEQ ID NO:11) reverse primers. The fold change (n-fold) was calculated with the following formula: (n-fold)=2^{-Δ(ΔCT)}, where ΔCt is Ct (target)−Ct (β-actin) and Δ(ΔCt) is ΔCt (experimental)−ΔCt (control). Fold change values of 1 indicate no change in expression in comparison to the control, while a fold change above or below 1 is considered as up- and down-regulations, respectively. The qrtPCR was evaluated in samples obtained from three independent experiments.

### ROS Fluorometric Assay

THP-1 cell monolayers (10⁵ cells/well) were established in 96-well plates, infected with MAB-S (MOI of 1:1 for 2 h) and exposed with TLR2 agonist or treated with Ph17 (MOI of 10:1) for 1 h. Five hours later, cell monolayers were stained with CellROX®) Green Reagent (Thermo Fisher Scientific) at a final volume of 5 M. After 30 min of incubation at 37° C., cells were washed with PBS three times, and then fluorescent measurements were taken over 24 h at an emission/excitation of 485/520 nm using the TECAN Infinite 200 microplate reader system.

### Western Blot Analysis

The THP-1 microphage monolayers from experimental and control groups of MAB infection/phage treatment experiments were lysed in cOmplete™ Lysis-M mammalian cell protein extraction reagent and then mixed with an equal volume of 2× Laemmli sample buffer (Bio-Rad). The denatured samples were resolved onto 12% SDS-PAGE gel (Bio-Rad) and transferred to a nitrocellulose membrane (Bio-Rad). Membrane was blocked with 3% non-fat dry milk blocking buffer prepared in phosphate buffered saline (PBS). After an hour incubation, membranes were exposed to the caspase 1 primary antibody (Santa Cruz a Biotechnology) at dilution of 1:500 for 3 h and later membranes washed three times with PBS to probe with corresponding IRDye secondary antibody (Li-Cor Biosciences) at a dilution of 1:5,000 for 1 h. Proteins were visualized using Odyssey Imager (Li-Cor Biosciences).

### Enzyme-Linked Immunosorbent Assays (ELISA) for IL-1β

Supernatants of THP-1 macrophages collected at 5 h and 24 h time-points from MAB infected and either TLR2 agonist exposed or Ph17 treated cells. In addition, we collected supernatants from THP-1 monolayers that were exposed to the heat killed MAB or to phages only (no bacterial infection). Samples were analyzed for the enzyme-linked immunosorbent assay (ELISA) for quantitative detection of human IL-1β secretion as per manufacturer's instructions (Thermo Fisher Scientific). The cytokine concentrations were quantified from the standard curves of sequential dilutions of the corresponding recombinant cytokine.

### Statistical Analysis

To demonstrate reproducibility, most experiments were performed in three technical replicates and repeated in three independent trials, unless otherwise specified. The statistical significance between experimental and control groups were assessed using the student's t-test in the Prism software 10 (GraphPad). The significant p-values were assigned as *p<0.05 and **p<0.01.

### Example 2

Identification of Mycobacterium avium subsp. paratuberculosis Lytic Phages with Extensive Host Range Across Rapid- and Slow-Growing Pathogenic Mycobacteria

In this example, MAP phages from diverse geographical regions were uncovered and to create a “lytic phage library” exhibiting activity against a range of animal and human MAP isolates. As described herein, the virulence of phage isolates against MAP was characterized and their stability assessed under various environmental stress factors and in the rumen environment. Additionally, the selected lytic MAP-phages were cross-examined against a wide range of fast- and slow-growing mycobacterial strains and data was collected on phage receptors. The potent phages of this study can be used toward development of highly effective phage cocktails for prophylaxis, early therapeutic interventions and possibly used as bio-disinfectants in dairy farms.

To generate the lytic phage library against MAP, a large set of soil and water specimens (approximately 1,000) were obtained from varied geographic locations of United States (Colorado, Florida, Hawaii, Minnesota, Nevada, North Carolina, New Mexico, Oregon, Texas, Virginia), British Columbia, Chile, India, and Philippines. Cleared samples were directly processed against MAP K10 strain using 1) the phage lysate spotting method on soft agar overlays of MAP and 2) bacterial spot plating assays from 7H9 broth to 7H10 agar. Alongside, these 1,000 samples were also screened in M. smegmatis for efficient mycobacteriophage isolation process. M. smegmatis is a model organism broadly used to study a range of mycobacterial pathogens including M. tuberculosis and M. avium complex, and it is generally used for efficient phage isolation process and propagation as well. See Shiloh and Champion, Curr. Opin. Microbiol. 13:86-92, 2010; Namouchi et al., BMC Genomics 18:530, 2017; Hatfull, Adv. Virus Res. 82:179-288, 2012. The fact is that the worldwide collections of mycobacterial species-specific phages are exclusively isolated using M. smegmatis model organism because every mycobacteriophage can infect M. smegmatis clade for reasons that are not understood. Another equally essential aspect of the phage isolation process in M. smegmatis is that this organism replicates rapidly (every 1-3 hours), and phage propagation yields to high titers in short time.

Six phages directly against MAP K10 and 307 phages (including these 6) in M. smegmatis were identified. Next, 307 mycobacteriophages were re-tested against MAP K10 strain with above methods. This is because the original samples may have contained very low concentration of MAP-phages that may have been missed during screening in this slow-growing bacteria. While 23 phages were found to significantly reduce viable MAP number (≥50%) using the bacterial spot plating assay, however, only 9 phages (including 6 that have been isolated with a direct screen in MAP) formed clear plaques on the top agar overlays of MAP. Furthermore, selected 9 MAP phages were examined for the host range using a collection of animal and human MAP isolates, and phage stability under different environmental stresses including the rumen fluid. It was found that these 9 phages exhibit a high virulence and are very stable in the tested conditions, no decrease in titration. These 9 phages were also tested in other slow- and fast-growing mycobacterial human pathogens such as M. abscessus, M. avium subsp. hominissuis, M. tuberculosis H37Ra strains and were found to exhibit a lytic activity in these species.

Phages Ore278 and Minn381 (two of the 9 phages discussed above) were tested against additional mycobacterial species and were found to have lytic activity against at least Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

### Example 3

### Characterization of the Host Range of MAP-Specific Phages

Twenty-three active MAP-specific phages were tested for their host range using a collection of animal and human MAP isolates, employing both spot testing and liquid culturing methods. Phages were classified as lytic if they completely lysed the host on soft agar (see FIG. 7A) and showed full clearance of bacteria in liquid culture (see FIG. 7B). The heat map shown in FIG. 7B illustrates the lytic activity and host specificity of each phage across six MAP strains. Fifteen of the tested phages, including Ore278 (“278”) and Minn381 (“381”) were identified as lytic, with both Ore278 and Minn381 being among seven phages shown as lytic across all six MAP strains. For phage isolates that produced plaques on 7H10 agar overlays of MAP lawns, phage virulence was assessed through titration and compared to the sensitivity of MAP K10 (see FIG. 7C).

Additionally, selected phages were tested against various mycobacterial species, including M. abscessus, M. avium subsp. hominissuis, M. intracellulare, M. chelonae, M. fortuitum, M. kansasii, M. xenopi, M. gordonae, M. mucogenicum, M. bovis strain BCG, and M. tuberculosis strains H37Ra and auxotrophic H37Rv strains. The most non-tuberculous mycobacterial species and M. tuberculosis strains used in this study displayed susceptibility to a subset of phages, including Ore278 and Minn381, as illustrated in FIG. 8.

### Example 4

### MAP-Specific Phages Effectively Prevent MAP Colonization and Invasion in Tissue Culture

In this example, the ability of phages to inhibit MAP colonization and entry into bovine epithelial cells was examined. MDBK cell monolayers were pre-exposed to selected phages at 10¹⁰ PFU for 30 min, followed by the addition of MAP K10 at MOI of 10. Control wells without phage served as MAP growth controls. To account for the potential internalization of MAP by epithelial cells, MDBK cells were lysed at 8 h and 24 h, and bacterial CFUs were assessed. Results indicated varying degrees of inhibition, with three phages, including Ore278 (“278”) and Minn381 (“381”), demonstrating significant early inhibition and complete clearance of MAP by 24 h when compared with control groups at corresponding times (see FIG. 9). Five additional phages, while not completely eliminating MAP, still achieved significant reduction in bacterial numbers at 8 h and 24 h time-points.

While illustrative embodiments have been illustrated and described, it will be appreciated that various changes can be made therein without departing from the spirit and scope of the invention.

The embodiments of the invention in which an exclusive property or privilege is claimed are defined as follows:

1. A phage cocktail comprising at least two bacteriophages selected from the group consisting of:
a first isolated bacteriophage having a genome that has at least 95% sequence identity with the genome of bacteriophage Pill582, deposited under ATCC Patent Deposit Designation No. PTA-127809, wherein said first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages;
a second isolated bacteriophage having a genome that has at least 95% sequence identity with the genome of bacteriophage Ore278, deposited under ATCC Patent Deposit Designation No. PTA-127810, wherein said second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and
a third isolated bacteriophage having a genome that has at least 95% sequence identity with the genome of bacteriophage Minn381, deposited under ATCC Patent Deposit Designation No. PTA-127811, wherein said third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

2. The phage cocktail of claim 1, comprising at least two bacteriophages selected from the group consisting of:
a first isolated bacteriophage having a genome that has at least 95% sequence identity with the nucleotide sequence shown in SEQ ID NO:1, deposited under ATCC Patent Deposit Designation No. PTA-127809, wherein said first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages;
a second isolated bacteriophage having a genome that has at least 95% sequence identity with the nucleotide sequence shown in SEQ ID NO:2, wherein said second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and
a third isolated bacteriophage having a genome that has at least 95% sequence identity with the nucleotide sequence shown in SEQ ID NO:3, wherein said third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

3. The phage cocktail of claim 1, wherein the first isolated bacteriophage is Pill582 or progeny thereof.

4. The phage cocktail of claim 1, wherein the second isolated bacteriophage is Ore278 or progeny thereof.

5. The phage cocktail of claim 1, wherein the third isolated bacteriophage is Minn381 or progeny thereof.

6. The phage cocktail of claim 1, wherein the phage cocktail comprises the first, second, and third isolated bacteriophages.

7. The phage cocktail of claim 1, comprising:
a first isolated bacteriophage having a genome that has at least 95% sequence identity with the genome of bacteriophage Pill582, deposited under ATCC Patent Deposit Designation No. PTA-127809, wherein said first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; and
at least one bacteriophage selected from the group consisting of:
(i) a second isolated bacteriophage having a genome that has at least 95% sequence identity with the genome of bacteriophage Ore278, deposited under ATCC Patent Deposit Designation No. PTA-127810, wherein said second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and
(ii) a third isolated bacteriophage having a genome that has at least 95% sequence identity with the genome of bacteriophage Minn381, deposited under ATCC Patent Deposit Designation No. PTA-127811, wherein said third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

8. The phage cocktail of claim 1, comprising:
a first isolated bacteriophage having a genome that has at least 95% sequence identity with the nucleotide sequence shown in SEQ ID NO:1, deposited under ATCC Patent Deposit Designation No. PTA-127809, wherein said first isolated bacteriophage (a) has lytic activity against Mycobacterium abscessus, Mycobacterium avium subspecies hominissuis, and Mycobacterium fortuitum, and (b) stimulates TLR2 signaling in THP-1 human macrophages; and
at least one bacteriophage selected from the group consisting of:
(i) a second isolated bacteriophage having a genome that has at least 95% sequence identity with the nucleotide sequence shown in SEQ ID NO:2, wherein said second isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae; and
(ii) a third isolated bacteriophage having a genome that has at least 95% sequence identity with the nucleotide sequence shown in SEQ ID NO:3, wherein said third isolated bacteriophage has lytic activity against Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

9. A composition, comprising the phage cocktail of claim 1 and a pharmaceutically acceptable carrier.

10. A composition, comprising the phage cocktail of claim 1 and an anti-mycobacterial antibiotic.

11. The composition of claim 10, wherein the anti-mycobacterial antibiotic is selected from the group consisting of clarithromycin, azithromycin, rifampin, rifabutin, ethambutol, streptomycin, amikacin, cefoxitin, and ciprofloxacin.

12. The composition of claim 10, wherein the composition comprises at least two anti-mycobacterial antibiotics.

13. A method for treating a mycobacterial infection, the method comprising administering to a subject having the mycobacterial infection an effective amount of an isolated bacteriophage of claim 1.

14. The method of claim 13, wherein the mycobacterial infection comprises infection with a mycobacterium selected from the group consisting of Mycobacterium avium subspecies paratuberculosis, Mycobacterium abscessus, Mycobacterium avium subspecies hominissuis, Mycobacterium fortuitum, Mycobacterium bovis BCG, Mycobacterium tuberculosis, Mycobacterium kansasii, and Mycobacterium gordonae.

15. The method of claim 13, wherein the subject is human.

16. The method of claim 13, wherein the mycobacterial infection comprises a pulmonary infection.

17. The method of claim 13, wherein the mycobacterial infection is associated with a pulmonary disease in the subject.

18. The method of claim 13, wherein the pulmonary disease is selected from the group consisting of cystic fibrosis, emphysema, chronic obstructive pulmonary disease (COPD), and bronchiectasis.

19. The method of claim 13, wherein the mycobacterial infection comprises a gastrointestinal infection.

20. The method of claim 13, wherein the mycobacterial infection comprises infection with Mycobacterium abscessus or Mycobacterium avium.

21. The method of claim 13, wherein the subject is a non-human animal.

22. The method of claim 13, wherein the mycobacterial infection comprises infection with Mycobacterium avium subspecies paratuberculosis or Mycobacterium bovis.

23. The method of claim 21, wherein the non-human animal is a ruminant.

24. The method of claim 23, wherein the ruminant is a bovine.

25. The method of claim 23, wherein the mycobacterial infection comprises a pulmonary infection.

26. The method of claim 21, wherein the mycobacterial infection comprises a gastrointestinal infection.

27. The method of claim 23, wherein the mycobacterial infection comprises infection with Mycobacterium avium subspecies paratuberculosis.

28. The method of claim 21, wherein the mycobacterial infection is associated with Johne's disease in the subject.

29. The method of claim 13, wherein the mycobacterial infection is a disseminated infection.


SEQUENCE LISTING (11 sequences)

SEQ ID NO: 1 - DNA, 41431 bp, unidentified
  source 1..41431: mol_type = genomic DNA
  misc_feature 1..41431: note = Pill582_Mycobacterium_phage (41,431 bp) (bacteriophage Pill582)
ggcccccacgccgccaccccatggcataggcgtcgccccccctggtccctcacaaaaaagaaaggcggcttgatggccggtcgcacagatcgcccgctgtccgtcgtcaaggctggcgagacgccggcgaagaaggcggcgccgaggaaacgcgcgccggccaagccgcggacgttggcgcaggcggcgaagctctcgcgtaagacgctgctcgaaacgctgcgcgacaagatcgccaacgccatggacgatccgcgcgcgcacccgcgcgacgtcgggaatctcgtcaagcagttgctcgacgttcagaaccagatcgacgcgctggccggcaagccggccggcggcaagaaggccgagccgtcggcagtcgcggacaccccgaatgaagtctgggacgaagacgcgatctaggtcgtcggccggcgcgaaggacgcgaccagaccgacgacgccgaagcctcgatcgaggccggccggcatggtgggcccgaagctgtctgaggttgcgcggcacatcgtgcggccggacggcattgtcgggacggcgtggccgcgcgtgcgggacacgtgcaagaacatcggttggacgttcgatcgctggcaggacggtctaggccggctgattctggcgctcgacggcacagggctctatgccgcggacacgtccgttatcagcatcccccgacaggtcggcaagacgtacctgatcggctgcatcgtgttcgccctggcgctgctcacgcctgggctgactgtcatctggaccgcgcaccggacgaagaccgcgaaggaaacgttcggctcgatgaaggccatgtgcgcaacgccgctggtgaacgcgcacgtgcggaacgtcagcgacgcgcgcggcgacgagggaatctatctgcacaacgggagccggatcctgttcggcgcgcgtgagaacggtttcggtctgggcttcgccggcgtcggaatcctcgtgctcgacgaggcgcagcggctcaccgacaaggccatggacgacctgattccgacgatgaacaccgtcgagaatccgttgatcctgctcactggcaccccgccgcggccgacggactcgggcgaagtgttcacgatgcttcggcaagacgcgctcgacggcgagtccgaaggcacgctgtatgtcgagttctccgcggacgagggcgcgcaccccgacgaccgggcgcagctgcgcaaggcgaacccgtcgtatccgcaccgcaccagtgagcgcgcgattcggcgtatgcgaaagaacctcaccgaagagtcatttctgcgtgaggcgttcggcatctgggacaaggtggttcaccggccggtcgtgaccgcggcgcggtggcgccggctggaatcgaccgggccggctgccggcgtgaagccgaacggattcggcgtcgacatgtcgcactcgcggatggtgtcggtaaacgcggtctggctcgacggcgaccaggcgcacaccgaggaagtctgggccggcgacgacacggacgccgcggtggcgtggatcgcggacgcgtggaagcgcgccggccggcgcacggtggttgtcatcgactccgaatcgccggctgcgtcgctggtcgtcgacctcgaaaacgccggcgtcaacgtctacgtgaccagcgcggcgaacatggccgctgcgtgcggcgccgtggaaaaccggctgaaagctggcactcttacccatggaggacaaatgtcggttacggacgcagtggtgaaaaatggaaaacgtcggccgatccgcggcgccggcggctggggctgggaccggcggaacccatcgagccagattcaccaggcggtcgcaatgacgctggcgctctacggagcaacgaagcacaaacgggcgacgcggcagcgtcgagccgagacaggcagagaggcggtcgtgctgtgacggaggaaacgacgtcggcgtcgaagttcacgttccgaatcccggagctgaacgacgacgtggtggacaaggtgaacgggctgtaccagcagctcgtcgatcgcacaccgcggaatctgcttcgagcatcgttctacgacggcaagtacgcgatccggcagatcggcaacctgattcccccggagtatctgcggacggcgactgtgctcggctggtcggcgaaggccgtcgacacgctggcgcggcgctgcaacctcgaatcgttcgtctggccggacggcgactacggcagcatcggcgggcccgacgtctgggacgagaatttcttcgcgacgaaggccaacaacgccatggtgtcgtcgctgatccacgggccggcgtttctcatcaacaccgagggcggcgccggcgaaccggactcgctgattcacgtcaaatcagcgatgcaggcaaccggcgaatggaactcgcgccggaatgccatggactcgcttctgtcgatcaccagccgcgacgccgaaggccacccgaccggaatcgcgctgtacgaggacggcgtgaccgtgaccgcggacatggacgacgacggcgactggcacgccgacgttcggacgcacaagctcggcgtgcccgtcgaggtgctgccatacaagccgagggaggaccggccgcttgggtcaagccgcatcacccggcctgtcatgtcgcttcagcaacgcgctctcaagggctgcatccgcatggacggacacgccgacgtctacagcttcccgcagctgatcttgctcggcgccgacgcgaagaacttccgcaacaaggacggctcgatgaagccggcctggcagatcgcgttggcgcgcgtgttcgcgctgcccgacgacgaggacgaacccgacgcggcgcgggcccgcgcggacgtgaagcaattcccggcgtcgagtccacagccgcatatcgagatgctggaacagatcgcgatgatgttcagcggcgagacgtcgattccggtcgaatcgctcggattctcgaatcgcgcgaacccgacgtcggccgacgcgtatatcgcatcgcgcgaagacctgatcgccgaagccgaaggcgcgacggacgactggtcgccggcgttccgccgatccatgatccgcgcgctggcgatcaagaacgggctcgaccggatcccgcccgagtggaagacgatcgactccaagttccggtcgccgctgtacctgtcgaaggcggcgcaggccgacgccggcgcgaagatgctcggcgcgggcccggaatggctcaaggaaaccgaagtcgggctcgaattgctcgggctgaccccgcagcaggcgaaacgcgcgctggccgaacgccggcgcgcaagctcggtctcgatcatcgaggcgctgaaccgccggcagcaggaagccgcgacggccggcgaagaccaggaccagggcgccggcgagccgccggccaacgagccgccggccgcgctgggccggccgacgctggtcggctaaatgtcgatcggcgttcctgagttccgcggcgcgctgctcgaattgggcagcggtctcgattccgacgtccgcaaaatgtcgacggcactcgcgacgctcgaccagggcgaggcgctgcgctacgtctccgacgcgtatcccgaaatcgtgacgccgtatctggccgcggccggcgacctgaccgcgacgtggtacgaggaccagccggcggcgatcggcgcgaaggcgttcatcgcacagccggccgagctgccggccgtcgagcaattggcggcgaacggacgctgggccctggtgcaacagtcgccgacgtcggcgctggaaggcaccagccgccggcagctgttcaagacgcaccgcgagacggtgctcaccaacgccgaccgcgaaggcgtgcgctgggcccgtgaggcgcgtccgggcgcgtgcggattctgccggatgctcgcaacgcggatcctcaccgaaggcgagctcggggcgcctggtctgtatcggtcgaagcgctcggcgcggcgcaacccgcaccgcttcaacgccgttcgaggtcacgacttctgtcgctgcgtcgccgttccgatccgcggcggatccgattacgtggtcccggactacgtgcattcgtggttggaggactacgaggccgtttctcgcgacgccgacggatacctccacccacctgggaagatcgccgcgctgatggaggaccgcgcgaagtcgcgcggcgaactgttcggcgtcgacaccgaatcgagtctgattcgccggccgcggcgccggcgcgggcccgaagtcgtcgacctcgacgcgccagctcgggcgatcgaggcgccggccggcgcggtccgcgagagtgtggccggcgcgcagcgtttcacgcaacgcaacgcgatccagttcgagaacgccgttcagccggtcgctgaccgcgtggcgcaggcgcaggcgatcgccgaacgcgccgacgaaatcgtgtccaccgcggcgcgcgtgaccggacacgtcaagcaggtgaccgacgtcgccgacaagttgctcggatcgcagtatccggcgatccgcagcgcgaaggtactcgtcgacgcggccgacaagggactccgctcggcgaaccaggtcaccggcggcgccgtgcaagtcgctcgggtcgccgaccggacacttcaggacaccgtgacgatcgcgcacggcgtgcgacaggtcgccgacgaggtaggcggattgctcgacgaaaccgcggcgatcgcgctcggcgcacgcgcgctgctcactgacgccggcgccgcggcgcgcaacaccgcggccgagctgcgcaacgtgcgcagcgtcgacgacctggccgagcgagtcaccgcggctgtcgacaccgcggccggcatccaggccgaaggcgccgcgctcgtcgaacgggcccgcgcaaccgtcgacgcgacacaggacttcggccgcgctgtcatcaccgagctaccggaagcgctgcgggccccggtcgccgacatgcaggagctcgcgcagaccgtgcgcaatctcgctgccgacgtcgagcacgccggcgcggacgccggcgccgtagcgcgcagtgtgcgccggctggtcgacgcgctgcgggactaccggcgcgctgagcgtgccaccgagacaaccgcggcgatcgaggcgcggcgcgggccgatccgggttacctccgaacggctcgacctcaccgacgcgacttgggaactgccggccggcgaaggccggctggcgatcgaggcaccggaacggcagctggcactcaaccggaagccggcgccgctggcgatcgagggcccgcggcaaccgcggatgctcaccgcggcgccggaccggccggcgatcgaaccgccggatccgcacgatatcgccgcgtggctcgacgccgaggacgagcactggcacgccgtccaatactggcgccgagtcgacgacgagaaccttcattcgctgccgccggccgagcggatcccagaccggccggccgcgccggaaccgccggccgctgtcaccaaaccacccgccgagatcccgaacgatccggcgcccgtgtccagcgtccaggccgtcgagcgcgccggcgaaaccgagctcgatcgcgcggtccgtgagttcgaagaggcgctggcgaccggcgacgaagcgctcatcgagcgcaccgcggcggcgatggaacgcgccgaaaaggcagagctggccgcggccgagcgcaaagccaaggccgctgcacgacgcgaggcggcgcgcagcgccgaccaggaccggatcttcgagctggtcgaggccggcgaggatccgcagctggcagaagccgaagtgctgacgtcgaacgccgacgggcgccggcggatcaagaaggtgctggccgagaatccgtcgttcagcgaggaatcggcgcggcgcaccgtccacaaccacattctcgagcggatccgccggcgcgacttcatggcgcaggcgcgcgccgacggacacgagggcaaaggattcgacgacctgctcgattccgtgttcgcgcggcgcgtcgacgaaatctacatcgaagccgagaacgcgacccgcgggcacatggtcaagagcaagtaccagctgacgttcgacccgaagcgactctggtacgtcaacgagaccaccgcgcgcaaatacatgtccgaagagttgggcaactggttcgatgagaacggccggatcacccggccgatcatgagacagatgattttggacggctcgacgaacttcagttcgtacaccgcgctcgtaggggacttcgtgcaatgacattccgagaagaagccgaagccgcactacgcgctggtcagaacgccgaaatcggcgacccgaacccgtacaacgggaagtcactcgcgctggcgaagtgctggcaacgcgggtacaagacgatgctcacgatccgcacggcggcgacgccggcgcgacagaagtacctcgaaggccggcaggatccggccggcgaaacgtagccacacacgacgcttgcaccgcgacatatgtcggttttggacatatgcgggttaacctggcgctatccgttaccgaacggaacggacccaccagacacccgaaccgggagcaagcagcatggcgaatcaggccaacaacgccggcggcgaccagtcgcaggccggcgagcaggaccagcacgacaaccaggaccagggcgcggccggcgggaacggcggcgattctggtaaccgcgaatacgagcagctgccagaggatcatccgctggtcaagcggctcgaagcgctcaaggccgagaacaaggttctgaagccgaaagcgaagctcgtcgacgacgccgaagaggcgaagaagagcgacgcgcagaggatcgcggatctacagacgaaagtcgacgcgctgcccaagcaggttgcggccggacttcgcgagcacctggtcgagttgcacggcatcgacaaggacgacgccgaactgttcctgacgggtgacaccccggaattgctgctgaagcaggtcgctcggctgttggagtcttccggcgccggcggatctaagcgcaagaactacgtccaaaaagagggcaaccaccagcgcaagccggcggattccgagaacggcgcattcgcaaagtcgctgttcggcggatccgacgactgacgccggcgggccccctcacacaactacctgagaagggtcaattcacatggctacgttcggtaccggaagcctgaagaacctcccccgcaacatcgcggacgggatggtgaaggacgtcgtgcagggctcgacggtcgccgcgctgtcggcgcgcaagccgcagcgattcggcaatgaggacatcatcactttcaacggccggccgaaggctgagttcgtgggtgagggtcaacagaagtcgagcaccaccggcgaattcgacttcgtgaccagcacgccgaagaaggcgcaggtcacgatgcgcttcaacgaagaggttcagtgggccgacgaggactaccagcttggcgtgctcgcgaccctgagcgaggccggcgccgaagcactggcgcgcgcgctggatctgggtctgtatcaccggatcaacccgctgacgggaaccgtgatccccggctggtcgaactacctcggcgccgcgagcaagcgcgtcgagctgacgaccgacacgatcgcgaatcccgatctggcgatcgaggccgctgtcggtctgctcgtcgcgaacgggcacccgaccccggtaaacggtctggcactgcacccgtcgatcgcgtggggactgtccaccgcgcgtttcaccgacgggcgcaagaagttccccgagctcggtctcggtatcgggctgagctcgttcgagggaatcaacgcgtcggtctccgacactgtgaacggcggcgacgaggccgatcccgacgatgaggacttggacggcgcgcgcgctgtccgcggcatcgtcggcgacttcgccaacggcatccactggggcgttcagcgggatatccccgtcgagctcatcaagtacggcgatcccgacgggcaaggcgacctgaagcgccacaaccagatcgcgcttcggctcgaaatcgtctacggctggtacgtcttcaccgaccgcttcgtggtcatcgaaaacgccgtcgcgtaacccgatccgaactcaccgctcggaaaggggcgacacatgcgactcatgaacgaacacggcgtcgtggtcaacgtgcccgacgagctcggcgaccagctggcagcgtccggcgcctgggacgtgctcggcggctcgaagccggcgcgcaagtcgacgagcaagccggcgaaggccgcggcgaagaaggccgtatccgctgccaagccggccgacgacgccgacgaccaggacgacgccggcgacgccggcgccgacgcgcagtagggcggtcgaccatgaccgagccgacaccgacggaaccggaacccttgctgaccgcagaggatttggcgccgtttgcgaccatcccgaaggccaaggccgacgagatgatcgaagacgcgctcggtttggccgaagtccatgcgccgtgcatcaacgatccgggattcgcgcagcgcagggccgcgaaagcgattctgcgcggcgcgattctccggtggcatgaggccggcgccggcgccgctgtcacgaagaacgccggcatctacgggcagaccgtggacacccggcaaccgcgcaaggcgatgttcttcccgtccgaaatcgaccagctgcgcaagctctgccggccggacgacgacaacggcggcgcgttctcgatcgacctgctaccgcaggacaccgtgacccacgccgaaatctgctcgatctacttcggcggcgggtgctcgtgcggcgcgatcctcacacagggattgccgctgtacgagaagaacaacggttgggcgtgatggacttccagattcccgagccgttcgaagtgacgcactggacgcgtccgaagatcggcacaacgccggccggccaaacgaaatacggcgaacccgttccgcggaagcgcaacgtccgcggattcgaaccggccggcgagcaagagattcggacggcgcagctggccggccgacaggtcacagagctcgtgatgctcaccgcgcacggcgactggccggccgattccgaagtcgagctctgggacggccgacggttcgaagtcaacgggcccgtgagggattacaacctgggcccgttcgacttcgaacctggctacgccgtcgatttgaggagggtcaacgatggggaagcttaaaatcccgatcagcgacgacaagaagatccggcgcagctctgaggttcgcaaggcgtgccagaagatcggcgccacgatcgccgtcaccgccggccggatcgccggcgacgcagacggttacggcgtcgaggaaagcgtcggatccgaccgcacgcgcgtcaacgtctacgcgcaacacaacaagaccgtgaaggcggaagccggcgcgacgccaccgcttcaacaggccgcgatgcgagtccgcaagtgaccgcaccggagatggtcggaccgaccatggaacccgcgatcgcgtgccgcgcatacctgatgcgccggctcgacgatcgcggaatcgacttgtctgtcggcgcaacgccgccggccggcaaaccgacgcgttacgtgctggtgaatcaggttgattcacgccggcgcggacccgtcgcggactacctgatccgcacgcgcgtctacaacgcagatgcgtacgaatgcgggcagcacgcaacgctgctgcacgctgcactgctgggcgccgctcaggcccgaatcgttttcccggacgtagggcagctgtgggtgaccggcaccgaacacgtcagcggaccgtccgacattacggacgacgacacgacgtcactgttcggccaagcaatttccgtcttctggacggtcgcactcaaaccgattgaagggaacacaccatgactacacccgtcgttccgtcgccgctgggcgattactcgcaggtgttcgccgcgacgccgagcggattgcagaccgccggcggtctctacatcgcgccggccggcaccacgctcccgaccgacgtcgacgaaccgctgaacccgttgttcaagtcgcttggctacgtgtcgagcgacggcgtgaccatctcgattgatggttcgacgacgccgatcgaagtctggtccggcgagcggatcggctcgctgcgcgacgcgttcgcgatcgagtacagcatgtcgctctaccaggtgctttcgccgcacgtgaacgccgtgatcttcggcgacggttccgtgaccaccgccgcggcgaccgaggaacacggcaaccggatgaaggttgccatcagctcgcgtatgccgaagatggcgtcgctcgtgctcgatgcgttcttcgaggacaaggcgattcgtcaggtcgccgagctcgttcagatgagcgatatcgacgacatcactctcgtgcacaacgagccgatggcgttcacgccgacgttctctgtgttccgcggacacaacggcgatcacgtcgtccagtacagcgacgacggtcagaagatcgccgcgtaagccggcgggcccgcgctccggtctggtgggcggcgcgcgggcccgtctctacgcaatgcacccaccaggccacccaccacacccaccaggagaagagaacatggagcacgacgagcaccaggaccagccgcagcacaccgagtttcagcgtgacgagacgccggccgagctgccggcgcctgagcacgccggattcgagggaaccgacaccgccgaaccgtcgacgtcggatcccgaactgtccatcgtcgaagagtggggcggcgactacccgcccggtacggcgttgttctgcgcgaagttcgacgccgacgacttcgacgccgactggggcgaagacgagtttcccgacggcgcgaccgtcgcgatccgtcgagggtccggcgttcctccgcagggctggatcatccggcacgcgcacctgtccgacctcgaacggacgaaggccatcctcgagaagcacgcgtcgccggacgcgttgcgaatcctctacagcctcaaggacgcagcattcgacgcgttcgtcgaggcgtgggggaaggacggtggcatggcaccgggaaaatcgaacaggtctgcgcggcgatccgccagcacgaggcggcggtaaggcgtgacgtgattgcgttggggtccggcctacgactcgacgacggacgtctgtcgtgggtcgacctccacgccgtcattttcgccgcaccgccgaacaccgcggtattccacgcattcgagcagggctggaacacaaccgattacctgctggcactgatcggcgacggaatccacgacctgatttggcagaagaccaaggacggcagcaagaaccggaagcggccgaagcggatcccgcggccgaagcgggatggccaagacggcaccgcgtcgaccgggctcggcaaggtcaccgtgatgacggtcgaagagttcgaacgccggcgagctgagcggcaacggcgctacatcgagcggaagaagagagaggcgcgggcgagctgatgcagggaacctactggctcacagtcctaccggagacgtcgaagctacggccgggtatcaagaaggctctggccggcgtcgaccaggacgccacgatccgcccgaagttcgatacgaagaccgccaagcgcgccggccaacaggccggccgcgagctgcaatccggtgtcgagtccgaaactcgtggcgggctcggcaagatgctccgaatcgacggggcgcgctcgactgggcagaacgccggccgcgagattaatgctggtctgcaatcggcgaatatcggttcgggcgcctcggcgcagctggaacggaacatgacgtcgggcgcggccggcctgggccggcggatcggctcgatgatcggtaccgggctcaaggtcgccgcggtgggcgccggcacgatcgccgcggccggcctgggcgccgcgctgcacgccgggttctctcgtctcaccgcgattgatgatgcgaagttcaaactgcaaggtctcggtaactcgacggaaaaagtcgaagcgatcatgcagaacgcgttggctgccgtgaagggaaccgcgttcgggctcgacgaggcggcgacgaccgcggcgacggctgtcgctgccggcattccggccggcgacgagctgacgaggtacctcaagctgaccgcggacaccgcggcgatcgccggaacgaacctggccgacgtcggcgcgatcttcaactctgttcagacatccggtaaggcgatgaccggcgatctgcgcatgttggccgatcgcggtctgccagtgttcacgtggttgcaggatgcgaccggaaagaccggcgaggacttccagaagctcgtcgaggaaggcggaatctcggcgcagatgttccgcgacgcaatcgaaaagaacatcggcaacgcggcgcaggagatgggcggatccgtccgcggctcgctgtcgaacctcaaggcgtcgttctcccgattcggcgccgagctgtccgggccgatcttcgccggactgaagccgctggcggtcgggctcaccggcgtattcgattccgtgacaacctcgatcaaaccgatcatggaggatctgaccgcgcgcgttgggccgtgggcagaggaaaccgcggcgaagatcaaagcgtgggccgataacggcggtttggagaagatcgtcgcgtggttcggccggctgcgcgacacgatcgcgagtctgcgcaccggcgaaggccgctcggatgcgatcgcgtcgatcgccgaatcggcgaagcagatcgggccggcattgcagaacgcagggccggcgctgaccgggctcggcgaatcagcgaaggcgttcgggcaggcgattgccgcggtcgggcccgaagtgctgacgtcgatactcgttccggcgctgaagctgttggccgtcacgctgaagttcgtcgccgataacgcgtcgtgggccgtgcccgtgatcggcgggctggtcctggccgtcgccggcctgagcaagaccgcgacggttatcgccccggtcatttcggcgttctcgaatctggcgcgtctgttcaacgcgccgttgataatcgcgcagacggcagcgatgaaccagcaggccgcggcgatgaaccggctatcggcgtcgctcggcaccaacaccgttgcgcagaacctcaacaccggcgcgcagacggcgaaccaggcggcgacagtccgcggccggatcgcagcgctgggagctgccgcggcgagcaaggtcgccgccggcgcgcagtggctctggaatgccgcgctcaccgcgaacccgatcggactcatcatcgccggcgtagttgcggccggcgtggcgatctgggcgttcttcacgaagaccgaaacgggccgcaagctctgggacaagatttggaccggcatcaagacgacgttcggcgtcgtctggggatggctcaagacgacactcgcgacggcgtggcagcagatcgggccgtcggtaatgaagatcgccgaagtcggcaagcaggcgttcggcgcgttcgggaacgcgatcaaagctctttggtcgttcatccagccggcgatcgtgtgggtcggcaagctgtggctggccgccgggaagctgcaattccaggccgcgatcggcgcgctgaagctgctcggctcgacgatcggctggctgtggtccaacgtcgtggttccggcgttctccggtatcgcgacggcggtagagacgtggtgggccggcgcgaagatcgtctggtcggcggcgacgaccgcggccggctggctcggcgacaaactctcgtggctctggcagaacgtcgccgtgcccgcgttcgagggcatcggctcggcaatctcgacgttctgggacggcgcgaagaaggtctggtcgctggtcaccgacgcgttcgacaaggccggcaagggcgttggcgtgctgaaagacgcgttcgtgaccgcgtttaacgcgatcaaggacgttgtgacaaaggtctgggactcgattaagggcatcatcgacggaatccgcgacgggctcggcggcgtcgtcgacaagctgcgcaacattccgggcgtcggcgcgctgatccccggacatgcggccggcagtcctcccggattcgccggtggccggccggcgacgctctcgcgtacgggccggctgcgcggtccgggcaccggcaccagcgactcgatcctggcgatgctgtcgaacggcgaaggcgtcgtgaaggaatcggcgatgcgcggcggtggcgccgcggtggtcgccgcgctgaacgccggctgggtgccgtcggccggcttcctccgcgcgatgctgccgggattcgccgaagggctcaatccgggcgccgactttctgcggaacacgatcatgcagcaatggccgtcgatcaagacgatcggcgggcgccggtccgaagacggatacggcgagcacagtagcggcaacgcgctcgacatcatgattcccgactactcgacgccggccggcaagatgctcggcgacgaggttgcttcgtggatcgcgaagaaccgcgacgtcctgggcgccgacggcatgatctggcgccagacgagcttcggttacggcggcgactggtcgaccggaaaaggcatggctgaccgtggatctgacacgcagaaccacatggaccatgtgcacgtcattctgggcaagggccgcggcgccggcgcgccggccgtccaggcggcgtccgcgtcgtcgctgtcgctgccggccggcggatccgcgtcgtcgctcggcgcctcgacgtcggccggcggtggatccggcatctaccgggccgcgaccgataaggagctcgcagcgtcggcgaagaagctcgattcggcgtctgaagctgtccggcaggctgagcagtccgtcgacgaccgcacctacgcgcgcgataaggcgcaacgccggctcgacgagctccgggcgaagggcaaggacaccgccgacgctgaacactcactggcgaaggcgaaccgcgagctcgacgacgcgaaccgccggctggcgaaggcgcgcgacaaggcgaccgaagtcgagcaggccgattccgagctgcgcagcaagggcgtcgaggacgcgaaggccgcgagcaagtcgggcaacggctacggcgatcttgggtccgcgatctggggcggtctgttggaaacgatcgggctcgacggttctgtgttctcgaatccgttcgagtggccgaccgtgaaatctgtcatggccggcgtgaactggctcggcaaggcgttcctcggcgacggttcgtccggcgacgccggcggaatcctcggcggcgtggccgaagcgaccgggctcgacgcgctggcgaacctcaacccggcggcgaacgaagttgccgcggcgccggcgtccgcggtggcgccggacaccacgacgcacggcgccggcgccggcgcagcaccagggccggccgtcgtgattcagaacgccggcatgtcgccggtggacgtctcgaatcggctcaccgccgacttcaacgcacgcacccggacgacgaaggttcactgatgacgcaacagtttctcggcgacgacgacttctacttcgacgaaatcaagcacccgaacgactatctcgggaatccggcctatgcgccggtcgacctcgggcacccgtcctggcagcggatgacgcgctgggaggacatgggccggtacggcaacattctgcgtggcgagtcgccgcagtgggtgtggatgcacccgaacaccaactggaaggtctggcacctgtccgggccacgggaaggcgtcgaaggcgtggtcctggccgaagggctcgacggcgttttcgacttggagttcgaacaccgatacagcaagggcccgtatctgatcggcgccgagcgtgaacgtaccgactacatgatgggcgtcgtcgacttcggggtgtggatcaatccgccggcgaacatcaaccggccggccggcggtccgttcgcgatgcacaagatcgaagacagttggcgccaatcgtggtcggacacggtgcccggattcctcggctgtttcacacggatgcacggctggcggtggattcccgcgatccagggttcgaagatccggcgcgacgcgcgcaagtcgccgacgtcgaacggcaaccagtcgacgcttctgtcgatgaccgggcacatgccctggccgttgttcgcgaaacgcgctgagactgaggtctggaagccgtcacaggaggacattgcccgcgacggctacgcgaagcacacgttccatattgcgaacaaggggacgttcgagtcggcgccgaagttcatcgtccgcggcggctcgcgcgacgacgtaacgatcgacgggaagaagggattcggcgtgcggatccaagacggcaacgagggcaagatggtcccgattccgccgttgctcgccaacgacggcgattacatgttcgtcgacaccgaccagtcgcggcagacgctcacgacggagaaggaaccgatcgacgggcagatctggaagacgctgcgacagtcgcaattcctggcgctactgctcaacgacaagttgcaatcgcacctgccggcgcagcggcgcattccgggcggtatcgacttcgacaacccgattccgccgcgcacggtcgcacacatcaccgtgacgcacacgaatccgttcggctcggtcgaaatgatcctgccgcagtattaccgggcgtcgtggtcctgatgtcaatgctcatgccagccggatacgtcggcgcaagcccgaaacgcgatccgatcgcgacgtggaaccaggtcaatgcgcgccggcgcgtgctcgacgaagaggcgaagtcgccgtcgctataccggctgtgggacaagcggatgaactacatcggcaccgtccacacccacaagtcattcgacgccgaagagatgcagcacgacaccggcgaagggacgatcgtgctgcgcgggtcggactggctggtgaactttctccgcaccgacgtacgggccgaagaggatctgaacttcaccgtcgacccgtacccgcaccggcgcaattggcgttggcgctggggattcaaggtgaccgacgtcgaggtcggacgctccgaagacggcgaagtgaccgtgacgctgcacctgatgcacaaccgcgagcactggaaacacattctgttcggcgcgaccgtgttcgcaccgccggaagctcagccgatcaaatgcttcctcctgccggccaactgtcggactgccgtcgcgacggccggcgcggcgaacctggcgcgactgttcaatccggcgctggccgtgtttacgaacctgctcaaccctggcgcctacgtcggcgctgcgctcgggctcggcgtgcctgggaacttctcgttcctgaaccacccgatccagatgcagttcgtcaacccgatcacggataccagccggctaacggtcctcatgtcgcgttggcaggacgcgcattccgtgactgaggcgatgctgcgcgacgccggctgtcaggtccgggcctatacctggctcgaagaggacgaagacagtccacacccagagctcgcaatgctcatcggcgagcagctggcgcgtccgcggcggaattgcatcgtgctcgcgtgcgaggaacgggcgcagtaccagggtattaccggcctggcgcctgatggcgcgatcaagctcatcgcggcgaccggcgacgatctgattaccaacacgctgtttcctgagtacgacgagaacggcgacggcgtaacggatccgctgatccgaaagtggttcggcgccgcgccggccgttccgtcgctggtgttccgcgacgggccgcgctcggcgatcgtgtcgtcgagtcaccacatgtataagtccaaggcgaaacacatcatgaccggcgggaagtcgccggcgtggctcaatcaggttcagacgttcctcatccgctatgcgatcagcaagatcgccgaagccgtgacgtcgctaccgggtatgccggcgcagctcgtgggttctgagggcatcgagaacgtctaccaaaatcagggcgacgacgtgtttttcgccttcatgcgctatacggacccggcacgcgagctgcgcgcggcggattacggtctgctcgaagagttcgtgcaaggcagcggatccgcgtacgtcattgcctcaccgctgacgattcgtcagggcttggagaagacgaagccgtatcacgcgttcaagaccgaagtgcgcaacgcgcgtccgcaccggatcttcctcgacttcgatatgggcgatccggcgctgttcgaaatcgacgggattctgtcgcaagaccatgtgtcggcgatccggctccgcgaggacgagacgacgccgaagacgttcggaatctcgatcggcgacgaccgcgaatccgattccggcctggctcgcgtgacacgctcggcgcaggagttttggaacggtctggcaacactattcggatcagggagcatgttctgatgaccaccagcaccgagcacgacgacgcgtacgaacggctcaactacgacattccgccggacctacagccggcgcaggaagcgatctatctcatcgaccagatgctcgacaacccgatcgacatgcacggcaatcactacgacctccgctatctgaaacctgttctggcgtaccacctggcgcgctgcgggctcggactcgtcgaggaccaggccgtcgtgaagcgacgggagtacccgaacgggttcgtcgagtgggtggcgctcgacgcgccggatctgccggcggatccgctcgatggactgtcgctggccgatatcgcgaaactgccggccgagcaacgcgacgcagcgatccgccggctgcaagctggggaggacgggcccgtcgacgatcccgacgaccggattccttggaaggtacgaacgaatatccagatcgacgaagaggcgctgaaatgaccacacccggactcaccgacgacgagatcgacgacttctacgccaaccaccggatcccgacgaccggcgacgccgtgatgttgtaccagtccatgcttcaggcaacgtggatgggcctggtcggcgacccggacacaccgccgatggtcggcgccacgatggaggccgtcgacggcaaggcggtaatcaccacaacggttctgatcgggccccgcggattccccggcaagaacgcgccaatgatcgatctgcactggccggttccgctcaacgaggacgacgaaatcgacctgccgaccgactggggcccagagcagaagaaccacgggttcctacacggtggcctggtctacgtgtgggacggcgtgagcgactttcacgcagcgctaccgggcccacagggcaaggtcggcgcgacaccgaacatcacgttcgacttcgagacgattcccatgtcggaacggacgccggaagtgatcgccggcggcgaccaggtaatccaaggtggcacaccggaaaatccgtacatcaagattcgcgcgctcactccgcagggcgagcagggcgagatgggccccgtcgagcagctgaccaactacgatccgaactccgcggccggcggcaagacggccggcaaggcgcttaccgtgctgccgaacggtaagtggggcccgtcggacctggcgacgaaaaccgtcctgttcggcacgattcccgaagcggcgttttcgaacttcagcggacttgcgcagcgcgcgccgattctgagctaccagctgccggtcgtcgacttcgactacgtgctccgcgtctcgggacacttcaagagctttggcgtcgagctcgactcggatccgcttacgatcggctgcgaggtccggctcaacgatccgttgtccgggcagctgatcggccgcggcaagggcaacatctcgagttggacccaggtcgggccgcacttctccgagccgagcgcgccgaccgtggccgcggcgccggacaacggcgtcgggctcgttccggccggcacaaccgccgtgattcacattgcgttggtgaacgacggactgttgggcgcgtacatcttcaaccgcaaggatgcgcagctcgactggatgatgattccgcagtaaggggcgactgatgacgacaccaacaccgaagaagccgttcgaaaagcggtacaagacggtcgtgcccgtgaagcggccggccggcgtcgaaccgctgaccgagctcggcgcagtcgacgagcacccggactacacgatggcgcgctggctcggacgggaatcgttcgagaacaccgcggccgacgatcgtctggatctggtcgagtacgccgagcggctggttccgctcgacgaagtggatccgcggctggccgacatgctcggcgccccggtcgaagagttcgagtggttcgagttctccgggctgggccggctcgatcaagatgcgttcgactacttcagcgcggaaatcctgtggcgccatgaacaatggctggcagctgagcgtgcctatctgaccgcgcgcgacgccgagctcctggccgataacgccggcggcgcctgatgggcggcaaggtctacgaccgccggcaactcaccgtcgaccgcgatccgctgaagcaactcgctgtcgagctcggcaagctgccacagccggatccgctcaagcagatcgaggccgcggcgaagttcgtccgcgacttcctgatcgacgcgttccgcgacgtgaccggaatcgacctgtccggctgggcagcgttcatggacgcgctgcccggattcgacgcgtcgaagatcatttccggcctgttctcgggctcgctgattcccggtctggacgcgtcgaagatcgtctcgggttcgttccccatggaacgcgtcaccggcctactcgaagcgctggcgtccggcgacgtcggcggaatcatcaaggctctgacgggtatcgacggcggcgacctcgacgacctcgaagactgggccggcctgatcccgaagctggtcggcggattcctgtcgagctcgatcattcccggactcgacgcgagcaagatcgtctcgggtctgctcggcgccgtgcatatcccggagctcgacgcgacgaagatcaccagcgggcagttcccgcaagcgatgctgaacatcaccaacattgcggccggcatcgtctcgggaatcttcaatcctgggcagatccccgcgatcgacgcgtcgaagatcgtctcgggcaccctcgacgcgctcgtgattcccgacatcacgcgcgaaatgtccagcgacgtccaggccgcgatcgacggtctgattaacgcgacccgcaacacacccggcgtgattgggcaggccgtttccgatatcgaagtcgcactcgctcggatcccgacggcgatcttcaacaggttcagcggcaacaacgtggcgcgcgcgtcggccgaccaggcgaacgcagcaatggcggcgctggcagacaccgtgtcggcgcagggcgcggcgatcaacgcgctgaagaacatcctcgacagcgcaggcggattcagcgcgaacgtcgagttccggttgccggaagtgctagagctcgacgttccgggtacctatacgtacgacttcccggtctggtcgaaggtgctcgacattgtggccgtgggcgccggcggcgccggccaaggcatgagcttcgccggcgtgtggggcgaaggcggcaaggccggccaatgggcgacgctgcggctggtccgcggcgtcaacgtcccgatgggcacgacgcaaattcagatcaccgtcggcaagggcaatccgagcacgttcggcgccggcaaccctggcgatccgacggtcgtcacgatcacgggttacggcacgctcaccgcggccggcggcgccggcggcagcgcgtccaacctcgacactcccggcaagtcccccggaaacatcacgttcgagggcgagctttatatcggtggcgcacaacagaacgggcagtcgtcgacaggcaacgcacccggtggcggtggcggtggctcgcgaatctcactcactacgggcggcatgggcggcgccggccgagcgtgggtaaaggccatggccgagctgccgccgcagttcaccgcgcttggcacgctcattgttccgacctaccggctgaacaccggcgtagcgcaaaccgattcgatgaccgcggccggtgtgtggaccaaagcgccgcagggcgccggcaatgtgctcatcatccgcgcgaacgcggcgttcaccacgtatgtctatctgcgggtttggtacgtcggcagcacgaccaactatgaaatcgggcgagtcacttcgggcgtgaaatcggcgtggaagaccggcacactcgcgcaggctgttccgttcaacgcgttctcgatcacgtcggactcggctcgcacgttcaccgtgtcgatcaacggaacggcattcgacagctacaccgacaccgcggcgtcctcgtcgatgggcgccaacttccgaaacggcggctggggttcctcggatcacaacgtccccggatccatttcgcaattcgcgtttctcgacaccggcacgccggcgcggatcacgtcggcgaacgtgcttccgtccgagacgatgacgggcacgtcatacgccaacctggcgacgctgggcccgtccgtcacgctcaacgttccgccgagcggcgaagtcctcatcgggttttcgtgtcgcacgtcgagcacgtcgagcggccgaaagacgttcgtcagcttcgatatgtccggcgcgaacaccgcgggccccgatgacacgatggacgccgaatactacgtcgcgagtgccaacacgtcgggaaccatgtcgaacgtgattcatctcaaggggcttaatcccggcacgacgacgttcaaactgatgtaccgaacagatgggagcggcaccgcagcgttctataaccgcaagatttgggtcgaaccgaaaccgtaggagggacaaccacaatgccactgaagccagtcgagctcatcaccgaaccgacgtaccaggcaatgcgattcgacggcaccgtcggatccgcgaccgacgtcgcatgggccgtgcaagccggacttgccagccggctactcgtccgcgccgatatcgaggcattccaagagggcaacaaccagccggaatggcggatgacgcttcgccggccggatgacgtcgaggtcgagatttacgccggatattggatcgtggcctggtcaaccggcctgatcgagggatacgacgacgcgagctatcgcgcgaaattcacactgccggaaggggaagagtgaccatggatgtacccgaagttcgtcagtgcgtcgatgaggcgtcgcacatgttcggcgccgtcgccgtgcgggccggcgtgaaccgctggggcgtgatgcatccgcaacacggcgggcactgggccggcgacgtcgaggtcaacgggtggccggccggcgcgttcgacgtcgaggcgggcgagtgatggccggcgcgaagaagacaccggccaagaaggcgccggcgaagacgtcggcgtcgagctcgacgagcaccaggacgaagacgtcgccggcgctcgcgcggcgcgtcgccgacctcgaacgcgaagtcggcgagctgcggacccagctcgacaacatcgctcgattcatcgccgccggcgtcgcacaacagctgcaagcgcagctgatggcgaacccggccgcgcagcaacagttggccgcggcgctggccgcggctcagggcaaccagccctgatgattcgtcggtggctggcagtcctcgacgcatacggcaccgattggccggcgttccatgagggacacccggaagatcgctgtcctgtgtgcggcgaggctgtcagcgctcacgtcggtatcgcgctcgcagacgccgaatgggcgaagtgggcgcaccggcgcgcggctgccgtggcgctgattctgaggtaaatcgctcacgcaccgcaccgcatatgtcgctatccgacatatgtcgtggatgatcgggatatggccgatcgtttcttcccgatgcgcgacgggacttacaccctgtcgtccgggttcggagctcgctggggaacacagcaccgcgggctcgacttcgccgccaaggacggaacgccgatctatgcggcgcaggccggcaccgtggcctatatcgggccggcgcaaggcttcgggcaatggatcgtgattgaccacccggccgcggacggcgccggcacaactgtctacgggcacatgtggaacgcgttcgcgaccgggctcaaggccggcgatcgcgtccaggccggccaactcattgcctacgtcggcgccaacgggcagtccacgggcccgcacctgcacttcgaagttcatccgaccgtgtggcgccaaggctcccagatcgacccgctgccgtggctgcacggctcccggaatcccggcgatgcgccggcacccgcgccggcggatccgccggcgtccgctccactaggaggtcagcgaatgcaggatccgttcaccggcgaagtctggtcgccgaaccgctcggtacggcagaagccggcgccgcggtggatcgcgattcacacgcaggaaggcggtcgaaccgcgcgcgatctgtgcgaaggctggctggcgaagcgtgagtctcaggtgagctaccacgtcgccgtcgacgaccgcgagattctgaaggtcgtcgccgagtccgaccgtccatgggccgcggcgaacgccaacgactacgcgtttcacgtcgtcgccgccggcagctacgccggctggtcccgcggcaagtggctcgaaaccgacgcgtccgacggcaagaacgaagacgtcgagctgaccaatctcgcgaaggtctgcgcgtggtggtgccagaagtacggcatcccggccgaatggatcggcgggcgaggcgtgccatggggcctcgacggcatctgtggacatgaggacttcggcgcttggggcgggggacaccatgaccccggacccggattcccggccgacgagctgattcggcgcgtacgcgcgctgctcggcggatccacaccggaaccgctaccgccggcgccgccggtggcgctgcctggcacgaatcccgaccaatacgccggcgttctgctctaccgcggccggcctgggcaggatccgcgacaggtccgcgtgctgcaaacacggctgaaacgggcctactcgaagctcgacgtcgacggcatcttcgggccgcacaccgaagcgtgcgtccgcgactaccagcacttgcacccgcccctggtcgccgacggcatcgtcgggccggcgaccgctgccgcactcgggctggtgttctgatggcgtaccaagcaccgcgcaacgtcggcgacacgcacccgctgatcccggcggcgaaacggaagctggccggcaactcatacggcaaggcgatcggcgacgaccgcagcgacgtctacacagaagcgttcggcgccgcgctcatccagtacggcaagaacgttcacgacctcgtgctggccggccggcgaccgggccccgacgtcaacgtcgagggcatcttcgactgggcgattcagcggcaaatggagctgaccgcaccgccggcgccgccggcgcccccgcgcgatcgcgcgctggcctacgtctggcgcggtaccggcggcatcatcgggcaagacctcgtgtcgctggtgtgccaaggcgtggccgacctggtggacgaagtgaatccaccgtgggcggcgaccatgggcggaatcccggtcggcgtggccggcggaatcggcgatccgagtatgtggtcggccgtgcaagcaacgcttgcgtggacacaaaccgacttcatcgcacggcacaaggttaatccgaagatccgcgtcgtgatcggtggctattcggccggcgcgatcgccgcggcgatgttccgcgcgtggctgctcgaaaacttcccggacaactacgtctgttcgttctcgttcggcgatccgactcgaccgttcggtggtggattcttcggccagccggcgccgtgggggcgcggaatctccacgatcagctacggcgatccgaccgattaccggcactgctggctgactcacgagggcgacatgtacgcccagattcccggcggcgtggtcggcgacatcatggacgacgtatacgaggaagtgacccgcttcgcgttccgcgacattctccaagtcgcaactcggatggtgtcggcaatcccgacgatcgccggcaaggccggcattccgctgccggccgtgttcggcgccctggccggcgggcccgcggggctgaccacgttcgcgattccgctgctgctgagctcgattcgcgggttcatccccggcggcaacgccgacgagctcaccggcaccgcggcggcggcgaaggccgcgacgatcgggctgcaattcctgttcgccggcaccgcaccgcatatccgctatcacatcgacgaggcatggccgggtggtccgacattcctcgacctcgcacgccaacacgtccgcgactggaccagccggccggccgcataacccaacaccgaagggaatcaacgaaatgttgaccaagtattcgccggcgcagaaggcgaaggcgaccgcggcgctgctctcgtcgctggtgctgttcctcggcgcgttcggcgcgttcgtcgccgacgtcctgccgaccggatccggcgccgcggcggcgatcgctgccggcatcgccgtcgtctgcgcagtgctggtgcgcaccgcgacgttcctcacgacgtcggcgccgacgctggaccagatcgcgaagaacctcgacgacacgatcgagctcgtgcgcgaagtccggccggcggcaatccagccgagctacggccggcacgcgcgcgaagccggcggcgacgacgccgacgacgacgagccgacgagctagggccgtggcggcatggaagggaccacggcaccgttcctgggcatcggatacgaacatctcgttattcatggcaacggttgtggttgtggcctacgtgatctgggaaatcgtgggggaagcaccgcaagggatggtgacgctcgtcggtttggccggcggcgcgttgttcggcgcagtgtccggcgataagcgcaagagggttcacgacgtcgagctgaccgcagaacgggccgaagcgaaggccgaccgcgcggtggccgtcgtcgaggaccaggaccagaagccggccgagccggccggcgacgacgagggggtggcacggtgacgatcgtcgacgacctatggtcctggccgttcctggccggcgtcgtggccgggttcctgctctcgcgtgcctggtcaatgttcaaggtgtggcagctcgaccggcgctacccgctgccgggtggccggcgtcgcgacgtgtgggccgctgccgccgtcgacgcgcgctgggtggccggcgtcgtcggcgccttgttcctggtgtggtcggtagtccagacgcaggagaacgccaacgagaacgcgcggatcgctgccgaagcgaagtcgttcgccgcggctgtccaacagtgccagtccgacttgatcgcgtcgatcatgggcagtcgcagtgtcacaacggaaaacgacctgctgtccgtggaagaacgcgcgctgctcgctgaagggcagcggctcggtcacgaatggctcggatcgctggtggcgccaccgccggatatcgaccggctcgggccgagcgatccggctcgatcgcgctacttcatcgagcgcacgcgcgtctacttcgaccagctggccgatttgaaccgccggatcgaggcgatccacgatgaacagttctcgaaccagcggacacggccggcgctgccagaccccaattgcggaacctagaagccggcgaccagcggaacggaatcgtccgtgtcgacggcgttgtcgaggaccaggacgtgtgcgacgtcggccgtcctgtcgagcacaccgccgatcgcgtcgctgataccgaggccgagcaagatcatcgccgcggcaatgatgctggcgccggcgcgcatccaggcgggccggccgtcgccgtcgccggctgccatcatggcggcgcggacttcggcgtcctcgacggccgtgtaaatctctgtcgtcgcgacggacgcgtggccgagcatcgtttgtactgcgcgcagattgcgcgatccgcggtaggcgcgcgtggcgaatcggtggcgcagcttgtgcatggtccagccgtcgggcagcgcgtcgccacacagccggcctacccaccgcggcgacaggtggccggcatcgtctcccggaaacaggtagccggtcgccggcgccccgtccgtgtggccggccgcaccgcgcgcgatgagctcggcgagctcgtctgacacgggcagcgtgcgattcttcccgcccttgccgtgcacgaccaggacgtaaccgtcgaacgattcggtgaggtccgacgtcgagacctgggcgacctcggcgcggcgcaagccaagctcggcggcgagtcgcaacatgagcatgacgcgcgcgtccgctgccatgagtgcggcgcggtacacccggtccggcgccggccgcggcgccggcggcgcgggtttgatggaaggcagatcggccgacggatccgtcggaatccggccgtcgcgacatgcccaaccgaagaacgacacgcacgtgttgcgatatccgcgacgagtttcgcgggcccaatgctcttgctcggcaaagaatcgcaccagcgacgcgccggtgacgttctccggtgacacgccgagcccgcgggcgatccggcgaatgtgagccgcacgtgtagcgcgcgtcgtcgacgggtatccggcagccgcaagcgagtcgagatagtcgtccagaatttgcatccagtcacggggcgcaggcacaggcgccggccccggagtgtttcgtccggtgatggtggccccctcgaaattacgagtaaccggacttacattgccgtttgccgcggcatctgaaacggttttcacggaccttattgtccggctaactaccgcgtcccgcctgggacgatagctgccggaagcgctcacgacgctttgtccaggactgaccacgggcccggaatcacgtcggcgttgcgctccgagtccacactcaccagggacttttttcgcgtagctcctaaacagaaggttgggggttcgaatccctccgggcgcacttcttcatctcctgccggcggatcttccggcggataccaaccaccatccccgtgttccagccaactaatcggaacgccggtggcaagggcccacgcattgagcacgattttgcggacgggcccttttcctttttcagcgttggagacaacagtccggccgcaccccatgagggcggcgagttcttcttgcttcaaacctgcttcaaggcgcgcgatcagcagacggtaggaaagcctgctcggcgggatccggcccatatcgaatgcttgtgacataccggcgattgtatgtacgtctgtcgcgtacgcgcaatattgccgttcgtcaacacttgcgagcggaaacggcgcgacgcgccgatcctggcaaaccgtcggtggtaacggattgcgtggcaaacgcaaaagttcgggcgtggcgcattttccaataggcgacatgtgtcgctatgttggtgctcatgaccccaaccgaaaccacagccgcggagctcaccgtctcggaagccgctgcggcacatgacattccgaagcgatcactacaccgcgcgatcgaacgcggtcagattcccgcgcgacgcgtcggaccgttgttcctggtcgacgccgaagctgcgcgtctctacggcgaagtgttcaaggcacgtcgagcgctcgacgcctacaccggccgcaccggctccgacaacgattccgattccgaatgaagggcacactcggcgccctgtgcgggctggccggcgacgctctgatcgccgtcgggttcgctctccgcgagcacgatcgcgggttctcggactacgaggccggcgacttcctgacgccggccgaaccgccggcgccggctgacgacctatcgattccgctcggcgacgtccgcgcagcactcgacgacctgtccgattcgaagctgctcaatatcgccgcgaccatcatcgccggctggaaaccgattctgctcaagaccaccggcgacctgaccgacgtcgacgtcctggtcgacgctctgcgcgatcgcgccacccaatatgccgtcgtcgagtcagacgacgttccgccggctgccggcgccccgaccaactcgcccgaacgcggcgagtgatggacggccggcgccccgctgataccggaacgccggccctgacacccgaaggaatagttcgaatgtcacacgacaatttacggccgatctggactcccgacgattactggaaggccgttcgcgaagcggccgaccacgccgaacaccgcggcgcgatcaacgaggatccgcattacgccaggtcagcgacaccgccggctgagcctcgtcgaggcgcaagtgcccggactgtgattcgcgcgctgctcgccatggcgtccggcgcggtgctggtcgcaatgttcgtgctctggctggcgccgccggtcaaggcggatacgccggactgcctcacgcagttctggttgattccgttccagtcgaatacgcgcacgatctgcgacgggccgatccagccggacggatcctggcagcgggcccgcgagttctggtcgccggcctacaccgcgccggcgcgctcgtcctgcactggtggctaccgctattcgagctgcacctactacccggagcgcttcgttccgcggaagtcggccggcgtcgaggtctatcccgtgcggccggaaacggtactccccgacgagccgggacacatcggctgatgcgcgcgtatttccgccgcggccggcaccgcgccaacaagttcagccacaccgtcgccgacgtcgagcgctgcctcgacgccgaagccgtcgagctcgaccgccggcgggcaaccgctctggtgctcgacggccgctgggccgaagtctgggggaccaaccgatgaccaccgtccacgacgtgaagatcgagccgcgctggttcgaccggatccagcagtccgagaagttcgccgaagtccgttacgacgaccgcgactatcaggccggcgaccggatccgcttctggcgcacggacctgcccgagtacgggcacagcatcgaacgccggattacccacgtgctgcgcggcgtcgacgggctcagcgacggctatgtggtgctgtcgctgtccgaccctcgactcgaccagctgcgcggccggctcgacagagcgctcgacgagaacagtcggctcgcacgctccaaccgcgggttgcgcggcgccaatacgcgtctgaggaacgcgcgatgagggcgatcgccggcggcgcgctggccggcctggtgataggcgtcctggtcctggccgtcggcgccaccgcggcggcatggatcgcactggcgctgtcggtggccgcgatcgccaccggcgccgcggactacgtgcggactcactgatggcggcgaacatggcgcgcgccggatccgacggccggccgtggccgggattcaccgtgcgcttctgcccggtctgctcggcggaaaccggaacccaccgtgaacccgtcgacgaccaccgcggcgaactcgtggtctaccacgaccacgacgaaccggcgaccggcgtccgctgccgcatggccggcgaacgcgccgcgattcgggccgtcgcgttctcgacgaccgacaccggcgcaagtccgcggacgaccgccggccgcaacctgcaccggccggcgggctcccgcgcggtcgacgaactgctcgaacgactcatcgaacaggagacacatgcaattcggaattgacccgaagacggttcagcgcgtcgacgaactggtcggtgtcctcgaccggctgaccgcggcgctcaacgcgaacaccgcggcgatcgtcgaggcgaaccggcagcgcgacgacctgatgaacgggccccgcggatgacggccgtcggactgccgccgctgaagtgtgagaactgcggatacgccgagcgctacagccggcagcaggtcggcgagcggccggcgttctgcacggttcacgagcgctggttgtgcgcaggctgccgcgcgttcatgaactgcgtcgacggcgggcaccgggtcgtgaaccaggcgccgcgcgccgacgtcacgaacgccgtcccgaagaacgatggcatttacgccaacatcgacgaggacgtctatcacggcgaccacctgagcctgtcgtcgagcggcgcacgcgatctgctcagcggcacaccggaagagttcgacttcaaccgtcgcgttccccgcgacccgaacaagaactacgacttcgggcacgctgcgcacaagatggtgctcggcaagggcgccaagctgaagatgctcgacccgaaggtccacgggctcaaggccgacgggaaaccgtcgagctctccgacgtcgacggctatgtggcggaaggccgcggctgacgcacgcaagcaaggtctgattccgatcgccaaagccgacatggagaaggcgcagaccatggccggccgcgtgtttcagcaccaggtcgccgcgcggctgttctcgaaaggcgcagcagagcactcgatctactggcacgacgacgcgaccggcgtccggttgcgctgccggccggacttcctgcccgatctgagccggccaatctgtgtcgattacaagaccgcgacgtcggcgaacccgaagcaattccagaaggccgtcgccgactacggctaccaccagcagcaggcgttctacgaagacggtctggccgaaatcgggctgaccggcgtcggattcctgttcgtagtccagtcgaagacaccgccgttctctgtgtcggtgtgccagatcgacccggaaatcgtcgagctcggccggcgccagaaccgcgtcgcgatcgagattttcgcgcgctgcatggagcaagaccgctggccgggatacgagggcattcaggccgtcgggatggccggctgggcagtgaaacagatcgaagaccagctcgaagagttcgagcttcaacccaccgcatgagaaaggcaccaaacaccatgaccagcagcgaaatcgacaagatcgacccggacgccggcacgctcgaatggctgccgccggcacaggcgccgtcggctgtgtcccgcgcgatgctccgtgagcacgcgcagatgatggccgacgcctaccagcttgcgaagggcatctgtggggacggccgcaacggtactggcctgatggttccgcagcgattccgcgggaagccggaagaggctgccgcggcgatgatctacggatccgagctcggactcaacccgttgcaagctgtccagcgcgtcgttcccatccacggaatgcccacgctcgaagcccgcacgatggttggtctactcaaggcgcgcgggtacaagatccgcaccgtcgagcagtccgacaccagcgtgaccgtggaaggcgttgcgccggacggcgaaacggcgtcgagcacctggactatcgagcgtgctcagcgcgccggctacgttccgacgccgagttcgccggactcgaaacgccggcccgacgtcgaggacgactgggtgaccgtctcgaagacgtgggacggcaaggtgaagaagtcggtcgtcggaaacatgaagtacatcaccgacccgcaggccatgttgaaggccaaggcgcagtcggaggtatgccgcgacctggcgcccgacgtcctcatgggtatcagctacacccgcgaggacttggaatccgagcgtcaagaccagttcgaggatcgccggccggccgcggcgccggcccggacgcagcgcgtcacggtcgacgagattttcgccgaagaggttccgctgtcgtctgacggtttcgccggcgacaacccgcccaccgcggcgcaggccgcaccggagccggcacccgagccggacacccaccaggcgccggccggcgacgtggcagaggaccagggcgacggggccccggatccgggcgaaccgtcgccggccgagcaatccgaaacggtagtcgagcctgagccggcgcccgagccggcgccggcgccggacactgccgcggcggcgaagtcgacgtcgaagaaggtcaacgccaagcgctcgacggcgccggcggcgaacccggacaagccgaaaacccggatgcggcaagcactcgagaagcgtctgttcgcgctactgggcgacgccggactgtcgggcgacgccgaccgcgacggccggatcgctgtctatcgctcggtgctcgaaaacgaggcgatcggctcgaccgacgacctcgacgatcccgcgatcgggaaggtcgctgaccagctctacgcctggcagcagcaaaacgaactcgacgacaagatcgccggcattctctcggacgctgcccgcgccgagtccgaatccgtggcgcccgctgcggatcccaccagtgaagggaacgagtgaccatgggactgattcaggaccagcccgaagacctgcccaccacgaacgcgctcgacgccatcgtggaccccgacgaagagcagggcggcgaacgcgccgaaatctatctgtccggcgcacacgtcgcgacgctcgacaacgcgccgaccggcgccggcgcccgcgtaaccctcatggtcgaactcgaagtcgtcgaggaaggccacaagtacaaggagaacggcgacgtcgaagtcccgatccggcgctgcaagcggatcggcgacatgtggcgccccggcacgccgaaaccgcccacgaaggaggaaatcgccgcacagcaggccgcggcgaaggcgaaggccgctgccgaggaagccgcacgcgaggaagccgaacgggccgaacaggagcacaacgagccgccgatgttcgacgaggacggcgaaccgatcagcgacgatgcgccgcgcccaccggacgacgacgaggcggatccgaacacccagttcgacgtggacgaggaccaggacgtcggcaacgtcgtgcagttctccgacggcggcaagtgagcacggcagacgccggccactcggcgcactggatcagcgagtgcgcggcgtgcttcgcgatgttccgcgactcacccgagacggccggcgtctgcccccgctgcggacatgaaaactactacagcgatgaggaatcccagtgggacggcgaaatcctatccacgtcatcggcgagccgatgcgaaaaggcgtcacgtggctatccgacgacctgatccgctcaggcgcccgcgacgacgctctgggatctgacgggttcgtcgtcgtggcctacctactctcgcgcgtcgccagcagcggccggcccgcgtgggagacctcagcggctcagatatccgagcaattcggttggggccgtaaccgtgagcgcgcttctcgcgcgatcgaccgggcagccaaagacaaacgtctgctgatccgtaactacctccgagacgggcagctggtgccccggcgatgcgcgtacgtggtgtgctccggcggtcgccagttcaccgatgaagagctgctgcggtggtcgaccccgatcgttgtcccctcgaaatcgacaccgggggaggacattctatgaaggcgcaccatgcacggattccgtgcaacattgcgcgctccgtcatgcacgaaaaccgtgcaacatcagcggtccacaccttgcacggaaaccgtgcaacatcagcgactttcgagacgcacaatcttgcacggttttttgtgcaacatcaccggcccgaatcttgcacggcagccgtgcaagatgtcatgcacggtttcagtgcaacaaaggacaacccacctacgtacgtaggtaaggacaacccgatcccgtcatgcactgcgtgcaagacggccgttctcccctaccgcaacttgacggccaatcaccttgaccaaaccagccaccgaggtaaccaactgaccgcacgcgcggaacgaggggtgtcgccatgccagcactgaccgcaaagcagatggcgacgaacttcgctatcgacgacgcctggtgtcccggatgcggacttcaccggcacgtccacggcgaacaccgcgcggactgcacgaccgagccggcgccgctgatgtgcgccgactgcgacgaaatccgactgccgagcgaacgcggggcgccctggcagtaccgaccagccacccgcacgtatcactgccccacccaccagaaaggcacccgatgaccctccaaacgccacagccggccgacgcgcgagcgtccgcgtggatcggcgcgcacgaacggatcgtggccgcgcagaagctcaaggtcgaccacaagaagcgtgttgtcgatccgctgcccgagctctactgggccaccctggccgaagctgccgtcctggctgagctcgcacgggccgaccagacgaccggattgcacgccggcggatacctggtcgaccagcaggagcggatcgaggaattcgggcgattggtggcgcgatcgcgcgaagcgcgccggcacccgcaccgcgcggccggcgacgagctcgtcgccgaggaccaggaggccggccgatgaccgcccgaaacgagaacgtcgcgatcttcaacccgcttgtcctgccgcttccggcatggcaccccgacgggcccgacggcacgatcgacggattcgtgatcccgttcgtgttccccgagacccgcgcgtccggccgcgagccgaaccggcgcgcgctgatccgtgtctacccggactcccccggttggcgggccgcgctgcggaactgccaagaccacgccgagacggccgagcaagcgcgcgcgttcgccgcggcatggatccgcgcggccgagctgttggaaaacgtcggcgccggcgccaaaccgaagacgctcggcgacccgacgcgcgaacccggcgcgctgccggccgacgtcgagcaccaggacgacgagacatgtccacacgacgatctgcgttgctcgtgcgcattcggcaagggacaccgcgcatgatcgccgtcggaatcgacccgtcactgacctcgaccggcgtcgccgtgctcgtcgacgggaagctcgcccactacggccgatatgggcgaccagggcagaacggcgcgagttacaccacgcgaagccggcgcgtccggcgcatgatccgcgaagtcaccaaggccgcgttgaccgccggccggcccgacgtgttcgtgatcgaggaacatccgtacgccgtgggcaaccagggcaacgagttcgaccgggccgcaatctggcatggcatctacggcaatctcgacgcgcacgaagtccccggcgtcgtggtcaacaactcgacgggcaaggcgtgggtgaccggcgccggccgcgcgtcgaaggaagacgtgatcgccgtgatcgacgactggtacgccggccaaatcgacccgccgttggcgaagtggcgcaagaccgtagaccaccccgacgacgtggccgacgcgatcggatacgccacgatgggcgccttcaagctcggcgacccgctgccgttcgagccgaaggaacgccaccgcacggcgttagccttgctgccgtggccgaagaaagcgcagttccgatgaagtgcccgagctgcggcgcctactcgctgacgtacgaccggcgcctacacgcgttcatctgccacatctacggcgcgaccatccccgccgaaaggattcccgcatgagcacaccaggacagcgaccagtgagccgccggcgctcactcgacgagcacttcgccgaaatcgaagagctcggcgccaagatccacgccgcgctgccggccgagctacggcagaaccaccagatcgaccgaacccggctgagccaaggcgcccaatgcgtctgcacgtgcggcacggtctgcacgtactgggttgcgcacttgcaggacgtccgcgaccacgccgagctctgggagcggatgcacgccggcgcatcgaacccgcaacggatcatgttcgggctcgcccagattccggccgacgagctcggcgcgtgctgcggacacgcctaccgccggcacctggtcgggctcgacggttgcgcagcgtgcgagcacatcgaacccgttcacgcgcgttgcaccggctggcgccggcagtttgaaggggcgccggcatgacgtgggaaccgattctggccgcgctcggcggatatctgttcttcgccggccgcgacgtgctgcgggccgtgatgcagtccgactggatcttcggccggatcctggccgcggacctacgcgcgatcgaccgagcgcaacgaagaaaggcgaagtgacatgtcccgaatctgggcagctctgccgcttctcgaagacccgctcgacccgaacgcgccggtgatcccgccggacgcgtgctggtggcgccacccgctggcgaggaacgaagacggtcacgagctgatcgttcgcacggactcgacgtcggacatgcgcgcggctcggctgggctgttgggaatggttcgacgggctcgacgcgcagctgattttcggcgtcgacgtgttccgcgtgcatcacacgttcgacggcgcccgatcccatctcgacgtcctagcgctcgacgttccgacgcgatcgctggcctggcgcaacctcaccgaaccgggcgactggcacgtcccgcgcgtcgacgagtacgtgattccgagcgacgcgttcgtccggttgccctacgccgtggcaccgcatcccgttcggccgtagcaccaccgctgaaattgtccagggccgacgtgtgtccaacttgacgacatatgtcgcgtactgaacaattgtcgtcatgaacaccgcacccgccaccaagaccccgaccttctcgatcgaagcggaatccgggacatacggaccgttcgacacctacgccgaagcccgcctctaccagcgcgaatggcagctgaccggcagcagcgtagtcccgaactgaccgccggccggcgcccaccacgggcgccggccaaacccgaaccccaccagaccggaaggccccaccaatgaccgcgatcgttctccgctccgaactccacccaaacggccaactcatgcaccacgtcggcgacctcgacgggatccgcgtcgaagctcaccagaacgagcaggcctacaacgcccgcaccgccggcgtgtggttcctaaccatcggcgtcgccggccacccgctgcgctgccacagtcagcaccgcaccctctccgacgcccgcgcgttcgcgaacgccgtcatcgccgaagctggcatcaacccgacccacgcgcagctggtcaaggctcacgactacgtggaagccggcccggttcttttcgccgacgtcgagctcgaaaccgccgaacccgcagatcagcccgaggccgcgggcccgatcgtcgtcatcccgtgcagcgcgcagaagctcgaccacgccgcgccggccggccagctgtacacgtcccagcacttccagctggtctaccgcgcagcgcgcaagctggccgacgaccagggcgcccgcgtgctgatcctgtccgcgctgcatggactggtcgaccccgaaaccgtgctcgacccctacgacgtgaagatgggcgaccgcggcgccatcgcgccggccgcgatcgccgagcagctggccgcgatcaacccgaccgccatcactaccctgttgccgcgtgcatacaggctcgcgctggtccgcgccggcgccgtcgtgaccgacctctacgccgacgcggccggtatcggctaccagcgcggcgtcgcgtcgcagatcctcgccagccggcaacacgccgtcgcgaccgacccggccggctacgtctacgccggaacgctgttctgatggcaacctctcgcgatccgctcacggtgacctgcccggtctgcgcgtctgagcccggcgtcgcgtgccgcgcgaagtaccttccgagcctgatcgtcgaacaccaagcgcgccgcgaagccgcggccggcccgcgtccgatccgcgtaatcggcgacgtgatctggccgtacgacgaaccgcattagcggaacaggcgccgcggccggcgcaataatgcgtggatgaaccacgccacgcgcgccgccggcgccgcaatcgccgcgaccctactcaccgccctcacgccggccaccgcgcacgctagcgaagccggctatctcgcacgcctcggcgtcgactacggctaccagctcaacgccgaatccatccccgccgcactcgaagccggccgcgtcctctgcgacgaaatgcgcgccggcacaccccgcgaccagctcaccgccagcgtcttccaggccatccccggcgtcacccaggaccaggccggcggcatggtcttcgccgcccacaccgagctctgtcccgagacgggcgagttcgacgcgcccgtgtaactcgacacgacgcgacattcgcgcgtagacgacatatgtgctcccccagctatcgtcgggggagcacgtttgtttctacgaccaggaggtttcctgacatggctccacgcgacaccaacaaggccgacaccaacgacaccagcgctgccgacgcctcgaccaccgaaggcgccacagccgccggcaccaagccggccgaaccgcagcgcgtcgtgatcgagcaggcccccgccgagcccgccgtgttctacggcgaaggcggcatcgtgcccggatccggcaacgccaccgatcccaacaagccgcacgtccaggccgactaccaacccttcgagtggtgacctgacccggatagttcgcgctgatggccggcgtcccacgcaacaccgcagtacgagacaggcaccgacgcgccatccaacgcggacacccgaactgcggtagcgagcaccacgactgcgcgcagaagcacccgcactgcggaatctgcggcgaacccatcgactacgagctcccacatctcgacccgtgggagttcgtagtcgaccacgttgtccccgtggcagcaggtggcgccgacgagctctggaacaagcaagccgcgcaccgctactgcaacagcgagaagtccgaccgattggccgaccagctgcccgtcggagtcacgttcgtaaccacccgcagctggtagccctcgaaaggacaacccagtgaccaaccagccacgagagttggagttcgcgaagctgcactgggtccagccggccgacaccaacgaggaaccatgaccacacgcgaacctcgacgaggcgccaagccgcaaccctgcaagcactgtggcgaacagatcgccgccgtagcctccagccgcgactacgtgcacatcgaaggcgaccacgccggcaagcaccgctgcgccatcgagccctacggtttcctcgccgagcccgtcgggacaccatgccgagccgacggaccgaacccctgcctcggcgccgaccgatgaacgccgaaaccatgtgccagcaccgcacccgccggcgcatccacggcagcatgtggcgccaccccgcgccaaccgtcccaccagcacccaggccgggtgaccccaccg

SEQ ID NO: 2 - DNA, 71684 bp, unidentified
  source 1..71684: mol_type = genomic DNA
  misc_feature 1..71684: note = contig00001 len=71684 cov=18.2 corr=0; spades = NODE_1_length_71684_cov_18.164964_pilon (bacteriophage Ore278)
agctcgagcccttttcgactacgctgatgagagtaggcggagtaggtggctggttgtcttcctcggacgcttcgtcatccaaggcatcttgcctaaaccgtagccacacagcagctccggcacccttaccaccactttgaaacagcgcgaagttgcccccgttgccagcgccaccggggtaagccccattgccgccaaacaccttctgttgcccaccggctttgcatttcatgcccttgtactcgtaggtaccaggactcttaccaacactagccaaaccgaatcgaaggtcaacaccaccggcacaaccaggcaccgtaactgttgctgtcgggatggaaatggtggtctcagtaccaggtttgccgaacgggtcaagtagtgaaccaccgtcaccgccacggcctagcttctcgatgttgacgattactccactgccgctgaaatctacacctctgtgccaagttccagcaacaaatgggcctgcctcaccgcttttgccatagaagccacccgtggcaccagcgcggcctccacccgcagctggcacaatcacataatcaatgtgctgcacccagtctggcacaatgcgcgtgccttcttctgtaacatacagatcaagtgggtcgtggtgtccggagtggtcacccaaatcaatcgcgaactctccgtaggggattgtgtggaccggggtgaacgagccctttgccagggtgctagccggcgcgtttggcgtagtctcactacgggtaaacgcaaaagcctcgatggtggctgccgtgtcaactggtgtgtagctaccttcctggtcagccaaaaggtacagcgtgccacccacgggcacccactgcacgaagtactcttcgccggcaacctgcgcagggcggttggccggttcaatctcatagtgcatgtacttgggtgagcttgatgttgccgtagggtcgatctgcccaacaatgttgggggaatggtggactagggttttggagccatcctcttccactctccacaggttgatgtacagctcagtgatgcctgagccaggataaccaatccagcgaataacacccagcggctgagacacttccacgcggtcactgattacagctgtattgctttgtgttgcagcgaagtacaccgcgctggtgttgccggtgaccttgtccaggttcctgttcgaccggcttgttttcagcaggccactaagaatcgaagtgttgttacggattcccaggacctcccaggcgttctggccttggcttgcacgctggctcaccaagttcaacacgttgatgatgtctgccaaaccagcaccgacgccgtccactacgccaacagcgccaccaacaatggcattggcgaacgcctccaaggcctccagcagggttccagggccaccccagccgagaatattcctctgtggaatattgagtagggcttctacaagatcggcaatctccaccaggaaagcgctagatccagtaagagcgttgaagacagagtccttcagcaagtgccaacggccgagaaggtcttgcagctcttcgagcaggccttcaacccactcagggagaatcttgttgcgtaaaaagccctttacgtcgtcgtaccgtatcaacccggctgtggcactactatcaatgtacaaacgaggccgaaccatgtcaacgtctgcgttaggaacttcatacgtgccagtgatttccgtccaaccaccagtttcgctgaatggagcgaacttggccacctcgtggtagccggtttcggtgtataccggagcaccgaattcgtctgtaccagtacgcttgtacaacaccaggtgcaactgaattggttcacctgtaccttcaaagtcctgccactgtacttgggctgtcatttccagacgttgccgagggtgaacaggagtctcaatgccccgcaaggctttcaggctgccgtcagcagtggtgcgcacactgcccgtgccgtctgcgctcttggtaatgccagtgtccaggtgccagatattttgtccctcaaccgattctggagggaatgcccctagggcccacacgttttgcggcgtatcaattagtgttgatatgggtacgccaccgaacagatgtgacggtatgagaccaatcagcttggctgcgttgattggaatctgcttgatgaagtcaatcaggccgtcgatcaggttgacatcaataccaccgctacgctgtacggtgatgtcgtcaacccacacttggccggtgttgatgctagtgcctacccacgcagcgattctgaagccgtcaacgttgttcggaacgacaatgtcttccgatgtgacagtaacaaatccacctgtggtaccgtggccggctggtatgctgacatggctagtgctaacttcagtttcaccattgaaccaaaccagctgcagctgtatatcgttggaagccaacggaatacccagccagcggagcttgccaccaatgacgaacttggtgcctggggtggctttgtagatcgtctgtgtccatactcgtttcgcgatgccattaccgatggtggtaaggctgcccggtgctgtgccggtggcgttgacggtgccgtcgaatgtccagcctgttgttgcactgccacccacgctccacactgtcgtgtccgcgctggcggcgggtgcaacgttatcgcctagggagcgaatctgactaccactgatgaagccgaggaaggtgctgccaccgttgacgattgcagtgagggcgttgataaacggcacgccaaggttagccagtagctgcagtacgggtttgagcaactcaaggttgccgatgccaccgatgaagtctaccagtgcatcccacaactcaccgatgccgctggtgctggtccactccaaaaggccaaacgctccgagcagctcaccgattgcgtcaaagaagccttcaaagctagtgcctagtccctctatggcatcccggaagttagtgactgcctcaatgaagtcttctgacagcccaaagtcctctgcccacagcaggattgaatcgaatatcacgtccgtaaactgaggcaacggaatgatatacgtagtgagcatgtgggttgctgcctcaacgaggttcagcgggaacggcgtgtcggggttgatgccaaagaacgccccgatgccctgaatgacgtacttgatgtctcccaactcaataccggtcggctcaccaccggcaaagaggatgaacaggtctccccagataccttgtagctgctctagggcattcatgttggcctgtgttacaccggcctcaagcttctgggtgtaagcgcttagcgtgtctacgctttggttcagccacgcaatattctttacaaagtctttgttagcgtaactgattttctgagagtcaatctctcgcagggctttagaagcggctgaccctgtgccaccgctgccgataccggccataccgcccgtcaaaaagcttgtcacttatggctccacgtactcaatcgggtcgtagttgaacgcaccctctgccatgacacctagctggactacgcctttctgctcatcccatgtgtggctcaacaccttgtgcttcacgttgatgtcccctgcccacggcaccggcccctgaatcctaatgatatctcccacatcaaaactgccgtgcggcgcgtttgggtgatagggatcaatcactaggctttggaactgtgctgcggggtactgcctgcggcttagtttacgcttcgcccacgcggccgcacgctcattcgagttaatgctgaggtcaacctcatccatcacacgcctgtagcggtctgggtctgcgttggatagcgtagagctgtataccttcccaggaaagtagccgttgatcgtgatgtctgaaaaccactctacctgtgtctcttgctttggcgtagcggctataacgttttcacccatgcggaagatcaagcttgcctggtctacgccaccggaagggtatgcaagcctaatgaacttctcaatcggggcctgcccaccctcccaggtagtggcctcccagtagtcgaacggaatatcacgggccagcctgttgatgtattccccacagtcgttacgatctactgcccgtatgaaaatcgcaaagaaatcttgtacgaactcttcgttttcgaagctgaagccgggcagcatttgcgtgcctgacaaagcaggcattgtcccgttcttgttggtgaccttaacccctaggttcccattggcatacgactgaatgtgagtccatattctggtcacaatctcgaaggggtccactgcgattgggttccaattctgcagccacggaatacccttggggtagttggcaaacccctcagctctgaggctcagaattccagtctgcgggtcaacatcactgggttgcaccagcgctgaggcccatatcttctcgttgccagaaaggtcctccttcagcgcatgaatccagtggccccatggcttgaactgtattggcccttggctattggggagctgtacgctcgggtctttggggtggaccttaaactcaatgccgcacgggccagacagcatacgagtcactatgggttccatggggataacgtcacgagcaatgatattgccgtgaatgtcctccaccatgaagtggaaccggtcgtagtaactcattgcctcactccaggatctggtatgcgtcgtgccacagcaagaccatcgaggtgttttcgttagtgtcggtagcgttccatgaaatctccttggcctcatggtcattgaagcgtacgtgttccagatatgggtcttcgccgatcaagtacgccgccacgctattgcccgcgctggtcactgcacgacgagaccatggatagctactgatttcgacaatctcaccctctggaatatcgtagtctagctccagctgatggtatccaaagttgatgattgggtgcttggccggtccttggatcaagaaccgcacccaactcggagcattgccacgagtaagcgttagagtctcaggctcattcagctcaaagcgatggtaccactcagtctcactgtaagcaaacgtatccagccggcgaaactctgcctggcactgatagtatagggagtctacgaccctctgcagctgataggccaacttgcctggacggccaaagaacatacgcgtaatgccatctgagccacagaagtacagtggcttggtggcaccccactccagcataacgtcctcagccctccactcacgttggaggtccccaagccccgggtcattgtcgaagttcaggaccactggatcctttagcagtgcagaaacattggccatctggcgattcttacggaagttgatggtcaactgcaccggagcgggcttcagcgtatcctggccaaaccgaataaagttgctagcctgagcctgtgagtcctgtacgtttacatcgtaattgccaatgtcaaagcgctcgatgctgtacatggtgtagtaacccataaccagattaccaatttggaactggcccggctccaggttcttagatggcataaccgaacgccccttgtccagatgctcgaatggtgaacatcatgtcatccatcgtgtctcgtgggtcttgcccaggcccttggtacacagtgttgttgttgatcacggttggccggtcactgtaacctctagcacccaattgcctaccaagcgtattgaattcagtcttgttctgtgggttgtcgctgctgtacgcctgaaccttgccgctaacctcatccagtaggtaccgtacgtcactggagccagtgaggccggtccagctctggatgaaacggccaacatacttggtagctatgccataggcttcttggccaatgtcaatggctgtgttcactgctgcaaggccctgagcaatgatgccagtgatagcacttgctgcggccaagcctccctgggccgcgctgccgccaccagtaggatcagctccacccattgcagcgatttgtgctgcgaaaccagtaatgttgctagcggtttggaagcccttttggaacagggtgatccattcctgcgcgttatcgattagacggttaacgtctttcgtgttctcgatgccccgcacaaaggtgtttgccatatcctgcgtagctgccacaaccttcaggccttggtcgaccacaccgaaaatgtctgtggcaatagacgaaatgccactggcaatcgtctgtgcctggtccagagccgatggcccttcttgcaggccaaactggttctgcaactgggacttgtagcctcccagagcttccgatagggccttagactcaggcgtacccagcttgtcctgatcggcaatgaggctgtccaagtgctgcagcacgggaaccactgtgtcgctagtcagctgggactgattgcgcatggcatcgcgcaaaactgggtctgtctctacaagatcagcaagcttgccgggcatgctaggcaggccgtttgtgttgccccagatgccccgcaaggccgctgcggattgccctgtgagggtcacaggcgtgccctgctggcccgtgtcacgggcgaacgctggcgggatggcccctggtgctgcggcctgcgcgaaagcgtccaacatctcgtttcggactgccacatgcacgtgattacggtgctctgacagcgttccggcgctgtactggtgtggcttgccttggaagagattgacgccaccctgatcatcactgtagatcagctcttggatatagggtgcgaatgtgtccctaacgtactcggcaaacttgcgcttgttctccgttgggacctgtacgcccggtatggcaaagtccccagcctcaccagacagatgccagctcttgccgctggctgtcatagcgttgctacgtttccctgatgtcagcttcaagccgaactggcttgcaatctgctcaaggtaacccaatgcgctgttagtgttttgagaaccaccagccggcagtccaggtggcggcgtgatgtcaccgctttgcttactgacagtagcggtttggcttgcatactgccaagcaccattgccaaccatgcgttcggcaatgtctgccttggctttaatcgggccaccaggagtgatcacatccaccatgccacccttggcggccgggggcgtggctgccttagtggcagtagtggtttgcggagtaactgtcggcgtgctgccgatggcatcaaggaaaggcttggctcccttgagcgctgcatcaaggtggcccttagctgcattctccggatgccaaggcccaccaatgctaacctgtgtgcccagttggctaccagcagtaaagttttcaggtgttaggccgcgctggcgggcgagacggaagtagttgtctacgttctgggcagcatcgtacatgttgccaccaccggcactttgccaagtgggcttatcgaactggaagatgccttcccagccagcggggttggaagccgtaggggacaaatggctttcgccatacgcctgcaccacaaaatcgcgggcctgctctggcgtatacccttctgacagagccttgttgtagatgtagttggcaatgtcttgctgagaggggttgggtcccagcttccctggtgctccaccacgtctgcctgccccagccatgcggctgccgctaccagtagcgccaccgctaccacgggcaccaccggcccacacggaaccgcctgggttccaaggccctagagcgctaccaccctgcttggctgtagtgtccttgatatagctcttggggaacagcctgccacccgacatgtcgttggcaaacttagcaacgctaaaggcaatgtctgagatctggttaccgaactcaagcaggtgcttgccaatgtccgagaacgcttgcaagtcacgtaaaaggccctcaatctgagtgttcaagggcgttgcgcccctagcacttagaccgtctgcagctgcaccagccacttcactggtaacgcctgcagcagtagccttttcgcttgctatgcccttggcccagtctgacgtaaacgtcttgccacggaacagtgtccagcccttgccggacaaagggccgtactttgctggcgaattgcctaggtagtcaccggcaagaccggccagctcaagcagtgcttgccgcacctcaggcgtgccttcaatcaagccttctgcgaatgcaacgctaagattctggccctgtgcctgggcacgcttaaccagctcgtccagctgggaggcaatttctgggttgtccttgagagccttatccaaggcttccttgttagacgccagatccaagaacttcttacggaattcagggtcgttctgtatctggttttgcagagcctcgtagtctggtgcaccaagtagtccccctaggctttccggtgctacagttttgccggctggaggcttctccggaatcgggccactgagctctggtgtgggcggcttgggggtattgtccggtgcagcaacagtacccgtttcggggtggggtggaccctgtagtgccggggctgggttagtctcgtcaccggcattggttcccggtgtatccaaaatcgtaggaacatgcagctttccatcaggcccaactatcttcgcgagaatgtcctgcgtggtagtgagcaggtccagttcgctttggatcttggccgggtcaccgtcccagatggactcgatagcgtcccttgggttttgagccttgagagactccaggattgcgtctttgggcagatatgcatcgatgattcgttgggccacatcagggctgatgccagcctgacgctgcagcaaatcctgtaggccctgtacggttctcgcgtaggcctcgtcggggtcagcctcaccctgaacgacaagctgcaccatctcacgacgaatcttctcgatattctcgaggagtgctcggccgttcttggtgttgaggtttagtgttccatccatattggccagtgcatccccggtggtgtctgcaaggtcaatgatgttggacatgtacgaggtcatttgttcaaatgcctcgttgtaggaactcaacgcatctcccccggggagcaggcccaggccctgcagcgcgtcaattaggctgttagccctgtcggctgcgctttgcgtcgaatcctttaggttgtctatggcatccgcgtagttcttgatctgacggccaccgttgacaaactgttcgcgaatacgcgttagcgtatcaatgagttcctgcgtgccacccttctcttcgacctcttcgaggaactctttgaactgggcgttggtgcccatgagtgcggcctcagcagatttggccttaatctctaacttctcaagggcttccttgagcgtaacgaactttggagtcagacggtcagtcgaatctcctaggtccttattggcattggcggcagcccgcgcctcagctgcccatttctccaccgtgctccacgcctggtccatggcgcggttcatcttgcctacattggcatctcgtacctctcggataccagagccaaggtcatgcatagcgtctgcgaaatcacgagcgttcttcgcaacgccgtcggccccgaacgtatcggcgatggcagcaaaggcctgtgagacccaacctgttaggtcacctatgatttccgtgatcttcaccacggcatccgacaccacggtgacaatgccggtaaacactttgccagtggcaatgcccacctttgccattacctcaaagaaggtaatgacgccaccctgatgttcttttaccttcttacctaaggcatctacggcctgtgtgaccttttgaatggcagtggcgataacgccagcgtctcctgcgtctttcttaccaaacaggggagcgagaaggtttgcacccaaacgggatacagcagcgccaaggttacgccagctgccaccgatggtgttgcccatggcctttgctgcgccaccggtcttgctcatggcgttgatgaagcggtcaaagctgacctctccattcgacaccatttcctgtgccttggtttgggttacgtcgtactcttcagccagcaacttaagcacaggcacctgacgagacacaagcagctgcatcacgtcaccagtcaacttgccctggatagctgcctggctgaaagcactaccaaggtcttgcattccaacaccagcaagggacgccgtgttggccgtggcgcttaggtacttgttgagatcatcgccaaccctgatgcctgcgttaattgcatcggctgcagtgttgaacgcctcttccaggccaaaggccgtgccctcaacagactgcaaggaagcctttgagattcgctcgatgtccttggcctgaacacccatggcatgcaacttggttgttgcatcctcgatagccgttaggcgtccaaagcctttggtgagaacaaacccaacaccggccaccgcagcagtagcaccggcagcagcagcagtgacacggcccagcataggcagtacgcctgctatgccactggagcctccaccgccgacattgttagcgccaccttgcggttggtcaacgtcaactcgcgtaggcaccacacgcacaggggcacgcagtgtaagtggcgtaagccgttgctgctctacaactctctcaaaggcccgacgatccagctccaagctgcttggagccaattgaataggggctctaagagtcagccggcgaaattgctgttgctcaagagaattctcaagcgcacgccgatctaggcgaacctctgttggcgatactgtgacagaggttttgacgttcgccttaccggcctgcttttgcaggttcgctacagctttagtggccctagtggtttcgttctcaaccctggtgccaaggccctcagcagggttgggaatgaccttctgccccagccgatcgatcattcgaccaaactgggacacaacgccaccagcgcggtctacgtccgacgcagcgttctgagcactccgaccaacatctgccagtgttgcctgtaagccctcaatatctttgacagcgccacgaacattgctggactcgtaagtgatacggactttaccagttacggtaccgagattggcggctccaccgcctgccgggctggtcatccgttgaacttactgaggtcgatcttatgcccctcggcactgagaaccttaggggcactaacgtctcgcaaattgcgtttgcccgcgcctgcggcattgggtgctacaggtggaggtgcatacgcgcttgaaacatctaggcccataagcttattgaacgccacgtgtcgagcttgcaatgcgaaaccgtctgtgcctttacggtttttcatctgcatccgcactgtcatctctgcttcctccacgcgacggtcaacgaatgtggcccagttccttagggcacggtcaaagtagaaggctgccagcggccatgagatgccccatagctcactgggtctcacccccatgtccttcgcctcgtgccatatcttgtagcactgcagcttgttcttcgcgaaacggttcgatcatccccaaaggcttgttcagctcttggaagaaggccatccggtctgcgaaatcgacatagccagagtacacttccccgtctttgagaggtgggatgggcctcttgaagtgctccagctgttctgccatgtcaagctcatagccgaacttggttatctttctacccgtttcagcgctgtccttgtcctcaaccaccacgacaccatcgtgaataatcttgggcttgatcgacgctgcttgcatcagacggccagtaacctctagaaagcgacggcgtttctcagggtcacgcaaggcctgccagatgttgtcctccaccttggcttgcgcctctacaggattgccgctggcgtcgatgtctgccggaaagagcttgcgggtgaagaagtccagttcctcaaccagatcgtgttcgatcaggtcaagtgtgtttaggtgcttcaccagcgccacctgccccgacgggcaatcgacgacgaacttgacgacccgcttaggcttccaagcactttcgttaaccgcgaaaaggtctttgacggtacgttttacggcctcgtcaacatcttgcggtgcttggtcgccggctgcagatgccaaaagagcagcaggagtaggggtttcccccactgaaacctcctgtgtcccaacaggttccttcgtgatggtatcagtggggtctacaccagggtaggacattggtcttcctttctcggaatgtcttgtcttgctagagggtaggacttagcccccgccgccgggcgcttccggggtggtgaacgcggtcggtgtgccgtagtggccaacttccacgccgaccttaccggcaacccgaatcaggtagtcggtatcggccgtaacggtgatggtggcctcgttgtcttcgacctccacagcggtactccaagtggtgccatcgtcggtggacaactggtaggcgtacttcgtgtagccggtcaccggctcccacacggccttggcagatgtaggcgaaagcacaaccaccacaacgttcttgggaggcagaatcggcagagcctccgactccaacgacaggaatgtctggctctcgtgctgcacaaagtcgtacaacagctttgtgcctggaattggcaggccgatgccgtccgcgctggtgacgaagaactggccgtcaccaaacgtgcccgaaacgtcaccgttgcacttggcgcggtacacgataccgacggtgtcgccgccgttgtcgttcatggccaagccacgcacctggaagtacgggcgtgcgtcatcggagcacttacggagagtaatggtccggttcggggccaccccctcctcgatgatctgtccaccggtgagaatggcccacgcctgcaggctgataccaccggcttcgatggtccagttgacctgtgctccattgccgtggctggccaacagaacgtcgtcgcctcgaaggtcagtgaagtcctcactgtcggcgaacgaaaaggtctgcgccgcagggagatcgaaaccttcttcagcgagctgggtgccctcactgtccatgaaggggtacagcttcacatctcgcagaccaaaggggtacttggcctgcgtagccaatgcggaagtcattgctgaatcacctttcgttgcaatttgattcgagctgtacccgctctgactttgttggttttgcactggagtacttctgcgtgtggtgcagttccccagtttccggatcgaagtaatggaacactacaactcccggctccctgtcggtgcaccagtggtctttgcagcgaatctcaacgtacaccttgccgtcgggatacacctttcgaacaccgtgcactttacccttgcaacggtattcgaccctggacacagcgacagtcatgttgtcagcctttggtttccggcttgtcctcaacaagctcgaatcggccgtccacacgcagcagatactgcagctgttcatcactgaacgaactcaacgggattcgtttcttgttgagggcgttccactcgaagtagtcgtccgacttgacaccagcggccttccactccttgggacccataatccgtacggtaccaacaccgttgtaccgcacgtacttgccccgaggatcagtacccttggtcgtcttgctgtagtcgtacgccggctgattttcctgcaaccgtacggttgtcggcaacccctgacgagggtcagccgtctcagcggccttgtcgtcagctttggtgctgagatccacgtcaagtttggtcggcgggttgagcaccggcttgtcctctgccacgtcttctcctaaatcgatgggactgagtagatttggtagctggcgtgtcgcgtgatagtgttgaagccatcatccaccaggtcgggtgacctgccgccaagttgcacgaacgacagcttgtaaccatcattccctgcaacgtctctgagccctctgagaacgtcatccatgcggtctaaaaccctagtgaccttattgaagtcgttactcagctcaattgggtagtgtacccacgcagtaaggttgcgaggcccgcgctcgggttcactaccccacaggggcttcgcctcgtcctcccagcgcaagatagcgaaagctgtaccgttagtagggcgctcttccttggaccagttgtggaacaccgtattagaattgattccatagccgttcagggtggcgtcggcaatgagagcgttgaacaaggcagctctactcacgaggcacccccgtatccccaagcttccctaggcgagactcaatgcgttgcatcagaatcttgccaatatagttgatggtaggcaacaggattgcgtacttgccggaccaccgggtctcaagaaagatgccgtagaacacactgtgtgccatggtcaaatcaaacgcgttgcctgcgtcgattgaccgggccttggcgtgtagcccagcacgagcgttgccactacggtctgtccacggcgcgttcttcttagcgtaactagtggctaacggctcagctcgcttgaactcaaaagccatggcacgcttgaccttctggtcatgctctttcatggcacgggccatctggtcaatgcctgtgatctggatctgagcgctactacgtgccatatgccgggtccttacctacggccactatggcagcgcgcttctcatacccgttgtcaggcatgatcgttgtcacccggtacaccattccgttgtgagtccaggtgtccccgatggcgattgcggcgttgtatgcgccgacaagctcataatcccattggtgcagaagggctccctcagccgtccggagtccacctgatggttgctcaacggagaattgctgaggaggcctaggggtttgggcctcattgtcgtacacgccaccacccttgtgggtcttctcgtgcggtacgagaacgatagtgtggctatcgatcttgaggccaatattggtgcctttacggagtgcattgagcaggctagccttcatttcgaccacggcctttcaatgcttccgaaagtgatgttcttgtgacggttttggttagcactgggatccagcggctccatgccgtcagggttggtctgtatcaccgtatcccagtaggcaagcatttccttagcctgcctatggatctgggacaacggctttgaaaggtctaggtactcagcagtttcctgtgtacgctgataccaaaagaacctcacgacttgagctggcttcaaacccgtagagagcaattcctctacgtactggtctgtccaaccatagccaccatcatcttccgacagtttggcgtctgtcggaaggtgacggttgactagagctacaagctcactcgttgtcgttggcatcccttgtatcctggaggtactgggccagcgtggaccgcagctcttgctcgttgccacgagtagaaaggttgtgctgcttcagttctgccttcaggtcttccaccttcaggttgagaacgtgctcgtagatgtcctgatccaggctcagcgtaacgccgtcgtcctcgtcgtcctcaggctcaggggcctgcccctcacgaggaccaccggggccgaacatcttaccgttcgctgcaacctcgtgccccaggcaccgggccagcttgtaggccacctcttcatcagtccacaggtggtcgtttcgaatcactcgagacatgttgtcccccttcgatatttggtctgaggaaggccagtgaccagaagccactggccctcctcagtggaaccgattgctaggcaatcagatccagaccacgggtgtactgctcgggaggttcgtaagcgccgtcctccttgatctggacgaccacggcaccaccgcgctggcggatgccggtaccgaaagccctagtgtagtaggactcgatcagcgggtagccagcgtcacggcccggcagaatgcgcaggccacggtacaccgggttcttgtgctcccggaagcctacgaggttctgcagattgccggctccaccagcgccgaacatcaggaagtacccgtcaggaatgaacggctgctcgatgatcaggatgtcatcgtagctgccgctcacccgcagaccttgccaccagtcgggcggaagagtgccgatgagtcccaacggcccatcgaggaactgggcaggctggttgcgagagggcacgaaatcgtaattggcctcaacgccgttgttgttgacctggcccatccggaacttccggacttccttgatctgacgacggttcatcagacagatgatctgcgtaccgttttcgatgctgtaaccatgatggaacagatgctcgtacgcatcttccaggtcaccgctgtcgatgagggtattgccgctcacgaggtagtggtcgtgtgtgccgtcgaaggtgttgtctccgtaggacggcggcacggtgccatcaccgttgtagagtgggtacaccggatacgggttgttccgaatgtccgtctcgcgattgcggttgtcgaagatggcctccatgaccttccggaacacgaggcgctcgtcggcctgcagcatggcgttgttgaccgcacgcacctgacgggcgtccatgtctcgcaacgccttccaggtgaagcgggtggcccggtcgtagtccttgaagtcgaaaccaagctggaagtagccaagcttcagccgttcgccttcaggctcaccgaattccgatgcttcctcgaaggtagcttcgccaacctgaggcacttcctcgatttcctttgtaacctggtaggtcagtagggaaaccagcttgttccggccctggttccagatcgtaagggcctgctgcgcttcagcccacagcgcgttgaggtcgacgttgtcgttcgtcgtgtagacgatgtcgccctcggtgccgtaaccgcctgcggtgtcttctccgccgaacggaagtcgcgtcaggagtgagcgaagagaggggttgtcccgcaacatcttcaggtcactttcaagcagtgcagtgttcatcagtgctcaaccctcacaatcaggcgctgaccggacaccgtatggcctacgcgggtaaggccagtggttgaacctccggcggtcgtggtggaaacggtagcgttgctaccggtcatctgcggaacgtcggtgtcattgtaccgtccggtgaacttgactgcgtaggggccaccggcttcaccggtcaccacaacgtcaccaggatcgatgttgctcaggccttccagagccgtctgaacctctgcagcagtggcgttcccgggaatgcttgccgtcgggccagcgccgtcccagctgagggtgaaggggttctcgtcgtcggtgacggtaaccgtctgcacctcttcgttacccgagctgacattgcccaagtcgtccgaaaagtacacggtaccaggctcaccgaaatccacgcccggaacaccaagcgtaggaccgaattccacgatctcaccgctggtcataacgtcgatgatttcaccggccttgcgtgccttggtcaaaacgagcacgcccgtgatgccagagacaccagcgccaatgaccactcggccctgggcgtcatgcccaacgccaagcaagcgttcacgattcgatgccgagaagtcggatgccaggggagcccggtatcctccattcttcggatcgtacttgtcgtacctactcactgtatccctttcagtctcggtaggaacagattacctagcgccgctatcagatacggccagcgagcgccgggtagttttctgccagtgccttagtgtcggtttcgttcccggcacccccactggtcccgccctgaccgggctgaaagcctgtaacgccgccgtttccaccagcctgctgctgacggccaccggcgttaccaccctggttcccattgtctacagaagacttgtcttccttgaccaggaacgggtgctgcttggcaacctttggaagggctgccttcaacccctcaaccttgccctcgtcattcacggcaatgtccggattgatttgagctgcaacggtagccaccatctccacgtcgtgccatgaccatcggccatcgtcacgaatggcagcctcaatggcctgcttggcaactgtagcacgtagagttgcaatggtgttgtcgcgatcctcaacgtccttgcggagattggtgacctcgtcgttcttcttccgctcttcagcgtcgaccttggcctgtgcctctttacgggccttttcagccgccttggcttcctgggccttcttcttcgccaaagccttaatctccgtaggagtcaagccggcgaattcgtcgtcatcgtcgtcggtgtcgtcaccagtagacgaactggaggtgtcagccttgcctgtattgccttgggtgccaccggtacccgtagcgccaccggctgcgccatcgccagaaccagcgccgtcgccagacgtatcatcatctgtctcaccgccaaacggcgggatgggccacagggcgaaagcttcggaggatttcattgcgtctctaaccttttcagtggtcggcaggctcgcttgaaggcgatgataccccacggtcggccgcgcctgggggttggcacgcgaacgaaatcgccggaaactattgcagtcctagggcttcctgttccacttcacgctgaaatgcgttctgcgcctgtgccactaggtcgtccgttgcgctcgggtacgccttagccaccgcgttgatggcctgcatagtatccgtagacaaccctatgccaaaggggcacatgcggccgtgttctttagcgttagtaagtgctcggttaaatgcgggcctgctgtcgaaattgtccgggatcacatcacgccttcctgttgtaacttcttacgtttcctcatgagccacttgtggactgccttagatgttggcttggccccgtccccgttacgctccacgtccgtaaatgattctgccactagctcagccgggttgaatccccagtcattaggtttactcaccgagtagccgctgggggcaccgccaatgtgcctgtagaatgctttcttctcccctgtttcagggtggtagtcctcgatcacatctcgttcgccgagccacctggataccaattccttacggccagcaagggtgtggttatccttcttgtacgctggattggccttggcgtactcactctccataatggcgttcagatctttccaggaatccatacgcgtgtaacgccaatccaacacatggccaaactcgtgcgtgatggtggcatcccacgggttggtgcctgccccagtggtggtccaacgccttgtctcaccactcttcttgctcttctccatttgagcatagttgttggcgaacttgacgttgagacgcagcttcgatgaaccgtcccactccggttggacgtgagcataggccccgtccatagcatcgttgggccatatctcaacgtgagtcagtgtgtccagggcctctgggtacttggaaatgttcttctctaggccaagagcaatttccttggcggacgcaagatcaaggtgctgatcgtcccagttgttaagttgaaaatagtactcttccttgaggattgtgcgaatctcgaagatgtccttggactcagagatacgttgccgcacagtcttgaactctggctcaggttcaacgacaggggagccggcgactttcttagtgtcgctaggatctacgtgtgtggcagcactgacaatcttgtcttcctggatcagctcaaaatccggaattgcctgtccctcaggagtaaaggcctcgaattcccgaacgtcacgttgctccacatccaggatgtagtagtggcctcccgatggctcactacgctttacgccgccattgaccttgagccaccccatggtgacctgccgggtggaatcaatctctgctgccttggtgccctctttaaaccgtagcaacacacgcgtttccgtgttcttcaacgcagcagcagcctcgaaagcgtctgtagcagtagtggatacctgcgtcagtgggatgctgatgtcggtggagccggtgatcagccgctcaatgtcagcgccctcgagattgatagcgtggaatagctcttgggtgctgccatcagagtaagcaatgcgtgccgttagctctcgaccaacatccaaggccattgagtccgatgcgtcgacctcaaggtcatgtcctagtatggctttcttgccggctgaggacaggccacccctatagcctttagtctcaacaattctggaaccctcacgggccttgttcaggaaggcaagccgggcaacgttagccttccagatggaattggttgtcgccctaacggtttcgttcgcagcagccttagccaccaaaggtccagtccagcccttgggttgcgcggccttagtggtggctggttcgggcggcgacggaatcatgtcgaacggctctgggccacccttcaaggaaccaattgcctcagtaagaggtactgcgtcatcgccgccttcaatgaagttgtccaggtagtcgtctagctgcccgctgttgaacatgtgctccagagcatcgtcatcggcctcttcaggcgtgacgaaacaccggcagtgcggatggggcttatccggtaccttgtctatgggaaacaggccctgcttggcatagtgttcgcatgggtccttagggtcatccatatgaaccttgctcaggttccaacgcatggacgttacccacggcttttcctgtgcgtccctaatactttgggcgtggaacgcgttgttaatctcggtgcgtgccaatcgcttagcagcgtaggtcaccccacctggagtcttcgggtctattaggtgccgtacatctctggctagcttctcgaagctgtctccacgggccagcgcgttgttgatggcacggtcaaccatgccctgagacagcgctctagtcttccaaaccttcttggacagagggcgattggtctcgagcacacgagtcatggtggcttcaatgttccgttcggccgtggcgcgcaggcttgccgcgtactgtgcacggtcaacgggattgggcaaaatcttagaaagcaaacgctcttcgtcttttagggcagcatccacagctgccttggccgcgctgctctgggacgtcttgatgatgtccgacacgtcaccgtacaggctgttcatgcgctttcgaatctctttgtgagccaacgcaagctgtagccgtcgggtggctgcgctcttgctcttcacatccttgagcgaagcaagagtggtgtcaatgtctttggtagcctccaccagggctgcgaacagcctcttgtcagcacccttttcgacagccaagtaccggccaagccagttctcccggatagccttagtgttaaccttgggtgttctaggaagcggtagagtcactatggaacctcatccaccagctatggtcctgtggtgcgtaaagcttagtgtcatactgataaacgccggctgaggtcacgatccagtaatcccagttgcgttgaattgcaccaagtttgatgccgtcaagatcagtggcgctggggtgtgtggtgccaccggggtgagtgtgccagatgcccttgatgaattcaaccttgtcagatagcttctcaacaagctctttgacgtccatcttgaacgccctacggggagctaggcttacgttggtcatctcgaggatctcaccatcagtcatgatgaaaccacacgcctcaaacggtgctgcctgtatcgccctgcgtatcaaccaatctcgggcttcaatgtccatggggctctacttcttcactttgtatccagcggcagagtagattttatcaacagaggccctggactttccggcgtaaacccctctggtaatacccattacagcgctctttgccactggggccgaccccttttcggctctgcgcttagccctgcgctcttggtcggctttgatcattgcagcctttgcagccttcttgctggccttcactggcttgttacccttgcctggggcatcgtgagcacgcatggggtggctcttgctagaggcagacgacttcttctcgccacgcataatggccttgaagtctttatctgacacactggcattggcgcgccgagtggctgcagcacttgcacgcttgctacggccctctgccttggctgctgcaattgccttgccctgagccttctttgccctagcagccctagccttctcaagattcttgcgggctgcagccttctgctttgctgttgccatttttaccccttacttggtcacttccagccaatacgcaaacggctgcgtctttcgaattccctaagcaccctacggccctgacgcgcaggcttaccacgaagggtcttgctcttgaggttgctgtgaatccaatccagatccttactcgtcggcctttccccgctgagcaacttgcctgaagccctagtgatttgggacggcatctccctgcccttaccgataaccttggcgttcaccccacggatacccagcttggaagtgggttttgtgccgctccgcctagcggctctagccttctcgagatttcggcgtgaagcggccttctgctttgcggtcgccatgtgttcctacctcttacgtgttttcttgggcctgacgctgcgacgagactccaggctccaggccttcttgaggtacgaatgattacccgttgcaacgaatttggctctggccctttcggcctgccccttaaggctttccagcttgccctgtggcttctttcgacgcttgcgggccttctccaggttggcttttgctgctgccttctgcttggcagtagtcacctttcgcttaggcatcctacttctttcccttcttacggggaagctgccgcttccacttcttcctcccgctatcagagatgttgcggtgagccatctttcgggcaaggcgagggtgcttggcaaacatgtaacctacctgtgcctttgagctgaagcggcgcttcttcttgcgggccatgtcacacctcaaatgccagctgtgaaccgtttagtgttcctatgtcgctgaagtcgtcaaccccagcgtcagcctcgccactggtgttgcccatggccgtgtcggcaatcgcccgcgcctctgcaagcagcttctcggtgatacccggcttgtactcgtaccctagcttttccatttcctgtcgaacctcgtcgatcaggagcaggcctgcggtcttgagtgttaccaactcttcgattttggactcacgattgaccggcatgggatcgccgaacaccgtttcccatacggtgccattgctttccagctcttcaaaggcaggcaaccaaccttggatcagatcgtgcccaatctggtcccacttgccgccgatggtgagctgcttgtcctcattctcagagatgatcggccccatctccagagccagagcaatgccgctttgagctactgacacatcgactttgccgctagccatatctggcaggccaaggcccttcgcggcgtagtcgtccatccactgcatgtgctcctggaacggcgtcacgctggccacgcccgtaacccgttggaaagtatcgtcctgtccaatctccaccacattgccggggtgaagcttgtactcaccgggtttgccgtcagggccagtgggcgggccagcactggacacgtacacgcctaggccctgaagcactagggacagatcttcgtacgtggtgctctggttgagagcgttgatgatgtactcgatgccagccacttggctagtgccccaagtggacccgtccggtacgttgttcggaatgtggtacacggggatggtttgaatctgcttgggcagcagcttctttggcatcagaatcttcacgccctgcaacggcttcagctcgtctggcttcaggttgcggtcgtcccacccgccaagctcgaaaagccttgcctcactagtgatgccgccgtcctctttgcggtacgtctggcgacgggccaccagcttactgcggtctgggtaggcagccgtgctctcacgaggatcgggcaccaggtccaccaggtggcacccaatcaccctatccccgtcctcaattgggaagtagcagctggggtgcatggtgtgaatgctgattcgcgtgccacgctgtttgctcggatcagcggtgatgtgccagatagcatctccacgcgtcaacccggctttcttcgccgttacgaacttgcgttgcagctcttctcgcttgttcaggttgtcgaagtaggcccgcagctcagaagcgttgcctcccttaaggttccagtcgaacttgactgccaggaaccgggccgtggagtcaatgatcttcttcgttgagggcagatagatctctacgtcgctggagccacgcaccgtaacctgaaatgtctcagggcgattgtggtagaagtcgtcgtacaggttgtacgccttcacgcgggcctggtccatcgggtcaacaatgttgcccagtaggtttccatagcctttcagaaaggcgatggcgctgtcgtattgcttagggatatatggcatttatcgtgccctccgaatctttactccagcaagatctttggggcgcacccaagaccacttttcgctcttatccgacaaaggcgtagggtccttacgcaacttgtggcggttcaccctcacagcgaacgtggcagaattcccgaccaccagcaagcgagctaggctgcctttcttgccctcgggcataccctttcgcttagccttcctagccttctcaaggtttcgctttgaggccgcgcgctgcttcgcagtagtgaccctacgcttaggcatcgtcgtacaccccattcaggaagttggacagcgcgttgttgaccttggggaactgggtgcgacggcgctcatgcgctgcaggcctacggaactcttcgctcttgtaccagtccctaggcacctcaaccttagcctgctcatcgtcacggctgaatgttgctgtgtggatacgagttccgttcttaccgaatgtcccaaagtagccggcacaaaagcgccccagggcttctgggccgtggtcgtgatccttcaaaggcttatcggccctatcgcttccaggttcccgcatgtcgttcttgtcggggtaccggtacgcgagcatgtcttcgatagtcttcttacactttcgatcaaacatcagccgtggccggtactggcccaccgtgagaccgtcgaggctaacttccacgtacggttcatccacgacaaccctaggctccctaaggaatttgcgaatccagttgagccggttgcgcaactctccgccggtgccaccccgagcacgcacattcagtttcttctctaggattttggtgctgcctgggtcagccgggtcagggtatatgtacttgaggtttgacgggttaaggccacggtatcgaatctcatcagcgaaatcctctgggtccaagccagactcgtatatctcatcgagtacgttaatctcgccccacttgcccacctgaatgagtagccacacattgggattgttaaacccatagtcaacagccgcgtaggtctcccaccctgggttgtactggaggtctgtaacgtgaacttcctcatcgaattccttgaagacgcggccgacgaattcggtaaagtcagctgcaatctcctgcaggaacagctcttctggcaggtcggcgattcggttgagaatctccttgtctatcaccaggttctgctctttagccagcttgtgagcactgttgcgcgtacggcgtacctgctctagcagtttgtgcacgtggtcttcacgcgtgggagtcttgtacacgtggtagttgacccacgagggcatacgccagctctgccactctgtgttgtatggatcttgggcatgacgccacagctcgtagaaccagtttttgccctctggcgtagagccaaagtaggaccagccgtcaaagtctgccagcgtgggctgaatgtacttgggccagatgatgtttcgcatcttggcggcctcacacatgatcacgcctgaaaggccctcacccactaggcgttccggcacctgggcactcttggcgatgatttccagtgcgccaccccacagcttgatctgcatgaggcctgtctcagggctattgaaactacccttctccatagggatttcgagccggcggcaccagttccatatgacgcgaaaggtcttctctgactcctggtacgacggcccgacaatccagaagatacgcttcttgccccggtccaaaagcgtagaggctatagggcgagtgaagaacacctctggcagcagcttgtcagtaccggcgtagtagtccttgccaagacggcgtccaccggccacaaccttgtgccgtgcgttgctttgagcaatggtggcctgtgcggggttaggcttccagtcaatcagtgggtggctgtacagcttccacttgtccagtaccgctaccataggtgcaccgtaggcgtactaggctttacgattgccggctgggtagccttgcagccacaccacacgttgagggctcggcaagccggacggccgcagtcgtcgctacgccgctttcccgtaggcagactagccaagtggcgtgccatcgtgacttcctccttggctagtcctgaaaaccctactgagagctgcccttgtcatcaggccggtcgctgaagcaagcaaagcgcacgcggccaacgttgtccagagcgctggtgaagcgtagcttgcgccgaaagtagggatcgccggcacagacgaatctgaatgccttgctacgctgctcaacagagctgttcgtagtgacattggtcaaatgcctcgcctccctacatgattgtggcgctagctgggcagtggcccttcgctgcgcggtggggcgctcaccttccggcctggagtgctcgcttccggcgcggggaacttgcgcttgctcttgtgctggaacaccttgcgttccaggtcacttgtgttgtcgatcggatcacgttggattggattgacctgatcagcgccaccaatgaccttgatgccaccaggtcccttgcggactcgaagagccatgagacactcctgttctcgtacgcctttggtactctacacttttcgagcctacccgctcttttgccgtcacgcttgaggatacccgatgccaggccacggcgataggcccgcgctggggcgcttgctcagaaactactgggagtcaccaagtttcacatcaggtggaaggctggggcgctcaagggcatcagggacaatgtcaacgtccaagggctccataggcttaacggcgatggcctcgaacacctgctcaaagcctgccttagcctggaccgaaaccgtaagtggcgctttgccaatggtgcggtcaaggatctcggtagcggccttgatacgcacgttcggctctaccgtctcgtcaatcatgatgctgctcaaggtctctgctgcagccagcgcgttgctcttgagcttgtccaggctgcggtcgaagatggcctgcgtaaacgcctgcaccatctcgcgaggcacgttcttaggcttgaagattttgcctgtggcagtacggaatccgaacgacatgagttcctcgtcgtccaggtcatcaatggtgatctgcttggccaggaacagctggtagcggctcattggcgcactggtctctgcttcctctgtctcagtgatcagccggtccagagggtgcagccgtgccccgtgggttgtgcagaacgggtagcggttggctgccttacgggagcaacgtcttccgtcgtcgtcaagcttcttggtgcacaaggcccagccgaacttgtccacccggtacttgcggttaggatcgtagttagccaacatctctggcttgaacgagccacgcatgttcagcggcacatggtggtccagtgctgggttgcggcctccaggctcttgcgtgaaggttgccacctcaatgccgtagctggcgatgaactttggaggcccgtccggcacgccaacgtagcgaacctctggcaagtcgtcgttgtcgttcactgctttgccttttccggtatcttccattgggattcggtgccgccgcacaggccacagactaccagccggcggttgtcgctgattgtcttagtcttccagtgcttctggtgctcgcgacggcacttgcgattcatcatctaactcgtccacctcctcaccgtgacggctctactgccatattgcctgtgcctccttgttgctgtggccccagccatcatgtggcgcattgagttcacaggggtagaacaggccgccaagtgtcagtccacctgtgcagctaggcaatacgtgctgcgccttgggccaaccacagttgacgcaacgcctccagggctctactggccctatgccccactcccgtttgcccacgaagttttcgcacacggcttcatgggcctctgctcccttgccgtccactaagcatacagggcatagggtttcgtgactggcattgtcacgaggattaccacagctgtggcatttccgaatctcgttgttactcatcgtccttttcctcggactctcgcttctcggcccgcattctcattccaccatagcaggctgggtgccagcaacagggtccacgccactgttgccagcacccagaaccatagtgctgcacgagcatacgtcatgccacgaactcttcccgttcctgtagtttgttagccagctgcctaagctgccgtctcttgcgcagacattgggccacccggcacaggttgtagccgaaagcgatgctacacagcgcacttccagccaccgtgaccccgtacccccaggcattggttacatcggaaaatgtagcaagcctaaagaagaacagggccgtccatgctcccaaactcaccagcatccacagtcgtggatctgtctttcccagtgacatactcaccctagggtttctctcggttcgcgtatgtcaggtatcccctaccctgactaggtcagtagttttgccggctgaacatccccttcctcttcgcgcgcccctgcggttggtcgtacaggctaggctcaaacggtacacccgtgggagtcaaacccgtactggcacttgagtactcgtcggtacccaagtgcacgtttgcaaagcggccttcaccacttgcggacaagcttcttgggtggcgggtcttggcgataggagacattgcgttccgttggtgcatctgggctgaccaacggcggcgcagcgccgtacctatccggattcccaagtgtgtcaaagtccctgttgctatcaggatcagtaggatcagtacgcctgtctgccacggctcgagcgtaattgtcactggcgtctcctgccttgtctctcagtcgcggtggcaccatgttggcaccaaagtcgtcctgtggcctacgctggccttcaccgttggcgataccgggcgggaacattcctggcccccagctgtattcactgttgccgtcgggcaagtcgttgggattaagtggcactagagtttccttcctcttgagcctgctgctgccgtgcggccatgttcattgcacaggcgttgttgtccaagtggatatcaccacatttggcgcagcgcagtaaatcaatctgcgacgcaacgcttttcatcgggtccatgtgtcctcctcacaccagctgtcgatcttggcctccagcgcactgtgcagccagtccacggcctcaaagaccatgcaagccagcacaaaccgaatgtccttgagcaccttaaccactcttcctcaatgccttaggggcagtcatttgttcgccctatagaacgcctgcgcaaagcccttgggagtaatcgccctaatgcctgctctgtctaggcccaggtgtgcgtactccggtggacactgtggcctgtaccagccgtcagtgtgccggtactggtctcgctgctggttcgtgtgtctagccggccgttcttctactgtccgtaagggattgttgaagtagccccacagatcagtcttcttggaccacatggcctctgggtcaaaccaccaagcctcgaaggtgtagcgcggatgtccgaggaacttgcgcagatggccgaacgggttctccatggcccaaaacttggtgtagcccttgcacattgcagcccgaatgatgtcttcacaagcgcgtactggcactaggccttccgcaaagttgggtcgcagtgcgtatgtctgcctagtgttagccaagctgaactgtgtgcagggaggggcagcgaggatgccgtggatacgcacgagctttacgaccacatctgagacactagggttctgggacaggaacactacctccctgtcgctgatgttgaccttggttacgtcgtagtcaggcaacgtgatccgaatcacgttgtagcccttgatccgatacggctcactccacgcgcctgtgcccgcacacaggtccagtatggtcttcatcagtcctgctgtgtccctttgccttccgcctgcttgtctgccagcttggcctctgctgcctgcagtcgggccttaagagcatcatacgccgccgcgcgcaaatccgctagaggctcaacgggattcgccacatagtcttccaggtaccagatggctgacttgatatccctagcatctcgctccacctgtgtcctactgtcccacggctctgccttaccaccgaaagccacgcgccaaatgtacttgaaagctgtagcgagcctagggtcctgcatgtgccggaacacatcgatgcactggatgtcgtagaactcagcagtctcatggtcactcagaaacaccctaggcccgcgctggtagtgcggcggactcacagcgtcataaggcttggccacctcgtacctttccggcatggccatctctgcaacgtggccggggcagtttcccttgcggcattggtcacactgctgctgagcgatacgaatcacattgcggccgtagctctttgagtcgtcccacagcggcaacggggaaccgtattccgagatgctacggagcacctgttggaatgcagggtcatccacactgagctctactggaccattcacgatgtctccctctgtaatacggtcggctggattagagatcttctgatgtagaggatctccttcaaaactcgtgtcagctgcatgacttacggcgtcaacagcgttattctttacctgcgcatccattttcgatccctctcttttgtcgccggctgaaacctttagagttgcaatgtcgttcgtcatttgtctaaagcctatcctcagtttgatttgtctatttgcccaggtcagaggctcgcccgcgaaaagtgaggatggccgcaacgccctgaccagtgcaaatgggccgtatcatcagtcctcagtttcggctgacttcacagcgtctacatgaaaatgtgtgcgcggaaagaaatgcatgtgtgtgcattactttagagcacattgcgtattttcttccctctctacatcattctatttaaagtgaggactgatgattagcaaacttgaaaaatgccctgacctgggacgatgtctcccgcagcaactcatcacctttcatcatcacttccatttccccaggtcagcctgttgatcaacctgaggaaaccttctcttcagtcggcccgcttcacccttgcaacggttggtgcgaaactccaaccaagccatcgccgttggttgttcgctgtccagctgccgtcctgcacccaaccactgtccttcagcttgcgtgtgaacttagtcttactaagcttctcgcttggcgcgatgccgttggatgtgcaccacctttcgtactgtttgtacaagtctcgatctccaaggcacagctcgtcgggccaccgtggcttggggttctccacgtccagacggtcgttgcgtgcgtacttcttccaggcactgggtggtgacgggaccactgtctccttcaggaagtccgacagctcgcttaggctggacacgaattccttagtctcaaactgcagctcgtcgggcacgatgtgaacaccctccaccagaccgcgggaggcgaactctaccaacgttgctagcatggcaaaccggcactgcacaggcagatcgtcaatggcgttggggtcacgctcctcgtcggtgacctggtgcctaaagggtacgaccacaaatcgttcctttgtcgcttcgtccacgtctggcacctctggggcctggttgcagccagagatgatggtacacctaggcttcagcgtcaccggcttgttgatgttcttgagctggcacgacacctcgtctccacctgtgatacgcttgaataggcctgcgtctatatgcttttggcccagttctcccaccatcatgaccctgcgagacagggcctctgccagcgcggacttgaacgttgtgtcctcgaatacggctggttcgcgccaaatcgataggtcccctagggcgtggtccatcatgttctgcagggtgctcttgccggtatgtggcaaacccactaggaacacagcctttttggtgttcacgccgaggattgaaacccctagcagcttaagggtgtactcccacgcctctttgccaagctggctgcgcaggaatttgttcatgtactcccagaaccgtttgtagccttcacgaacctcagcgtcggggtggtctttctgctctcgcaacggaatgtacggcacatgcgtgttctgcgtgaccagctggtccttgatcacgtctggatcttgtacccactcaaagggttccgcgccagccagcacctcttccctggtgtggaacctgatcactccgttggccactggcagcaacattgggttgttgtccagctcttcgtacaacagtgcaatgtccggaaagctttgggcctgagccaggctgttgctcacgcgggccttgttgccgctttcaagcgcgaacgccacccacgccctggcctctgccagctctgccttctgggcaggcgtagcagcagctgtaccaccggcggcagccacagcagcagcagccagttgcaaccggcgctgagcatcccgttgctgcctggtcttcacgcgccggaacatatggcgcactagggtttccttgttgtccaccacccaacggccgtccctgaacagcatccacctaccgccttgggactcagggtagttggggacaaaccgcacgttgccacggttgagatgcaggaagtgctgtgcgttgccgtcctcgttgcgctcgtattgcgtaggccttttaggaggtttgcgttgcttgggaggctcccagttgcgacggcactcacaggggtccagagtctctaggtactctaggcctttgcgcttgaactgaacgtactcacccttgatctttcgcagcgtgccgtagcgggaccgggcaatctcgtacttggtttcggcaacgctccgaacctgatctgccgctacacgatccgcccagctctgctcgagaacgtggcaggctgtggcccagcctgagtgtccttctgcccctaggcgcaacaggctccagtgcgcacggataagcggctcgtggtggtcgctggcactgtccacgtcttccaggtgccggttgagggcgttggtcatgatgtcgcacatgtcttcaccgtcgacccagcgcttcttctgccagttgcgtagctgtgcattagtgctctgcatgtcgatcacagcgccaccggcgttggcaacgccgttgcgagtcaagaacttccaccagcgctccggcagtacagccagctcttgcaccctagggatacggtcgttgacgtggatgccgtcaggggcctgtcccctatcgtaccaacggtattgcatgccgtcggggtgcatcgaagggaacaccaggccgtaacggtgcactctctgtacgatttcgatactatcgctggcgcggcctgtggatggcagcaccggcttgcccatgtactcgtagccaactggagtcaggaaccagcggatgccgctcttgccgtccgagcgcgccgagcttgtccacgtatttggtagtggcccataggttttgatcagctcttccaggtgagccgcgccgctcttcccgtcgtagtcgtcaacgtcaatccctatgacctcgtaggcctcaccgtcgttggtgacctcgttgccgggccgtaccgcaatgttgcccttgcgccaccgcttgtcctgtatccaggcatctatgtcatcgcgtgtggcaagtcggccgttgcgccccgtaaaccctagaccctgttcgtcgtcctctttgctgggtggtgacttcttcttgttgggaggcaacggcagaacgttccaccccttcaaggtgtactccaaggccgctttcgcgaatggtgctgccgctccgatatttttgggtgcgttcaccgccatggtctcactttcccgggatgcttagagtgccattgtttgtcaagctcattctcctcgtgtgaaccccacttaaggttctgaagacggttgtcgcttggtataccgttcaggtgcctgcactgagtcttgccgggagggcaagggcctctataggttagtaaaatgagttggtgaacagcgtagcgtttggacctaccatttctcgtgatatgtacgtacgcatagccatagcggtcaagtgtcggagtcagctcaccctgtggccctaggattgtgccgtcttttaaggcaaagtaattgggtgcgtaaggaatctctctgggtttcaagtgaatctagccatcctcgtctagatgattttgcgcacggcgtgtgttcggccgtgaggttgacgctctgtgatgctctgacctgcgagaacgtgatcgcgaagtttacgctgatcaggggttatgtccaccaccatctttggggtgcgcagcccgcgtgcgacaccagatgatgtgtttcctaaggtcagagcaccacagagcatgtcagccggcgactacagtagcaacgctagggctttcagtgcccacagcaactcgtacaaagcctccctagtggactcgtcctccacccctgcaatcgagtctgccacagaattgaaacgctctgcaagcgtaggcccttcaggcattacaccctcctcacgatttccactacgctcactgcagtgcccccaagatgtccggcccggtccagccttccggcttgaggaccttaccgtccgtgcggcgaacgataggccctagggacccatcaactttggacaggttggagcgtgccacctcgtgagccgccaaccgtgcccggtcctctccaacgtaggaaagcaatgtgccccaggcgatcacgatgatgtccagcaggccgtccacaatctcgattaggtcattgccgtcctctgcgtcgaagtattccttgacctcttctcggagcaaatctttacgcagactacgcaactgctgcagctcggcgtagctgatcatgccctcgccgctttcggtgccaatccactgatcgatgggaacctctgcaagcttatggaactgggccgtagcttccaacaggttgtggaatcccgatggggcactttcgccttcgatgatggtcatgtcgcccatcaggcccagctctttctccggcaccggcttgtcactgctgtagaacgcctctcgccatgcctgcgtaaggtgctcagccggcactcccctgtaccgtagcaacattagagcaaactgcttgagcttgacggtctggacctcaagcttggccacgcgctcttccagtgtgctggtagggttttcggtcactagcttctcctaacggtgatggtaacgtacgactggtcacgctggtccattgcgttgtggcgttggatagctacggtgttctgctctccgttgggagacactgattctttggccctttgcaactctgccactagttcgtcaacgctcgggtctccgtacaccgtgaacttgtagctgtggtcccgagtgattcggttactcatggtatctccctagtgttttgggctgcctcttgcatgtcggctgccagctgtcgcaacgcctgaaccctgtgccaggggtcatcgacctcgtccagtatgcgcaacaaatccctagcggcactttggaactggacgaccatgacgttgaccagctcccggtagcgctcggctgcctgctggtacgctgtggcattgttgagcgtgcgactgatctcttcaggggtgaggcttgaggccgtgcccaatacgctcaacgggatgataatctggatgtcactcactgctattgctcatttcacatcgaaggtaatctggttgctgccctacgttgtccgcgaacacaatggccataatgccaaccaccgtgatcaccaccacggaaagcgtagcggcagccacctttacgatttcgttcatgcgctggcatcctccagctcttcctcgtccaccggctcatccccaatgacaccgtacagctctggccaagccacacgcttcaggccgacaccctgcgggccaatgcaccggcgcatgaacatcaccgcgtggcggtcagcgtctcttgcgtggtccggcaggccgtcttctgcaatggctcccaacgctttcagccggtcgtccgttacccgcttggcctgtgatggatcctgcttgaacatgcacctgtcggtgttacgccataggtacgcttcaagtttggcggtgatgcgcaccggggacaacagctctcggctcttctcctttcgttctacacgtaagatgaagtcttccaccacgattgcggcccctggccacgcctggcacatcttgaacagctggaagctggctgcgtcctcgtcgccagtggtgaccatctccccatgctcccaaataacggcgttgcgtagtatggtcgctggatcgtagctgaacacgtcacggcccctgatctgccgtggcaacacaagcaagctccagcccgtggtgccgcctgggtcaaacgccagaatcggtacccgagtaaggtttacgcctgccaacggccttgccttgggcctgtcagggtagtggattcgcagatcgcggcgcattgggccgtagtatccggccggacgctcaaccgtggactgcctgtatgacatagtcttcctacccctttcagcctgcggcctgaatcgtagtgtctaccacgtacacaccgtcggcctcttcgattgctgccagcacgccttgtggtgtcttcacctctgcgatggtatcccgtagccacgcaatggctttgcgcttgtagctgggcatctcctccagaatgtccttgtccaacttcagttccaggctgatcaccagccgtgtctcttggatgccgtgaaagtgggtctcgctcaccagctttcggccgtgaatatcaggcatttccaacctccactcgctgcgcgggcttggttccctggtacaggtacgttgtgctgtctggcttccgatagtacacctcttggatgcctttctggaagtcgttgaacagcaactcctttggatagggcttgccgtatgtgaaggtacgcatgtgctttggcccacctacacacgttaccgttgtcttttgacccttcttagccatcgatgcccaacctttcgtcgctgcgctgaagctcatgcgccacactggaactacgcagaattgccagcgctgctgccataccttcccgacggtgcttgttcttcaaccagttagcgttgctactagtttcgccggttttgaccacgatggactccatgactttccggcgctggtccatctgctcttcgatcttgctgattagtgtgcggccaccgtagccaccgaaactcttagccagagtagcgcttttgaccgtctggtcctgctggatgcggcgtaacgtatcgccctctcgcacaggggctttacctgtcttggccctcttcttggccttcagcgccgccaccttggccagcatctcagcggcctcagcctccagctgttcggcggtcttctcctgctcagacatcagcatcccctagggtgacaagcagatcctccagcgtcggatggatgaagtccaccatcttgtacatcaggctagccttgcaaggattgtcgagcaagatgtgtgtttgcttgcccgcgccgacggcccaccccaactccaggtgagcgtcacgttcgcacggcagcaccagcaccacaatgtcagaggcgaacagagcgttcatgtctcgctggaagcccttgtctgccaaggggtgagcgatgatgtccaggtactcagttgtggacacactgtcgtggttgtcgtctacaatgtccgaccacgcaaaccccttctcaccgttggggttcttgaagtcgtacacactgtagccggcaacccttagggcctgaacgacattgggttggaattggtttcgccagctcgaagcaacgtagacgtgcggagtgggaaacatcccctgatttgccactgtatctcctagggttacttggttattgaaccgtgggtgccaaggactcgaaccttggtgtgtgccgctcaccccaccggcttacggagcagccgggtttggaacctgcagtgttggtacaacgttgttacccaacgggaaacagttgagcgtctggcctgtctcagccatcttgttgacacacgcggcaatggcaatgcgtaggtctgggattggctgtttggccaattcctctgccgcaagacgtttctgctctgccgtcttggcctcttgctcggccctagtggtgtcggcacgagcagcgttgatggcgttgatcttctgctctgtgttgtcgtcatagtcgatcaccggcacgctcaggtccaggatggtgacctgatcactcttggcttgcaactgttcaagagcatctgccgtcaccgagctgagcgtcactggctttggagcaggccccgtgggcttgccgtccggcccaacaggtggcttgggatcaccagccagaggatcaaacgccatgaacgcctcgttgagcacagtacgcagattgcgctctacaagattggaccgcacgttgtcgaacgtcttgtactgctggaacagctcgtcggcttgatccggcttgatttcccagcgcacattcacgtctgccatggcagtgctgttgttccctaggcgcacttgaattctgtggtcgccgttatacacgtcgttctgaatggcggcgtccatctcatgcaccgaggtccacggcagaatgaaatgcaatccgttgctaatggtttcgcctgtcggccgattccactgtgtcacaatgccaacctgtcgcgtacccacaatcgtggtgcaagccaacaccaaactgatcaggaacaccaaagttgaagtgccaaggccaatcagagctgctgggttacggaacgtacgtttctcgtagcgtccgtaggacacttcccgttctttggtgatcgggaagaacaggaaccatacggcaaagccaatcgtcacgagcaggacgaaggccaaccaaagagttgcccacatctggtttttcaccaaccttttctgtgttgcttagggtttcttgggttctggcagtgtgatcttgcttgggtcctcccctctgtacagttgatcgtaaagcgtacctctgctagtgatttcggcatcggcacgcgcaatgtctcgcatggggatcagcatggcctgctcaataccgtctggcccgaacgatgcctgaaagggcacaatccgaacacctagctcttcaagctgattcaggatcggcttgggcttggcgtgcaagaagttctgcagggcctgcagtcgcaacgcagcctgctcctgctggtactcagaaggcttggccttcttgaacttccccacttctggcctcagttgttgtcgaggcatctggccaggctttcgctgtgggtcaccgaaagttgcactggcactacgcaaagtctctgccaacttctgcagtttggccgcagcatcttcctggggcgtcgacggcccgttcaccacctgtaacggtttcttagcgttactaggaagctgggtcttgggctttggcttggcagccggcgtttgtgcctgtgttgctttggctttcaccctttgcttttgctttttggtcttgcgtcccatggccttacactttctggcagacagctgccgtcaggtcccgtatgtcgctgttgctaatcatgctgtactgtggtccccagttacgcttggccgctccccaggccgcaagcacgccgaacaggtcaagccagtagttgggcagcacattgaaggcctccagaacgatactgccgtattcgtcttcgtcacaggcgtgccgtaggctagcctccagtatggccaagtgccgtgcgcacaccaacgggtctgtgccaagaggtgaattctcttgttggggcatcgggtccatcgtaatgctctcggacacttcgccatccgcaatcgccctcgtggcaggcgtttcgcgtgggttgtcgtacgcgtgcatcgaggcagcccacaccttgtagctccctaggcccacaccaagttcagcagccaccagctcgtgcagcatcgtgaactcaaacacgttgaaaggcaggatacggaaagcgttattggacctcatgactgtggtcatgtgcaacgcgtaggcgtgctccccgtcagggcagggcctgagcctgaagaacataccgaatgtgcaagggatatccccactttggcgcacagcgtcttccggttgccacacaaccgcagctgcctgtctggagtacggtgaatggcgtagccgctcaacgactccctcgatctggttcaggcccggtcgtggctggaagatcctaaggccgtaatcacttccaggaacggtgagcttgtcgtcactgaagttgcggactttcggctcgtaaaacgctatgtcctcaacacgattgttgcccgccatcaaccaggtgaagcgagctacggccgtcacgatgttcaatggcctgttagggttggtgaagatacgggacctgatgtctgtcatgggaattgtttggtagtcgcatagctcccaagacgaatccttctcggttgcaatgtacatgccttggttcatgacgtgtgtcagggtcgtgctgatgagtgtgtctgcatcgctcacaggcccatacctccttggctcttgggcactccaggtgctcggagccacgtttcctcccgcttgaagctggcgtccacgtagccgtactgaagaatccccatgtctcccaacagctgcagcaagcgcttgtaggtgctccagtcctgtggcacatcaatgatcaagtgcccgtggcccggtgtgctgctgggaacgtacacgtgtgggcagtccagatccaataggaccctatgcttcccttcgctcttgtagatacgagcaccccgtatccggtcgtcaaaggggtcaacgatctcgtggaccggtgccgtgccaacaacgtagctgccgatagcgtttgccccgtcgtcaagcggtacgttatctgtcactaccttgtcgtggggcagctctcgagaggcactgtcccccaggccaaagtccacaacggcactgtgaacgttctcccagccgcgtagttcctcaagggccttctgaaatgccttgtctactacttcagaacgataagtcatcctgtgtttcatcctcttccggattgattacgatcaattccttgacctgaatggccaagcctccaaagtgagtcacgtagcctgtcaccaggattacgtccacaccagcgcgggcgttggcgataagaccggcgtactgtgggtacttcttccggctcacccgcaagcgtattggctcgtcgtactcgtcgtcgacgtccagggttgcgtacttcagtaggtgcggttcatctagctcctctaggattttctctcgggtgaggccgtctcccccgtacttaagacgctgctgcacagtgtctttgtaggtcttcttgcgcagcacgcccataaagactacgtactcatcctcttccacaatatcgatgctgacgtgtgttggctctggaacaccaggaaatttgcctgcctcaaattccctacgtatgctatcaagtaggcgtttggtggccagcacgccaaacgggtcatcgtcttctgcgaactggcgcatcacttttactctgccgtcaccaatgccagaaacggctgtaaggctagaccactcaagagcctctgttttgtgttcatcacgccactggacaatcttctcagcggtcttgaggccaatgcccttcacggctgtaaagccagcgtacaggctgccgccgcgcaagctccagcctgcctgtgactcatgcagatccaggctattgaccttaatacccttgcgctcggcctccacgatcaacttcctacggcgctccaagccttctgctgtgtcgggacacttgagtagctgccctaggtagaactcagggtagtacaccttaagccacatctgacggtaggccgtatcgctataacacacagcgtgactgatgttgaacgcgtaccctgtagcgttgaccatcttctgccacacacccacggccttctcgcgtgaaacgccgtgagacgcagcaccttccacgaacctttcgaggaactcgttgaagacacttgagtcttccttggccgagatgatcttcctaatgcggttgacctcagccggcgcgaatcctccaaagtccctaaggattagaagaatctgttcctggtaaacgatagtgccccaggtccattggatgtggtcatcaactgtggggtggaccggctctggcagctggcctgtgctcttggccttgatgtacgccgtatcgtcgccaccagggcggctcagtgcgtttacgtctaccaggtccataaacttatctggctgtacgcctttgagcacgcgccgtgttgccacaccctcaaactggaagatgcccaacacgtcaccagcgcgaaacgcctcgaaaaccttagggtcatctagtggtatctggtagaactcctcgatagtcatgccaattcggtcacgcacccactgtaggtgttccagtgtcgctcggatgccggtaagcgtagttaggctaaggaagtcaaccttcagaagacctaggtactcactgtctttcttgtcgtacgcaacgccgcttccgcgctgggcaccactctgcttttcgtaggtggctgtaatctgagtcaacggtacgccactgatcacaatgccagcggggtggataccggccgtacggtagttggactgcccgttgatgccctccagccgtggcgcgtactgcaattcaggtgtacgcctaactgtttcggcaacgtcgtcgtagctatcgaaggtgtcaagcagtgtcatggagaaccgtgggtggccgtcttgccgctccaccagcttacccttgatgttctcaatcttccactctgggatttcgtatacgcgcgctacgtcatccaggctgttcttgcccttgtaccgggtgaacgtcagaatgttagcaactctatcgtatccgtacttctcctgggcgtattgaataacctcccaacgcctctcgtcctcaaagtcgatatcgatatctggagggtctgtacgcgtagggtccaggaaccgctcaaagagcatggggtacagcagtgggtcaatctcggtgatacgcaaaagccagcaacacagactgccggctgcactaccacggcctggacccactagaatgccattgtctttggcccaacgcaccaagtcgctaaccatgagaaagtagtcgatataccctggtacgctacggattacgccgaattccatgcgcaggcgctcttggtacttcttaggttcggcctgcatctctttgctagtgcgcctacggaacttgaacccctcggcaagccagaacttgaagagtttctcagtctcctgctctgacagtacaggtttcgtcaccggaacacctcgttgaacgtgtacaccctattggtcctgatgtacctgtagaacgggtggtggcctgcgtactgctctttgcgcccatgtgctgctaggtctttggccggtatcgtggccccgttgctatagaccacgtatgccgggctgtcccagtctacacagcgttcagcctgtgcggcccgcagacagtagtcctgcccctctttgacgtggcagatgatgcctgccacgccccgatctggcttgaaggcaaagaagtccactgcctccacccaaccgcctagtctcgcaatgactaggatgtcccagcccctacgccttaggagccgtttaactttctcaactagctcctcgtgtgcgtccacacgctctacgtccatggctcccaatctctttcccccggtacatatttcggtggcggcgctttgggcagctctacattgcattggtctgcaatgtgcgccgtggcctcaacagcggccttggctcccgcagtgctgagtcccgtgcccacaaggtctctgtatacctctctatcggtttcagggtacgtgagcagtatgtcgtactcccagttagcgtcagtaacttcaacagtgctttgacggtgagccgcgtgcaagatgcgctgcatctcattgtcattcgggtaggggtaatgcacgtcagcagtagcagccattcgtgctcccgttgtgttgctaagttcttcgaaacacgggttgagagcacgagtgcgagcgagaccagaaaatcgctgggtctcaaggtaatacctgtcgccaaatatcgcctggaacctctcaattgcacgcctggctcgttgaatgtccccgtctctgggttccaatcgtttgtcacccaatgactttccacccaacaggatgcaagagatggggccgtccgcgcagcccgacagagcaatgatgccacgattgtacttcctcagtactggtgcgtgaacggtgggaaacttagatttgctagtggtcccgagtgttttccagctctcagtaatgatgcagttcagattgtgtagtccctcatcgtctttcgcgagaaggatcatgtggaacttgcgtggctggttctctgaggcaacgtaggcctccaggccaaagattggcttgatttccctacgcttacaatgcttttcgagctggacccaactggagcagttaccgtgctctgtgagcgccaacgcggtcatgccaaggtctgcaactctgtcaacgtgaacgcccaccggcccgtacccgtccccgtacgaaaaggtcgaatgtgtatgcaggctaacccaaatcacgttgtattccttgttcaattggattggggcactaggggagccgagttaccacgagtcagttaccgccccgactcccctagcgccgtatgggatcagacttcccagggctcaccctcgtcgtctgagtcatcagccggcaactcttcgtcttccgaatcgccatcggttgcatcgtcgtccgagtccatgtcaccgaacgtgtcgtcggcgttgccctcgtcgccgtcgtcctctgcggtgtcgtcgtcggccgtcaagatgtcgtccgaatcgtcctcgtcctcatcgtcatccgcagcacgaggcgtgacgaagcggctgatttcggcccgttcgactccctggtcgtccgtacgcatcttcgtcacgaagctgattgggcggccaatcgggttggtcttcttgccgatacggacaatcgggtagcgcttcttggcatctgcatcgtcaacggcaaagccaatctccttgaaagccttgcggattgcggccttctgggcttcgctgccgtcggtgagggcatggaggaactggttgacccacgggctgccttgctgggtgagctgaagccacttggagacaccggctcccttgaacttgccctcagagatctcacagatcaccatgattcgctgctcacccttcttctccccttgggtgaccttggacagcagcatcttcttcacaacgcccttgtacaggcccggctttggcggcgggccatcgtaccaatcaccgcctgtgttggtcgccgagtcatcgtaatcggtgggcggcttcatcaccttgggcatcagttccctactttctcatcggttttctcagcttcgccggcaaaggccttttcaaccttcaggcgaatgtctctgagagtcaagtcctttgtgaacggggccagcgccatcgttcggtccttgcctctcaagctttgaatgtcctgccagtatatatagcgatgtgtctctagcgttgtgcgtggcttcttagtcttcggattgattactgccttaccgtctttcatcacctgcttgcgctcggtgcgaacctgcatgtaaccgtacgaggtcatctgtgctgccacttgcattgcaatggagtagcccttgccgtgaatctcagggcagaagaagtcctcaccgtcggcatcggtttccttacggtcccatgccaaccagagcaggttgaccggcagctggttcatcttcttgacgtagcgtagcagtatcttctggttcttgcggtaggtgggccagtcctctacgtccaggcccttctcatccagcaacttgtcgaacaacaatccctgtgcgtcactaatggtatcgatcacgatccagttgaacgggatgccggtgccgtcctcggcccactcggatagcttgtcgtaccactcttccagctggtcccaatgatggatgatcacctgtcttgcattgctacccatgcgctttgcactgatgcaaccctctggctcacacgtgaggaacagcaccttgtcgtcgctgcctcccagcaccgtcttaccacagccagaggccccgtagaccatgaagtttgggtgcggcgaaatggcatccaacgcaacgatatctggatcctgtacagcttcatcccatgtcggcataatagtctcccttactcagttactcgtgaggcgaattgcgatgtgctactaagagttactcgtacgtcctcgagcgtggccacagtgtaccacgaatctacctgtcgcgcaacagtttcggtgactctgcaccagagtagtactcttggtacgggtcttctttgcggaacatgtggctcttggcaaactctacgtctccaccgctttcgtggatctggcaaaggtcgaaaaagctacagtcccatttgcagtcacgtgttggtgtcttgtagatgggcagccggcgagacctgaactgctccataaccaaagcctcttggccaatgcgcttgatctgtacgttgcgttcggcactggtgcgctcaatgcgctcccggtggaagtacggggcaggctgactcttggagacctcacccagcacaaccaaattcaatcccctagcaagggattcgtactcttctttcttggccctcgaggagatgtagtctccgatatcgcctttgccacgctggtctaggtatgcctgaatggctttgacgtagtcttccttggtaggaaggttggtgtacttgccatcagcgttacggggcctttggtccggcttagccttacgcaggaagttgtagaccagcacacgaactgactctcgaggcccaatcaaccccctttttcgcagctcgtgtgtgccgatggcaaggtaaccaccgttctggtcatcaatgtaaaggtgatctacctctatggttttcatgaacttgtggtccacgtactcgatcagacctgtgatgtgattccgaacgatcaggtctatggtccctacgtattccacaatggggcaaggcctcttggcctgcttgcgtaggtcatcagggtgtggaatacgttggtggaactggtcttctgtccacaggacttcccagtgctcgtcattgccgtagtgttccaggtagtgcccgagcatttcgtggcccagcacgcgggcgtcctcccaagtgccctcaatctcgtcgtcgacgaacttttcgactttcactgagtcccatagctcttgggtaatcttgtcccaggtctcgcgtgggtcgattccgcgcttgcggcctggaatgtagtgctctgctagggcaaggtggccaaggcttccgaaaaccagtgggccagtagagaagttgacaggcacgaggtgctcgccatagcgccaccaccagcgcaaggggcatcgcttgaagtcgtgccgttctgagttgcgtagcaacggaacgttatccatgagttaccatcctcttgttgttaggggtggaagggcgtagacttgaccctatcccagtggcctttcagaaccttatgtgttagttccttgtccgcaagctcccacttgcagccaccgtacccggtcccgtttgattggcggaaccactacctacactagcgaactactcgctgacgtttcgattcggtcaggggtggcctccccctacctgcactactttgtctagcaacaccagtcggccttgcgagccttccggtgccctagttggcgtaagccctagggattagggtactttcgttgctagcggggacagacttttgctttgccggagtcgaaccggaaacggcttttacaccgttgctacctcccaatggtagtgctcttgcctgagcgctttggtggtcccttagggagaccaggccatacgatctgacgacgtgctgccccaggtgttccgtcgccttttgagccacagaatgcaccacgccatcgcactttcacgtgcccgctacttgtgctcttcagcccctacgtgattcgccaaccacgccttgggacctccatggataccgcacttgtatgcctactcgcactatgtatccctcccatgtaccaaccatcaggcttgcagcttattagcgtttgagtcgctgcgtacacgactggcctcaccgtatcggcctggcccgctctgcttgcctgcgtcccaccaccgttcagatgggcgaaggtactggattgcctgagttacctaggattactaggcgaccggggatcaaccggcctttcagcagcattgctactgcctatcgatcaggctcgaagtatattcaactgtgtaagaacaattcccgtgtaccggggcagaggctctgtgctactgggtcttccaggcttggagggactgtacttgactcttcgctggtgcgtaccccggtcactggagggcaggtcctgccctaaggagtagagagacagacgttttcagcctgccgaggggctactcaatacgcgctgagtgtcgatgtctctctaccctttagagctaccgaactggggagacattgcgtggctgtacggggaggccgtgcaatcttcttcaccagaggatggtagtggggctacttaatccccaattcggtagctgccctagaggaccggatcagaaggtcgcgctgaccttgtcgtcctcgtcctcgtcgtcgaacaacgagtcctcgtcgtcctcgtcgtcctcgtccgcagcagcggcctgggtgtcctcgtccgacgcgtcgtccgaatcgtcgtcttcctcagcgtcttccagctcccgctgggcctcttccaggagcttgcgggccttctcggccttctccttggccttggccttcttgagctgagcgtccatgtcgttcagcagttccttggtggcagggtcttcgtaccaccgagagagactgaacttgacggcgcggcagtcctccggcgaaatggtcttgccggtgagctctttgaccttgtcggccaacgactcactgatcacccggtcgcggggagtcggcgtgtccttgaacacacgaggtccacgagcgccgccacggcccgtcttttcggtcgtctcaacagtcatgttgtgtctcttcccttttcgttcttccgggatgccccggtggcctgacgacgacaacgatacaccaccgccagcggcctgtcaaccccgtttcggggcgaatttgttagcagcagctgtatcacgcaggtaccgggcgagggtgcctggcttagtagtcatcccgtcgttgacacgcgctggtgctccggccctggcccgcacaaggtgctcacggtcggattccgttgcgactctacggttttccagttccctgatcagctccagggtggcccgacgggcctgcagacgattaaagagccgctcgttcacacgccacgtgacccaactacctgcgacgatgcccaagaggccagaacctactgtaagtaagaagcacgtcatgctcagctctccaacaagtccttagctgcctctggtgtgtccaagatacgcttaagactacggcccgtcttacgggtggctcgtaagatcttctcgtcgatagtgcccttggtaacaaggttccagatagtaacgctatgtatcctggagatgcggtgaatccgatcttctagctgcacttgatcatccacgttgtgggtactgtccaccacgatcatgtcgtcggcagcatccagggtgatgcccacgccaccagctttcgtattcagtagcaacacttccggactcggcttaccagccggcgtctgcccagattggaattcctcttgaatccgtagtctctcatctgcactggtggcaccggtgatcttgtaattcggtatcttgtactgttctaacaagacttgcgcaaacaggttgatgtgcttagtgaactgggatgcaatgatgatcctgttgccgcttgggtctgggccgtaaatgcctctctcattaaggaactggagtatccagtcgaatttgttcgagggcatatcagggtggtaattgccctgttcatctagtctcccatagctattcgcaaactgcttaagccttgtcatctcagccaacaacgaattggctagcagcgtaccaccttcgatgttggccatggcctgggtcttcatctggttgtaggctgtacgttgcttgccgtccatcggaagccatactgcaacggggttgttcttgtcgtaccacccagccagcttggcttgtgccttccggtaacctagccttgccttgggattggccttctgctcctccgcagcgcgggctacggcgtccagtagctctttgggaggcttcttctttgggtacagcggtgtgccgccgtattgcttatccggtagctccggtagcactttcttcttggtgcgccgaatcatgtacggctttaatgcagctcgcaagtcgcgttcactgcgcagctttccgataacgtccgtatcgaatacgtcgtcgtggtagacctggaagtacctgtagacccacttccagtaagagttagggccaccattggcgttgtactccacagggtcaatccagttgaggatgccccagatgttctcgtgcttaccacgaaagggcgtaccagacaacgcaattcgcatgccgtcgtcacgaatgtcaagcagacgtaggcctctgctctgagcactttgcttcttcaagtttcccgtagcaccagctagggtttcgtgggcctcgtcaacaatcacggcagcccactgtatgtacagcagaggcttcagggcctcacgtaccggggtgatgatcttgttgcccttgtcgtcatggacgtagcgccccttgtcatcgcactcaatcttgaagcgtaagtagttaggagatacgataacccactggcgttggcccgcgcgctggctggaggtctcccacttgaccgtatctcgtagcaacgctatgcgagtacgcttgtctgcattgccggtgatgatacgcacagggtcatctggtgcaagatcgtgtaaccagcgccgtagctcagctggccatgttacgtacgcagcggttttgggtgccacaatcagcacggaacccgtagtcttgttacctgttacagctgcaatggcctggatggtcttaccgagtccaggttcatcggcaatcagtacgttcttatgtcgcatggcaaaccgtgcgcctgccagctggaacggcctactgtgaagggcaactccgaggtctgattgtggcaccctaaaacctacctatcttctagagccgcagtaagagcggtgatgatgttggtgcagtccacacgggtggccctgctaagattcatcgaccaaattcctttttgtacgcctccatgatttcctcatgagtggcaaatttgccctcattctctttgcgccactgacgttttcgcgccgaatctcgttcaaacttttcgattacggcctgttgatcgtaggtcattttcactacggccaaatcttcgccctctgaattgcattggacatagaagcgttcgttcttgtgctcatgcttggctttgaactcatcaagatcgttgcgggacaagccatttgaccaatcaccacattggttgcattcgatcctaaacgaatcgcacgaaatcatgaattcacgtgttgactcaataatcatataccactacgcctttcgcgccggctgcgagcctgaactccactgtgttcactcgtggttctccgaatctccagtgagcgcagcccagaccatgatacaagcagccttgaggcactggcaccgtgtcatcttggcgacaacagtacggtccctacgttgagtctgaatgtggatggttaacagacgagccatcacatcacctttcgcttggccagcacgaacccgtcccagcccttggaacccgttgtctggataacggatacgtcaaatgcaggctgacggccgaggaactctatgaattcccgcttgtccggcctgctagggtccagcaccctgccgtcgcgaatcacgttgtccaccaccacggtaacgtcttctgtgccaaggccgtacgcgtacttgaagtaagcctcgttggactccttgtcggcgtcaatgaagatgaagtcaaacgagtttggctccagcttgggcaagatgtcgtgtgcatcccctaccaacacactggttctgtggtggattccagcgtgatacaggttcacatgtgcaaccttggcgtgccgtggctcatactccacggtggtcacgtacccgtcctccccgacggcgcgggctagggcaatggtgctgtagcctgccagtgtgccgatctctaggacgttcttggccttggctgtacgaactaacagctcaaggaacttcgcttgctgcacggatacagcaatgtccggaagctccatgtcagaggcgtactcccgggcctctttaagggccttgtcttcccggatgcactgatccagtacagcgtctacgtcgttgggatacactgcggtcatgcgttgcctaccttgatgattgtgtcgagaatgacagaacggaagcctgccttggctcccatgaagtgaaagatcttccgatggcctactggcgctatgcgaaagttcttcgagttgctcttgcgaggttgcaggactacgtcaaagtccagcctgttgtactcgtgcgagaaccagaacagctgacggccgatcatctgctccatgatgaattccggtgtgtccgtatgttcatcgaatggcagccggcgatatgtttctactagattgggtttgttcttgttggggaacgccttgaacaagtccaggatgtccggccacccctcaaccttctctaaagctacgctggcacagtgtaggttttgacgacggtcaaagatccggtactctccagcttcatatgcgttgcctttgtatgaaacctccagcctctcatcgtttctggttgctatcactgttgtgtggcgaatcccgtccaccaacggggcctcgatattgccatgccagtcattgcgtttcgctgcaacggcaatcttcattgccttaacaactggttcctcggctggtaatgccatgggccatcctctgtgtgttccgtagggagtctagggcgtcgggcctaggccctaaccctgccccggatcataacacacgagagcctgcttggcacgaaaaatgagatagccgtaggactaacactgagatgctcaggctagtaacccaaggctgttatccatcacgagtaagtgggtgccggggccttgaaccccggtggctgccagtcacccgcaccagactagttggtagtcaacaactctggcatatccatcacttgagcaagggcctgcacgctcttgctgtccaccttggcaaactcaccgtccaccagcttggcagcgttgccctgaacacgggagcggccgcgcagtggcgcgtagtgggttgcgtaggtgttgattgcctgtaccaccccaaggccagtacctttccaggtattcgccatagggtcactttggtacacgctgttcaggtgttccctcttggactcacgaccacgaacggtcctggtgctgtccttgttgggatcacccggtggaatgatgatgtctagcacctcaatccacttcttgttgggcacttcaatggcagccagcttgtggaagaactccatagagctgtctgcggtttggtgaatgatcgacagggcttgccggacctcagagatcttgtgggtctcaaggcttccggacgtgtgacgtgccttgtactgtcgccctatcttggctgcccttgcgttggccgccgtcatcgtgttgtcgcacacgatcaacaacgtttggccagcgaaagtagtagccagactactgtctaggctcgtatagcagtgaacgtatggaacaaagtccattccagttgtgctgtcgtgagccgtctcaggaagagccacctggacacaggcctgagcgccattgcgcaacaggattgctgaccaaatgtcgagagtgtccccaatcacgttggatagcaactgcaagagccactcctcgtagtcgtggatcttgtagttcttgccggggtgtcccagctccaggtagttgtcgctacggaccaggccccgtttgtcatggacctctaccagtttcatgggaacgttgaccggacggccctcgtggaacactgggttgccgttgctgtccatacgctgaacccatactgacggttcaggttcgtagcgacggcgtccctggtcgtcggtcaagggctgtcctgcctcatcccgtagcaactcacatggaatcatgtttgccgtacgggtaagcattggcttccaattgaatagccgccgtacaacatcgtcatacgggatgccctcggggtaatgattcggctcctctccctgcatggactccatatagtgccaactctttccacgctgattggtgtgcccgatcaggcagaagttgttgtaccactccatggtttctgcgcccattgtttcccatcctctttctgttgtggtgcttggagtctccctcgtggctccaagtcgtgtccgtcagggactcgaaccctgatgtctgccagtcggactcctgtgctgtactagtctaccggtcgttcacgttctgccagtagtgatctggcccaagcgatatgggcttcgtcattcagccggcgtctgtgctgcgaaactctgtaggctcgcaacaacttccgcagtttgttcactgagcctccctcttgtaccgctcgaccggggcgttcatccctcggctgagcataccgtcctcacactcgattgtcaacaccccagagtcgttgagaatccttgtgattcggccacgatgtggcagctcatctgccggtggcatgtgaggtaggctgccatcgctgagctcgcgggcgagcacgaattccgtttctctatgcaactcagtccatcccctttcttgatacgccactatggcgctcgtgcccgcgctggactcgaaccagcgtgtatgcctttcgggctgcccagtctactgggacttgttgtcttgctgtgccactcgttcgagcatggcatcaacttgggtgtggtagtccacgtctgccataccctcacaggttggctcgtgctcgctccacagcaggggagcctcgtacgcgaaagcgccaaagcctcccatgggaattcggttcacagggtcaccgacaatcccggccttccgcagggcctcgaacactccgacatgttcgctgtagtccccgtagatgaagacattgcctttacccggctgaaggttgtagttgcgcaggcagactgtggccgtagccagaggctcaccgtactccgacacaacacgtagtacagtgctgccgtcggcgtacttgccccacttcaggactgcctttgtctctgggacatactgggtcccaaaggtaaacgtacgcatttccatcccctttcgttcggtagacctggtggcccaccgtcgtccctgccagaggaatcgaacctctgttggtgtgtaacccccgaacacgccaaaaccgttgcagggtatctctctacttggcacaatccccgtagccacagcagagtgctttacttacgcagcatcttcacctgagttgacaagggatatcggtaggccctagcgcgctggccctcgtgaaagggcacgggcctcaggtacacgacgacccaatcgccatcaaccacaggctctctggtcacctcccaatcaccatctgcgtcggtgaacatctcgcccaccttgagccgatgaatgccagaggtcttcctcactgcatctgctactcgcatttccatcctctttcatagtgccccaagccgattgctcaggactcgtgccgccccaggactcgaacctgggtgtatgccattgcggctcttgtgactacggccagccgtgctccacattggctctgaacttattcgcccagaggatatgcacgtctagcactctcggtgaatcagttttgaagtcaccgcagatgcaacggtacggttttgccgggccaaagcagccggcgaaatcgcatacgttgctattggttccatgctcgctgcagtctctcatcggataatgtccgttcccggtggcagccaacccggctcatcgggcagcacgttgtggtcaaacaccacatacgtttccttgccctgaagcgtctcctcccggtagtagccaccgctggaagagcatgagtagctaccgcagtaggtgtagcgtggcacgtaacccgctggtgtccagatcaggcgcaaacgctcccagctgccgtcaggcctgcgcggcccgtcgcagatggtgcgcctttggcttcctaggaaaccccaacgaattgtctcgcagccaacgcctggatctgccttggccacattggttacggcggcgttgtggattgctgcgagagcgaagaacaggcccatggtcacaagaaatgcaaccacgacttctttcacattacgtttcatgttccatcctcgaatcttgatgtaaggcaacgggttgttgccttcgtgccggtagtggactcgaaccactattaaccaaccgtggccggctgcctagcgctgctaggactttgagaggttgatcacaacgatgttctcgcccttgaccagttcctccaggtgtgcaacacgttcttcaagcgagaggcctactgcctcagcagcatcttgcgcatcttcatcgtcgtcaatgttggcgtacggatttccgcttggggcactcaaacccatttcgcgctcttcatcggaagccttaatgggcctaccaatcactttggttacgtgaccccgtgctccaaacatgagcttaccgtcatcggtaatgtaaatgcgtagtacgccaatctgaccggcctcttgaaacgccgacaaggctgccttgcgaaggtacgcaaggtcattgcctgaaatagcgataacgtgcttcattccatcccctttcttgtagtaagccatagtggctcaccccgcgcctgccgacggcttgaacgtcggtgccttccagtcaggcttgccgttacagtgtgtcatcgtccagtgctgtaacccatataggctcaccgggattggctgcagcaagtgctcgtcgacgctcaacgtcaatccggtcctcccctcctgcatagcagtaccagctgcagtagcggcattgccagtagtcctccccggttcgcccgctgaatctacatgacacacctttgcgtttgcattcggggcagcgtatgaacttgaacccccttggtagcggcattgttccatccttctcttgctaggccgtagaacgcctacggctttgtgggtgtcagggactcgaaccctgatgtctgccagtcaccctgccctgctttggggctaggccctcggagcactatcgccgacggtccactccggcatgctgtaaccggattgatacaggttgttcagcacttcctgagcggtcatgcgctgcacctgttcccgtgcctcttctggagtacgggccttgatacccgaaacccagacctcagtgtccccgaacactccagggacccgtacgggcacattgtaatcgcgaacacggccctcgagctccatgacctcgaaggtctcgttccagccgtagatacggcgctcatagtcgccgcacatgtgggtttcttcagcgtaggaattgatgtgttcgttcagctttcggaaatcacgcttgactgcctccagccgctcaccttcttccttatgcagttgcttgtacgactctgcctctttcaaggccgagtacatcgtccggattgcctgccgcaaatcgcgactggcctccttgggcgtaccgttgactgtgaatgcctcccactggcattcgtgatccaggtccaagatggacatcaagctggttgtcatgtaccggtggtggacatacgcaggaacgttgtcgccgtacttgttttgcttccagccagtaaagatgtactggcagtcaatgtttcggccgtcccagaagtatgaccaggtgggcaaaccttcgaagaagtcttggtacgtagcaggtttggtatcctgctctgtctcctcgccgtctgtggccgtggacacaacgagccaaccctcctgctgaaaagcagcaatgtcgatcacatcagtttcgtagtccaaagcccagttcccgtcaggaagcttggccaccacgtacagcgtaccggtgccaacttccgtattgcgaactgtcccaccgattccagccttctcgcaagctgccgacagatcgatcagctcaactgcattcacactgttctcaatggtggtcatttccatcctctttcgttaaggcagtggccccgtgccactgtcctagtgccgctcaaggactcgaaccttgatgtctgccagtgcggctcgccctgtcatgcgttacgacgggcctcttctacgatgtccaacaggtggtttgcaatgccctcggcatcagccaaggtcagcttgacacttgcgtgccgtttcccgttgggacccatgatagtccacttcagccaatcaattgagtcatgacttccgtagattggatcgatctccagggtgtgtttggtgcctgaagtgctttcaccatgtgccaataggatatcggccatttcagttcctttcaagttccccgagtggtggggaccaaggcccttgattgaacttgaccttcacaccctcctcatcaaccatgatccgaagggtatgagtatcagggttcttgagataacccctgatcgcctgcagctgcaaaccgtcaatcttgtacacattcaccattttcccatcctctctaattgccggctggtttgccgggcaaagtgccctgctgaggaatcgaacctcagtacgccttaccagggctacacgattgggttgtcagtggtgcttggcccgcgctgcctgcttgacggccaactccaaggcgttggcgtggcgcatagcaaacgccacatgtgacctggcgctcatggtgctgtagtagcttcgtcctacagccagcgcaagttcgaaagacgtttcgtctctctgtattccggccacgattcgggccacctcgtcgtcgctccacggctgaccgtggtttgccgccgccttcagtgaccgggcctgaagctctcgagtgctgttctgccccttgcacttccgaacgcgggcaaacctcgtcggcttctcggcctgaatcgacttgacaaccttcatctttcgtgctgccatttccatcctttctgtgaggtcgtcccgtacgaacgacctagcgggcctaggggaatcgaacccctacgaaaaccgttggccctccggtgattacgtcaccagtcgttggcgtgctgcagttcgtagccctccaggtcaccgacgctgtcgtcgtaaaccgatggattgctacgccaatcgctgcgaagacgctggccggagccgttgtagcaggccccgcacttggagcagtcaacgtctcgaccaccgctggacacaacctcaatgccgcactgagcacaatcccaagaggtgattgagtaagagcctgcgttgcagtcacactcggtgacgttaacgtccacgtggcagttgcggcaaagaaagttgcgcgccttgattgtagccattgttcccatcctctctagcaaccactttggttgcctagtgggacgggaggaatcgaacctccccgaaaaccgttgccccgatttagtttactgtctccctttcaggtacaccatggctttgactacctcttgcagatcttcttgtgtgccaacgttatccaagatgttggccatgtactcttggtaccgggcgatttcgtcacccggcacccctgctgcctccaacattgttgagtagcagtcgtacacagtttgaagatcagactcttcagcggccatcagatggccaccgccacaggagtgccgtcggccgcaaccacgaatcgctccatggggtaatcatggaagttcatgtggtcgttgcacgggcaatcccattcggactcatacaaatccatgttctgctcggaagtgccagtgccaaagtcgacaccggcaacctcgttctcgaagtagtccatggcctgcttgacgttgtcaaacttcttgtgtccgtaattgaaaggaccgtagtgaatgtgtgcgcagatcattgcttccatcctctcgtaagggccatacgaactgtatggctttgtgccgctcaaggactcgaaccttgatgtctgccagtgcggctgagagcctttagctctcgatggactgtatgaacaccgacaaccaccgcgctgcggcgtggacctgatccggatgccccgaatcgaacttgacgaacacgttgccctgctgatctgtgaggtacagatcaccggccccaccggcctttacggaaagcagtgtgtcagcacagttttcctcggcccacctggggttgttcagcaacggcttgccaaacccgactgcagcagcagcaggatcaacggcctggtactcgaagttgctcatgtcccatcctctctgttgtgccgtagcggcgctacagctttgtgcgctccctggactcgaaccagggtgtctgccagtagcgcgttggactcttagcttccgtaaccttttcgcccgtctcgaagagcgtctggtttcggaagccttgagtcccaccattcaccaggaagacgctgcagccagatagtgagctgtgtcgcaatgtcttccgtagtcgcattctgaaggctggagtgcaactgatccacgtagtggtccctgtactcgacaaggtttcctgcctggatcagcaaggcatgtgccaaatcagccttttgaacattggtgagtgccatcgtctttctcctaaccgcattcataggtgaggtttgacttatagatgcgtgccttgaaccggccaacggccgtccggcaaatcgcgtagtcggaatccttagggtcccaacacacgaaggtgccgaacagccggtggccgtgtccgtctacccaactaacgttgggcctaggcgcgggcatcaacgcttcctgcgcttgatctggatggcctgttcgttagcctctatggcaagaacgattttggcctccagctcatcagggaacttgtctccgtacacccgtcgcaccagccgcacggccccagaaaaccgtgcccggtcctcttggaggctcgagatggtgatgtaactgggatgccatgcaaccgtttgcggttgactcagcaaatgattggccaagcactccacttcgtggagcatctcggccttgacttcgtcagcagtggtgcgggtttcgacgaacatggttaccatcctctttgtccgtaaactgttgtggctcaaccggactcattgtgccattggagcactcaaactgattgagtttgcttgagtgccctaatggcacaacgagtttgaactcgctgtgccgaggatggatggaaaacgttgtaacacattgacctgtagcgacaggtgctccggccccgcgtgaggggcttgggtggtgcctgctcttgctgcatgaaggtgtaccgtcacaacgtatttcagcctcatcgggctcaattccctacactccctaggttcagtcaggtcacaccgtgtccggcttcagcgggcaccccgtaggatgactccggtactgtctcacgacgatccgttatcgctggttgaattccctaggtactcttgggcaagagcgccttcggcctatgttgcactaaacagcccgtcccaggatccacagcgctgctccatgtcgcagctccaccgtttacacgaatggtgtagaccctctaggtcttacgtttatgtcccagtggggtggagcctttttcacgcgtcgggcgaagagctggtagtcgtttcgccaagggtgtgctcgcgtccgcccgtgcgggcggctgggttgtggtggggtggcatgggtcgccccaggcaaaagagtaccagaaacgggcctaacttgctacttttcggcagcgccaccagggaatcttgtccgaatcttgcctataccgtgaccaaatctttatctgtatcactggacatatgagctggtcagggcatcagccggcaacctgaccataggaaatgcaagaggcccgcagctggcggacactcagtacaagcgtactgctatgcagctacgggcctctcagtgccttagggacactacagcttacgggctttaaataggatcgccctcagagacagcctcagacaggcgttgtttgtagtcggctagtctggccctaaggtcaggcttttcggccgtctcagggcttctgaggccccgttttccacggtttgcccaggtcgctgcagcgttcacctggggcaacagggcactttgggcagacagcagcatttgccggccagtccagcgttgttcgataagcctatctgtctctaggctttccatgccctcagcttctgcaacttcgcgtaacacgttcggtaccagctcgtttgggtccgcgccgagcttcagcgcaagtacggccaccagcgtctccagaaccaccgtgtacgcaatgtcgtcaggattgctcacttttcaggcccttgatcttgtcaggagaaaagccttgccactgtttacctttggccaccaccaacggaagtgctgtgcctgtcgccagggccttgacttcttggtgtgcaacctcgttggtgtcaatgtcgatgtactcgtaggggataccctctttgtccatcaccttcttggtgtgctcacactgcacgcaaccaggccttccgtacacccgtgccttgctaccactcatgctaccctccttgttcccacgccccgcatgtactccacacacgggttaaggtcatatcctccatggggttccatgttgcccatgaacatcacgccgccgatgatagcaaggccgatatcgatggtgccatccaatggatccattaggaagtcaccgattctctgcaacagaccggcaggcccaccggcccagctgttttcagcggcaatcttgtagatagcactacggtttaggccgacctcgttgtcaggattctcggaatacaggtcaccacggcggctgactactttccaccagctcggcgtattcgttaggcgcacatcactgatgccttgtgttcccggcttgggtggatcagtaacccactctgccactacatctttctcgcggtatgggtcaccgaaggtgaggccccgtgtgaggttggcgtacctttgctgcagcatcgggtctgtggtatccctgagtagcataaggatacggttgacaatgatggccccttggctgaatccctcgatgccccatggtgttccaagtggaaacggtctggtcggctgcccgttgccgtccaacagaaccgtctgcctcagcacgtttagaactgcgctgataccagtttggttacggaacggtaagctcgtgttgtcgtaaccaataggctgccaccaacatacaccttcgctttccagcttagaggcaacgtacgcgcaagggccaacgtacatgctgctcaggtgtccctctacagtaatgatgatggggcgcacggggtttggtgccttcttgaaccccatggcgatttgagtcggcaagtcaagcacaccagttatgtaggtacccaactctagccggccttgcaagaccagacgctgctgcatttcgataacagcagcttccatctcgcggtcaaactcgtttgtatcagccaaatgccctgcgtagctacggtacattgcgcgcatgtacgccttggcacggcgaaccgtatcatcaattacggagtggtcccctacaccccaaccaacccaaatgccgttgactctcatcctgtttcccccaggctcttctcttgctcctgtgcctctttgagtttgtggcgcacgtacaccagcgacggtggagtctcggcctcaataccgtgctcccggagcaaccgcatgatgcggaagtcccacgaaacatgttcagttataagcatttcgatttcaacgagacgttcaaagcgctcatcggacagcttttgcagcttctcgaagtcttgacttagagcttctaggcgtctgatccatgtctgatcacctacctgctcaccctctttcttcgtcttgtccgcggtggcgctgttctgcttgcgattggcataggtagagtagatgcctcctacagcagaaatggcagccaccgtgactagcgcagcttgggtgatgagggactctaacaccttcaattccgttcccgctccctgcgccggtactctgccaacagctttcgcatttgacggaaacgttgtactgaggcgtaggcgatgccgacactcaaccccacggaagttactgctatccaatagggcttgtcaccagggccatgcgtggcaaccatcacaaagagcgtataacatagggccgcaacgaaaagcgagaggtggccaaagatgccaagcctcaggctgagttcaaaccatcgttcacggttgagtacagcaccggccaatgccatggcactaccaaccacgaacggtatgttgagagcaactaccgttgcataccctactgtcttatacagtgctgattctggaggcagccctaagatcatctgggcgatcgataccacgagaagccctgacagacaacccgtttggtatgggtgcctgtcagggcgttcgcgtagctgcagctcaaatgtcacatcaccgtgaagctttttgatctgagccgctaggtctttcggctctggagcagtcacagcttattgacccaaacctcattggacttgaggaagttcagcgctgcagtgaagaaaaccactgcaatgctaaagattcgttgagtggtgtcgtccatgaagttcgacagaggcgtaagctcgttgagcatcacaaccagagcaccaatgagtgtgatgatggtgtggtagtacgcctttgccttgtccagccagttcacggcccctggcttctcagccagcaccttgacctcagccggcaaaacttctggctcagtaccggtctggtcttgaaccacaacctgattcggtagtggctcaatgcgaatcttgaccggcgcgggcgagtcaccggtcatgtccggtggtagtggctctgccatgggactatccttccgggaaccgttgtgcgataatggtgttcttgaggtgctgttgagtttcggtgaggtacttgtcctcaatgcgaatcagcactgcctgagcgagcttgtcgccacgctcactgttgcgggccaccagcgcaatgctgctcggatcacctagcactgcaagacgttcgatgtgggcctcgtgtaccatgccgtcgtcgttcttgatcagctcgtgcaacttccagatgtcgccttcaccgtcagcgcggtaacggctctgagagccaatctgattgaacaggcagccgtggatttccttaatcatcttgacgattgttggatcgctcatccaatcttcctctccaagtagtaaattgaggagtcgcggacccatagctgtggtgcgatcgtacctcattttacggtctggcaagccattcagcccgccattgacgtactttgtagcttgctctaggtttctggcgtctgctgcttggttcatcggcctctgaacagtccagtaccaggtcacggcatgaaaaccgtactccggcttggacagttcactagggttcatcacaaagtaatctggagtagacactatgcctttggagtgtgcccactcagagaacttggtgtagttgtaccggcctgtaacttgtatggggccacggcctttgaagcgctttccatcaccgggctgtgtattgcccaggtcagtacggccttcgtacttgctgccgtcggcaatctcttccatgtacttcaggctcagtgactcatgcccaacctgtgtgcaccacatagcgattcgcgcttcagtggtgcactcgcagtcacgcagcgtgcgggcaaccaacggtacgtagtactcgtagtcaacaccactcacgtcaagcatggcctcagacaacagagccgtgaagttgactccagtagtgcccttatgctccttgaaccgactgaaacctgtcgtgatgtcaatcttgcgagcaatgaagtcttccacacgcttccacgtagtagagccagcaggcccatacgtgttgtatcccatctggaagtgcatggaatcttttggactacgccaatcgttgccccagtagaccataccctcgtagtagtccaaaagctctcggacatatggggtttggtcacccttgattatagatgaaccattccagccggctcttggatctggtgctgcataaccgagtgggtggtcactccagttataatcaaaggcagtgcctcctaggtggttactggttggcactttgtttgtaccggtccatccgccctcgtccgtgccgccacggctgttgtaaacgctctcgacataccagttgagatcagccgcaaaggctttcaggatcaaggacggttggcccttttgaagtggaatgactacgtccgtgccgggaatcggcttgagatccaagtcatttggcccacaatcgggccaaccgttttccgtgatcatgtttagccctctggcagctcgatgtatgcggaagtgatagcgacagtagagccgagcgtgatactcgtggtgttgaggatcaggtcagcatcagtagtgcctactgtcccttgagccttcaccgtgtcgccggctgagttcaggatacggaagaacgttgccgtacctgtggcatcagccgaggaatccgctgtgatagtaccgaatacagcccgcgctgcgctgccagtgtcggagtagctggcgaaaggcgatgaagcacatgtcagttcagccagctgagtgttactggacagtgctgcatccgcattgctcggcaccgtgccgctgtacagccggattttgggtgaagcgcctagggcctcagccaaacccgtagagttgagcattccggccaccacggcctgggtgagaaagaactgttttgccatgctactcagccaccacccgaatcgttgcctgtgccgggccaccgatgttctgaggttcccagcggtgaaggatccacgcctctgcgcgccatgcctcttcctccaggccagcagcccgtaggatctctacggcattgacggcctcgtggaccgtcttgtcctcccaaccgggatacagctcagggtgagccgcaacgaaagcatcggcaaggtctcttgcctgctccagattgcggcctggcccgggagatgcagcgtcccggattgcctccaattggctatccattagtgttcctttctaactcagcaccactgcgatgctgccccagttgttgttcacgtagtgcgtgaacgttgttgatacgtcagaaatgctgagggccagtgtcactaggttgccgctagacgtatccaatagtgtagcaccaccgtcaatttgcctgaatgttccattgaaggcaaagcactgcacgatcaattggtttgccccacaggtgaccgattgtgttgcattagtagagtttccgtttgtgccggaagttgtaccggcagaggtcattcccttaacagagatgccaccggcgatataccaaccagagttacttgctagtgttacttggattgtggggttagccgacggcacgtttttatagctatacagctgcactcgcgcgttggccccggcaccagtccacagctgagtgccaagcagtgtcatgttgttgccagcacatgtgactgtgatgctctggtccgtgccacggtccacgcagaagccaacaatgatgtcatcacctgcatccgctaacactgtgaagctggcactagagtttccgagtgacggggacgtcatctccccgtagttgttgtactttggcccctgtattgctatgggactcagacgcaaagcctgtgcagcccagttcgtagtgctcggcagtgttccaccaaaggtagtgttcgcgttggcatctcgcaccatcattgcgaaactggagcctgttttccagaagcgctctgtgctaccgctattgacgctgctgaaggtggagccaccgccgttgcacaggctccagctgaccatgccactgggcgggcacgtaactgactgtgctacagtagcgctcgcacgcccagaagtagaaataatgggatgcagggcttgcgcgtccttgtaagacacagctgttgccactgcccagcgagagccactaagggccaccgagatactgtgtgaaccattggatgtagacttgccaacgtagcagcataggaacgcattgctgctctgtgtgctgctgagccgctgaattgacatgcgccgcaatgggttaccgtcaatggttaccgcaccgatagtggcactacggtcacgggcaaaccagacaacaacgtcttccccagcagatgatggggtgatggtgaacgcaagagggttggccgtaaccacattggtgcctgcactggctgcggcaaacgttacctcgttggctacgatagccgttgacacaaagctggtggcacgcacttgcggagtaagtgagccttcctgtgccacaccaccactgaatgaggccttgtgcaactttgtctgtgcaccaagcgaaccctctggagccatccagcccgatagagaggcaacggctttcttcgcagaagcctccaggtcaccatcataaggctgttctccggcaaagccagctaccaatggggccaattgtgctgcaacactaccttgctgccgcacttcgccctgtgccaccatgcccgccacgatcaggcgcacttccattgctgcctcagcccgaacttcagctgttgcaccaaagtacgcacgcccaagagcggactccaggttgccttcaggctgcagagcaccagcgaaggtggcttcagcaggttgcgcttctgattcaatagtggcagtaaagggttggttaccgaccatatccgctgtggctggcaatacctgtgactctaggtctccctcgtgtggctgctcggccgaaagagatgctgacgccggtttgagctctgcctccacggcagagaagttttcggcccaccaaccaacggtcataggtcaatctccaatggcagagcaatagagctccaacgatttgtgccgctggaggtagctgtgatcgtgcctgtgcttcgagttgtgctgatgcagatcatgggataggtgccagcacccttcttgcggttcttggccccagtaatggtgctagggatgtagattgggccaccgcccgcgcctgcggcaaacacctggagaatctgctcgttctctcctactgtcacatcttgggtctgtgtggtgccaaccccgtagttagtcatgggagtaccgacatcagtcacgccctcgaaggctaggcaaaagcaggtatgccagccaccagaaccactgaaacgcactgtttctgcttctccagtgccagcaccggccgcacgataaatagcttgtccaccatagccggcagtattattgttcagtacagggcctgccaccagctccatatcttcaccggcatactctgccagagtgacactaccagtccggtcccagcaaaggcacatgtacacgtcagctccctcaggggcagcgaatgtgccagtgccagtgccattgctgtacatcaggcttgaaatgccaccccagccaagattgataggagctggtgctccgacgtacgggtctgttggtaccgtacaagctacgtatggtacagggtccatgctgccagggtctaccatcgacgcgatttcggcgaaaaccttagtgaatataggcacaccaggaagatcaggttctgtagtctgtgagttctcaaagccagctatgacagtgccggtagagcggttgtacatcgaaaagcactcatagccagtccaactacccgggtgactgttgaagtcaccaacgtcatatgtccctaagccgtatccacattccggtggcgctgatccacccaaaggaatagggcagaacacgctttcccacaactcagtggtttgctcactcaggattgcatagtctcgcatatgctttgcccactcaagcatgtcagaaatgttggaaacgatggccccggcagcaccggcaaagcttgggtgaacgccagtggtgttctgtgtacggccacctgtgaggccaccgtgaccagctgcgtacggggatggcatgcttgccgtagtaggccaacttgtttcagtcaacccaagtggctgaatgatgtcttctagcaccacattacgaatcggcctgccccgaatggctctcacgatcagcccgagtagcaggtagttgccgttggtgtaaaagaactgtgtcccaggttcaaacattggagtgccgccacgaataatgttgagacgcgtagcctcactaacagcattgccagggaacaacagcaggttgagcagaaggccccagtctgcctgctcactcgcaatgccagagcgcatcattagcatatgccgcactgtgatcttgtttgcattagggactgtactcaatacgtactcatccgtatcgaaatcggccagcttatcttccagggacagtaagccatcttcaatacacttgagtactgctgttgcagtgaaagtctttgtaagactaccgattctgaagtggtcatccaatgtcaatgcccggccaccataagtggccaagccgtaggccttacggtaccagcccaaatcagactctacggcaatggctattccaggccccattcccttagacaggcagtcttcgcataacgcatctatattgttgcgaattgcaagtggcaatgggtccatgccaggcacatacacgttagtctccactgagattggatctgacagttcactttccagacctgcgccgtcaatgttggtagccgcaatgtagtgctctgtggcgctaggcaaacccgtaaaggtgtggctcccattcaatggcaagacattagggttgccttttgtccactcctcaagatcagtggagatgtagaagttacgcccggccaatgccattagtccgtacctccctctgcctcaacggtaatgaagctcgagcccttttcgactacgctgatgagagtaggcggagtaggtggctggttgtcttcctcggacgcttcgtcatccaaggcatcttgcctaaaccgtagccacacag

SEQ ID NO: 3 - DNA, 56361 bp, unidentified
  source 1..56361: mol_type = genomic DNA
  misc_feature 1..56361: note = contig00001 len=56361 cov=23.2 corr=0; spades = NODE_1_length_56361_cov_23.246116_pilon (bacteriophage Minn381)
gctcctacccacaccgcttcgacggtgccgcagcccaaatcaaaggcttcttcgacaagctcgacgtgtggcgcgccaaacccggtgccagcaccgatatatggcctgtctcttatacacatctccgagcccacgagacgtagaggaatctcgtatgccgtcttctgcttgaaaaaaaaaaaaaatttttttttttttttttttttttttaatgatacggcgaccaccgagatctacacctaagccttcgtcggcagcgtcagatgtgtataagagacaggtgtgacccagggggtagggaggttgtagtgccgttcgccaacacgactgttcggaatcgtcgccgcgctcaggttcgtcagcgtgacggtgatgcgttgtgcgcgttgcggataacggctgattgccaggcgcttggcggggttatcgactatgacgctcgcccacctcatcctcggtcgttcgaggttgaccacatcgtgagttctgttgaggctgcgcgactgggctggtctcaggaagattcggatgggctggataactgccaggcagtgtgtcggcagtgtaatcgcgcgaagtcgtccggggatcgtgctgtgccggcagtgaggaagtcatacgtgaatccgcgctttgcctagaccttgcaccgggttgtcggtgcgccacgctgacgtcgggcggttaaccggcgggtccgagagggaatctaatggctgagtacagcactttgaatgaggcgatgcaggccggtgatgagctggcggaggcgaggatacggtatcgcctactggctgaggcgtttgaggatatgccacagctccgatcgcaattgaacgctcagattgagcgggcgaaggctgagatcgtgaggctgacggcgcttaaacctgagtccggctcaggtacggcgaagcctggcaaggttgtggtgttcgatgccgaccggttccggaagtcgggttaaccctgcgccgcttgtcgatattgcccgccagtgtttcattccggatgacatttcgcatacgcgctactacgagcttattgctccggaactccccggcatgggggtggcgtttgatcgctggcaggaagacatttggtacgcggcactgggtctgcgtgaggatggcacgctggcgtgcgatgtgatgggtgtgaccttgagtattgcgcggcaggctgggaagacctggggcatcatggtcggcctcattgcaatctgcttgtcgcggccggggactctggtggtgtggtcgtcgcatcatgatcggacgtcatcggagacgttgacgaagattgctggcattgtcgagaagccggctatccggccgaagatgcgtccgcttcatcctgtggtgcagtctgacgacaatcggggtgttcatttcgcgaacggttcgcggattttgttcggcgcgcgggcgcaggggtttggccgcggtttctctgaggttgatattcaggtttacgacgagtgccagaatctgaaggagtcggcgctgacggacatgttggcggcgatgaacgtgtctgagattgggctagcgttcttcatgggcactccgcctcgcccgcaagaggttgcgttaggtgtccatgatgcgttcaagcggcgacgtgatcgcgcgctggagcagaagaagcggcgccccttcaagggtgtgtacgtggagttcgccccgcagtctccagatggcgtggtcgctgacattgacgcgcagggtttctgggagagtttgacagaggctaatccgtcgttcgggcatcgcgtcgggaagtcggcgattgagcgcctggtggagaatatgtcccccgaggatgtgcgccgcgaggtgttcgggatttgggacaagacgatcgaggtttcgtcggtcgttccgtccgatcagtggcggtcgctgttctgcgacgtggacgaccttggtgacgtttcagcgttcggggtgagcgcaaccaggtcgggatggttctgggttgttgcctgctggtcgggtggcggcgacggggttcatgtcgagattgcgctcggtacgcagtctgaggttgaggcagttgatttcctgcgtgcgcatgcgtcccgcaagacgccaatcaagcatgattcggttggtgcggcgaaggcgctcggagaaaaactgaagcaactgaggttcaggtcgtcggtctactcgtcgaatgaagctgtggccggtaatgcgttgtgggtgagtcttgttgatcagggccgcttcactcacggcggccagtccgagcttgatgtggcggttcgtggcgccacgcgtaaggatcgtccgtctggcgggtggatgatggtgccgcgcgccgagccgtttgacattggcccagcgatagcgatgtcggcggcggtgtatgcggcggtgacgtcgaatccgcgtaactctggaggcgcatcgttcgcctgagattgggatcgatatagaagggaggtggctgcgatgctcgatgatcgcgagatacgcgatgtgatcgctgctatgtggcagattcacctctctgagcgttcgtggcttgaccgtatcggcgactacgcgaagggtatccgaggggttccagaggtcccggagtcggcggagcaggagatcaaggacctcgcccggctttctgtgaagaatgttctgggtctggtggttgattcgttcgctcagaatctgtctgtgacgggttaccgttcggcggatgcgcaggacaatgatccggcgtggcggatttggcagctgaatcgcatggatgcgcggcagtcgacggtgtatgttccggctctgacgtatggggcgtcatacgtgacggtgaccgccggtccgaatggcccggtgttggcgccgaggtcgccgaagcagattctgacggcgtatgtcgatcccgttggggatgagtggccgcagtatgcgttggagatgtgggttactcaggttgatgcgaagctgcgtcgccggggccggttgtatgacgacgagttcgcctatgacctcgatcttggcgaggtggcggattctgatccgcatgtcaggagtgcgacgctcccgatcagcgttgctgttgaaggtgatcccgtcgcacacggggcaacgtatggcggtgagccggtgtgtccggtggtgcggttcgtgaatggccgcgatgccgacggcgcgatcgtgggcgaggtggcaccgctgattcgagatcagcaggccatcaactcggtgaactttgaccgactggtggtgtctcgattcggggctttccctcagaaggtgatcacgggctgggcggcgaccccgagtgagattctggctgcttctgctaagcgtgtgtgggcttttgaagatgaggatgttgacgccaagtcgttgcccgctgcggatacgggacagtacaacgacattctggacgagatgattcagcatgtcgcgatgcgcgcccagatttccccggctcaggtgacgggcaagatggtgaatatgtccgctgaggcgttggcggcggctgaggctaatcagcagcggaagttgcagtccaagcgggacagtttcggcgagtcgtgggagcaggttttgcggctggcggctgagattgacggtgaccaggagacggcggctgattctggcgctgaggttgtgtggcgggataccgaggcgcgggcgttcagcgccgttgttgacgggatcacgaagcttgtcgcggctggtgtgcagttgcgggacgtgattcatttggtgccaggattgtctcagcagcagatcaaggcgatcaaggattcgattcagcagtccactgtggttgatctggtgtcgagcattaggcagaccgcgctggcggcccagcgtgatccccaagtgggggatatagcgggtaggaccgttgccgaatccgactgacgccgaggcattgcggcaggtgctgtcagacctcgcgacgctgaacactgcccagttggtgcagttgtggcggtcattctctgacctgtcagggtttgagcagatcgtgtctgaggcgctccctgatgtggtggcgccgcagattgcgggtgcgtcgatggtcacggcccagtggtacaccgaaacggctccggagctcgcatacaaggcggcgccgatggttgagccgataccggctgagcggattcggagaacggtgtcgtgggcgtttcacgcaccagggcagtcctctccgctggaccgtctcgctggttctacccagcggatggtgtttgacgcttcacgggagacggttcttgaaaacctggaaaacgagatcgcagatgctggagcaccattcccggccaggactagatgggcgcgctatgcatcagctacagcgtgcccattctgccggatgctcgccacacgcggtgcggtgtattggtcgcaagaatcggcgggggcgtcaatcaagtaccacgaccactgtcgatgcattgccgttcccgtgcgccccggccagtcatatgagccgcccgcgtatgtcgagcaatgggagaaggactacaacgctgccgtgtccgccgcaagaagcgatcggaagtttgaaggcgagtacggcgcggtagatccgaaagacattctccggcacatgacttccgcctgacaccaggtgaaggcgcggacgacctgcgctatcggaaacggtcggcccactccaataacgcggaggttatatcaccatgcccgaagaggctgaaaacaccgccgaagacggcgcaacaacccaacccggaaacggggacgaccaggcgaattcgttcaaaccgatcacatcgcaggacgagtttgaccggatcatccagcagcggatcgcgcgcgaacgaagcaagttctccgactacgacgatctgaagtcgaaagccgaagaactggacaagattcgcgaaggcgagaagaccgagctgcagaggctcaccgagcagctgcagtcagtcagctcgcgagcagaaaaggcagaacgcgaccttctcgtgacgtcggtggcggccgagaaaggcgttccggcggccagcctcacgggtagcaccaaagaggaactggaagcctccgctgatcagctgatcgcatggcgcgatcagcaattgcaacagcaagccccaaagctcaaaccgcctgccaagaacctgaaatcaggaaccacaggcactgagaccgcggacctggacccgaaagcagcagcagctgaagctctgcgtcggatgcgggccggcggttaacccaaccatcgaaacccgttcgaggatcggcctcggcggaaaaccatgaaaggaaggccatcatggctgacatttcacgttccgaggtcgcgaccctcatccaggaggcctacgcaaacgacctcctggcgtccgcgaagaagggatcgactgtgttgcaggcgttcccgactgtcaacatgggcaccaagaccactcacctgcccgtcctggcgaccctgcctggcgcttcgtgggtgtcggagtccgcgaccgaacctgagggtgtgaagccgacgtctgaggcgacgtgggccgaccggaccctggtcgctgaggaagtcgcggtcatcattcccgttcacgagaacgtggttgatgacgcatcgacctcgctgctggaagagatcgcggctcttggcggccaggcgatcggtaagaagctcgatcaggctgtcatcttcggcaccgacaagccgtcgtcgtgggtgtcgcccgcgctgctcccggcggctgtcgcggcaaaccaggactacacgatcgttccgggtgacgcgaacgaagacgacctgatcggctgcatcaaccgggcgtcgaaggcggtcgcggcagccgggtacatgcctgacacgctgctcgccagcctgggattccgtttcgacgtggcgaaccttcgtgacgcgaacggtaacccgatcttccgtgatgagtcgttcaacgggttcggtacctacttcaacgccaacggtgcgtggcctgtcggtgtcgctgaggcgctggtggtggattcctcgcgagttcggatcggtgttcgtcaggacatcaccgtgaagttcctcgatcaggccacggtcggatcgatcaacctcgctgagcgtgacatgatcgctctccggttgaaggcccgcttcgcgtacgtgctcggcaacggcgctaccgcggtcggcgacaacaagacgcccgtcggcgctgtcgtcccggatggcagctaaggcgatagcgaatgtcactggcggatatcgatgacctgaagttggttctcgggcgtgaactgacgccggacgaagccactcgtgcaggtcttctcctcgaagaggcgtccgatctcgttgtcggatacctcgggtgggagtcgattcctgacgtgattcctggtgctgtggtgagggtggtggcgcaaatcgccgctaccgccctcactgcacctcaatcgccataccccgaaggcacaaccgcatctgctggcccgtactcgttcaagtggggcggtgattcgtcaaacttgtatcttacgaacgcgctcaaactgcgtctgcggccataccgcatatcgatgaacagcatcccgctgggttccgacaggtacatgccgtgacgttccctactccgtacacggtgacgcactatccgcacgtcggtgactcggcggatggtttggggaacacggtcccccagttcggttccggggtgtctgttccagtgatccaacttgctccgcacgtgcaggtggtggggacgtattcgattgtggaaaccgaaacgatcgatgttgacctgtacttgccgcccgattcaccagtgaaggtgaaagaccgtgtggggtacgggtcagatgtgttcgatgtggttgcggttcgtgactggaacatgggttttcacggttgggcgccgggtttggtggcagaacttcggaaggtgtgatgaatcgtggctaacggtccaacgaggaagaaccctttggcgaagttcggtgtgcggcttgatgatttcgacaaactgcctgaggtgaacgagggcgtcaacgagttcatggacgaggttgttgacgcgtggaagaacaattctcccgtgggcaccggcgcttaccgtgattctgttcaggtgacggaacgttccacgaacaagggtcgcgggaaggtcggtgcgactgatccgcaagcgcatctcgtggagttcgggtcggcgcacaacgacgagtacgcgcctgcccagaagacagctaaacagttcggtggcaacgcgtatggcgactgattcagcgccgagtatccaccgtgtgttggtggcgtggctgtcccctttggggaaggtttctactcgccgtttgtcgggtgatccgttgccgcatcgtgtggttcgtcgtgtcgatgggcgtgacgttcccgaggaaggcagcgattctgctgtcgtgtcggtgcacacgttcgccgcgtctgatgaggccgctgagaatgaggccgagttgacgcatcagcggatgctggagctcgtcgttaacccgctggtggagataccgctcggcggtggtgttgttgcgcgtatcgactacgcgcgtgtactgatgaaaccggtcctcgtcgagtatgacgacgacggccacctggtgcggcatgtgggccgctacgagatcggtgttcagtacatctaattgaagtttcagccctgtcaaaggggcctggcggtaacgccgggtccctttttgttcgccggaaattttcgcaatccggtcccttatccaaatgagaggagcgtccctaatgacgcagccattgaccggcaccgactggagcgccggcggattcactgacattcacaagccgttcatcgagcgtggcgggctgcaggcggtgttcatccgcgacaatcgcggtgccgcgactgacatgtcgccgttcgaggatgattgcgtgacggtgaagtggtcgccgtttgcgcaggacggcaagcttcgcgatgacctgttcattcgccggaaggtgaacggcaagtacgagtacaacactgacccgaatgagggttggtggcacatcggctgcaaccctgaggatggcggtgcggagcgtgaaccggatgtcacctctgacgatctgatggtgttgcagtcgaagttcccggtcgattctgaggtgacggaaaagtcgtactcggtgcggttcgtggcgctcggtactgctgatccgctgattcatcggctggagtcggaacttccgttgtgcgacaacgcgggtaatccgctggtggctcttcccggtacccctgactacggtgagggtccgctgctggacgctgactcggcggagtaccagctgctgctgctgtacgcgcgccgcacctcgggcgggttcatttaccgcgctgagggttatccggcggtgaagctggacgaccaggcgtcgaagcagcgttccaagaccgatcctgacgcggcggacctgacgtacaaggttctgccgaatgagtacttcatgcggcctgacccggcggggacgatcgcccttgttcccggctacttctatgtgtggatgggtggccccggctgggctgagcagtattcggacggcagctagccggtaagtcgtcctgccgggtgggttggtttggggctggcacccacccggcaggcacccacataaagccagcccaacccctcaaccccgaagcccccccctttttaaggaagcccctgatgtctgtgaagaaacctgaaaacaatggtgccgccgcgcgtgaacaggccaccgagttcgattcgccgttcgctgatcgtgttctgcgcttcgatgacggcagcaccatgacgatccccccgcacccgaaccttcggatgctcgacgatgatgcgctggaagcgtacgaggcgtacctcgaagagatcgaaacctatgaccgggaacctgacctgtatatcccggagcagaccgtgaaggaccgcgacggcaacgagatggtcctgccggcggagacccgtcccggcgcggtcaaggggccgccgtatttcaaggacggtaagcgtgtgtcgccgccgcgtgaggtgcggatcgttcaggtcgtgttgggcatggacaactacgaggtgttgcggtcgaagcagatcaacggtcgtcctgcaggtgcccgggatgtgtggcgggcgtggaccgagcaaggtttcacgatcgcggaacgagctgagtccgactcgaaaagtgatggaagctcagtggttctggagactgtacccgagacagatagcgagtgatctgcggcgcttcttcgggctgagcgtcgccgattggcatcagggcaggttgtccagtttggagttgctggacctgttcggggttcggttcgtggacaacgctgaagaacgcgttcgggagttgtatgtggatttcgcgccggtcaatggcgcggtggcgcgggctgttcgcgggggccgctggtctgagtcggagttgattgcggcggaaacatacaacgagatcgcccggttcagggcgtcattccatgcatcgagaagccgtaaagcggcgtatgagccgttcgcttttgaggacccggttgatcggttggagaaagcgaaagcgtcggttgaggcgcacgagttgcagcgtgaggttgaggccgatctgttcggctggtgacgggaggtgagtgtctgatgtgtgctattgatggatgcgatcgacgagtcttctcccataagaacgggctgtgcgcaacacatgaccgatacctgcgccggtttggcattgcggagccgacgctccagcagagattgtttgcgaaggtggacaagtctgcgccaggtgggtgctggctttggacgggcggaactactaaccatgggtatgggaggtttaataacctttcgccgcaccgtctttgctatgagtgggcgcacggagagatcccgcccggtatggagatagaccacatctgccacgtcactctgtgtgtcaacccggatcatcttcgcgttaccaccgcgaagcagaatcgtgagaatagatcctctgggtggggcaggagcggagtgcgtggcgtgcgtttcaaggctggcaagtgggaggcggtcgtagttcataaccgtcagcacatttactgcggacggttcgagagcaaagagtccgccgctgcggccgctaaggcgaagcgaatcgaactgttcactcataacgacggtgaccggggagcgtaatgcctatctacgtcaacattatttcccgtcttgatgagcgtgctgctgcggtggcggcgaagaacattgagcgtgagatggaggcggctggggcgcgcgcggggtcgtctgctggccgagcgatcggcgagaacgtcaccaaggaagctgcggctgcggggcgtaatgctggcgagcagttgtcgcgtgaggttgatcgtgcgacgcgtcaggccgggtctcgcattgttgacggtttttcgtcgcatggtgtgtcggcgggccgggggtttggttcgtcgtttggttcgtcgttgagttcgtcgttgcctgtggcgggccggttttcgtctgccatgtcggggtatgagggtgctgcgtcgaaggctggcgcgttggctggccgcgcgttgggcacggcgttcaccgcggccgcgacaggcatcatcggagcagccagtgttgccctgttcaagggtttcgacaggtacaagtctcttgatgcgacatcgcatcgcctttccgcgatggggaacagcgccgagcaggtcaagacgatcatgtcggatatcaacgaggtcgtcgttggcactccgattgcgttggacgaggcggcgaaagctgctacgcagttccttgctggtggggtgaagcagggtcgcccgttgcaggcggcgttgactgcgattgcggacgcggcgggtgcatctgggcagaagttcggcgacctggccgtcatcttcaaccaggtgttcaacaagggcaagctgcaggccgaggagatgttgcagctcaatgagcgtggcatcaatgttcaggcggcgttgcagaaagagttcggcctgacgagcgccgagattcagaagatgtcgaaggacggcacaatttcgttcggcatgcttgtgcaggcgattgagggccagttcggtggcatgtcgaagaagctggccgacactgttgacggcgccttgtcgaacatgaatgccgctgtgggtcgtgttggggcgaacttcatttcggcgttgttcggtgacccgctggacacgacggagggtcctggcgcgcttgccaagtcgatcaacaatgtgaccgacaagctgaatgacctgaacgcgtggatcgtcgcgcacaaggacgacatcaagcgcaccttcgaggaagctgccgagactgcgcaggatttgtgggatgcgctgtcgagcgtagtcgaaatgctcgaccggatcggtatcagcgttggggacgtggtgaccgcgttcgtggcgtggaagtccatagctggcatcacggccctgacgcaatctctctcaacggtgagcacgacattggccggtcttcccgcgacggccgataagtcggctaagggaatctctgccgcgttgtcgcgtgtggcggtcccagcgtggctggcgttcctggttgcgcagaacggccctgagattgaacaggccattcagaacgcgattccaggtgcggatagctggaatcactccaatacgccggatcagttgggtcgcagtgcccgtgagtggtgggaccgcaacattcagggcggcacgggggttgatccgcagccgtctccgcttcctcagctcggcggcgggtctggacctggcacgccaacggttggcggcattccgattccagggcttgtgggtacgaactcgaacggtccagcgtccccgttcggtaaccttcccggtcaggttccattggatgtttccgtggaggatcgccgcgggcgtcgtggtggcggcgcgcctggttcggatggggcacccgcggatggcccgttggctgatctgttcccgggcgcggtgggggctgctgatggtggtagtggttctggcccgaagttgccggatgcgcctgtgttgccgtatgacacgacgctgccgccggggattgctggtatgccacccgacgcggccgtgttctccgctgagtcgtcgtatctggatgcgcgtcacaaactggcggagaagcgtgcccgcgccgcccaattggagcagtccaccgaagccaccgagcaggaccgcctcaaggcccgcaacgatgtgatcgaagctgaacgcgaccttcaggccgccgagatgcgcatgagtgatgcccgcgcgaatcagtacgagaagctgacgaagcaaaccgacaagcatgtcaaggatttggggcagatcggtgcccagcttgatcaggatttcggtatctcgaagggtttggcggggatcgcggagaacatcacgaagttcgtggcgaacctcgctgcggcaccgttgttggggcagttgcaggccatttcggcctataacccgacccagggcgggcacgggttgatgggtgtgctcggcgcgcagggtgtgttcgggccgcagtaccagaacaaccagtatgaccggggctcctacccgtccgccggtgcgaccggtgtgtccatgacgccgatcggtgcctatcccggcgacgcggcgctactcgccaacgttccggcgggccggtacgcgcaagtccaagcggccgacctcacgcagggtttgggtgactgttccagcgctgttgaggatctggtcaacattctcgatggcaggtccaccgagggtaggtcgctgtccacgcataacgctgatcagtggttgcagtcccgagggtttgtgccggggtcgggtgggcctggcgatttccgtgtcgccttcaacccctcgcatatgcaagccaccttgcctggtggcacgccgttcaactggggcagcgcttccgctgccgcgcggcgtggtatcggcggcacgggcgcggatgatccgtcgttgacgtcgcggtattaccgtccggtgacgtcggttcctggcgggtcggcggcggcggcgggtgctccggggttgtacagcccgcagaacaccaaccctgcgttgaataacccgccggctccggtgtcgtcgggtgcgtgggcgacgaatcctgcgccgctgcccactacgggcggtggtggcggcccgatggccgctggcgcaccacaaggtctgttcactggcgggccgacgaacaccaccaacatcggggcgaacgtcgcaccgtatgccgggtccggttccggtggtatcggcatggacggtggtggcgcgcttggcatggcggtgcaggccggtggtatggcgctggacgcgatggccccgggtgcgggtcaggccgcgcagactggggtgaagctgatcaaccgtgccatcgagtacggcggtcaagtcgccgcgatcggcgcccaagggttgatggaaacgttcttgcctacgggtggttcggatttggcgaacaacaactggatcacccgcattgccggggggattgctggtgcggccccggcgttgccgaacctggctggccaagcatcccagcagcgcaaggacattgatccgcaagccacaggccagggtcaaacccaagtcaaccagggtggcgacacgaacatcacggtcaacaaccagcgcgccaccgaagacggtacaggccgcgacatcgcgtatcacctgcaaaaccagtacgtcatgccgggagggtaaatggctaagaagcattaccccgccactggtgtaaccccgcacggatggtatgacctcgccaagggtgaaaagccgatgatgtggctcgacgcctacgacgggtcgatcactttccacatgatgggcgggatggcggtccctgaccgggttgtagccccggagatggtgcacctcacatcactcaaggggttgatcccgccgtggaagcacatcgaccagaagggcgccaccgaggacggaatcaccaatattgatgcgctctacgacccgattgaggttgaggtgggggtggaatgccgtggccggtcgccgaagtggacgcgcagggtctaccgcgatctggtcgcgtcgatcgacgcgaagcaggaatcgacgttgaacttcctcacccacgacatggggcactggtgggcgccggtcaggtggttccagggcgcgccgcaagcaccgctggagatcggcaagcggcagcgtgaaagtttgcgcctgcgggccgattcggggttctggcgtacctacgactacgcggcgagtttccagttcgagtacgagtcgatgaccgacacgttcaactatgacacgtcgggcacgcaggacctcggcgcggactggccgctgtactacgagggtgacggcggcgggtacgtctacgccaatggtgaccaggcgaggtggcgggacgacccggacgatccgctgacaacggatacccgcgaggtggtgtgcgggccgtacaaagacttcgacaccgacaccgacaatcaggttgtgtcgatggtgctcggagggttccaagagtggagcctgcctgatagtggggctaatgacctgtgggctcgcatgggccgcgacagcaacggagactgggacggtaatggcatccgcatgcgggtgcagggcaactggatcaagctgtcgaggttcaacaacttctcgcagacggtgatgtttcagcggccgcttctggtggccccgctgattggggagaagttcaccctggttgccgggtatgagggcgatccgcgcatgttcaaagtgttgcgcaatgggttgccgatcttgtcgcacaaggaaaccggcactggtagcgagcttggcccggattatcggggtattgggtttggtatgcaggccggtggcgcgttgatcacgcaggcgacaccagctccggtgcggaaagtgtcggctggcgacaatgcgaatgtcacccagtcggggtttgtgtcgatggtcaatgttggtgaccagccgatgtattgggatgcgaccttgtttggcccgggcacgttccggttgtatgacggtcccggcgcggatgagtatgtggagtttggtccgctgctgcccaatcagattgtgttcctacgtaccgacccgcgctcacagacgacgttggtgcaggatttgacgtcggtgccgccgtcgccgcaggagttgaacattttccagcaggcggtgaagtcgttgttgtcgttcttctcggaacggaacgcgttcaccgaccagattgggtcgctgtttgggattgttcccccgcagggcaatttctataagtacctgtcgggccggttcagtgagaacgcggcgatccccgcgaagtcacctggcgaaccggcgcagcagttctttgtgaagacagaaattgttggtggcaacgctgactcgaaggtgattctttcggggactccgttgcgccgctacccgatgtagccactggccgatacctcggtgaggggtgaatttgtggcgcctgtgaaccaggaaaggaggggatgacggttgtcgaagtttgaacgcgaaacagccgcatggcaatccgccctccagtccggcgacccgaacaggatcgcacgaaccgcgcgggcgttgacggaacgcaaatcgaaggtagacacgtcgttccggttcacggtgtgcgacaagttttggcagccgatgggcgctgtcggtggcgacctgatcgaggcgtcgggtgctgacccgcgcaacgatgttgaaaccggccggatcgtcctcaaagggaacagccctctcatccctttgttcatggactgcaaaaagacgatggtcggtgtcatcgtcgagacagccggtttgcggtatgcgttctacacgaagaaccacacctacgagtaccgcgacagcgcatggaccggcaccgctgaactgcgcggtatccgcgacatcctcaattactacgtgatttggccgtcgtggtggctgccgattcaggcacagccgttctcgcacgcgatcttcgtgtgggcgctgcaaaccgtcgtggagaacatggtcgcagaatgcgctctgcggttgcagtccgggtggctggagttcatcaacaacggcctgtcgttaaacccggatatccgggcatggttcggcaccgttctgcaagccctgtcgcgggacgggctgtcggtccaggcgttcacccgcatgctgcgaaccccggtgtatgtgtcacgcaccaatccactgctggacacgtcgccgatggtggctcgcacagtgcggatggaaaccgttcaggccgtcatcaaggacgttacccagtcgtacggtgtggatacccgcatggatttgtggcttccaggtgatccgcagcctgacaggtgggcgaacctggaccagcctacctacgtgttttccacagtggaccggtcgcagatcactggtccgacgaaaaccgtgctcgattcggtgctgcgcaccacgattgaccttggcgggtcgctgggggacatcttcaaacctgtcatcaagcaggttcccggcatggacggcgtgttttatgcgcccgcgttgggtgtggatttcgagcagccatacgcctatttcgtggcccccgagccgggtgaggacaccggcatcgatgcgtgcacgatcactgaccacacacctgagggttggcagcacactattggtggccgtagcccaaagtggttgaacgacttgatgaatgccaccttcgcatggctgatcgactcgctgatgatcgttgttggattcaccggcataccgtccgatctgctgtcggggttcctgaacaacagcttcctggcgttccagttgattcagcattacgaccgccgtgacgaagttggcccgtaccatccggcgatcgagcggttctatccgacagcatcagcgccgtacaacatcgaaacggtgttcgcattcatcaacgccttgtttgattcgcagggcaagacgacggcgacggtgcagttccgcaacggtgcccagtatgcgttgggtcgggacgtttttcgcggcggcctgatgtcgctggtgttcatgtcacgtacccgaatggtgactgactacatcgaaaacgtcatgtggcgggtttcccaggatgagcggaaggcgatcgcgcagttgggggatggacgcaagtcggaggccccgttggcgaagcatcagcggttcatcacggggatttttgaaacgttgtcggtcctcacgctgtcacctcagggataagcagcggtcgtcctttctttctgtaactcgcccaatgtgaatggagcgtgccttatgtcgtggcctttgaatcctgctgggactcactatttgtttgaggggatcgtggagattcctgtcgatcctacggcgggtgcggcgattctccagttgcgtccgcagggcggtatcggtgttggtgtgcccgcgattgagaagggtgatccgggtgtgccggccacgttcgatgcgacggtgaatctgacggagctggacccggacgatccaaccccggcggaggcgtcgttcactgagatcacgccacctggaacatccacgccgggtgtgtaccggttgaaccttgcgttgcacgccggcgcgaagggcgcggatggtgaggcggtgtgggacccgacggatgttgatccgtcgccggttgcgggtcaggtgccggtggtgaattcgactgctgatgggtttgtgttggcggcgcagcgtgtgggggaccggtatgttccggcgtcgatcaacaacactgcatcgggtaacgcgaactcgactttggctcaggtgtcgattcctgcgcagccttttgattggcggccgcgtgtgcaggggtacacggtggtcaccggtgagggtgcggatgttcgggttgatttggtggcccggttgaacggtgagactggcggcaatgtgatcggccggtgccccggtgtggcgcaatcggagcggctgacgcttgtttcgggacctgcggcgggctcatcggatgggtttgaccgtgtggcggccggtacaccggcgacgatctatttccggtgtgaacgtcaggcggggtcggtgacgtacacgacttctgcttctacgtcgatgttttcggttgaggtttggccgctgtcatgacgtcatcgtttgatccgttgccggagtgggcgcatgcggtgccgtctgagccgggtattcacccggagcagtcggcgttgcagtggcagcgtccgttcactgttcagcagctgcttgagattggtgagcagttcatcgagcagtttttggcgtgggtggtgcgcgcggtagctggggtgttcatccctggtgaggcgtcgttcgatcagttgcgtgattgggctttgaacatccccattctcggggacattatcgaggcgatcaccggccttgtgggtggcggggttgaggaactgacccagttcttcacgaacgttcgaaacttcttccagtcgatcgacttcaacgatccgagtttcaacccgcttcaggctgcggcgcagttggtgaacatcatccttgcgccgctgcgcaatttgctgcccagtttgttgacgattctgccgatcggtggcatatcaaaccaagcaccgaacattcttcctgccccgaagttccctgaggggtcggtgggcgagaacgcggattgggttgtggacccgtcgcattctcgcagcggggatggtactggcgcggcgaaagtcattgccgatggcacgttgaaggcgctgcggtcggggcagaatgttggcgatttcttcgcggtgagcgaagggcagacaatcactgcccgggtgttcgtgtctcacgacgattatgtgggtacgggcgcgccgatccggttgcagctggtgccgtacatcgacggcgttgcacaggcccctgtggatttgaacgcgtacgccccccaggacgcgaacttggcgtggcccggtaaggagctgtcgggggagtatcgggtgcccgctggggtgactggtgtgcagacccggttcgtggtgaccgaagacgccactgcgggcacgttctggtgggatgacgccgaggtcaagcagaccggcgttattcagcagtcgtgggtcgagggtcttccggagattctgcaaaccttgttggcgcgtgtgcagttgacgattgacacagtggtgtcggcgatccgcggcggcgtgcagaccgttgagaacacgctggaggatttgttcgacgctttgcgcaacatctccccggagtcaatcgccggcatgctcggcccggagaatctgcgggaaaccatcgagaacatcgtcaacagcattgtcggtggcctggtaggccttccgggtattggtgctggtatcgccgacctgttcaacgtgttgcaggagatcgcctcgcgcgccagcttggggttgttctcgtgggacatccttggcatcaggaccaacaagcccgtcgatagtggtttgttgccgtcggagcggtccaacttcccgctgtcgaacgtcacgacgtggctggaggccacgcagagcaattcgctcatcggtgttgacttgatcgaagagtcgatgccgcttggcgtggtgtcgtggatcggctacggcctttcggggatcaccgagttctacgtcaacatctggaaggtcgacttggcgtcgggcgactggacgctggtgcaccattccccgaacatcgtggggcttttgggcggcacggccgcccccggggagttcatctcctacgagttggatgacccggttcccgtggtggcgtctgaggcgtacgcctatgagcttgtcccggtgggcggtacgcattatgtgcgtggccgtgtggcggacttgccgaatcatccgacgtcgcagattgtgtcgctggcggccaccagaaacaacacgtcgccggatagcccgccgtcgtcgattgcgaaggcgtcggtgacccgctcgggcgatgtgccgtgggtgagcatcgccgtggatacaggttccggcggtgaccatcacgatccgttgaaggtctaccttggcaccgcggccacggtgttcccggttccgaactgggtgaactacatcgacccggttgcggtgggcgctggtggtggtggtgcacaaggctgggcattgggcatcaacggtcaggccggtcagcctgggaagttcaacgccaccacatgggtgcgcggtgagcatttcggcgacaacgccatcatcaccctcgacccgggcgctggcggcgtgggcggtcctggtgacggcgcggccggcggtaacaccacgttgtctatctccacgcccgggggtgacacgtattccattgtcgccgagggcggcgcggcgggcaccactgaagggtttttgtcgaaacctgttggccgaggcccgggcacgttcacgttcaatggccaggactatgtgggcggcgttgaccagaaggtcatgggcggccacggtgcgcccgctggtggtgccggtaacggcggcaagggctcgttggcggcctttcagtccggcggcaatggcgctcctggtggcggctgggtgttcttccggcccgacccgctgcctgaccctgacccggatttgacgccccccactgctccgacgttggtggagctggtcgattcaactttcagcactatcacgattacgtggtctggagcaacagacgtatgacaatcaaagggtatttcgtttacgcgaaagagaaggacgcttcaggcgatttcgttcagttgaatcccgacccggtgttgccgccgtatgggacaaacggtttgaagtcgaacaccacgtacgagttttatgtgaagacggtggacaacgccggctggctgtcggacccgtcggatacctacgagttcaccactcccgcgcacactgcgggtgatttgttgtcgccggaggaccaggcgatggtggatttgattgtggaggagtcccgcgcggagaccggccagccgggggtgatgttgcagatcaccggcccgcgcgggaactatgcgaaggcgtatggcaccaccgtgggcggcacggttcgcccgttgacgttggatgaccacttccgcatgggttcctccacgaagatgttcaccgcgattgcgtttttccaggccgtcgacaaaggcctcatcacgctggatgacactctggagcagtacgttccgggtatcccgaacggtaccgcgatcacgatggggcacatgctgtccatgcggtcaggtatcgcggagtatacggcgggtatcaacgcgctctggatcacgctgtttccgacgtggccatggacgggcgcgaaggacttcctgggctctatgaaagggccgtcaaatttctatcccggcaccgactacctgtatacgaactccaactttgcgctgatcgggatggttctagagattgttgacccggcccatcggccgatcaagcagatcttcaaagaagacatcatagaccctcttgggcttacggaaacgtcatggccgccgatcggtccagttccacccccagcgtcgatcgctgacacgttcaacccgaacttcctcgacgctgccggggcgctggcgacgaacatcaacgactacacgaagttcgcggaggcgttgcgggacaacgcgatgggcctgtcgcccgagtcgtatgacgcgtggctgtcaacattctggaagcatcccacggggtgggacccgtacgcgaacgggttctacattccttccgagtattactacgggtacgggatagagtcgttcggaacgtggttcgggcatccgggacttttttcgggtggctggtcgtccacgattttctttgagcgggactcgggtgcgacattcacgctgcacgagaactcgaatacctccaaccccccggccgcgggctatacccgcatttgggtgcgggtggcggagtatctgtatcccggaacgattacgaatgaccagaactggccggtgccgccggagccggtggatgttgggtttgatgccgtgtcgggggctggggctggtgtcggtagcgccactgtgaacttcaaggcctccgagggggccacggtgttcgcggtggtggcgtgggaccgcgcgggctcagccccgtcggccacgtatggcggcgccggcggtgtacttctcgggtccgtttcgcacgatggcgatccggcgaatggaggattggcgattttccgcatggagaacgcaggctccggcgttgctcgccagatgaaggccaccggcccgggctgggtgagtgcgtatgccatttcattcaacgatgttgtgtccgtgggaacccccacgttcgcgcacggcaacggcactgcgcacagccagtcggtgacggtaccgagcggggtgacgctgcaggcgttctcggccggggccgggggggtgtcgtcgtccaagctgacaacgattctgggggcgcgcttgcgcgcggagcagtcggggatcgccccgcccctgtgtgtcaacacaaccacgaggacggggacggtgagcgctacatcgtcgcggccgaacaggtgggctggcatggcggtgaacttgcagattgggggatgagcgtggctgttggctggtgggctgagtcccacgtctcgtttggcgtcaccctcgcccccgagttggggtttacctatggtggccccaaggaggagtttggcgtcaccctcacccccgagatcggcatggccgccgtggcgcacaaccgtgcgagtttcggtttgtcggtgccggtttcgctggggatgggggcggccagccacagcaaggcgtcgttcggtctggtgttcgcgccgtatatcgcgatgcgtggtccggccgcgttcgagccggtgtttccgtccgaggatttgtatccgtcggtgtcgctgttcccgacgccgcgcgcgcagacccccggtttcgggttgtcgttcacgccgagcctggggttcgaggccgcgccgaagtttgcgcggtcgttcggtatcgaactggacccgcaggtcggcatgggtaccgcactcgggttcacgaagggcttcgggatcgaactgtccccgcaggttggaatgtccggcgcggagcggtattaccgcgagttcgagctgacgttgaccccggaaatcggtatggacgccgtgggtaatgacggtgttgacccggtggcgttcgacgcggtaaccatgtcccagcaagcgacgtcgacgttctcgttcaaccacacggccaccgccggagcgtcggtactggtgtcactggtcgtacagggcagcgacacgatcgcctccgtcacctacgacggatcagcgatgacgcttatcggcagccaggctctaaacaataacgctggcagtggttcccaacacttgtatgtcattcatggtgttgctggcgggtcgaaacaggtgacggtcaacaagcccaccggcttcgggtgggtgggcgctgtcgcggcctcgtacctgaacgcgaccaccaccggcactgtgcagaagtcatacggaaacagtggttcggcaagcctgtcggcgtccgcgcctggagacggtggccgggtagtcgtttcgttcgccaacatggggaaccggacgtttacaccctctggcggaacgaaccgattctcgggttcgggcctgttcccgatcctgaccatcagcgacgcgacgacggccacgaacttcacggcgacaagctcgtcgggcacatgggccgccatggcggtcccgctcaatcccgtataactcgaaaggaaacaatcatgggcattcccaatgcaactcacaaggcagcgtcggacgccatcgccggtctcggtgactggatcagtgtgcataccggagctgctggcaccacaggggcgaatgaagccacgggtggtggatatgcgcgggagcagacgtcgtggacgtcgggctccacgggcaccaacaccggcgacgaggttgaaatcttcgtggcggcaggcacctacgtggagggcggcatctggtcggccagctcgtcgggcacgttcgtcggttcggaagctttcgacgacggtgacgtggaggtgtccggtacgggggcgagcatctccgtgacgccccgcatagtcgcctgaaatcctggataggggaactgttttgaacatcaaaactgatcatcagatcgtcgcgttcggcaacgacatgatgggcttgtttgaccgtgacggcacgttgattgtgcaggccgcccgcgtggtcggcgggtgggaggtcaccgccgaggggcggcccccggcgaccgtgttggatcggtcttcggcgatcaccgaaatgatcaacaccgccctcgcggtgcttccgggtgacggttattcgtgcctggtgccgaggggtttgcgggcgcaaccttaggagggggtttggtatggcttattcgaagcagtcgtgggagaacgttccctcgacgaacaccccgttgtcggcggaccgtctcaaccacatcgaggacggtatcgaaggggcgcatgaggggctggacgataaggccgacctcgcccacgaccacgttttggccgatgttaccgatgtcacctctactggcgcggctattgctggcgcggcggataacgatgcagccctggaggctttgcagccagagttggacaacaagatccacgagatcgtcgactactacgcgaccaacgagttggacgttcaggtggacgcttccgacgtggtgtcgggcacgctgagcattaatcgcatccccgtgggtagtagtggttccacggtgtgtgttggtaatgattcgcgcctgtcggaccagcggacacccttggacaactcggtgaccctggccaagattcaggacggtgcgatcaccaacgcgaagatcaataccggcgcggcgattgcgaaatcgaagctggcttcggatgtgcaaacctcactgggtaaagcggattcgtcggtgcagaaatccggcagcgcgtccgggatgtggatgggcactacccttcctggtaccggcacggcgggtgtgttgtacgtggtggtgtcgtgaaagtttggaacggcacggcgttcgttgaccccaccgcgttcaaggtgtggaacgggtcggcgttcgtcaaccctgagttgtacacgtggaacgggaccagctttgacaaggtgtggccgtcgtttgaaccgttcagcatctccagcgaagaccccggctacgaggatctgatcgacgagccggtacccgagggcgcatccggttgctgggtcacactcggcggtgcgggcggcggcggcggctccggccgcagatccaactccggctaccgctacggcggcggcggcgggggcggcggtggctacatcggccgcgtctggattccacgcgcgtcgctcggctcgacgtataccctcgtccggggcctcggtggcgccggtggagcgcgggcggcgggatcgtccaacggcaataacggcgctcccggcggttcgactgtgttctcgtccggcagcgtttccctgacggctagcggaggggcagcaggcgcgaggggaactaactcgtcgtccagcggaagcggcggggccggcggtacaaccagcatctccggcatatccgcaacaggctatacaggtggcaaaggcggcaacggcggtagtaacccaactagcggggagagccgttcaaacggtgcgggcgctggcggtcggggggctggaggcgtcctgtccaacgacaacagcttcagcggcggtagcaacggaaccagctccggccccgcggggaacggcggcggggggaccgccggagccataaacacgggcggatcaaacgcaggtagcggcggtgacggctacgtcctgatcgagtgggaataactcgctaacggttcgggtcaccagcagcgcggagttgatacacacgctgcttggaaatcttcagggcgcggccaatgtcatgccacgtgatgccgtggacagtcatcgcctcgtagacgagggcagccagttcggcatcaagctcggcgatagtcgctgcgcgtttctgccggttggcgatcatgcggtcgatgattgtcacatctaggagtgtatctcaaagaaacacttgtgcacgtggtcaaacgcggttagactcgcgttcatcaacttgagacaccgcccggcggggcgataggcctgagaaaccaaccccgccggacggcccacccccaacaggaggcccaccaatgctacgcaccaccaccgcgactgtcttcgcaatcgccgcactcgccctcggaatacccgcagtcgctgatgccgcacccgcccactgcgcgaatcacggcaccggccacgggcagatctacaagcacgcatgcgccaccggcagcggcggcgcaggagccgactggacatacgccacccacgccgacggcacacccaagatggacggcaccaaacacatctacaagtgcgtgcgccactgcggcggcggccgccaccacgtcgaaaccaccgacacctggtgacccgccatgaagatccacgttcaatcccgcggccccgccggctggaacgcaacagtcctcttcaccacaggaaccgtcctgactgtcgctgacgaccaaggtcgcaggcacctgatcgacacgtcccgcgtcacggtcaggaggctgccgtgaccaagcccactgtgaaacgcatagccggggctctcggaaccggactcctcggaggcatcgcactcaccagtgtcctgtcctggatgttcgccacaggcaaccccgccattgacttcttcatcgaacgcgacaccctgttctacttctaaacccaccccagaaaaagccccgccacccacttggtgcgcggggtttttctatgccccgaaaggaaccccggacatggaccgtctcggaatcatcctgctcaaactgctcggaccactcgccgacaggatcgccgaccgcatcgccgacaggatcaccgagaacctgcccgacctgtccaacttggacgaccagatcgtcgcgaaactccccgacctgaccaacctgccagcgcaagtcatggacatcatcgacggcgcgctgcgctccatccccgtcctcggcggaatcctcgggagcaaacggtgaccacgaaagatcaagtcgcccaaatcaccatcgccgaagccaaggcgcgcggctacacccgcagcgaatgcctggcgatcatgtccaccttctaccaagagtccggctggaacgacaccatctgggacccgacccacaccacctacggcattgcccagcaggacggctcctacccacaccgcttcgacggtgccgcagcccaaatcaaaggcttcttcgacaagctcgacgtgtggcgcgccaaacccggtgccagcaccgatatatggctgaacatctgctggatgcagcaggcccccaactggcccagcgctgactactggtacgccaacggccgccgcgcctacctcaccgaaatcaagtcacgcatcgccaccgtcaccccatacctcgacaagtactggcccgccgatggaggtaccgccgtgcccgacgaaccacgccccgacttcaacgagtttccgatctggtcgaacaacaacagcgcccgcagcggcaagccaaccatgttcctgatccacacccaagaaggcggcggcggggacgctgccgccgagaacctggcgaagtggttccagaacggcaacggcgtctcgtaccactacacgatctcccaagcgtccgatggtggtgtgacggtggtcgattgcgtcgacactgaccgcgccgcctggtctgtgggcaacgccaacagcatcagcatcaacctgtgcttcgcggggtcgcgagcatcctggatgcgggatcagtggatgaagcagtccaacgcaatcgacgtcgcagcctacctcgcggtgcaggacgcgaagaagtacggcttcaccccgctcgtggtgccaccgccgtacacgaatgggcgacctggcatctcggaccaccggtgggtgaccgacgtgttcaagtggggcactcacaccgacgtcggagactggttcccgtgggactacttcgccgaacgggtcaaccactgggccaacggtggcaagaccgagcctgaaccgcccaaggtgaaacgcttcccggacgactggagtgaccgcgaaatcctcgtcgagatcctgcggcaactgcgcggatacaacctcactggctggccgcagctcggcggaaaaaccctcgtggacgcggtagcagaactgttgggccactgatgcgcatcgacggccaatacgtcggcctcggaccaggggacagatccgacgagatccgcaagatcaaagcgttcatgcggcgcaagttctcctacgccgcgacgctggccgacaccgagttctacgacgaggccatggccgcggtcgtcgccgagatgcaatcccggtacaacacggctgggcagctgcgcgacgggctctacatcccggggattgtaggggccgaaaccaagtacgtcatgggctacctaccgcgccccgtcgtggatacccgtcccgtgctgatcaccgtgtgcggcaccggcgtgccctggtgggtcggccccgacgccgacaccgcacgcgccgtcgaagaccaatatctgtggcaacccatcggataccccgccgcaccgttcccgatgggccgatccatcaccgcaggaatcaccgaggcgcacaaccaggccaaccggtggcgcgaacgcatcgaaacccacgggaccgcactggcgggctattcgcaaggcgcggtggtcctctcggagctgtggatgaaccacatcgcacccgaagacggctccctgcgatggatgaaaccccatgtgcgtaaagcggtcacgtggggcaacccgaaccgcgaactcggacacgtgtgggctgatcacggcggctccccaatggccccatccaacacccagggcgtgtcctccaacggcatgcgcaacacccccgactggtggcgcgactacgcccaccaaggcgacctgtacgcctgcaccgaacccggcgacacacaagaggtccgaaacgccatctggcagatcgtgcgcgacctcgacctgttcaccggccccgattcactgctggcccaagtgatcgaactcgcgcaagccccgctgccggaaacgatcgcgatcacccgggcgatcctcgacgccggcatgttcttcgcgaaacgcaccggcccgcacgtggactacaacccccagcccgccatcgactacctacgcacataggaggcacccatgctgacacgttcattctggatcgacgccgccgagcgggccatacgcacattcgcccaaaccgcgatcgccaccctcggcgctggggcagtcgacctgatgaccaccgactggatatcggtgctgtccgtgtccggcggcgcggccgtcgtatcactgctgatgtcgatcggcgccgaacgccgcggaaaccccggaacggcgtcggccactagagcggtcaccaccgcatgatctgggaatcggtgcgcgaagcggtgaacgccgcgtaccagcctgacgacggtatcgacctgataggactgctcatcatcggactgccctccaccatcgccgccatcggaacagggatcgtcggcgtactcaccgttcggggacagcgcaaaggccgggagcgcgcacgccagatcgacgcgaaaacctatgagattcacgagcagaccgtcaacacccacgacaccaacatgcgtgacgacctcgacgagatacgcgatctggtgcgcgacggcttcaaacaggtccaacgcgacatcagcggactgcgggaggagctgcgaaccgaacgactggaacgaatcgaaggcgacaaacgccgcgaccggtaaccaccaggaaagaagggcgcacgaatgtcactactggccgatctcgcgggcctgcaaccccgcacatgccccgcatgcgactgggcgggcgcccggtcgaaacaggaacgcgcagagataaacacggcggtggagtccgccaaacgcggtgaggttcagttcaccgacgtgctgcgagtactcatcaaacacggcatgcccgacatgaatccgcaatcgtggcggcaccacgcgaggaaccatcatgtccctgactagcgacctacgtcaggtccgcatatccgagggtgtgcgcaacaagattctgatcctcgacgtcgaacggctccccggcatcaccgaacagtactggtgggacaggggcgacctgaagaaccggtatgtgcagtacgagacggtgacccgaatgccgcgcaccacgatcgtgtgcgccaagtggtaccacgacgccgaggtcattcaactcgcggaatgggacagtggtggccgcaaacggttcctgcggcgcgtgcataatctgctgtcgcaggctgacattgttgtcgggcactacatcgacgaggcggatgtgccgtggctgaaaggcgacctgcacatcgaggctgggttgccgccgctgccgccgttcaaaacggtggacacgctgaaggtgttgcgccgtgagttcaaatccggggcgccgttcaaagggttggatgcgttctgccagatcgtcgggctgtctgcgaaaactgatcgctacgaccggtttgcgatggaacgcgccgtgacggagaagagcgccgtggaccgggaacgtctcatcgcctactgtgttggtgacgtcattgccacgcaggggttgtacgacttcctgaggccgcacatcaagaatcatccggcgctgtttgtggacggcgaggacaagctgacggtgtgtaaccggtgcggcagtgaaaccgtgttgattccgcgccgatatgtggcgaacgtgctgacctacacgatgcgccgctgcaccagctgcggcgcgcattcgcggttgtccattgagcctgagcgcatgagcgttgtgcggggggtgtgacgtgaatattcgtgtgtgtacgttcctcgatcacggtgtgacggtgggattcctgtgggacgcgatcaaggcgtgggttcgtcgtgatgtctgctgatcctgttcgcggcgcgatccaagccagcctggacgcgatgggagacggttggcaggtggcccactatgtggtggtcgtcgggttggaacgcatcgacggtgaccgcatggacttgggtgctacgactgtgatcacacctataggtcaggcggggtatgtcaccgatggtttggtgaaccgttattgggatgagtcgtctggtgagtgatccgcagttggagttgtggcggtcggtgtggctggcggtcgtggcggggatgatcgtcgcgctgctggtttacgtcctggcttaatccacgccttgtgaagcatcgaacttcaggagaagttacggggccgccccgcttgcacaactctccagtgcaagcggggcggccctctttcgtcatgtctacagtcagcagcctccgacagggccagcgatttcttcgcacggatttcgaggagctgacccaacccactcggcaccgtcgtagcagacggaaaacagcaccctattctgagtccacgcagtaaaccccggaccctccggacttccatacccggcataaaacgtcacgtcgaggactgactcatcaggcgtgacttgaacgagcgtctctcccggatactcggacagttctgcctcgatcagattgcgccatgtggtccagtcattcttacccattggcttcttcccatcgtttcttcagctcgtccatgaactcgtcaaccacgtccatcaggacgaacgtcaccatgaggaacggtgctatccacgtggcgatcgatgcgtaatacaacacgcgccccactgtgtgcatgggttcttcgtcgctgccgagcatctcgtcgaatcgtgattcgtaccaagcgaggacactcatcggcttgcttcttcatccatgtttgggcatgtgtggtgcactgggggaagggtgtcgatcactgtctccccgtctttgaacggttgaccgcaccggccgcaacgatcatcggtgttcatcagttgcacatctcgcatccgtggccggtcgggtaggtggttgattcgcctatgtggctcaggttccgactgccagtgggctgcgtcaacgcgacctcgatggagccgcaagcggtgcacatgccgtaaacggtgtcatccgaattaacctcacaaccaccacacatcacagcttcgtagcggtcagtagctgcgggcatttctgtgtctctgcatctaggccgcacccccgaaaggctgcatggcggcgtcgatttcgttcaaatcgacgcggacgaggcgggcaccagcgcggtagccggtcagacgcccgtcggcgatcatttgccggatggtgcggggtgttacgtcaagatatgcggccgcctcagccagtttgacgaagcggcgaggggttggtgcgtctcccatctcagtggcatcctttcgagcgcatcagaaatcttatgcgtcgagccccccatgttgtcgggtgacgctgtatcgaaggatactccgagttgtgcgcttggcgaaaaacttgcgcctcggtgtgtcgtcatcgggcgtgtggccgctgttttggcaggcggctcaccgccagcatcctggcggtttcccggtgaaatcccctatagggttacctttagggtgatcccctctgaggcttatagcctctgacctgtgcgccgtgagggtttcgaacccccgacccgctgattaagagtcagcggttgataggctgcataccaggagaaacgttgtcaaacccgcaggtagacacccgatactgcgcaattctgcataatgctgcgcagcaccgtagggtgaaccgtagggtgacccccgggagggaaacgatggcaaccaagaaacgcagaacccgcggagacggagcgttcttccaacgcgccgacggcaaatggatgggacgagtagaactaccccccgaccgcaacggcaaccgccgctacaaatgggtgtcctccgttgaccgcaacaccgccatggccaaactcaaacaactccgccgcgacgtcgaagagggccgcatcgccaccacctcatccacaactgtggagaagtggatgctgcactggatcgacaacatccacgccaaacgtaaagtccgccccggcgtcctcaacgactaccgggccgccatccacaaccacatcaacccgatcctcggcgcgaaacgcatcgacaaactcaccccgcagcatgtgcgggacctgcactccgagatcggggcctcccgcaccgccgagctggtccatgtcatcgtccagaaagccctggacgatgcggtagcggagggtgtggcgaccaggaatgtggccgcattggtcgacaaacccgagtaccggaagaagaaacgcaacggcttcccggcggacgtggcgcagcacatcattcacaccgcgttccaggtgtgcgacgaaccggatgcggtgcggatcgccgccggtttcctgacgggcgcccgccgtggggaactcctcggcctgcgctggccctacgtcgacaaccccgctcagggatggatcaccatcgcttggcagttgcaatcggaaacccgcgtccacggctgtggggatcctctacccgaaccgtcaccgctggcccggcccgaccgtatgcccaagaaacccccgtactggccttgcgggaagacacgggcatgggcatgcccgcagtcccggtgggacctgccggcgcatttcgagtatcaggaatgtgaggggtcgttgttgttcacccggccgaagacggacgctggttggcgtgaggtgccgttgttgccgccgttgtatgtggcgatgcagaaactccgcaccgacaatccgcatgacttggtgtggcacaaggaggggaagccgatcgatccccgttcggactacgacgtgtggcgtggcgtgttccgcgctgctggggtgatcggtccaaccgagtcgttgccgccgcacaactcgcggcacaccacctcgacattgctgcgcgcagcgggtgtggatgagcaaacgcgtatggagatcctgggtcatgcgagtgtggatgcgcagcggatctatgcgcatgcggaccgggcgaggcatctagaggccatgcaggggctgtccgaactgctcccatcgacgtttgcgtaggcgaccgactgtaaatgcgccctgccgagggattcaccaatccccggcagggcgctttttttgcgttctggcggggagtcaatccgtcatggtccaagttccgcagccgctcgtgcggaacacgatccgatgatccccgttgattgtgccggtccacgacgcgacaccatcgggttggatgttcgcgcggacagtgccggatggtgcttcaccttcgcggagcgtttcgccgccgcggtagtcggcgatgctgacgaccgcccacgtgcagccgggggagccgggtgggatggtggcggtgtaggtgccccagtcgtatccgtctgcgccgcccatgttgtgggtgccgtcgccggggatggtgcggtacgggttgggccgtgtagtggtggttgtggatgtggcggtttgtgatgcgcttctgtcgtcgtcgtcgttgttgttgcgtgcggagacgatgcctacgacggcgagcacagcgagcgcggtgaccatcaccttccctggtgacacagcgcgatcattggtggtcatctggtagtgggctttctgtgttggtggctaactttcgcgcactggcgttatctgatcgtgacattcccatgtttgggcttcctgtgtcgatcctggcaatgatccgttagcgtctacgcatccggttgcgaggggtgaccggtgctggtgatttcggtaggtgcagcccatgtttgatgacgaactcgacactctgctggtgcggattctgaacgcgatggacgagtgtccgccaacaacatggacgttgcgccgggcacgtctagtccttgcggcgttgacgtgcccggacgctcctggcgatgtggtcgcgaatctccgccccggctgtttcgccggtccgaggttggcgcggttgcgtcgtgtcactggtcgcggcgtctaggtcgccctcctggtcttgacgcgcttcgcgcggtgttcgcgtcgtctgcgtagtttccatgacatttcgtgcctcctttagtcgtcgccggacttcggcgagaagttcgtcgtctgagtagcggactatcgccggctcgggtagcggcggtggaatatctgactgttgaaatccggctatcgccagagcttcgttgacatcccattggacagctcgggcagcggcggccacggtggatgcggtcgttccgattgggatcagtgtccctttgttgatctgccaccccgtttccagttgcttccaccgtcctgcgctgacggcgggcttgtcgctgcctggtggcgttgtgcgccgtgaggcttcgcgctgagatagcccgacgcgctctctgtgccgcttgagttctggcccgaatggccagtcttcgcggtgttccttgttctcgttcacgcctacatgttcgcgtgcaaacaggtgcaaagtccactgcttgcacaaccctgattctttgcagttacgcgcttgtagttttcgaacattgcaggtcacagcattgttggcgcgaactgagcgcgaactattgcggtttgcacttgttcgcagtacagttggcggcatggtcaaacagtcctacggggtgtggcaggaactccgggtcatccgtgagcgcacaggttggtcatccgccgaactgtcccgcgaaagcggagtctccgccccttacctctcccagcttgagaacggtgaccggtggccgaacgccaccgtcaccaagaagctcgccgtcgcgctcaaggttcccgtctccgtattggagcggccagccgagcagaaaaaccccgccgcatagaagaagcccccacctgtgtgcagcaggtgagggcagaagacacccgagaggaaagctcaaatgtctgaactacagcttaccggagaccagtcaccattcgacgccggacgcatcccgtgcccgcagggcggcgaggaccggtggtctgcccgctggctcatggagcagatggggtacgacaactggcagaacttcgagaaagtcgtcgaacgcgccaaggtttccgcccacaaccagggtttcaacgttcggaccctttttactgccgtcagtaaaaagggcgccggcaggccgcagtccgacttcctcgtcactcggttcgcggcatacctgatcgcaatgaacggcgatccacgcaaaccggaagtgtcggccgcacaggagtacttcgccgtcaagacccgtgaggccgagactcgtccggccattccggacatcaccacccctgaggggttgttggcgatgacggagatgttcgcggacaccgcgcgcaagctcgtcgccgtcgaggccgagaaaaagatgttggccgccgcgattgagcgggacgcgccactggtcgcgaaggctgaggcgcataccggttccgatgccgacattcatcggcaggagttcgcgcgtgaggtccaggcgtggggcgtcaagcagaacgtggccatcaagcaggctgacgtgttgcggttcctcgggcacatcgggttgttcatccggggggaacgttccgacactgggcacgccacggctgacgctcagcgtcgagggttggcgttcacccacaaaaatgttgcgcgcaacggatacgcgtatgcggttgggaagttgacgcctgctggtcaggactacgcctggaagcggatcaccaagtacgtcgccgatcacggcacgttggtgctgccgcgcgagctgcggggcggtgatccggcgtgagtagcgacccgtcatgcaaaggcagccagagccgctacaacaaggggtgtcgatgcgacatgtgcaaggcggtcaatcgcgagaaagcacgcaagagacgtgagcggaaccgtgaaaagctgcagggttcgtatacgggcttcacccacggaatcggcggctacgacaattggggatgtcgctgcgaagtttgcaaggccgcggcgcagaagaacagggctaaatatcgtgcaggactgaagactcgtccacgcgatgaggttccgcacggaactgccaacggctacaaccactgggcatgccggtgcgatccgtgccgaactgctgcatacgctgcgagatccaagtatgtcgacgcagctgagtattacgcaaagcacgaagcgctgcaaaacgagcgaagtagaactttcgcgcatcaccatcgagatacctggactggtccagagctggaaatccttagccgcgagactctttcactggccgacgccgcgcagatgctcggacgaacgctggcggcatgtagggagatgcggagaaagcttcgagtcgatccgagaaagaagggatacctgtgaagttcaccggtgactacctctaccgggttcgtgtgatctcctacccgaagggctctttcgagtgcatcgacgaggccgctgactactggatacccacccccggctggcgtcctccggggtggcgcccggttggcaactacacccagatcatgggcaccgacgagttcgtgtggccggtaacgaacaaggtgtacgggtcgcactcgacagctaagaagcgggccgaccttctcgagtcctatggggctactgcggtggttgagcgttccagccggattgtgtggcccgaatcatgagcttctctttctactcaaaacctcagcgtctgatcaaaaagtcacacggcggtgtgaccatcggtctagggaactacgacggaaccgacctggcctacctcaatgttgccggttaccgaaatgacgccgatgtccttctcaccgccgatgaactcacagatctgatcgaccagctgaccatcatccgcaacgcgatgagggagacgcgatgacgtttcattcacgcccgaggcctccgattcagcatttcccgaaaccgaagaaacctttgttccagtcgaaaccgaaggatccgaaatgagcactcccagatgggccacgttcaaagaggccgcgtcatacctccgcttgaaatcagacgtgctgatacgggaggcagtcaaaaacgatgggttgaaggcttatccgatcggtaacggtcgggaggcgcgtgttgacctgaatgaggttgatgagtggatgaagtcgcgtagttatgagccgaggtccgcgtgagtacgtctgctcctaagcatcggagtgtgtgccaactgtcgggtgaagtgactcgtccgtcggggttgtggaaagcgttggcggagttcgacgcccgtcagttgcgtgaggctgcggagttggaggcgttgcgtgaagaaaacgcgcggttgaggtgccggctacaagaactgggggagacagcgtgagtggtctgctctggattctcgtggccgttgtcgttgccgctcaggttccccaggtggttctgctgatggccccgcgtgcgttctgggacggcttgtacgacagccgaccgacagcggcgtgcttcctgtggggatattcccaccccttcggcccggattggagtaaccggtgaatcttgttgagcgtttgaatgccaggtttaacaacgtgattcatgacgggctcgccttggtgggtgctgtggtggatccgtggctggccaagcttgagcgtcaggccatgagcaatgcgttggggcgggatttcggcttggactacgcggatggtcttgcggctgcggaggctgaggaagaagtccacgaacccggattcgcctctgtccgcggcaatgccccagacccgtcacccgtctcggtgggtgacactggtcccggcgcgggcatggttcccccgcctccccccgcgccgggaccttccaaatgcacctgccccacagtggaatgcgaactcctcgctgaggacatctgcgatgaggctgaggaagacgaactcacgggcgacgactggatgagcctgagggagtacctggagaccgcgaccgcggaagaactcgccgccatgaggcaacagcgcgtgacggccgtcgagttggaacgccatctgagctatttcacgaccgcgcccggcgcgtacggggtgaaccccgcagtcgtcgccgcatccctgctcgacaggtaccgcatcaccccgagatagataggaacacaggactaatgaacctgaagacagcacgagccgcgatcgaagcgatcctcgccgctatccccgatgaagagttgccttcgttcgaccgtatggagcacggagccgacggacgagtaaccgcctggtatggggacactggccggatcctgggaagcgctacccgtggtggcatccgtaattccgaggtgtaccggaccggggcgtcgtggagcgccattgaaggcgagatggcctaccgggccgacaagaagcttctggagaacaacgacgagcctgctggcttgttctgagctaggcgggccgccgccccgattgcgcgggacgacggccctaacaccggaaacacacaactaaggagacagttcccgatgtcaatccaagattctaaaccctcatggtgggacaaccaccaaacaaactgggctgacctgcccgtcaccaccaacccacccatggctgacctcgacctattgaaggagttggaggacttggcggagctggtgttgatccacacggagagtgtgtcttggttccgcccgttcctgccgccggtgcattgggagaacgagccgacgatctgggagcagatgaacggcgacgctgttgtcgggttgttgcgtgactacctcaccgagggagacgcagcatgaggcgcaacgagaagtcctggcggtactggtggacgatgccgctgctgatcgctgcgggcatcatcggccccggactcgccgcaccagaagccaaagcagacatcacatccgatgccttcatcatggcactcgactccgaaggcatcacctacagctccaaacccgccgtcatcaacgccggcaaagccatctgcaacatcctcgacaccggcgccaccatgtacgaagcatcaatcctcgtacacgacaactccaacctgaacctctacgacgcaggttatttcgtgggtgccgcaaccgcatcattctgccctgaacacctgagcggaactgggtgggcgtgatggcgaactcaccgttcatccaactggcagaagtccacaccagcgactggcgttcccgcgcgatctgcacccacaaggacggcgacatttggttcctcaacgaatccggccactacaccgccgaccccgcccgccgcatctgctggacctgccctgtccaagcgccatgcctcaaattcgcgttgcaacacaacgaggccggcgtgtggggcggcttctcagagaaggaacgtgcccgcatcaagcgtggcgagctggccccggtgaaaccggcacggttcaccgagaaggaatgcttgcagtgcggtgaggtgttcgagccggtcacccgcagggcaaggttttgctcgcagaaatgcaagaagcgcgcctcgaatgcgttgcggtcacaaccgtccctgaagatctgcacgcagtgcggcggcgagtttatggggacgtatgcgaagacctgctcgaatgaatgccgacgggcgcagaggtggggcgcgtgagcatcgattggttcgccgtggagtgcgccgtgaacggaactcccatgcggcttaataccgaagagcgccgaatgctggtgcggcggcgcccgaaactccccgaagtggagttggcgcgtagggcgcactgcacggtccgcaccatcgaacgggacagggctgaactgcctgcagcaaagttgcaatcctgcccggtgtgcggggaggacgcgtgggtcacgaccgatggcagcatggaagcccacccagacaggctgtttcaggaatgcccactgtcggagacggattgggaatcccgtatcgctgcaacagtcatctggttgtctcggcgtatccgtagcggtgactccctgcccgtgtgggcctatctgacaagcctcccggaaaccgaacgcactcaactgttgatggctgcccttgccggtgtgccagatgttgaggacccgttcgcgtggatcacagaactggagtccgttgcatgaccgacctgtctcatctcctggtcatcatcaacgaggaccggcactcgtggcgagacaaagccctgtgcgcccaagtcgatgtgggggacatgtttttccccggtaagggggagagcgcgaagccggcgaagagaatttgtgcccggtgtgaggttcgggtcgaatgcttggagttcgcgttggcgaatcgcgagaactacggggtgttcggggggttgtcggagcgggaacggcggcctctgctcaaagcgatcgatggtgaggatcaggtggcatgagcaacgggaacaggctcaccccagagcaggtgcagacgattctgttgatgactcgtgaggggtggtccgccaagcatattggggaagtggtgggttgttcggctcggacggtggttcgggttcgggcggctggtgatgcccgtttggcgtcgccggatcagtttgtgccgttgagccaggagcagaaggattccgcccaatatttgcttgacgacggcgccccgtatcgggaggttgcccgcacgttgggtgtgagccggacaacggtcgaaaagtatttccctggttacgggtggtcgaagaagcaggctgctgagttcagagctctggtcaggaagttccgctggttggaggcttcgtgatgtgcgtgtgtgggcacaacaggtcgtggcaccgatacgcgtgggacaagttccggggacggtgggacacgggttgtgacgccaccaactaccacggccccgccgggcatgaacgctgccactgcactgaatatcgagacaaggacgaaacatgatccacacgaggaagtcaccacgaacattcgacactcgaatcatcgaggttggcgacaatgtgaaagccggtgcagcggcgctcgactgcaagaagccaagccccgccgccgcgcaagccgctttcgatgacggacgttgtctcctcgacgagctgctcacagaaacgacctggataccggacaggccagcggtgcagaaaatgtatcggtcgttggaggaagccgtcaggttgatgaaccttgctccgaataacaagagggttcagggcgcggtgattgagattgcggatcgggttcttggggcctcgatccgactggcggacaggtggacagcatgagcaccgaaacccagaccctcacgtgggaatggttcaccggttttgttggccccggcaggtggcgtgcagaactccccggtgatcggcgcaacgcgtggatcaatccgtccgatgtggctggtgatttccgttggtctgtggaggacaacacgtgtgcgcgggttttggcgtgggggtatgaggaaacgttggacgccgcgatggccgctgccgctgctgctgcggaggtgaccgaatgagtgacccgactgccacactcgccctctgcaaatggttggaagaccggctaaagcaatggaaagcagaagccaaacaacaactcgggttactggcgggggagcggaaagccgccgtcgtcgccggacaagtcatcggacacgtgtcgatggcgaaaggccgaaaaaccgccaaagtcacatccgaaacagcactgctcgcctacgtgaaagcgaactaccccaccgaaattgaagtcgaagaacgcgtacgacccgcgttcctcaaacaactcctggacgacgcagcgaagaagggcgcgttcgttgacgtcgatggggttgtgatcgatgggttgatcgatgttgttgagggtgacccgtatcccattgtgaagttgtcggatgactcggatgtgacgatcgctggtttgttggctcggggtgctctcggggtgtccgggttgaaggagatcgaacaatgaccctcaaaacccgtcccccaaccggcgctgtcccatggccgctcatcctcgtcgaaggtggtgagaaagccggtaaatcgtgggccgcagcggtcctttcatcctcggagaaggtcggccgcaccttgtggatcgactgggctgaaggtgccgccgacgagtacggggcaatccccggtgcccgctacgaagtcatcgaacacgacggcacatggacctccattatggagcaagtccgcgccgcccgagacgaagcccagcgtgccatcgacgccggggagaaacccgttgtgctggtgatcgactcgatgaccgcggagtgggacgacctcaaagagtgggtcgataacaaggctcgccggcgtgaggcaaacctgaaaaagcttgagcgcgatcctgaagcggagatcgtcatatcaactgacctgtggaacctcgccaccgcccgccacaaggaactcatgcgggtcctgatgcggttccccggaatcgtagtcatgatcgcccgcggcgcggatcaggtggctatggaaaacgggaaacccacctcgcagcggacgtggaaggttgaggggcagaagaatttggcgttcgacgcctcagtgtgggtgaggttgaaccggggtgagcatccgcagatcatcggtgcccgttcggtccacgccggcatcattccaggcgaagacaagccccgccgtgtcccggatttgacgctggaacaactggtgttcgacatcctcaagtgcgatccgaagaccgctcatgtgcgggagttggagtcggttcaggatcgtgtgttggagctgatggatttgatctcggctgcggagtcccgggatgtgttgaccgggttgtggcgggacgcgaaggccggcgagttgttgaatgttggggtcgctgatggtcctacggttcaggaggcgttggcggcgcgggctcaggagttggaagccaggcaggcggacgcctcatgagccttcctgatgctgtgcgacgtgaacccacctgcggatcatgtggcgaggaaacttactttgacggcgattccttcatctgcgacccctgccagatcttcttccacgccgatgacctgagtgcgtcgttcctagatgttggcgcggatacgtgcggagagccctgccagaacttctggcacggatctgaccgcataaagccaggtatgtccttcgaatgtggtacttgccaacttccagcaggtcacttgtcggggcattggacggactgccatctcatcaatggggccgcgtcgtgagccgccggttcacgggctttcccccggaagtcaaggaactgatctggtctcgtgctcacggtcgttgtgaacgctgcggagagtacgcctcagacgctactgcacaccatcgcaggccccgggcgctcggcggctctcgacgcgaagacaccaacgtggcgtccaacgggctgtgggcctgtggtgcttgtcatcgttgggcggagtcctatcgggcgcaagcgttcgccgacggctggcttgttcgtcaaacccaatcccccatccagattcccgtcctgtaccgcgggcaatgggtgttgctcgacgacgacggcaacacctaccggatacctgaccctgtggaggcaacacaatgaaccgtcctgtgttttatgtggatcagaaagaggacttggagaagggcacgttctggtggacggtggccacttcgaatggtcggacgatcctgacgtcggagatgtacagcaggaggcgcgacgccaggaaagcagcccgaagcttcatcgggaacattggagacgttcccgtgtcgttccggtacttcaacccaaacagagaacaggtggaagaacgcttctccgttgggatgtggatgagcgattctcgggagtggtacgcgtgatgtacacggtttcggggacgtggccccactacatcgtcaccggtggaaccgaaccaccgaaatgcttcaactccaccgtcaccgccgtcaaatacctggaacagattctccagcaaggcgacaccatcaactggcaggtcccatgatcaccgttgtctgcgcggaatgcagccgcaccaaaggctgccccatcaccgccgaattccccactaccgaacaagcgcaggcattcatccgccggcaccacgccttcgccgaccaccgggcacacatcccagaagaggccgccagtgaccgactgtctgttgtgtgaccatcccaggtcttctcatgccccccggtgccgggtccgcatgggtgtcaaccgggacgacatgaacacctacacgatctgtttgtgcccaggattcgaaggcacagaagaggaggaagacacgtggctggactgaagtggattcgactcgaaactaccctctttgagaatcctaagttgctctacctccaagaggacaagcagtacagaacgatcgtttcctacctccaaggtatgtgttactcgggtcgacatgggctcgcagggttcgtcccgaaagccgctctcagggtcatcggggcgacgcaatctgatgccacacgtctagtaagagaagggctttggaaccccgccccaggtggatggcagatcaatggctgggatgagtaccaactgtcaaacgaggaagccgtaaggcgcagcgagaaggccaaaaaggccgctgccgccaggtgggctcagaggaacggcaggcagttcgatgacctatcaatctgatgctccaagcattgctacaagcattaaagggttgcagtccttggggttatgcaagtcgagcaatgcacgtacgtacgcacgtactcacaaagaactactcacctttggtctttcttcttacgttagtcaacgcgcacagggggtagaaagattttgatccacaaaggggaggaaccaaccgccttcggcgatgtcctgacggacatcggccccgagttgcggccacacgactgcggcaggttcgacgtcaggcgcactcgtcacacagaggaccgccaaccgattccgcgatggctgcggctcaaggtttacaagcgagaccacttccgctgcgtatggtgcggatcatctgaccgtctagaactcgaccacatcgttccgtggtcagctggcggcccggacacgatcgacaacctgagaaccctgtgcaaaccgtgcaacagttaccggtccaaccgcgggtactcgctggatttatcagctcgtggaatctcgcatggacgggagtgcgtgaactgcaacccggaacgggttctagggcatccacaagcggaagggatcttctgcacacggtgcgagatcaccgcagtgggtctttcgcatcgcccgcttgaaactgcctatttccgaggctatgtgacgctcgatgaggccaaggaggcgatgtgggatcgctggcgtgaggaagacgatctcgaaggcggtgctggctgatgaaggattggcgtggcacgacgatccatcaggaagcgttgaaggttgggtgtcgtgattgtcgggctggggttggtgagccgtgtgtggtgcgggatgggaaggggcgtgtgttgaaggtgttggaggcgtttccggctcattcgcacaggatcgctgacagccgttctgcgggttcccaaggcactgacaccgaccttgccccgaaagtcgctccacgtggcgtacagcccccgcaatcaacaccaggagacaagtgatggcgcagcgaaaaggcggattcgactggatccggtcaaactacggtgtcccggcgaagcgtggaatgcgagtcatcttcgatggacgacccggacgcatcgtgagtgtggacggcccgtacctgatgcttcacctggatggtgatccatgggactggcgcactcgtgtacatccgacgtggcgcatggagtacctgacatgacgatgttcgtttcgagcccggatgatccgcgtgtcctggaggcggtgtcgtgcaggtcgtgtgacatctgcaaagcccccaaaggcaaaccctgcagcaacacgattcgtccggggaagccgctgcccggtcgggtcatccacttcgggcggctcacagacagaaaccgagaaccgaaaggcgacgaatgaacaaccccgagttgcgtgcagtactcacagaagccctcagtgaagcgctgaagcggctgtggaccgaccctgaggatgctgccgaccaggcggcctgggatgcgctccccggaaagctcgctgatgccgttgcttctcttccgggtgtggcggtaatccaactacccgaacccaccgaggctgtgacgatgcaggactgcgcgggatcggggaaaccgttcaagcccggcactctaagccgcgacggcgaggtcgccaaatgccctgcgtgcgggaccaaccgctacgtccgtgacgacggcagtatcgagccccatcaggtgcccgttgctgcggttgtagccgagggggaagacaagtgagcggcgacatcaacgccgaaggcttcatccgctacggcggtgactgcacttgcggcgcgatctacacctatggcggacacgcggaacctggctcatttgatccgttctgccccgaccacggagaggctgcggttgtggctacaggggaggaagcatgagcgacgacgcattccgggcatccgtaaaagccgcagtggacaagtacggcatcaccgccgtggcagccaagtgtcagacaagcctcctggtcgtacgcagatgggccgaagggttccccccgtatggcgccgctaaggcatctgttctgaaggcaatcgattctttggaggaagcgtgagcagcgaagcccagaacgtgatcgccaacgccctgcgtgagcacggctacaacggtgcctcgtgcggtgccgagtatccccggcacgagttctgcacatgcggctggtccggtcccggtgagggcgtgcatgagcagcatcaggccgaggaaatcgacagagccctcggaggactcaggcgggaaacccgcgtaatcgagagcatcttcgagctgggcgtgccagagcctgcaacccgattcgttacccactggatggagatacctgatgagtgatgttgttgagcgcgccaaggctgcgctggtcgactacgaagtggcgaaggggtctcgggtcgcggtcgcaccgggccggtcctaccggctgctcgccgaattggtagccgaggttgagcgtcttcgccccaggggggttgagactactgctgatctcgaatggctcccagaggattcctgagattatcaggggttgattcgatgatcgtcgccgtttctccaggtaggcagccgatctgacagcgcacacatgtttccgattaccgacactcgtagggagatgacgactatgccgaccacagagcatggatcagacgtccagcacttgagccctgaacaccgcgatcgtgcttggcgcgataggttcaacgcccggtggcactatgactacggcgggtggattcgtaccaggccgcaggatgaggcgtcgaccttcgctttgatcccaaccaaacactacggaccgttcactgaggatcactcgtgtcctgcctgcctggtggtacacccacctgaggattgccccgtcctaagtggaaacaccgacatgttggttgttttcgattacgacacctcgcccaacaaggcacaagcggatacagctgacgatgaccccagataacgtaattctcacccacgacggaggaaccctgcagaagacgagtaggggtacctggtattgggccaacgatgaccaagacgagagccttccagggggccttatcgacttccttcccgcccgcgtgctctacatccctacagactctttggaggaagcgtgagcagcgaagcccaaaacctcatgatcgaggtgatcgatgcgcacacgtacaacggcacagacaggggggtcctcggcgagcaccgtgtcgagtactgcatctgcgggtggtcggaggaaggcgacggcgtgcacaccgcgcatgtggcttctgaggttgataaagccctcggaggactcacccgtgaagagcaatgggttcctgtagaggaatccggacaccgctgggcgggtcggagcagggaggtcgcagaggtctgcctcaaggacttctcggtcacgggaatctgccacgacccggaggtcgactctccgcttgtccgaattgagcacgaggcccgctgggtgtcgggatgcgagcaaggcatgagcgatctctgcgcgtgcgatcccagccacggcaatgtctgcggctgcggaatgagggtgtgcgcgccctgctgttacgccgtgcacgacacctgcccctgctcctgcccgggctgtttacccgagggatggtccgacgggacgtcccattcccacacgtgcggaaaggaaccgtcgtgattcaggttcattgccgggagtgcaaccgtgtgtgggaccagtcgtgcgaagactgcgctcagtggaaagcggatcgtcactcgatcaacacggggcatacggatattcacatcatcccggacaccacaccaccgcggcctgtggtggatcaggggtgggcggaatggctcacgaaaggaaaaccatgagtagctacaagatccgcgtcgaagcgaaagccgttgagttctacgagatcgaagccgagtctgaggatgatgctcgcgatcgttggcacgagggtgagttcctgcacagcgaggtctatgacgcgtacgtggacagtgtggagctgatcgatgagtaatgagggcacaactcctgagcgtgcagctctggttgagcgggccgcgcaagccatctacgaacaaacctccgccggcaagttgtttccttgggacaccgtgactgaagcgcacaaggtgcagtggcgggccatggcggatgctgcgtttgatgtcctgatcgacgcctgggctcctccgttttgaccggccgcaagatcgtgtcccctgccgatcacattgaccgggccaaagaggaagccgccgcgggggattaccaagcagcgcaggtccacgccctgatcgccatcgcacaactactcaacacaaaggaccaaccctgatggccaaaattcagatcaactacaagtccggaacttccatcgtcgcgaaagccgacgagttcaccgtgaaacgagacaagtacggcgtcaagtcctacgagtggacgaacatggtgccccgacccctggacttcaacgccgacgacgtcgaatccgtgtgggagctgtcgtgaccttgtccgtgatcctcgccgcccaggctcggttcatccacgagagccctgtttgtccggcgtgtttccagccccgcacagagcattccaccgactgcaaaggacaccacaaatgagcgtctacgcactgaagcaaccgcgtccagacgggggagagtggatccaggagcacgacagcctagaggatgcgcttgagttccagtcgcatagcggcggcattctcgtccgccgcgaggctgtacctgggcagcctggactgtggtgggtagaggtcaacaccgaattgcccagcgatgtcgggtcggttgtgcagtctgaacccaaccaggaggggatcactgatgtctgatgctcgtgtgggggcgtggatcgcggcgtgggacgcgctcaacgccgccaccaacaccctcaaaaaatgccccattcaagaccccgacgagtatcgggcgttctgccaactccaagcagacatctacgcccacctcgccgacgtcccggcagaggtcggtgccgccgcagcggaatggcttgaacaccgcgagaaggaactacgggaacagaaacgtcgggaacagaaagaagatatgttcaggaaggcgttcgacaagtgagcatggacttccacctccccagggctgaccaactcaaactacaagaggcgctcggcggaatccccacactcatcgaggacctcgccgtaaccatcacacgccaagcccgcgtacaaaaacccggactcgggaaacttcgaaggcggaaagctgaggcgcgcatcccgttccacatcggcgcagtagaagccgcagacgaactgcacaacgccctgatcaaatgggtcaggttcacctgcgacgcccgacaaaccccgtacaccgaatccaacgatgacatcactctggcccgctggctgagacgcaacgtgaccgccctcgccctcatcgaaggctccgaagaatcctggcctgaaattcaccaccggattgacgagtgccgcaagcagatcgacttgcccccagaggatgacatcgtgatcgacccggaacgggtccgccaggccaaccgtcaaatcctcaccgccggccagatagaaaagatcgccccacgcctcggcgcactcggtgcagggctgaacaaacgtcgggtccagaccctcgtgaaaagcaagcgactgcggccctgcgcagtcgacggagaagtccggttctaccgcctaggagatgtgctcgacgcgcaccacaggcaactaccacgctccaagaaaacaacgtctgaacaggtaaaattagcagaatgagccgggtctttcgggtgcatctcaacgacgtcctcgctgcggaatgctgccaccccaactgctacgcgccagcccttactgacatagccagtcatgtgccgttgtgtgagcggcacatcatggttgtctaccgggaagccaatctcatgctcgccagccatagagctatggaacaggcatatgaacttcttccatcagaggctgagttcatcccaggcccatgccctcgctgtggggacagtggtctacttgcccacttagcgaacgggtttgtggtctgcaaggcggcagggtgcgactacgaacgatccatggtggcgttctgcactgaacggaagacactgatgggtgtaactgccgcaactgatgacgtcgtgtactacatgcgactgggaaaccgtgccaagatcggtaccagccgaaacctcaaggcccgcatcggagtgattcagccagaggactgcatgggctacgaaccgggggaccggaagctggaacgtaaacgtcatgaccagttcaagcacctgagggtgtctggtgaatggttcatgattggccccgaccttgtgcggcacgtgaactcgctgcggattgcatgagcacagcatttagttgctatgtttgtcgcaattgccttaacggcaaaattgcgccgtaaccttgctgatttgacaaacaaggttcgatattcgtgctaggctgtcgccgtaggcgcaggtcacacttcctcgcctgttaaacgccccggaacctaccggggcgtttcgcatttcgggaggtgaacccccgttgcccaccttcgccacaccacgatccctcaacgaccgcatcaccgacgctctccacaacgttcgcctagcccgcgaagacggaaacccgagcattgttgaggctgcggagaaactgttggaccagttgttggatcgcgttccccgctccaccagccaggagtagttgccgtgccgctcaaacacctccgcatttgcgacacctgcgatcgtgtccgtttcgccccctgcggcaaagcatgccgagtccccaacgatatcgatcctgactcgtggcgaatcaatctgcaggacggtgcaggaacgatggagaacgccgggttcatgtgtgtggagtccaaggaggccgaagaatgaccggcgcccatgcatcactgctggggttgttggcgttccttgttttccatcactacacggtggttcgtgagctgcggtacatgcggttgcagaacaccaagatggagacggacctcatctgcttgcggaacaggttcggtctcctatgaccgccccggtcgacgtccgcgacgagatcggtggcgaagggtgtgccgacagaatcagtgacggcctcgcaggcgaatatcccaaatgagcagcctcacagacctcacggacttccttaaccgcacgctgaacaacctggttcaccccggcgacgaaaacaccaaacccttcccgatcctcctgccgggactacgacctatcagtgtccccccggaactcgccggccagttcgctgaagaatcaggtctaccgcacctcgataccccgaaactggtcgcggaagcactcgccgcggcgatcacgcaaaactatgtgatcctcacacgcgaagaagcagaacagctgcgccagaaagcggccgacgcaccgaccgggcaccgcgtcatcaacatccgaaccacacccacagccccgcccgtgctgtcgatcaccatcgacaaaacaagcaacgacgtcatcgttcccaaacgagccttgcggaaagcgaccgaacagtgatccacatcgaagttgacgggaaagtgctcatgcacgccgatcccggccagtggaccaccacgccacctgatgttcaagcggtccagaaagctggacccaacgagccttggatgctgccgatcatgaccgcactcgcgaaggcggccaccctcgcgatggccggggcgaaacacgaggacaccacaatccgcgtgaccacacgcaagaacggctggacgatggactgcaccaatggatgaggcagcccgcgcccgccaggagctgcgcagatccaacgccgcccagccgcaccgaaaccggcaccgcgaacgcaaaaccggacgaaccacagaccgcaacatctgctactgcggcgacgcggactgcccagactgcggcgaatggtacgagtgacgaactgagcccacacatgaccgacgtcgtgattaacggaacccgatacgttcccgaaaccaccaacggaactccaatcggaatcggagtcaccacccgcaaccggaacaccatcgccgacgagacaatcgcccacattcgccgccacacacccaacgccaaactcgtcatcgtcgacgacgccagcgacgaaccatacccagcagcgacctaccgattcactcaacgcgcaggcattgcccgagccaaaaacaaatgcctcgaactcctcaacggctgcgagcacatcttcctgttcgacgacgactgctacccgatcgccgacaactggtttcagccttacatcgactcacccgagccgcacctgatgtaccagttcatcgacctcgccggcggacggaaactcaacgacgtcacgaaggtctacgacgacggccaacacttcgccctgtccggggcgcgtggctgcatgatctacgcacaccgcagcgtcatcgaacgcgtcggtggcctcgaccctgaattcggcggttggggctgggaacacccatcgtggtccgatcggatctacaacgccggactcacctcgttccgctacggcgacgtctgcggatccaacaagctcatccactccatggacgagcacctggaagtaaagcgttccgtcccgaccgaggaacgcaaagccgtcgccgcccgcaatgccgagttgtactggcagcaccactacaccagcagccaccacatccccgtagtggaacccgaccggcgtgtggtgctcacttgcctgctgtcgaacaaacctgacccgcaacggggcacacgcatgcggcccgacgtcaaactgctcgaaacgttgatcacctccatcactggaggtgaaaccgttgtgctgtgcgacaacccactcacccacccgcaggcgtcattcgagcgagtcaccagcccagtcgataacccatacttcgcgcgctggtacctgtactaccaatggctccgcgccaaccccgacgtcaaatgggtgtggtgcgtagacggcaccgacgtcgaaatgctcacccctccgtgggaacacatgcaacccgggaagctgtacaccgggcacgaacccgccgttgtggggatcgactggatgcgcaacaaccacaaagccacacacctgcaacagttcatcgacacccacgccgaccgcaccctactgaacgcggggatcgtgggcggcgaccgggaaaccgtcatggcattcgcacacgacatggccgccgaccacgaagaccaacttcggcgcgtctggcacaaagacgacgccccgggaacaatcatcggcgacatggcgacactcaactacgttgcctataccaaacacgccgaccaactcatccacggaccccaggtggtgacggtcttcaagaccaacgaacgcaacgactggtcatggtggaggcacaagtaacaatggtcgaaaggcccatggtcacgatccaccgacgcaccgtgcacaagcagttcaccaagcagatcgcctgggagaaagaactacaggcataccgcacgatgccatgggccacgcccaaactcatcgacttcggacccatgtggatcgaagtcgaacgctgcaccccgatcctcaacatccaccccaactggtcccggcgctacgccgagccgctgtgggatctgctcgccgccatccacgccgccggctggtggcactgcgacccctgcctgatcaacgtcgtcgtacaccccgaccgcggcgtgctgctcatcgatttcgagaacctcacactcgcgaccggaaaccgctcctatgacctctacggcgcacgcgccgccggtgtcgaacccgcctggcccggactcggcccagacggcgtgtactggaacggaccatggccgtcgtgccccggaccctactgggacgaatgacaatggagcggagcatgaaacccggcgacaacgtatgggtcgacttcaacggactcgaacacgaaggcaccgtcgagaaaatccaatccagcggctgggtcagatgctccatcgccatcgaccccgaatacgactacggcagcatcacaccacgactcgcaccacacatcaccgtcgccgtgaaaaccacacgcataaggccacgatgaaccacaccatcggcatcgtcgctcacaccaaacgcgccgaacaagcacaccggctcatggaaaccgtgggcgccgcatacatgagcatcgacaacggcgcactcggatgcgaaagcaaccaccgcaaagtgtggcaacacctcacccgccacaacacagactggctcgtggtcctcgaagacgacgcgatgccgtgcaacaacttccgcgaccagctcgacgcagcgctagcagtggcgcccagcccagtggtcagcctctacctcgggcgggaacgaccccgcgaataccaacaacgcatcgccaaagccgctgacaccacagcacactggctcacctgccgacgactactccacgcagtcggaatcgccatacacgccgccctcgtgccgaacatgctcaacaacctgcccaacggcaaacccatcgacgaagcaatcagcgcatgggcacgccgccaaagccacaccatcgcctacacatggcccagcctcgtagaccacgcagacgagccgtcgctcaccggccggcgcgggccagcgaccgggcgggttgcgtggaggcacggcggacgtgactggtggactgccgaggcacacgcgctctgacccggccagcaaacgagcgcgccagcaaacccgcaggtcagaaggggtggggaggccccccagcgaccctccgacc

SEQ ID NO: 4 - DNA, 22 bp, synthetic construct
  source 1..22: mol_type = other DNA
aacagagaggatttcgtttccg

SEQ ID NO: 5 - DNA, 23 bp, synthetic construct
  source 1..23: mol_type = other DNA
tttgacctgagggtaagacttct

SEQ ID NO: 6 - DNA, 17 bp, synthetic construct
  source 1..17: mol_type = other DNA
tgcccgtctgggtgaga

SEQ ID NO: 7 - DNA, 19 bp, synthetic construct
  source 1..19: mol_type = other DNA
ccggtgctccttgatgaga

SEQ ID NO: 8 - DNA, 19 bp, synthetic construct
  source 1..19: mol_type = other DNA
cagtccaagctcgattgcc

SEQ ID NO: 9 - DNA, 19 bp, synthetic construct
  source 1..19: mol_type = other DNA
ctgccgaaagttgctcact

SEQ ID NO: 10 - DNA, 21 bp, synthetic construct
  source 1..21: mol_type = other DNA
catgtacgttgctatccaggc

SEQ ID NO: 11 - DNA, 21 bp, synthetic construct
  source 1..21: mol_type = other DNA
ctccttaatgtcacgcacgat